(19) United States
(12) Patent Application Publication
(10) Pub. No.: US 2006/0051763 A1
(43) Pub. Date: Mar. 9, 2006

(54) Detection methods

(76) Inventors: Anu-Maria Loukola, Veikkola (FI); Sharron Gaynor Penn, San Carlos, CA (US); David Russell Rank, Palo Alto, CA (US); David Kagen Hanzel, Palo Alto, CA (US); Graham Casey, Moreland Hills, OH (US); John S Witte, Burlingame, CA (US)

Correspondence Address: AMERSHAM BIOSCIENCES;PATENT DEPARTMENT, 800 CENTENNIAL AVENUE, PISCATAWAY, NJ, 08855, US

(21) Appl. No.: 10/529,193

(22) PCT Filed: Sep. 25, 2003
(86) PCT No.: PCT/US03/30359

§ 371 Date: Mar. 24, 2005

Related U.S. Application Data
(60) Provisional application No. 60/413,583, filed on Sep. 25, 2002.
(60) Provisional application No. 60/491,842, filed on Aug. 1, 2003.

(51) Int. Cl. C12Q 1/68; C07H 21/04; C07H 21/02

(52) U.S. Cl. 435/6; 536/23.1

(57) ABSTRACT

The present invention relates to the prognosis, diagnosis and treatment of cancer, particularly prostate cancer. Polynucleotides having single nucleotide polymorphisms (SNPs) and haplotypes are provided which are of utility in the prognosis, diagnosis, prophylaxis and treatment of prostate and breast cancer.

### CROSS REFERENCE TO RELATED APPLICATIONS

This application claims priority to U.S. provisional patent application Nos. 60/413,583 filed Sep. 25, 2002, and 60/491,842 filed Aug. 1, 2003; the disclosures of which are incorporated herein by reference in their entirety.

### FIELD OF INVENTION

The present invention relates to single nucleotide polymorphisms in nucleic acids involved in encoding enzymes in the testosterone biosynthetic pathway and to methods for detecting such polymorphisms. The invention has utility in the diagnosis, prognosis, prevention and treatment of disease, particularly those relating to prostate cancer and breast cancer.

### BACKGROUND OF THE INVENTION

Prostate cancer is the most common non-skin cancer in males all over the world. Currently, there are no means to predict how aggressive an individual's cancer will be. Thus, many patients are given unnecessary drastic treatment with severe side effects and possibly others do not receive treatment effective enough.

Incidence of prostate cancer shows strong age dependence, being a disease of old men, and strong race dependence, being almost twice as common in African Americans as in Caucasians, while Asian populations have the lowest risk (Cook et al. (1999) J Urol 161, 152-155; Hsing et al. (2000) Int J Cancer 85, 60-67). The third well-known risk factor is having a family history of prostate cancer (Cerhan et al. (1999) Cancer Epidemiol Biomarkers Prev 8, 53-60; Kalish et al. (2000) Urology 56, 803-806), and several studies have supported the presence of predisposing genetic factors.

Genome wide linkage analyses have pointed multiple chromosomal regions showing linkage in prostate cancer families and several prostate cancer candidate loci have been suggested; HPC1 in 1q24 (Smith et al. (1996) Science 274, 1371-1374), HPCX in Xq27 (Xu et al. (1998) Nat Genet 20, 175-179), PCAP in 1q42.2 (Berthon et al. (1998) Am J Hum Genet 62, 1416-1424), CABP in 1p36 (Gibbs et al. (1999) Am J Hum Gen 64, 776-787), and HPC2/ELAC2 in 17p (Tavtigian et al. (2001) Nat Genet 27, 172-180). Recently, a candidate cancer-susceptibility gene, RNASEL, was cloned at the HPC1 loci (Carpten et al. (2002) Nat Genet 30, 181-184) and two possibly deleterious germline mutations segregating in prostate cancer families were discovered.

The growth of prostate cells is dependent on active testosterone (Ekman (1995) J Urol 101, 22-25) and strikingly, prostate adenocarcinomas can be created by testosterone administration in rats (Gupta et al. (1999) Cancer Res 59, 2115-2120). Testosterone seems to be a strong tumour promoter for the rat prostate, even at doses that do not measurably increase circulating testosterone (Bosland et al. (1991) Princess Takamatsu Symp 22,109-123). Consequently, genes involved in the testosterone biosynthetic pathway, e.g., CYP17, CYP3A4, and SRD5A2 (FIG. 1) are good candidates for being involved in the initiation and progression of prostate cancer. Several polymorphisms have been discovered in these genes and some of them show association either with increased risk or progression of prostate cancer (Table 1). Nevertheless, there is no evidence of higher testosterone levels in prostate cancer patients.

Approximately 55 different Cytochrome P450 genes are present in the human genome and are classified into different families and subfamilies on the basis of sequence homology. Members of the CYP3A subfamily catalyze the oxidative, peroxidative and reductive metabolism of different endobiotics, drugs, and protoxic or procarcinogenic molecules. As an example, CYP3A4 is responsible for the oxidative metabolism of an estimated 60% of all clinically used drugs. Up to 30-fold interindividual differences in expression has been detected, causing variation in oral bioavailability and systemic clearance of CYP3A substrates, such as HIV protease inhibitors, several calcium channel blockers and some cholesterol-lowering drugs. Variation in CYP3A expression is particularly important in substrates with narrow therapeutic indices, such as cancer chemotherapeutics and immunosuppressants. Variation in CYP3A expression can result in clinically significant differences in drug toxicities and response.

As with prostate cancer, breast cancer also shows age-dependency indicating a possible hormonal influence on the disease risk. Endogenous oestradiol synthesis takes place in the ovarian theca cells of pre-menopausal women, in the stromal adipose cells of the breast of post-menopausal women, and in minor quantities in peripheral tissue. These cells, as well as breast cancer tissue, express all the necessary enzymes for this synthesis, including CYP17, and enzymes that further hydroxylate oestradiol, such as CYP3A4 (Kristensen et al. (2000) Mutat Res 462, 323-333). Thus, polymorphisms in these enzymes may also be associated with the risk of breast cancer (Kristensen et al. (2000) Mutat Res 462, 323-333). Furthermore, CYP3A4 is also involved in the activation of many mammary carcinogens, such as the polycyclic aromatic hydrocarbons and heterocyclic amines (Guengerich et al. (1991) Chem Res Toxicol. 4, 168-179). According to a recent study (Zheng et al. (2001) Cancer Epidemiol Biomarkers Prev 10, 237-242), high CYP3A4 activity may be a risk factor for breast cancer risk.

Single nucleotide polymorphisms (SNPs) are the most common type of genetic variation in the human genome, and are expected to be helpful in identifying human disease genes. In addition to occurring frequently, on average every 500-2,000 bp (Li & Sadler (1991) Genetics 129, 513-523; Chakravarti (1998) Nat Genet 19, 216-217; Cargill et al. (1999) Nat Genet 22, 231-238; Halushka et al. (1999) Nat Genet 22, 239-247), SNPs have a low mutation rate when compared to microsatellite markers, both of which are characteristics that may have particular advantages for association analysis. The utility of SNPs is not only in their use as markers for discovering additional functional variants and for the general evaluation of a specific gene in the context of a given clinical phenotype but also in their potential functional relevance. However, rather than finding a single SNP with drastic effect on the phenotype, more likely it will be multiple SNPs in relevant genes, either linked (i.e., grouped as a haplotype) or independent (perhaps on different chromosomes), that contribute to the phenotype.

Recently, several studies have shown the utility of haplotypes, i.e., a combination of SNPs with alleles physically assigned to a chromosome, in association analysis (Daly et al. (2001) Nat Genet 29, 229-232). Studying haplotypes might give the analysis more power but traditionally demands either samples from multiple generations or tedious molecular haplotyping. Alternatively, several algorithms have been developed for inferring haplotypes from genotype data (Clark (1990) Mol Biol Evol 7, 111-122; Excoffier & Slatkin s (1995) Mol Biol Evol 12, 921-927; Stephens et al. (2001) Am J Hum Genet 68, 978-989). These algorithms have been proven to work with a very low error rate (Drysdale et al. (2000) PNAS 97, 10483-10488). In a sense, haplotyping is equivalent to performing a study in a family or other select group of people. It helps to get back the power of linkage, and can be regarded as a crucial step in association studies using random individuals.

WO02/055735 discloses specific nucleic acids useful for identifying, diagnosing, monitoring, staging, imaging and treating prostate cancer and breast cancer. Similar compositions comprising prostate specific nucleic acids are described by the same applicant (Diadexus Inc.) in related applications (WO02/42776, WO02/42499, WO02/42463, WO02/42329, WO02/39431, WO02/239431, WO02/38810, WO02/38810, WO02/236808 and WO0224718).

Diadexus Inc. have also disclosed a method of diagnosing, monitoring, staging, imaging and treating prostate and breast cancer by means of specific nucleic acids, in a series of related applications (WO01/39798 & WO00/23111 & WO00/23108).

WO01/53537 (DZ Genes Inc.) describes isolated polynucleotides containing at least one polymorphism useful for the diagnosis of disease, particularly prostate and breast cancer.

Single nucleotide polymorphisms associated with prostate cancer are disclosed in WO01/83828, as are methods for using these SNPs to determine susceptibility to this disease.

In order to improve the lives of prostate and breast cancer patients it is essential to develop prognostic markers for cancer as well as markers allowing general assessment of disease risk. Patients need to be categorized into those needing immediate, extensive treatment, and those who just need watchful waiting. As a result, prostate and breast cancer mortality could be reduced and unnecessary side effects caused by invasive treatments could be avoided. There is therefore a need for prognostic molecular markers for aggressive breast and prostate cancer to aid predicting, diagnosing and monitoring these diseases in individuals. Furthermore, there is a continued need for improved methods of treatment of both conditions in patients. The present invention addresses these needs and provides improvements over the prior art in the form of novel and specific nucleic acids, microarrays and kits useful for the diagnosis of breast and prostate cancer.

### SUMMARY OF THE INVENTION

According to the first aspect of the present invention, there is provided an isolated polynucleotide selected from the group consisting of a nucleotide sequence comprising one or more polymorphic sequences of SEQ ID NOS 1-34. Suitably, a fragment of the isolated polynucleotide comprises a polymorphic site in the polymorphic sequence.

In a second aspect of the present invention, there is provided an isolated polynucleotide comprising a sequence complementary to one or more of the polymorphic sequences of SEQ ID NOS 1-34. Suitably, a fragment of the complementary nucleotide sequence comprises a polymorphic site in the polymorphic sequence.

Preferably, the polynucleotides of the first and second aspect comprise DNA, RNA, cDNA, or mRNA

Preferably, at least one single nucleotide polymorphism of the isolated polynucleotide is at a position selected from the group consisting of position [CYP3A4_IVS9 +187] of SEQ ID No. 1, position [CYP3A4, 1639 base pairs after the stop codon] of SEQ ID No. 2, position [CYP3A4, 945 base pairs after the stop codon] of SEQ ID No. 3, position [CYP3A4_{—}5′ region −747] of SEQ ID No. 4, position [CYP3A4_IVS7 −202] of SEQ ID No. 5, position [CYP3A4, 2204 base pairs after the stop codon] of SEQ ID No. 6, position [CYP3A4_IVS2 −132] of SEQ ID No. 7, position [CYP3A4_IVS1 −868] of SEQ ID No. 8, position [CYP3A4_{—}5′ region −847] of SEQ ID No. 9, position [CYP3A4, 766 base pairs after the stop codon] of SEQ ID No. 10, position [CYP3A4, 1454 base pairs after the stop codon] of SEQ ID No. 11, position [CYP3A4_IVS3 +1992] of SEQ ID No. 12, position [CYP3A4_IVS9 +841] of SEQ ID No. 13, position [CYP3A4_IVS12 −473 of SEQ ID No. 14, position [CYP3A4_IVS12 +581] of SEQ ID No. 15, position [CYP3A4_IVS12 +586] of SEQ ID No. 16, position [CYP3A4_IVS12 +646] of SEQ ID No. 17, position [CYP3A4_IVS3 −734] of SEQ ID No. 18, position [CYP17_IVS1 −271] of SEQ ID No. 19, position [CYP17_IVS5 +75] of SEQ ID No. 20, position [CYP17_IVS1 +426] of SEQ ID No. 21, position [CYP17_IVS1 −99] of SEQ ID No. 22, position [CYP17_IVS1 −700] of SEQ ID No. 23, position [CYP17_IVS1 −565] of SEQ ID No. 24, position [CYP17_IVS3 +141] of SEQ ID No. 25, position [CYP17_{—}5′ region −1488] of SEQ ID No. 26, position (CYP17_{—}5′ region −1204] of SEQ ID No. 27, position [CYP17_IVS1 +466] of SEQ ID No. 28, position [CYP17, 712 base pairs after the stop codon] of SEQ ID No. 29, position [SRD5A2, 1356 base pairs after the stop codon (3′ UTR)] of SEQ ID No. 30, position [SRD5A2, 849 base pairs after the stop codon (3′ UTR)] of SEQ ID No. 31, position [SRD5A2_{—}5′ region −870] of SEQ ID No. 32, position [SRD5A2_{—}5′ region between −2036 and −2030] of SEQ ID No. 33, and position [SRD5A2, 545 base pairs after the stop codon (3′ UTR)] of SEQ ID No. 34.

More preferably, at least one single nucleotide polymorphism is selected from the group consisting of [CYP3A4_IVS9 +187C>G] of SEQ ID No. 1, [CYP3A4, 1639 base pairs after the stop codon, A>T] of SEQ ID No. 2, [CYP3A4, 945 base pairs after the stop codon, A>T] of SEQ ID No. 3, [CYP3A4_{—}5′ region −747C>G] of SEQ ID No. 4, [CYP3A4_IVS7 −202C>T] of SEQ ID No. 5, [CYP3A4, 2204 base pairs after the stop codon, G>C] of SEQ ID No. 6, [CYP3A4_IVS2 −132C>T] of SEQ ID No. 7, [CYP3A4_IVS1 −868C>T] of SEQ ID No. 8, [CYP3A4_{—}5′ region −847A>T] of SEQ ID No. 9, [CYP3A4, 766 base pairs after the stop codon, delT] of SEQ ID No. 10, [CYP3A4, 1454 base pairs after the stop codon, C>T] of SEQ ID No. 11, [CYP3A4_IVS3 +1992T>C] of SEQ ID No. 12, [CYP3A4_IVS9 +841T>G] of SEQ ID No. 13, [CYP3A4_IVS12 −473T>G] of SEQ ID No. 14, [CYP3A4_IVS12 +581C>T] of SEQ ID No. 15, [CYP3A4_IVS12 +586G>A] of SEQ ID No. 16, [CYP3A4_IVS12 +646C>A] of SEQ ID No. 17, [CYP3A4_IVS3 −734G>A] of SEQ ID No. 18, [CYP17_IVS1 −271A>C] of SEQ ID No. 19, [CYP17_IVS5 +75C>G] of SEQ ID No. 20, [CYP17_IVS1 +426G>A] of SEQ ID No. 21, [CYP17_IVS1 −99C>T] of SEQ ID No. 22, [CYP17_IVS1 −700C>G] of SEQ ID No. 23, [CYP17_IVS1 −565G>A] of SEQ ID No. 24, [CYP17_IVS3 +141A>T] of SEQ ID No. 25, [CYP17_{—}5′ region −1488C>G] of SEQ ID No. 26, [CYP17_{—}5′ region −1204C>T] of SEQ ID No. 27, [CYP17_IVS1 +466G>A] of SEQ ID No. 28, [CYP17, 712 base pairs after the stop codon, G>A] of SEQ ID No. 29, [SRD5A2, 1356 base pairs after the stop codon (3′ UTR), A>C] of SEQ ID No. 30, [SRD5A2, 849 base pairs after the stop codon (3′ UTR), A>G] of SEQ ID No. 31, [SRD5A2_{—}5′ region −870G>A] of SEQ ID No. 32, [SRD5A2_{—}5′ region −2036(A)7-8] of SEQ ID No. 33, and [SRD5A2, 545 base pairs after the stop codon (3′ UTR), T>C] of SEQ ID No. 34.

Optionally, the polynucleotide is the complement of any of the isolated polynucleotides hereinbefore described.

In one aspect, the polynucleotide comprises part of the CYP17 gene, the CYP3A4 gene or the SRD5A2 gene.

Preferably, the isolated polynucleotide further comprises a detectable label. More preferably, the detectable label is selected from the group consisting of fluorophore, radionuclide, peptide, enzyme, antibody and antigen. In a preferred embodiment, the fluorophore is a fluorescent compound selected from the group consisting of Hoechst 33342, Cy2, Cy3, Cy5, CypHer, coumarin, FITC, DAPI, Alexa 633, DRAQ5 and Alexa 488.

In a third aspect of the present invention, there is provided a method for diagnosing a genetic susceptibility for a disease, condition or disorder related to prostate or breast cancer in a subject, the method comprising analysing a biological sample containing nucleic acid obtained from the subject to detect the presence or absence of one or more single nucleotide polymorphisms at a position selected from the group consisting of position [CYP3A4_IVS9 +187] of SEQ ID No. 1, position [CYP3A4, 1639 base pairs after the stop codon] of SEQ ID No. 2, position [CYP3A4, 945 base pairs after the stop codon] of SEQ ID No. 3, position [CYP3A4_{—}5′ region −747] of SEQ ID No. 4, position [CYP3A4_IVS7 −202] of SEQ ID No. 5, position [CYP3A4, 2204 base pairs after the stop codon] of SEQ ID No. 6, position [CYP3A4_IVS2 −132] of SEQ ID No. 7, position [CYP3A4_IVS1 −868] of SEQ ID No. 8, position [CYP3A4_{—}5′ region −847] of SEQ ID No. 9, position [CYP3A4, 766 base pairs after the stop codon] of SEQ ID No. 10, position [CYP3A4, 1454 base pairs after the stop codon] of SEQ ID No. 11, position [CYP3A4_IVS3 +1992] of SEQ ID No. 12, position [CYP3A4_IVS9 +841] of SEQ ID No. 13, position [CYP3A4_IVS12 −473] of SEQ ID No. 14, position [CYP3A4_IVS12 +581] of SEQ ID No. 15, position [CYP3A4_IVS12 +586] of SEQ ID No. 16, position [CYP3A4_IVS12 +646] of SEQ ID No. 17, position [CYP3A4_IVS3 −734] of SEQ ID No. 18, position [CYP17_IVS1 −271] of SEQ ID No. 19, position [CYP17_IVS5 +75] of SEQ ID No. 20, position [CYP17_IVS1 +426] of SEQ ID No. 21, position [CYP17_IVS1 −99] of SEQ ID No. 22, position [CYP17_IVS1 −700] of SEQ ID No. 23, position [CYP17_IVS1 −565] of SEQ ID No. 24, position [CYP17_IVS3 +141] of SEQ ID No. 25, position [CYP17_{—}5′ region −1488] of SEQ ID No. 26, position [CYP17_{—}5′ region −1204] of SEQ ID No. 27, position [CYP17_IVS1 +466] of SEQ ID No. 28, position [CYP17, 712 base pairs after the stop codon] of SEQ ID No. 29, position [SRD5A2, 1356 base pairs after the stop codon (3′ UTR)] of SEQ ID No. 30, position [SRD5A2, 849 base pairs after the stop codon (3′ UTR)] of SEQ ID No. 31, position [SRD5A2_{—}5′ region −870] of SEQ ID No. 32, position [SRD5A2_{—}5′ region between −2036 and −2030] of SEQ ID No. 33, position [SRD5A2, 545 base pairs after the stop codon (3′ UTR)] of SEQ ID No. 34, position [SRD5A2_IVS2 +626] of SEQ ID No. 35, position [SRD5A2_{—}5′ region −8029] of SEQ ID No. 36, position [CYP3A4_IVS7 +34] of SEQ ID No. 42, position [CYP3A4_{—}5′ region −1232] of SEQ ID No. 43, position [SRD5A2_{—}5′ region −3001] of SEQ ID No. 44, and position [SRD5A2, 1552 base pairs after the stop codon] of SEQ ID No. 45.

Suitably, the nucleic acid is DNA, RNA, cDNA or mRNA.

Preferably, the single nucleotide polymorphism is selected from the group consisting of [CYP3A4_IVS9 +187C>G] of SEQ ID No. 1, [CYP3A4, 1639 base pairs after the stop codon, A>T] of SEQ ID No. 2, [CYP3A4, 945 base pairs after the stop codon, A>T] of SEQ ID No. 3, [CYP3A4_{—}5′ region −747C>G] of SEQ ID No. 4, [CYP3A4_IVS7 −202C>T] of SEQ ID No. 5, [CYP3A4, 2204 base pairs after the stop codon, G>C] of SEQ ID No. 6, [CYP3A4_IVS2 −132C>T] of SEQ ID No. 7, [CYP3A4_IVS1 −868C>T] of SEQ ID No. 8, [CYP3A4_{—}5′ region −847A>T] of SEQ ID No. 9, [CYP3A4, 766 base pairs after the stop codon, delT] of SEQ ID No. 10, [CYP3A4, 1454 base pairs after the stop codon, C>T] of SEQ ID No. 11, [CYP3A4_IVS3 +1992T>C] of SEQ ID No. 12, [CYP3A4_IVS9 +841T>G] of SEQ ID No. 13, [CYP3A4_IVS12 −473T>G] of SEQ ID No. 14, [CYP3A4_IVS12 +581C>T] of SEQ ID No. 15, [CYP3A4_IVS12 +586G>A] of SEQ ID No. 16, [CYP3A4_IVS12 +646C>A] of SEQ ID No. 17, [CYP3A4_IVS3 −734G>A] of SEQ ID No. 18, [CYP17_IVS1 −271A>C] of SEQ ID No. 19, [CYP17_IVS5 +75C>G] of SEQ ID No. 20, [CYP17_IVS1 +426G>A] of SEQ ID No. 21, [CYP17_IVS1 −99C>T] of SEQ ID No. 22, [CYP17_IVS1 −700C>G] of SEQ ID No. 23, [CYP17_IVS1 −565G>A] of SEQ ID No. 24, [CYP17_IVS3 +141A>T] of SEQ ID No. 25, [CYP17_{—}5′ region −1488C>G] of SEQ ID No. 26, [CYP17_{—}5′ region −1204C>T] of SEQ ID No. 27, [CYP17_IVS1 +466G>A] of SEQ ID No. 28, [CYP17, 712 base pairs after the stop codon, G>A] of SEQ ID No. 29, [SRD5A2, 1356 base pairs after the stop codon (3′ UTR), A>C] of SEQ ID No. 30, [SRD5A2, 849 base pairs after the stop codon (3′ UTR), A>G] of SEQ ID No. 31, [SRD5A2_{—}5′ region −870G>A] of SEQ ID No. 32, [SRD5A2_{—}5′ region −2036(A)7-8] of SEQ ID No. 33, [SRD5A2, 545 base pairs after the stop codon (3′ UTR), T>C] of SEQ ID No. 34, [SRD5A2_IVS2 +626C>T] of SEQ ID No. 35, [SRD5A2_{—}5′ region −8029C>T] of SEQ ID No. 36, [CYP3A4_IVS7 +34T>G] of SEQ ID No. 42, [CYP3A4_{—}5′ region −1232C>T] of SEQ ID No. 43, [SRD5A2_{—}5′ region −3001G>A] of SEQ ID No. 44, and [SRD5A2, 1552 base pairs after the stop codon, G>A] of SEQ ID No. 45.

Optionally, the single nucleotide polymorphism is selected from the complement of any of the single nucleotide polymorphisms described hereinbefore.

Suitably, the analysis is accomplished by sequencing, genotyping, fragment analysis, hybridisation, restriction fragment analysis, oligonucleotide ligation or allele specific PCR. Preferably, the analysis is accomplished by hybridisation, the method comprising the steps of
i) contacting the nucleic acid with an oligonucleotide that hybridises to one or more isolated polynucleotide polymorphic sequence selected from the group consisting of SEQ ID NOS 1-36, 42-45 or its complement
ii) determining whether the nucleic acid and the oligonucleotide hybridize;
   whereby hybridisation of the nucleic acid to the oligonucleotide indicates the presence of the polymorphic site in the nucleic acid.

In a fourth aspect of the present invention, there is provided a method for diagnosing a genetic susceptibility for a disease, condition or disorder related to prostate or breast cancer in a subject, or predicting an individual's response to a drug, the method comprising adding an antibody to a polypeptide present in a biological sample obtained from the subject which polypeptide is encoded by a polynucleotide selected from the group consisting of SEQ ID NOS 1-36 and SEQ ID NOS 42-45, or the complement thereof, and detecting specific binding of the antibody to the polypeptide.

In a fifth aspect of the present invention, there is provided a kit comprising at least one isolated polynucleotide of at least 5 contiguous nucleotides of SEQ ID NOS: 1-36 or 42-45, or the complement thereof, and containing at least one single nucleotide polymorphic site associated with a disease, condition or disorder related to prostate or breast cancer together with instructions for the use thereof for detecting the presence or the absence of said at least single nucleotide polymorphism in said nucleic acid.

In a sixth aspect of the present invention, there is provided an oligonucleotide array comprising at least one oligonucleotide capable of hybridising to a first polynucleotide at a polymorphic site encompassed therein, wherein the first polynucleotide comprises a nucleotide sequence comprising one or more polymorphic sequences of SEQ ID NOS: 1-36 and SEQ ID NOS: 42-45.

Suitably, the first polynucleotide comprises a fragment of any of the nucleotide sequences, the fragment comprising a polymorphic site in the polymorphic sequence.

Suitably, the first polynucleotide is a complementary nucleotide sequence comprising a sequence complementary to one or more polymorphic sequences of SEQ ID NOS: 1-36 and SEQ ID NOS: 42-45.

Suitably, the first polynucleotide comprises a fragment of said complementary sequence, the fragment comprising a polymorphic site in the polymorphic sequence.

Suitably, the position of the polymorphic site in the kit or the microarray as hereinbefore described is at a position selected from the group consisting of position [CYP3A4_IVS9 +187] of SEQ ID No. 1, position [CYP3A4, 1639 base pairs after the stop codon] of SEQ ID No. 2, position [CYP3A4, 945 base pairs after the stop codon] of SEQ ID No. 3, position [CYP3A4_{—}5′ region −747] of SEQ ID No. 4, position [CYP3A4_IVS7 −202] of SEQ ID No. 5, position [CYP3A4, 2204 base pairs after the stop codon] of SEQ ID No. 6, position [CYP3A4_IVS2 −132] of SEQ ID No. 7, position [CYP3A4_IVS1 −868] of SEQ ID No. 8, position [CYP3A4_{—}5′ region −847] of SEQ ID No. 9, position [CYP3A4, 766 base pairs after the stop codon] of SEQ ID No. 10, position [CYP3A4, 1454 base pairs after the stop codon] of SEQ ID No. 11, position [CYP3A4_IVS3 +1992] of SEQ ID No. 12, position [CYP3A4_IVS9 +841] of SEQ ID No. 13, position [CYP3A4_IVS12 −473] of SEQ ID No. 14, position [CYP3A4_IVS12 +581] of SEQ ID No. 15, position [CYP3A4_IVS12 +586] of SEQ ID No. 16, position [CYP3A4_IVS12 +646] of SEQ ID No. 17, position [CYP3A4_IVS3 −734] of SEQ ID No. 18, position [CYP17_IVS1 −271] of SEQ ID No. 19, position [CYP17_IVS5 +75] of SEQ ID No. 20, position [CYP17_IVS1 +426] of SEQ ID No. 21, position [CYP17_IVS1 −99] of SEQ ID No. 22, position [CYP17_IVS1 −700] of SEQ ID No. 23, position [CYP17_IVS1 −565] of SEQ ID No. 24, position [CYP17_IVS3 +141] of SEQ ID No. 25, position [CYP17_{—}5′ region −1488] of SEQ ID No. 26, position [CYP17_{—}5′ region −1204] of SEQ ID No. 27, position [CYP17_IVS1 +466] of SEQ ID No. 28, position [CYP17, 712 base pairs after the stop codon] of SEQ ID No. 29, position [SRD5A2, 1356 base pairs after the stop codon (3′ UTR)] of SEQ ID No. 30, position [SRD5A2, 849 base pairs after the stop codon (3′ UTR)] of SEQ ID No. 31, position [SRD5A2_{—}5′ region −870] of SEQ ID No. 32, position [SRD5A2_{—}5′ region between −2036 and −2030] of SEQ ID No. 33, position [SRD5A2, 545 base pairs after the stop codon (3′ UTR)] of SEQ ID No. 34.position [SRD5A2_IVS2 +626] of SEQ ID No. 35, position [SRD5A2_{—}5′ region −8029] of SEQ ID No. 36, position [CYP3A4_IVS7 +34] of SEQ ID No. 42, position [CYP3A4_{—}5′ region −1232] of SEQ ID No. 43, position [SRD5A2_{—}5′ region −3001] of SEQ ID No. 44 and position [SRD5A2, 1552 base pairs after the stop codon] of SEQ ID No. 45.

Preferably, at least one single nucleotide polymorphism is selected from the group consisting of [CYP3A4_IVS9 +187C>G] of SEQ ID No. 1, [CYP3A4, 1639 base pairs after the stop codon, A>T] of SEQ ID No. 2, [CYP3A4, 945 base pairs after the stop codon, A>T] of SEQ ID No. 3, [CYP3A4_{—}5′ region −747C>G] of SEQ ID No. 4, [CYP3A4_IVS7 −202C>T] of SEQ ID No. 5, [CYP3A4, 2204 base pairs after the stop codon, G>C] of SEQ ID No. 6, [CYP3A4_IVS2 −132C>T] of SEQ ID No. 7, [CYP3A4_IVS1 −868C>T] of SEQ ID No. 8, [CYP3A4_{—}5′ region −847A>T] of SEQ ID No. 9, [CYP3A4, 766 base pairs after the stop codon, delT] of SEQ ID No. 10, [CYP3A4, 1454 base pairs after the stop codon, C>T] of SEQ ID No. 11, [CYP3A4_IVS3 +1992T>C] of SEQ ID No. 12, [CYP3A4_IVS9 +841T>G] of SEQ ID No. 13, [CYP3A4_IVS12 −473T>G] of SEQ ID No. 14, [CYP3A4_IVS12 +581C>T] of SEQ ID No. 15, [CYP3A4_IVS12 +586G>A] of SEQ ID No. 16, [CYP3A4_IVS12 +646C>A] of SEQ ID No. 17, [CYP3A4_IVS3 −734G>A] of SEQ ID No. 18, [CYP17_IVS1 −271A>C] of SEQ ID No. 19, [CYP17_IVS5 +75C>G] of SEQ ID No. 20, [CYP17_IVS1 +426G>A] of SEQ ID No. 21, [CYP17_IVS1 −99C>T] of SEQ ID No. 22, [CYP17_IVS1 −700C>G] of SEQ ID No. 23, [CYP17_IVS1 −565G>A] of SEQ ID No. 24, [CYP17_IVS3 +141A>T] of SEQ ID No. 25, [CYP17_{—}5′ region −1488C>G] of SEQ ID No. 26, [CYP17_{—}5′ region −1204C>T] of SEQ ID No. 27, [CYP17_IVS1 +466G>A] of SEQ ID No. 28, [CYP17, 712 base pairs after the stop codon, G>A] of SEQ ID No. 29, [SRD5A2, 1356 base pairs after the stop codon (3′ UTR), A>C] of SEQ ID No. 30, [SRD5A2, 849 base pairs after the stop codon (3′ UTR), A>G] of SEQ ID No. 31, [SRD5A2_{—}5′ region −870G>A] of SEQ ID No. 32, [SRD5A2_{—}5′ region −2036(A)7-8] of SEQ ID No. 33, [SRD5A2, 545 base pairs after the stop codon (3′ UTR), T>C] of SEQ ID No. 34, [SRD5A2_IVS2 +626C>T] of SEQ ID No. 35, [SRD5A2_{—}5′ region −8029C>T] of SEQ ID No. 36, [CYP3A4_IVS7 +34T>G] of SEQ ID No. 42, [CYP3A4_{—}5′ region −1232C>T] of SEQ ID No. 43, [SRD5A2_{—}5′ region −3001 G>A] of SEQ ID No. 44, and [SRD5A2, 1552 base pairs after the stop codon, G>A] of SEQ ID No. 45.

Optionally, at least one single nucleotide polymorphism is the complement of any of the single nucleotide polymorphisms as hereinbefore described.

Suitably, the oligonucleotide further comprises a detectable label. Preferably, the label is selected from the group consisting of fluorophore, radionuclide, peptide, enzyme, antibody or antigen. More preferably, the fluorophore is a fluorescent compound selected from the group consisting of Hoechst 33342, Cy2, Cy3, Cy5, CypHer, coumarin, FITC, DAPI, Alexa 633 DRAQ5 and Alexa 488.

In a seventh aspect of the present invention, there is provided a method of treatment or prophylaxis of a subject comprising the steps of
i) analysing a biological sample containing nucleic acid obtained from the subject to detect the presence or absence of at least one single nucleotide polymorphism in SEQ ID NOS 1-36 or SEQ ID NOS 42-45, or the complement thereof, associated with a disease, condition or disorder related to prostate or breast cancer; and
ii) treating the subject for the disease, condition or disorder if step i) detects the presence of at least one single nucleotide polymorphism in SEQ ID NOS: 1-36 or SEQ ID NOS 42-45, or the complement thereof.

Treatment may take a variety of forms depending upon the nature of the cancer. Hormonal therapy is a widely used treatment for patients with metastatic carcinoma of the prostate (Goethuys et al. (1997) Am J Clin Oncol. 20, 40-45). Such treatment may, for example, involve androgen deprivation by surgical (e.g. orchiectomy) or androgen suppressive agents such as estrogens, (e.g. diethylstilbestrol), antiandrogens (e.g. flutamide) and luteinising hormone-releasing hormone agonists (e.g. leuprolide). Radiotherapy using radionuclides, is such as ³²Phosphorus or ⁸⁹Strontium, can be an effective treatment for the disease. There is also growing interest in the development of vaccines (Slovin (2001) Hematol. Oncol. Clinic N. Am, 15, 477-496) or the use of gene therapeutic methods (Ferrer & Rodriguez (2001) Hematol Oncol Clinic of N. Am 15, 497-508) for the treatment of prostate cancer.

Suitably, the nucleic acid is selected from the group consisting of DNA, RNA and mRNA.

Preferably, the sample is analysed to detect the presence or absence of at least one single nucleotide polymorphism at a position selected from the group consisting of position [CYP3A4_IVS9 +187] of SEQ ID No. 1, position [CYP3A4, 1639 base pairs after the stop codon] of SEQ ID No. 2, position [CYP3A4, 945 base pairs after the stop codon] of SEQ ID No. 3, position [CYP3A4_{—}5′ region 31 747] of SEQ ID No. 4, position [CYP3A4_IVS7 −202] of SEQ ID No. 5, position [CYP3A4, 2204 base pairs after the stop codon] of SEQ ID No. 6, position [CYP3A4_IVS2 −132] of SEQ ID No. 7, position [CYP3A4_IVS1 −868] of SEQ ID No. 8, position [CYP3A4_{—}5′ region −847] of SEQ ID No. 9, position [CYP3A4, 766 base pairs after the stop codon] of SEQ ID No. 10, position [CYP3A4, 1454 base pairs after the stop codon] of SEQ ID No. 11, position [CYP3A4_IVS3 +1992] of SEQ ID No. 12, position [CYP3A4_IVS9 +841] of SEQ ID No. 13, position [CYP3A4_IVS12 −473] of SEQ ID No. 14, position [CYP3A4_IVS12 +581] of SEQ ID No. 15, position [CYP3A4_IVS12 +586] of SEQ ID No. 16, position [CYP3A4_IVS12 +646] of SEQ ID No. 17, position [CYP3A4_IVS3 −734] of SEQ ID No. 18, position [CYP17_IVS1 −271] of SEQ ID No. 19, position [CYP17_IVS5 +75] of SEQ ID No. 20, position [CYP17_IVS1 +426] of SEQ ID No. 21, position [CYP17_IVS1 −99] of SEQ ID No. 22, position [CYP17_IVS1 −700] of SEQ ID No. 23, position [CYP17_IVS1 −565] of SEQ ID No. 24, position [CYP17_IVS3 +141] of SEQ ID No. 25, position [CYP17_{—}5′ region −1488] of SEQ ID No. 26, position [CYP17_{—}5′ region −1204] of SEQ ID No. 27, position [CYP17_IVS1 +466] of SEQ ID No. 28, position [CYP17, 712 base pairs after the stop codon] of SEQ ID No. 29, position [SRD5A2, 1356 base pairs after the stop codon (3′ UTR)] of SEQ ID No. 30, position [SRD5A2, 849 base pairs after the stop codon (3′ UTR)] of SEQ ID No. 31, position [SRD5A2_{—}5′ region −870] of SEQ ID No. 32, position [SRD5A2_{—}5′ region between −2036 and −2030] of SEQ ID No. 33, position [SRD5A2, 545 base pairs after the stop codon (3′ UTR)] of SEQ ID No. 34.position [SRD5A2_IVS2 +626] of SEQ ID No. 35, position [SRD5A2_{—}5′ region −8029] of SEQ ID No. 36, position [CYP3A4_IVS7 +34] of SEQ ID No. 42, position [CYP3A4_{—}5′ region −1232] of SEQ ID No. 43, position [SRD5A2_{—}5′ region −3001] of SEQ ID No. 44, and position [SRD5A2, 1552 base pairs after the stop codon] of SEQ ID No. 45.

More preferably, at least one single nucleotide polymorphism is selected from the group consisting of [CYP3A4_IVS9 +187C>G] of SEQ ID No. 1, [CYP3A4, 1639 base pairs after the stop codon, A>T] of SEQ ID No. 2, [CYP3A4, 945 base pairs after the stop codon, A>T] of SEQ ID No. 3, [CYP3A4_{—}5′ region −747C>G] of SEQ ID No. 4, [CYP3A4_IVS7 −202C>T] of SEQ ID No. 5, [CYP3A4, 2204 base pairs after the stop codon, G>C] of SEQ ID No. 6, [CYP3A4_IVS2 −132C>T] of SEQ ID No. 7, [CYP3A4_IVS1 −868C>T] of SEQ ID No. 8, [CYP3A4_{—}5′ region −847A>T] of SEQ ID No. 9, [CYP3A4, 766 base pairs after the stop codon, delT] of SEQ ID No. 10, [CYP3A4, 1454 base pairs after the stop codon, C>T] of SEQ ID No. 11, [CYP3A4_IVS3 +1992T>C] of SEQ ID No. 12, [CYP3A4_IVS9 +841T>G] of SEQ ID No. 13, [CYP3A4_IVS12 −473T>G] of SEQ ID No. 14, [CYP3A4_IVS12 +581C>T] of SEQ ID No. 15, [CYP3A4_IVS12 +586G>A] of SEQ ID No. 16, [CYP3A4_IVS12 +646C>A] of SEQ ID No. 17, [CYP3A4_IVS3 −734G>A] of SEQ ID No. 18, [CYP17_IVS1 −271A>C] of SEQ ID No. 19, [CYP17_IVS5 +75C>G] of SEQ ID No. 20, [CYP17_IVS1 +426G>A] of SEQ ID No. 21, [CYP17_IVS1 −99C>T] of SEQ ID No. 22, [CYP17_IVS1 −700C>G] of SEQ ID No. 23, [CYP17_IVS1 −565G>A] of SEQ ID No. 24, [CYP17_IVS3 +141A>T] of SEQ ID No. 25, [CYP17_{—}5′ region −1488C>G] of SEQ ID No. 26, [CYP17_{—}5′ region −1204C>T] of SEQ ID No. 27, [CYP17_IVS1 +466G>A] of SEQ ID No. 28, [CYP17, 712 base pairs after the stop codon, G>A] of SEQ ID No. 29, [SRD5A2, 1356 base pairs after the stop codon (3′ UTR), A>C] of SEQ ID No. 30, [SRD5A2, 849 base pairs after the stop codon (3′ UTR), A>G] of SEQ ID No. 31, [SRD5A2_{—}5′ region −870G>A] of SEQ ID No. 32, [SRD5A2_{—}5′ region −2036(A)7-8] of SEQ ID No. 33, [SRD5A2, 545 base pairs after the stop codon (3′ UTR), T>C] of SEQ ID No. 34, [SRD5A2_IVS2 +626C>T] of SEQ ID No. 35, [SRD5A2_{—}5′ region −8029C>T] of SEQ ID No. 36, [CYP3A4_IVS7 +34T>G] of SEQ ID No. 42, [CYP3A4_{—}5′ region −1232C>T] of SEQ ID No. 43, [SRD5A2_{—}5′ region −3001G>A] of SEQ ID No. 44, and [SRD5A2, 1552 base pairs after the stop codon, G>A] of SEQ ID No. 45.

Optionally, at least one single nucleotide polymorphism is the complement of any of the single nucleotide polymorphisms hereinbefore described.

Suitably, the method counteracts the effect of at least one single nucleotide polymorphism detected.

In a first embodiment of the seventh aspect, the method comprises treatment with a polynucleotide selected from the group consisting of polymorphic sequences SEQ ID NOS 1-36 or SEQ ID NOS 42-45, or their complement, provided that the polymorphic sequence, or the complement, does not contain at least one single nucleotide polymorphism at a position selected from the group consisting of position [CYP3A4_IVS9 +187] of SEQ ID No. 1, position [CYP3A4, 1639 base pairs after the stop codon] of SEQ ID No. 2, position [CYP3A4, 945 base pairs after the stop codon,] of SEQ ID No. 3, position [CYP3A4_{—}5′ region −747] of SEQ ID No. 4, position [CYP3A4_IVS7 −202] of SEQ ID No. 5, position [CYP3A4, 2204 base pairs after the stop codon,] of SEQ ID No. 6, position [CYP3A4_IVS2 −132] of SEQ ID No. 7, position [CYP3A4_IVS1 −868] of SEQ ID No. 8, position [CYP3A4_{—}5′ region −847] of SEQ ID No. 9, position [CYP3A4, 766 base pairs after the stop codon] of SEQ ID No. 10, position [CYP3A4, 1454 base pairs after the stop codon] of SEQ ID No. 11, position [CYP3A4_IVS3 +1992] of SEQ ID No. 12, position [CYP3A4_IVS9 +841] of SEQ ID No. 13, position [CYP3A4_IVS12 −473] of SEQ ID No. 14, position [CYP3A4_IVS12 +581] of SEQ ID No. 15, position [CYP3A4_IVS12 +586] of SEQ ID No. 16, position [CYP3A4_IVS12 +646] of SEQ ID No. 17, position [CYP3A4_IVS3 −734] of SEQ ID No. 18, position [CYP17_IVS1 −271] of SEQ ID No. 19, position [CYP17_IVS5 +75] of SEQ ID No. 20, position [CYP17_IVS1 +426] of SEQ ID No. 21, position [CYP17_IVS1 −99] of SEQ ID No. 22, position [CYP17_IVS1 −700] of SEQ ID No. 23, position [CYP17_IVS1 −565] of SEQ ID No. 24, position [CYP17_IVS3 +141] of SEQ ID No. 25, position [CYP17_{—}5′ region −1488] of SEQ ID No. 26, position [CYP17_{—}5′ region −1204] of SEQ ID No. 27, position [CYP17_IVS1 +466] of SEQ ID No. 28, position [CYP17, 712 base pairs after the stop codon] of SEQ ID No. 29, position [SRD5A2, 1356 base pairs after the stop codon (3′ UTR)] of SEQ ID No. 30, position [SRD5A2, 849 base pairs after the stop codon (3′ UTR)] of SEQ ID No. 31, position [SRD5A2_{—}5′ region −870] of SEQ ID No. 32, position [SRD5A2_{—}5′ region between −2036 and −2030] of SEQ ID No. 33, position [SRD5A2, 545 base pairs after the stop codon (3′ UTR)] of SEQ ID No. 34position [SRD5A2_IVS2 +626] of SEQ ID No. 35, position [SRD5A2_{—}5′ region −8029] of SEQ ID No. 36, position [CYP3A4_IVS7 +34] of SEQ ID No. 42, position [CYP3A4_{—}5′ region −1232] of SEQ ID No. 43, position [SRD5A2_{—}5′ region −3001] of SEQ ID No. 44, and position [SRD5A2, 1552 base pairs after the stop codon] of SEQ ID No. 45.

Preferably, the polymorphic sequence does not contain at least one single nucleotide polymorphism selected from the group consisting of [CYP3A4_IVS9 +187C>G] of SEQ ID No. 1, [CYP3A4, 1639 base pairs after the stop codon, A>T] of SEQ ID No. 2, [CYP3A4, 945 base pairs after the stop codon, A>T] of SEQ ID No. 3, [CYP3A4_{—}5′ region −747C>G] of SEQ ID No. 4, [CYP3A4_IVS7 −202C>T] of SEQ ID No. 5, [CYP3A4, 2204 base pairs after the stop codon, G>C] of SEQ ID No. 6, [CYP3A4_IVS2 −132C>T] of SEQ ID No. 7, [CYP3A4_IVS1 −868C>T] of SEQ ID No. 8, [CYP3A4_{—}5′ region −847A>T] of SEQ ID No. 9, [CYP3A4, 766 base pairs after the stop codon, delT] of SEQ ID No. 10, [CYP3A4, 1454 base pairs after the stop codon, C>T] of SEQ ID No. 11, [CYP3A4_IVS3 +1992T>C] of SEQ ID No. 12, [CYP3A4_IVS9 +841T>G] of SEQ ID No. 13, [CYP3A4_IVS12 −473T>G] of SEQ ID No. 14, [CYP3A4_IVS12 +581C>T] of SEQ ID No. 15, [CYP3A4_IVS12 +586G>A] of SEQ ID No. 16, [CYP3A4_IVS12 +646C>A] of SEQ ID No. 17, [CYP3A4_IVS3 −734G>A] of SEQ ID No. 18, [CYP17_IVS1 −271A>C] of SEQ ID No. 19, [CYP17_IVS5 +75C>G] of SEQ ID No. 20, [CYP17_IVS1 +426G>A] of SEQ ID No. 21, [CYP17_IVS1 −99C>T] of SEQ ID No. 22, [CYP17_IVS1 −700C>G] of SEQ ID No. 23, [CYP17_IVS1 −565G>A] of SEQ ID No. 24, [CYP17_IVS3 +141A>T] of SEQ ID No. 25, [CYP17_{—}5′ region −1488C>G] of SEQ ID No. 26, [CYP17_{—}5′ region −1204C>T] of SEQ ID No. 27, [CYP17_IVS1 +466G>A] of SEQ ID No. 28, [CYP17, 712 base pairs after the stop codon, G>A] of SEQ ID No. 29, [SRD5A2, 1356 base pairs after the stop codon (3′ UTR), A>C] of SEQ ID No. 30, [SRD5A2, 849 base pairs after the stop codon (3′ UTR), A>G] of SEQ ID No. 31, [SRD5A2_{—}5′ region −870G>A] of SEQ ID No. 32, [SRD5A2_{—}5′ region −2036(A)7-8] of SEQ ID No. 33, [SRD5A2, 545 base pairs after the stop codon (3′ UTR), T>C] of SEQ ID No. 34, [SRD5A2_IVS2 +626C>T] of SEQ ID No. 35, [SRD5A2_{—}5′ region −8029C>T] of SEQ ID No. 36, [CYP3A4_IVS7 +34T>G] of SEQ ID No. 42, [CYP3A4_{—}5′ region −1232C>T] of SEQ ID No. 43, [SRD5A2_{—}5′ region −3001G>A] of SEQ ID No. 44, and [SRD5A2, 1552 base pairs after the stop codon, G>A] of SEQ ID No. 45.

Preferably, the polymorphic sequence does not contain at least one single nucleotide polymorphism which is the complement of any of the single nucleotide polymorphisms hereinbefore described.

In a second embodiment of the seventh aspect, the method comprises treatment with a polypeptide which is encoded by a polynucleotide selected from the group consisting of polymorphic sequences SEQ ID NOS 1-36 and SEQ ID NOS 42-45 or their complement, provided that the polymorphic sequence, or the complement, does not contain at least one single nucleotide polymorphism at a position selected from the group consisting of position [CYP3A4_IVS9 +187] of SEQ ID No. 1, position [CYP3A4, 1639 base pairs after the stop codon] of SEQ ID No. 2, position [CYP3A4, 945 base pairs after the stop codon] of SEQ ID No. 3, position [CYP3A4_{—}5′ region −747] of SEQ ID No. 4, position [CYP3A4_IVS7 −202] of SEQ ID No. 5, position [CYP3A4, 2204 base pairs after the stop codon] of SEQ ID No. 6, position [CYP3A4_IVS2 −132] of SEQ ID No. 7, position [CYP3A4_IVS1 −868] of SEQ ID No. 8, position [CYP3A4_{—}5′ region −847] of SEQ ID No. 9, position [CYP3A4, 766 base pairs after the stop codon] of SEQ ID No. 10, position [CYP3A4, 1454 base pairs after the stop codon] of SEQ ID No. 11, position [CYP3A4_IVS3 +1992] of SEQ ID No. 12, position [CYP3A4_IVS9 +841] of SEQ ID No. 13, position [CYP3A4_IVS12 −473] of SEQ ID No. 14, position [CYP3A4_IVS12 +581] of SEQ ID No. 15, position [CYP3A4_IVS12 +586] of SEQ ID No. 16, position [CYP3A4_IVS12 +646] of SEQ ID No. 17, position [CYP3A4_IVS3 −734] of SEQ ID No. 18, position [CYP17_IVS1 −271] of SEQ ID No. 19, position [CYP17_IVS5 +75] of SEQ ID No. 20, position [CYP17_IVS1 +426] of SEQ ID No. 21, position [CYP17_IVS1 −99] of SEQ ID No. 22, position [CYP17_IVS1 −700] of SEQ ID No. 23, position [CYP17_IVS1 −565] of SEQ ID No. 24, position [CYP17_IVS3 +141] of SEQ ID No. 25, position [CYP17_{—}5′ region −1488] of SEQ ID No. 26, position [CYP17_{—}5′ region −1204] of SEQ ID No. 27, position [CYP17_IVS1 +466] of SEQ ID No. 28, position [CYP17, 712 base pairs after the stop codon] of SEQ ID No. 29, position [SRD5A2, 1356 base pairs after the stop codon (3′ UTR)] of SEQ ID No. 30, position [SRD5A2, 849 base pairs after the stop codon (3′ UTR)] of SEQ ID No. 31, position [SRD5A2_{—}5′ region −870] of SEQ ID No. 32, position [SRD5A2_{—}5′ region between −2036 and −2030] of SEQ ID No. 33, position [SRD5A2, 545 base pairs after the stop codon (3′ UTR)] of SEQ ID No. 34, position [SRD5A2_IVS2 +626] of SEQ ID No. 35, position [SRD5A2_{—}5′ region −8029] of SEQ ID No. 36, position [CYP3A4_IVS7 +34] of SEQ ID No. 42, position [CYP3A4_{—}5′ region −1232] of SEQ ID No. 43, position [SRD5A2_{—}5′ region −3001] of SEQ ID No. 44 and position [SRD5A2, 1552 base pairs after the stop codon] of SEQ ID No. 45.

Preferably, the polymorphic sequence does not contain at least one single nucleotide polymorphism selected from the group consisting of [CYP3A4_IVS9 +187C>G] of SEQ ID No. 1, [CYP3A4, 1639 base pairs after the stop codon, A>T] of SEQ ID No. 2, [CYP3A4, 945 base pairs after the stop codon, A>T] of SEQ ID No. 3, [CYP3A4_{—}5′ region −747C>G] of SEQ ID No. 4, [CYP3A4_IVS7 −202C>T] of SEQ ID No. 5, [CYP3A4, 2204 base pairs after the stop codon, G>C] of SEQ ID No. 6, [CYP3A4_IVS2 −132C>T] of SEQ ID No. 7, [CYP3A4_IVS1 −868C>T] of SEQ ID No. 8, [CYP3A4_{—}5′ region −847A>T] of SEQ ID No. 9[CYP3A4, 766 base pairs after the stop codon, delT] of SEQ ID No. 10, [CYP3A4, 1454 base pairs after the stop codon, C>T] of SEQ ID No. 11, [CYP3A4_IVS3 +1992T>C] of SEQ ID No. 12, [CYP3A4_IVS9 +841T>G] of SEQ ID No. 13, [CYP3A4_IVS12 −473T>G] of SEQ ID No. 14, [CYP3A4_IVS12 +581C>T] of SEQ ID No. 15, [CYP3A4_IVS12 +586G>A] of SEQ ID No. 16, [CYP3A4_IVS12 +646C>A] of SEQ ID No. 17, [CYP3A4_IVS3 −734G>A] of SEQ ID No. 18, [CYP17_IVS1 −271A>C] of SEQ ID No. 19, [CYP17_IVS5 +75C>G] of SEQ ID No. 20, [CYP17_IVS1 +426G>A] of SEQ ID No. 21, [CYP17_IVS1 −99C>T] of SEQ ID No. 22, [CYP17_IVS1 −700C>G] of SEQ ID No. 23, [CYP17_IVS1 −565G>A] of SEQ ID No. 24, [CYP17_IVS3 +141A>T] of SEQ ID No. 25, [CYP17_{—}5′ region −1488C>G] of SEQ ID No. 26, [CYP17_{—}5′ region −1204C>T] of SEQ ID No. 27, [CYP17_IVS1 +466G>A] of SEQ ID No. 28, [CYP17, 712 base pairs after the stop codon, G>A] of SEQ ID No. 29, [SRD5A2, 1356 base pairs after the stop codon (3′ UTR), A>C] of SEQ ID No. 30, [SRD5A2, 849 base pairs after the stop codon (3′ UTR), A>G] of SEQ ID No. 31, [SRD5A2_{—}5′ region −870G>A] of SEQ ID No. 32, [SRD5A2_{—}5′ region −2036(A)7-8] of SEQ ID No. 33, [SRD5A2, 545 base pairs after the stop codon (3′ UTR), T>C] of SEQ ID No. 34, [SRD5A2_IVS2 +626C>T] of SEQ ID No. 35, [SRD5A2_{—}5′ region −8029C>T] of SEQ ID No. 36, [CYP3A4_IVS7 +34T>G] of SEQ ID No. 42, [CYP3A4_{—}5′ region −1232C>T] of SEQ ID No. 43, [SRD5A2_{—}5′ region −3001G>A] of SEQ ID No. 44, and [SRD5A2, 1552 base pairs after the stop codon, G>A] of SEQ ID No. 45.

Suitably, the polymorphic sequence does not contain at least one single nucleotide which is the complement of any of the single nucleotide polymorphisms as hereinbefore described.

In a third embodiment of the seventh aspect, the method comprises treatment with an antibody that binds specifically with a polypeptide encoded by a polynucleotide selected from the group consisting of SEQ ID NOS 1-34, or SEQ ID NOS 42-45, or the complement thereof.

According to an eighth aspect of the present invention, there is provided a method for predicting the genetic ability of a subject or an organism to metabolise a chemical, the method comprising analysing a biological sample containing nucleic acid obtained from the subject or organism to detect the presence or absence of one or more single nucleotide polymorphisms at a position selected from the group consisting of position [CYP3A4_IVS9 +187] of SEQ ID No. 1, position [CYP3A4, 1639 base pairs after the stop codon] of SEQ ID No. 2, position [CYP3A4, 945 base pairs after the stop codon] of SEQ ID No. 3, position [CYP3A4_{—}5′ region −747] of SEQ ID No. 4, position [CYP3A4_IVS7 −202] of SEQ ID No. 5, position [CYP3A4, 2204 base pairs after the stop codon] of SEQ ID No. 6, position [CYP3A4_IVS2 −132] of SEQ ID No. 7, position [CYP3A4_IVS1 −868] of SEQ ID No. 8, position [CYP3A4_{—}5′ region −847] of SEQ ID No. 9, position [CYP3A4, 766 base pairs after the stop codon] of SEQ ID No. 10, position [CYP3A4, 1454 base pairs after the stop codon] of SEQ ID No. 11, position [CYP3A4_IVS3 +1992] of SEQ ID No. 12, position [CYP3A4_IVS9 +841] of SEQ ID No. 13, position [CYP3A4_IVS12 −473] of SEQ ID No. 14, position [CYP3A4_IVS12 +581] of SEQ ID No. 15, position [CYP3A4_IVS12 +586] of SEQ ID No. 16, position [CYP3A4_IVS12 +646] of SEQ ID No. 17, position [CYP3A4_IVS3 −734] of SEQ ID No. 18, position [CYP17_IVS1 −271] of SEQ ID No. 19, position [CYP17_IVS5 +75] of SEQ ID No. 20, position [CYP17_IVS1 +426] of SEQ ID No. 21, position [CYP17_IVS1 −99] of SEQ ID No. 22, position is [CYP17_IVS1 −700] of SEQ ID No. 23, position [CYP17_IVS1 −565] of SEQ ID No. 24, position [CYP17_IVS3 +141] of SEQ ID No. 25, position [CYP17_{—}5′ region −1488] of SEQ ID No. 26, position [CYP17_{—}5′ region −1204] of SEQ ID No. 27, position [CYP17_IVS1 +466] of SEQ ID No. 28, position [CYP17, 712 base pairs after the stop codon] of SEQ ID No. 29, position [SRD5A2, 1356 base pairs after the stop codon (3′ UTR)] of SEQ ID No. 30, position [SRD5A2, 849 base pairs after the stop codon (3′ UTR)] of SEQ ID No. 31, position [SRD5A2_{—}5′ region −870] of SEQ ID No. 32, position [SRD5A2_{—}5′ region between −2036 and −2030] of SEQ ID No. 33, position [SRD5A2, 545 base pairs after the stop codon (3′ UTR)] of SEQ ID No. 34, position [SRD5A2_IVS2 +626] of SEQ ID No. 35, position [SRD5A2_{—}5′ region −8029] of SEQ ID No. 36, position [CYP3A4_IVS7 +34] of SEQ ID No. 42, position [CYP3A4_{—}5′ region −1232] of SEQ ID No. 43, position [SRD5A2_{—}5′ region −3001] of SEQ ID No. 44, and position [SRD5A2, 1552 base pairs after the stop codon] of SEQ ID No. 45.

Wherein the presence of a polymorphism at one or more of the positions is indicative of the subject's or organism's ability or inability to metabolise the chemical.

Preferably, the analysis comprises detecting the presence or absence of one or more single nucleotide polymorphisms selected from the group consisting of [CYP3A4_IVS9 +187C>G] of SEQ ID No. 1, [CYP3A4, 1639 base pairs after the stop codon, A>T] of SEQ ID No. 2, [CYP3A4, 945 base pairs after the stop codon, A>T] of SEQ ID No. 3, [CYP3A4_{—}5′ region −747C>G] of SEQ ID No. 4, [CYP3A4_IVS7 −202C>T] of SEQ ID No. 5, [CYP3A4, 2204 base pairs after the stop codon, G>C] of SEQ ID No. 6, [CYP3A4_IVS2 −132C>T] of SEQ ID No. 7, [CYP3A4_IVS1 −868C>T] of SEQ ID No. 8, [CYP3A4_{—}5′ region −847A>T] of SEQ ID No. 9, [CYP3A4, 766 base pairs after the stop codon, delT] of SEQ ID No. 10, [CYP3A4, 1454 base pairs after the stop codon, C>T] of SEQ ID No. 11, [CYP3A4_IVS3 +1992T>C] of SEQ ID No. 12, [CYP3A4_IVS9 +841T>G] of SEQ ID No. 13, [CYP3A4_IVS12 −473T>G] of SEQ ID No. 14, [CYP3A4_IVS12 +581C>T] of SEQ ID No. 15, [CYP3A4_IVS12 +586G>A] of SEQ ID No. 16, [CYP3A4_IVS12 +646C>A] of SEQ ID No. 17, [CYP3A4_IVS3 −734G>A] of SEQ ID No. 18, [CYP17_IVS1 −271A>C] of SEQ ID No. 19, [CYP17_IVS5 +75C>G] of SEQ ID No. 20, [CYP17_IVS1 +426G>A] of SEQ ID No. 21, [CYP17_IVS1 −99C>T] of SEQ ID No. 22, [CYP17_IVS1 −700C>G] of SEQ ID No. 23, [CYP17_IVS1 −565G>A] of SEQ ID No. 24, [CYP17_IVS3 +141A>T] of SEQ ID No. 25, [CYP17_{—}5′ region −1488C>G] of SEQ ID No. 26, [CYP17_{—}5′ region −1204C>T] of SEQ ID No. 27, [CYP17_IVS1 +466G>A] of SEQ ID No. 28, [CYP17, 712 base pairs after the stop codon, G>A] of SEQ ID No. 29, [SRD5A2 1356 base pairs after the stop codon (3′ UTR), A>C] of SEQ ID No. 30, [SRD5A2, 849 base pairs after the stop codon (3′ UTR), A>G] of SEQ ID No. 31, [SRD5A2_{—}5′ region −870G>A] of SEQ ID No. 32, [SRD5A2_{—}5′ region −2036(A)7-8] of SEQ ID No. 33, [SRD5A2, 545 base pairs after the stop codon (3′ UTR), T>C] of SEQ ID No. 34, [SRD5A2_IVS2 +626C>T] of SEQ ID No. 35, [SRD5A2_{—}5′ region −8029C>T] of SEQ ID No. 36, [CYP3A4_IVS7 +34T>G] of SEQ ID No. 42, [CYP3A4_{—}5′ region −1232C>T] of SEQ ID No. 43, [SRD5A2_{—}5′ region −3001G>A] of SEQ ID No. 44, and [SRD5A2, 1552 base pairs after the stop codon, G>A] of SEQ ID No. 45.

Preferably, the method further comprises predicting the response of the subject or the organism to the chemical by their ability or inability to metabolise the chemical.

Suitably, the chemical is a drug or a xenobiotic.

Suitably, the organism is selected from the group consisting of bacterium, fungus, protozoa, alga, insect, nematode, amphibian, plant, fish and mammal.

In a ninth aspect of the present invention, there is provided a vector comprising a polynucleotide selected from the group consisting of a nucleotide sequence comprising one or more polymorphic sequences of SEQ ID NOS 1-34 or SEQ ID NOS 42-45.

In a tenth aspect of the present invention, there is provided a host cell transformed with the vector hereinbefore described.

Preferably, the host cell is selected from the group consisting of, bacterium, fungus, protozoa, alga, insect, nematode, amphibian, plant, fish and mammal. More preferably the mammalian cell is a human cell.

In an eleventh aspect of the present invention, there is provided a method of metabolising a chemical using the host cell as hereinbefore described.

In a twelfth aspect of the present invention, there is provided a method for making a host cell resistant to a chemical, the method comprising transforming a cell with any of the polynucleotides or with any of the vectors as hereinbefore described.

In a thirteenth aspect of the present invention, there is provided an isolated haplotype selected from the group consisting of CYP3A4_Hap4 and SRD52_Hap3.

Preferably, the isolated CYP3A4_Hap4 haplotype consists of Allele T at [CYP3A4_{—}5′ region −1232C>T], Allele C at [CYP3A4_{—}5′ region −747C>G], Allele G at [CYP3A4_{—}5′ region −392A>G], Allele G at [CYP3A4_IVS7 +34T>G], Allele T at [CYP3A4_IVS7 −202C>T], Allele G at [CYP3A4_stop +766T>G], Allele C at [CYP3A4_stop +1454C>T], Allele T at [CYP3A4_stop +1639A>T] and Allele C at [CYP3A4 stop +2204G>C].

Preferably, the isolated SRD52_Hap3 haplotype consists of Allele C at [SRD5A2_{—}5′ region −8029C>T], Allele G at [SRD5A2_{—}5′ region −3001 G>A], Allele G at [SRD5A2_{—}145G>A], Allele G at [SRD5A2_{—}265G>C], Allele T at [SRD5A2_IVS2 +626C>T], Allele G at [SRD5A2_stop +1552G>A], Allele G at [SRD5A2_stop +3059G>A] and Allele G at [SRD5A2_stop +9301 G>C].

In a fourteenth aspect of the present invention, there is provided a method for diagnosing a genetic susceptibility for a disease, condition or disorder related to prostate or breast cancer in a subject, the method comprising analysing a biological sample obtained from the subject to detect the presence or absence of a haplotype as hereinbefore described.

In a fifteenth aspect of the present invention, there is provided a method of diagnosing a genetic susceptibility for a disease, condition or disorder related to prostate or breast cancer in a subject, the method comprising adding an antibody to a polypeptide present in a sample obtained from the subject, which polypeptide is encoded by a haplotype as hereinbefore described, or the complement thereof, and detecting specific binding of the antibody to the polypeptide.

In a sixteenth aspect of the present invention, there is provided a method of treatment or prophylaxis of a subject comprising the steps of
i) analysing a sample of biological material containing a nucleic acid obtained from the subject to detect the presence or absence of at least one haplotype as hereinbefore described, or the complement thereof, associated with a disease, condition or disorder related to prostate or breast cancer; and
ii) treating the subject for the disease, condition or disorder if step i) detects the presence of at least one haplotype, or the complement thereof.

Preferably, the method comprises treatment with a portion of the isolated CYP3A4_Hap4 haplotype as hereinbefore described wherein the portion of the haplotype does not consist of at least one allele from the group consisting of Allele T at [CYP3A4_{—}5′ region −1232C>T], Allele C at [CYP3A4_{—}5′ region −747C>G], Allele G at [CYP3A4_{—}5′ region −392A>G], Allele G at [CYP3A4_IVS7 +34T>G], Allele T at [CYP3A4_IVS7 −202C>T], Allele G at [CYP3A4_stop +766T>G], Allele C at [CYP3A4_stop +1454C>T], Allele T at [CYP3A4_stop +1639A>T] and Allele C at [CYP3A4_stop +2204G>C].

Optionally, the method comprises treatment with a portion of the the isolated SRD5A2_Hap3 haplotype as hereinbefore described wherein the portion of the haplotype does not comprise of at least one allele from the group consisting of Allele C at [SRD5A2_{—}5′ region −8029C>T], Allele G at [SRD5A2_{—}5′ region −3001G>A], Allele G at [SRD5A2_{—}145G>A], Allele G at [SRD5A2_{—}265G>C], Allele T at [SRD5A2_IVS2 +626C>T], Allele G at [SRD5A2_stop +1552G>A], Allele G at [SRD5A2_stop +3059G>A] and Allele G at [SRD5A2_stop +9301 G>C].

### BRIEF DESCRIPTION OF THE FIGURES

FIG. 1 illustrates the Testosterone Biosynthetic Pathway.

FIGS. 2A, 2B, and 2C show the location and allele frequencies of selected SNPs in CYP17A1 (FIG. 2A), CYP3A4 (FIG. 2B), and SRD5A2 (FIG. 2C), together with the major haplotypes. Solid black triangles refer to the locations of novel SNPs while white triangles denote locations of known SNPs. All haplotypes with frequency ≧23% in at least one of the four sub-groups (European Americans(EA), African Americans(AA), cases, controls) are given, along with their case and control frequencies. Composite haplotype refers to all the remaining rare haplotypes pooled together.

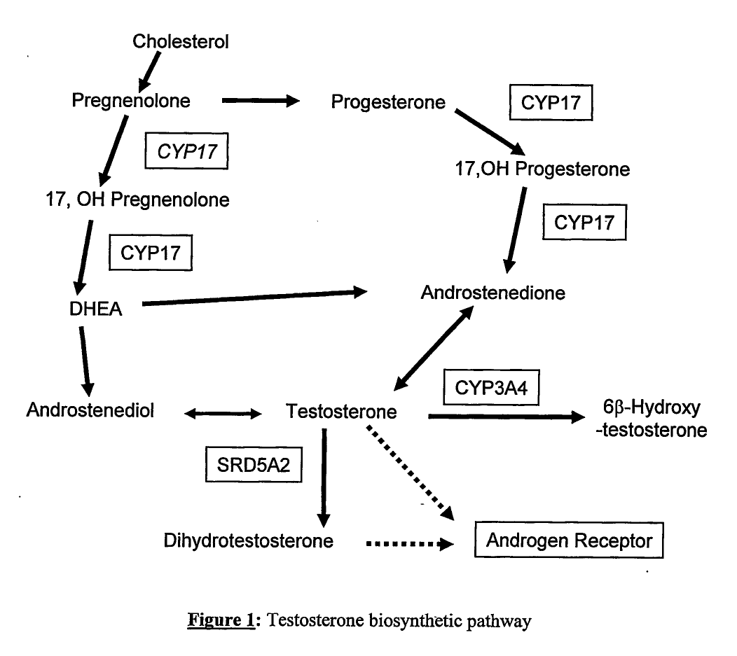

### DETAILED DESCRIPTION OF THE INVENTION

Approach

A two-phase study was undertaken of CYP17, CYP3A4, and SRD5A2, to evaluate the relationship between their genotypes/haplotypes and prostate cancer. Phase I of the study first searched for single nucleotide polymorphisms (SNPs) in these genes by re-sequencing 24 individuals from Coriell Polymorphism Discovery Resource (Coriell Cell Repositories, Camden, N.J.), approximately 100 men from prostate cancer case-control sibships, and by leveraging public databases. Eighty-seven SNPs were discovered and genotyped in 276 men from case-control sibships. Those SNPs exhibiting preliminary case-control allele frequency differences, or distinguishing (i.e., ‘tagging’) common haplotypes across the genes, were identified for further study (24 SNPs total). In Phase II of the study, the 24 SNPs were genotyped in an additional 841 men from case-control sibships. Finally, associations between genotypes/haplotypes in CYP17, CYP3A4, and SRD5A2 and prostate cancer were evaluated in the total case-control sample of 1,117 brothers.

Subjects

A family-based association study population of 1,117 men (637 cases, 480 controls) was recruited between January 1998 and January 2001 from the major medical institutions in the greater Cleveland area and from the Henry Ford Health System in Detroit. The study was approved by the collaborating institution's Review Boards, and informed consent was obtained from all participating men. Characteristics of the study population have been described (Casey et al. (2002) Nat Genet 32, 581-583).

Men diagnosed with histologically confirmed prostate cancer at age 73 or younger were invited to join the study if they had a living unaffected brother who was either older than the proband, or at most eight years younger than the age at diagnosis of the proband. This age restriction was selected in an attempt to increase the potential for genetic factors affecting disease, and to help make certain that the controls were not unaffected due simply to being of a younger age. To help confirm that the controls were not diseased, the prostate specific antigen (PSA) levels in their blood was tested. Individuals in the study with PSA levels above 4 ng/ml were retained as ‘controls’ unless a subsequent diagnosis of prostate cancer was made, at which time they were reclassified as cases. Keeping them in the study was important because automatically excluding men with elevated PSA levels regardless of their ultimate prostate cancer status can lead to biased estimates of association (Lubin & Hartge (1984) Am J Epidemiol 120, 791-793; Poole (1999) Am J Epidemiol 150, 547-551). Information on the cases' Gleason score (a measure of prostate cancer cellular differentiation) and tumor stage (TNM, tumor-node-metastasis stage) was determined from their medical records. The study population was comprised of 90% Caucasians (European Americans), and the remainder primarily African American (9%).

Polymorphism Discovery

Polymorphisms were discovered by sequencing individuals from prostate cancer sibships (67 cases and 43 controls for CYP17 and CYP3A4, and 51 cases and 41 controls for SRD5A2). Of the 110 individuals sequenced for CYP17 and CYP3A4, 106 were Caucasian, 2 were Hispanic, and 2 were African-American. Of the 92 individuals sequenced for SRD5A2, 84 were Caucasian and 8 were African American. In addition, the 24 individuals from the Coriell Cell Repository Polymorphism Discovery Resource (Collins et al. (1998) Genome Res 8, 1229-1231) were sequenced against the three genes.

PCR primers covering coding regions, splice sites, 5′ and 3′ regions, and parts of introns of CYP3A4 (reference sequence No. 39), CYP17 (reference sequence No. 40), and SRD5A2 (reference sequence No. 41), were designed using the Primer3 program (http://www.genome.wi.mit.edu/cgi-bin/primer/primer3.cgi). PCR products were sequenced using energy transfer dye terminators on the Amersham Bioscience's MegaBACE1000 (Amersham Biosciences, Sunnyvale, Calif.) using standard protocols. Sequence analysis was performed by assigning quality values (Phred; University of Washington, Seattle, Wash.), assembling contigs (Phrap; University of Washington), automated identification of candidate heterozygote SNPs (PolyPhred, University of Washington), automated identification of candidate homozygote SNPs (High is Quality Mismatch, Amersham Biosciences, Sunnyvale, Calif.) and by operator confirmation (Consed, University of Washington). All polymorphisms were confirmed by Single Nucleotide Primer Extension (SNuPE) assay (Amersham Biosciences, Sunnyvale, Calif.)

In addition to novel polymorphisms discovered in this study, several publicly available SNPs from the dbSNP (http://www.ncbi.nlm.nih.gov/SNP/), Utah Genome Center (UGC) (http://www.genome.utah.edu/genesnps/genes/), the Human Cytochrome P450 Allele Nomenclature Committee (HCANC) (http://www.imm.ki.se/CYPalleles/), the Human Gene Mutation Database (HGMD) (http://archive.uwcm.ac.uk/uwcm/mg/hgmd0.html) and the Human Genic Bi-Allelic SEquences (HGBASE) Release 8 (http://hgbase.interactiva.de/) were searched for CYP17, CYP3A4, and SRD5A2. For the Androgen Receptor gene, several publicly available SNPs from dbSNP, HGBASE and the Androgen Receptor Mutation Database (ARMD) (http://ww2.mcgill.ca/androgendb/) were included.

Genotyping

In Phase I, 276 individuals from prostate cancer sibships were genotyped for 29 SNPs (11 novel, 18 known) in CYP17, 33 SNPs (18 novel, 15 known) in CYP3A4, and 25 SNPs (5 novel, 20 known) in SRD5A2. The individuals included 153 cases and 123 brother controls, 70% European Americans and 30% African Americans. The information from the 276 men was then used to determine initial case-control frequency differences and haplotype tagging. The results were then used to determine which SNPs should be genotyped in the remainder of the study population (i.e. in Phase II of the study).

In Phase II, a total of 24 SNPs were genotyped in 841 individuals, giving information on a total of 1117 individuals for Phase II.

Genotyping was performed utilizing the Single Nucleotide Primer Extension (SNuPE) assay on the MegaBACE1000 (Amersham Biosciences, Sunnyvale Calif.) capillary electrophoresis platform (Amersham Biosciences). The Primer3 program (http://www.genome.wi.mit.edu/cgi-bin/primer/primer3.cgi) was used to design PCR primers to amplify regions containing the SNPs of interest. PCR fragments were purified with 0.5 U of Shrimp Alkaline Phosphatase (Amersham Biosciences) and 10 U of Exonuclease I (Amersham Biosciences) by incubating at 37° C. for 40 min and at 85° C. for 15 min. The single base extension (SBE) reaction was set with 1 pmol of HPLC purified SBE primer, 2-4 μl of SNuPe Premix (Amersham Biosciences), 2-4 μl of sterile water, and 1 μof purified PCR fragment, and incubated at 25 cycles of 96° C. for 10 sec, 50° C. for 5 sec, and 60° C. for 10 sec. For phase I of the study, SNuPe reactions were set in 96-well plates at 10 μl volume and purified with AutoSeq™96 Plates (Amersham Biosciences) prior to injecting into the MegaBACE1000 system. For phase II of the study, SNuPe reactions were set in 384-well plates at 5-6 μl volume, diluted with 3-4 μl of sterile water and purified with 1 U of Shrimp Alkaline Phosphatase (Amersham Biosciences) by incubating at 37° C. for 45 min and at 85° C. for 15 min prior to injecting into the MegaBACE4000 system. In cases where low signal was anticipated (due to faint PCR), SNuPe reactions were desalted using a custom 384-well filter plate incorporating modified size-exclusion technology (Millipore Corporation, Billerica, Mass.). The Scierra Genotyping LWS™ (Amersham Biosciences) system was utilized for the tracking and management of samples and laboratory activity for Phase II of the study.

Specific software (SNPriDe) was developed for the automated design of SNuPE primers. Using a purified PCR fragment containing the SNP of interest as a template, a third, internal primer was designed so that the 3' end anneals adjacent to the polymorphic base-pair, and during the SNUPE reaction a fluorescently labeled dideoxynucleotide (terminator) was added onto the primer. A separate software package has been developed (SNP Profiler™, Amersham Biociences) that automatically processes the signal data and outputs the maximum likelihood SNP genotypes. The system includes a user interface for editing and verification.

Three SNPs, SRD5A2_SNP20 (V89L), SRD5A2_SNP22 (A49T) and CYP17-_SNP29(−34>C) were analysed by restriction enzyme digestion (Cicek et al., unpublished data).

Proofreading Genotype Data

A large number of haplotypes inferred during initial rounds of haplotyping implied erroneous genotype data. A phylogenetic study of inferred haplotypes was performed to reveal the relationships between different haplotypes. All haplotypes differing from another haplotype by only one SNP, and being represented by only one individual, were subject to inspection. Genotype data for the individual at stake were reanalysed by SNP Profiler™ (Amersham Biosciences) to exclude the possibility of an incorrect genotype. Rounds of phylogenetic study of haplotypes, followed by reanalysing suspicious genotypes and inferring new haplotypes were applied until no more incorrect genotypes could be found. Three to six rounds were applied for each of the genes.

Haplotyping

Alleles within each of the three candidate genes were in strong linkage disequilibrium with one another. Thus, for each gene, haplotypes were estimated using the resulting genotypes, by disease status and within major ethnic groups using the software PHASE. This program uses Markov chain Monte Carlo to estimate haplotypes, imputes information for missing genotypes, and incorporates a statistical model for the distribution of unresolved haplotypes based on coalescent theory (Stephens et al. (2001) Am J Hum Genet 68, 978-989).

Haplotypes and haplotype tagging SNPs were first determined among the 276 men genotyped for Phase I of the study, where tagging SNPs was necessary to define the most common haplotypes (e.g., >5%). After completing genotyping on the entire study population (Phase II of the study), the resulting data were used to estimate haplotypes.

Association Analysis

Case versus control allele frequencies were first compared within major ethnic groups. Then the association between the resulting genotypes/haplotypes and prostate cancer risk was evaluated by calculating odds ratios (OR, estimates of relative risk) and 95% confidence intervals from conditional logistic regression with family as the matching variable, using a robust variance estimator that incorporates familial correlations. This is a standard approach for analyzing sibling matched case-control data, although sibling sets without any controls do not contribute any information (197 cases total here) (Breslow and Day (1980) IARC Sci Publ 32, 335-338). In the analyses of CYP17, CYP3A4, and SRD5A2 a log-additive coding was used which treats the most common polymorphism (or haplotype) as the null-risk referent group and assumes that the relative risk of carrying one polymorphism (or haplotype) is the square-root of the risk of carrying two. Since haplotypes were estimated for these three genes, the probabilities of observed haplotypes were used in the analyses (Schaid et al. (2002) Am J Hum Genet 70, 425A434).

To control for potential confounding, age was adjusted for in all regression models. In addition to looking at the main effects of each SNP or haplotype, the analyses were also stratified by the case's disease aggressiveness, where high aggressiveness was defined by TNM stage≧T2B or Gleason score≧7; and low aggressiveness by TNM stage<T2B and Gleason score<7. All statistical analyses were undertaken with the S+software (version 6.0, Insightful Corp, 2001).

Polymorphism Discovery (Phase I)

A total of 34 novel SNPs were detected: 11 in CYP17, 18 in CYP3A4, and 5 in SRD5A2 (Table 2). In addition, 11 SNPs were “rediscovered” from the public databases. Including these 11 SNPs, 53 SNPs were selected in total from the databases: 18 in CYP17, 15 in CYP3A4, and 20 in SRD5A2. These were chosen based on the intention to obtain an even distribution of SNPs across the genes and the availability in the databases at that time (January-April 2001). Twenty-one SNPs were chosen from dbSNP, 27 from GeneSNPs, 12 from HGMD, 8 from HGVbase, and 2 from HCANC (the total number of SNPs listed here exceeds 53 as several SNPs were present in multiple databases). Table 3 lists all 87 SNPs (34 novel, 53 from databases), with their origins, exact locations and allele frequencies.

Among the 34 novel SNPs, 26 (76%) were discovered in both the Coriell and case-control populations. Three SNPs were only observed in the Coriell data, and the remaining five were found only in the prostate cancer sibships. Of these five, three were relatively rare (allele frequencies 0.2-1.5%), suggesting that they may not have been discovered in the Coriell population simply due to its small sample size (n=24). Nevertheless, the other two SNPs that were only found in the prostate cancer sibships (CYP3A4_SNP12 and CYP17_SNP42) showed higher allele frequencies (7.5% and 21.8%, respectively), suggesting that they might be specific to the prostate cancer case-control population.

Genotypying and Haplotyping

Phase I

The 87 SNPs were geneotyped in a total of 276 males from prostate cancer sibships (29 in CYP17, 33 in CYP3A4, and 25 in SRD5A2). Eleven SNPs gave ambiguous genotyping results. This might have been due to unoptimized genotyping reactions or primer self-priming due to secondary structures and unspecificity of PCR and/or SNuPe primers, especially within the Cytochrome P450 gene family. Of the remaining 76 SNPs, a similar percentage of those novel (41%, or 12/29) and known (38%, or 18/47) had allele frequencies>10%. However, 19/47 (40%) of the known SNPs were found to be monoallelic in the 276 men, suggesting that they are either extremely rare, population specific, or artifacts.

In light of these results, the 11 SNPs with ambiguous genotype results, the 19 SNPs that appeared monoallelic in all samples tested, and an additional four that were seen only in the Coriell Diversity Set but not in the prostate cancer sibships were excluded. Also excluded was one SNP because >15% of data was missing (due to a low success rate for PCR and SNuPe reaction). Finally, 12 SNPs were excluded because their minor allele frequencies were less than 5% in all of the following four subgroups: European Americans, African Americans, cases, and controls (Table 3). Following these exclusions, a total of 40 SNPs remained for consideration in the Phase II association study (14 in CYP17, 16 in CYP3A4, and 10 in SRD5A2) (Table 3).

Using the preliminary genotype information, haplotypes estimated with a frequency ≧5% in at least one of the four major subgroups (i.e., European American, African American, cases, or controls) were identified. Each gene had a single “common” haplotype, with a frequency ranging between 42 and 51 percent (not shown). Haplotype tagging SNPs were identified and used as a basis for inclusion in Phase II of the study. In addition, non-tagging SNPs exhibiting suggestive case versus control allele frequencies were considered (Table 3). Altogether 24 SNPs were selected for Phase II.

Phase II

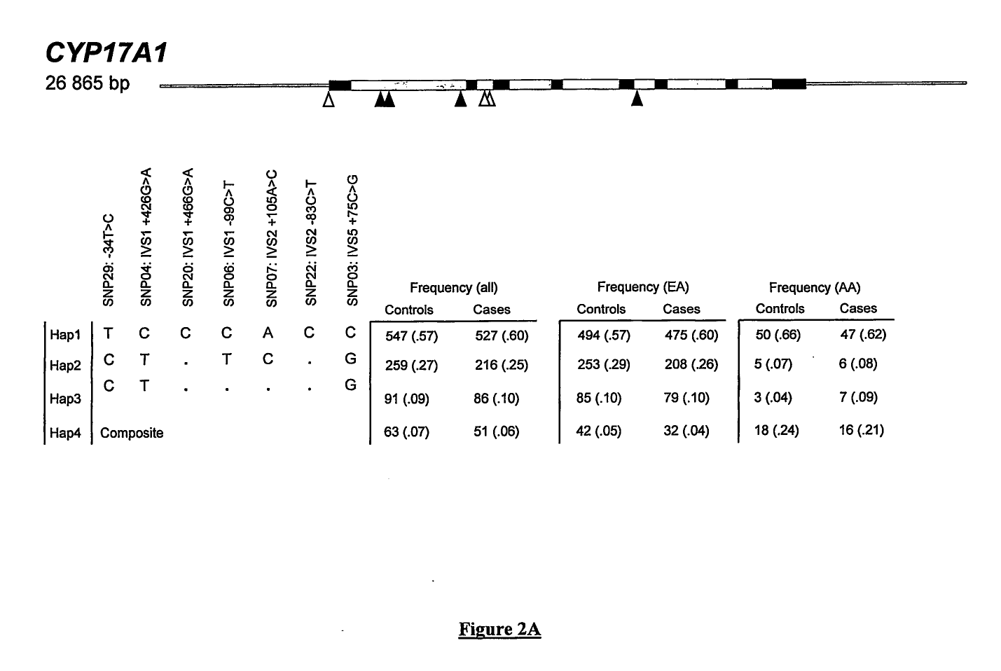

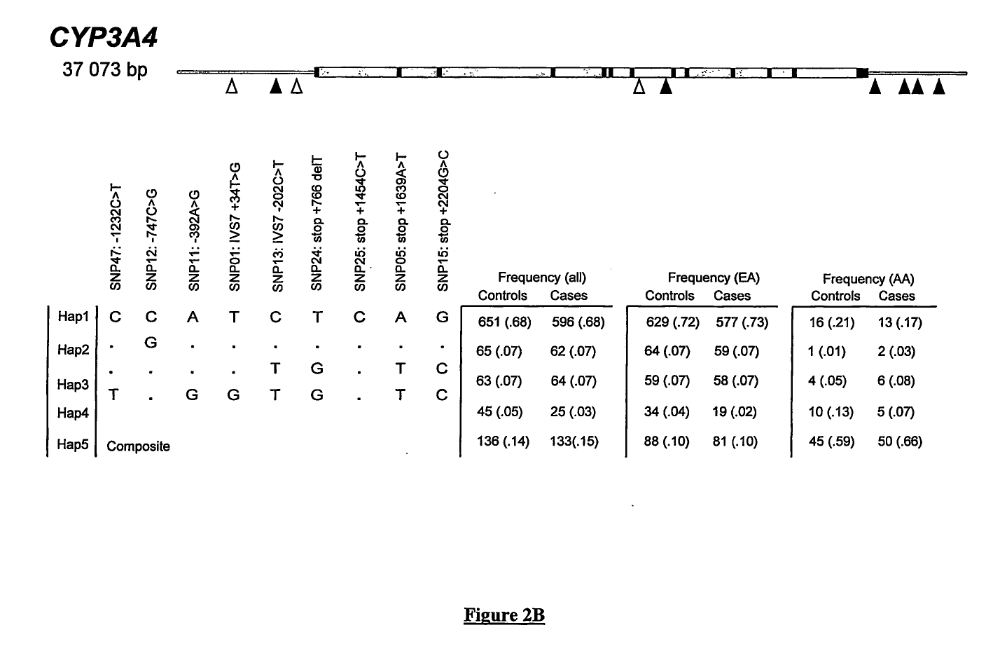

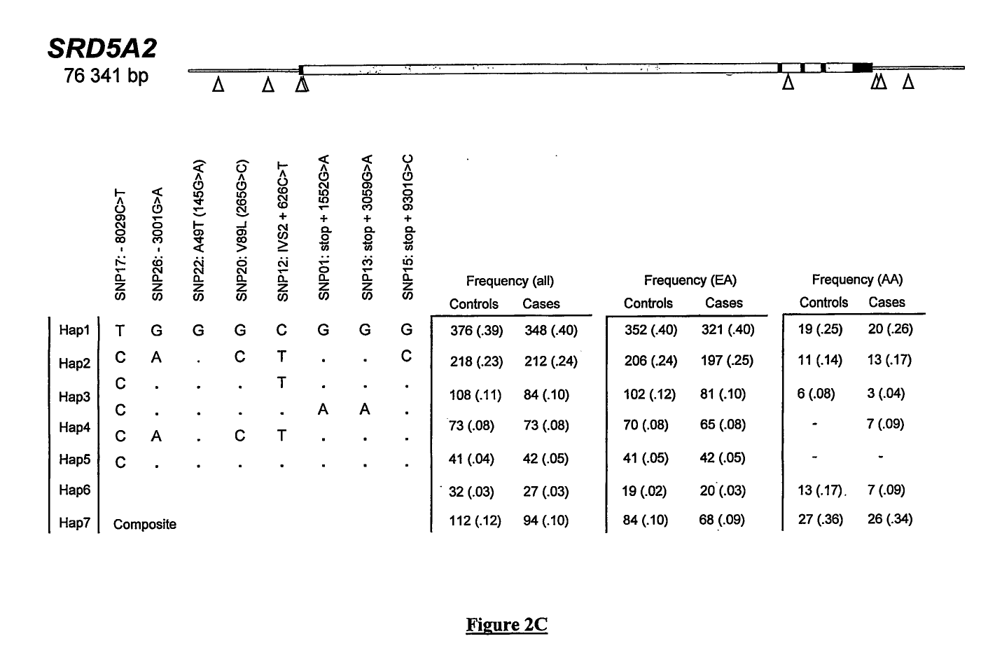

The 24 tagging and suggestive SNPs were genotyped in an additional 841 men, giving information on a total of 1117 individuals for Phase II. Case versus control allele frequency differences by ethnic group are presented in Table 3. Haplotypes estimated with a frequency ≧3% in at least one of the four major subgroups of the study population were identified. The major haplotypes for CYP17, CYP3A4, and SRD5A2 along with their frequencies are presented in FIG. 2.

Association Analyses

In the association analyses, no associations between CYP17 genotypes/haplotypes and prostate cancer were detected. When looking at CYP3A4, SNP1 was found to be associated with an approximately 50% reduction in risk (OR=0.53, 95% CI=0.29-0.99; p-value=0.05) (Table 4A). Furthermore, the haplotype analysis revealed an association with an approximately 55% decrease in prostate cancer risk and CYP3A4_Hap4 (OR=0.46, 95% CI=0.21-1.02; p-value=0.05) (Table 5A). Two SNPs in SRD5A2 were also found to be associated with an approximately 50% increase in prostate cancer risk: SRD5A2_SNP26 (OR=1.57, 95% CI=1.08-2.30; p-value=0.02), and SRD5A2_SNP20 (V89L) (OR=1.56, 95% CI=1.08-2.25; p-value=0.02) (Table 4A). These SNPs, however, 5 were in almost complete linkage disequilibrium.

When the study population was stratified by high and low aggressiveness of prostate cancer, several interesting associations emerged (see Table 4B and 5B). First, five SNPs in CYP3A4 showed statistically significant associations with low aggressiveness: CYP3A4_SNP11 (CYP3A4*1B) (OR=0.20, 95% CI=0.06-0.67; p-value=0.009), CYP3A4_SNP47 (OR=0.19, 95% CI=0.06-0.62; p-value=0.006), CYP3A4_SNP1 (OR=0.21, 95% CI=0.05-0.86; p-value=0.03), CYP3A4_SNP25 (OR=6.54, 95% CI=0.99-43.10; p-value=0.05) and CYP3A4_SNP15 (OR=0.41, 95% CI=0.22-0.79; p-value=0.007). Second, an association was observed between CYP3A4_Hap4 and low aggressiveness (OR=0.06, 95% CI=0.008-0.50; p-value=0.009) (Table 5B). Finally, an inverse association was observed between SRD5A2_Hap3 and high aggressiveness (OR=0.52, 95% CI=0.29-0.91; p-value=0.02) (Table 5B).

Table 6 provides annotation of CYP3A4, CYP17 and SRD5A2 genomic sequences.

All of the SNPs disclosed in the present invention have utility in the prognosis and diagnosis of prostate and breast cancer.

Although this invention has been described in terms of certain preferred embodiments, other embodiments which will be apparent to those of ordinary skill in the art in view of the disclosure herein are also within the scope of this invention. Accordingly, the scope of the invention is intended to be defined only by reference to the appended claims. All documents cited herein are incorporated herein by reference in their entirety.

TABLE 1
Known polymorphisms in CYP17, CYP3A4, and SRD5A2 associated with increased
risk for prostate cancer or increased risk for progression of prostate cancer.

| Gene | Polymorphism | Association with | References |
|---|---|---|---|
| CYP17 | −34 bp T→C | increased risk | Lunn et al. (1999) Carcinogenetics 20, 1727-1731 |
| | | | Wadelius et al. (1999) Pharmacogenetics 9, 635-639 |
| | | | Gsur et al. (2000) International Journal of Cancer 87, 434-437 |
| | | | Habuchi et al. (2000) Cancer Res 60, 5710-5713 |
| | | | Kittles et al. (2001) Cancer Epidemiol Biomarkers Prev 10, |
| | | | 943-947 |
| CYP3A4 | −392 bp A→G | increased risk, progression | Rebbeck et al. (1998) J Nat Cancer Inst 90, 1225-1229 |
| | | | Paris et al. (1999) Cancer Epidemiol Biomarkers Prev 8, 901-905 |
| SRD5A2 | 145G > A (A49T) | increased risk, progression | Makridakis et al. (1999) Lancet 354, 975-978 |
| | | | Jaffe et al. (2000) Cancer Res 60, 1626-1630 |
| SRD5A2 | 265G > C (V89L) | increased risk, progression | Nam et al. (2001) Urology 57, 199-204 |

TABLE 2(a)
Novel SNPs

| SNP | | | | |
|---|---|---|---|---|
| identi- | | | | |
| fied in | | | | |
| Seq ID | | | | |
| Number | Novel SNPs | Location | Comment | Sequence |
| 1 | CYP3A4_SNP2 | Intron 9 | | ATGAGAATTTCTGCCACATAGCAGAACGACACATGTT |
| | | [IVS9+187C>G] | | TGAATGTTATAAGTGGTAGTTGGAGGCACTTTCTAGA |
| | | | | GGCATGCAGGCATAGATAGCCATGTT(C/G)TAAGAG |
| | | | | TAAAGGGCAACCCTAAGCAAACCTGGCATGCTAGAAA |
| | | | | GTCAGTCTGCGGTCTGTGGATCACCTACATCAGATCA |
| | | | | AATGCCAATTCTCAGCCTCC |
| 2 | CYP3A4_SNP5 | 1639 base pairs | Possible association | TGATAGAAGCCAGGCTTCTCACCTTTGCAGAAGGGAG |
| | | after the stop | (OR 0.51) 95% Cl | TCATGGATTCAGAAAGGGAGAAAACTAGCATGAATCC |
| | | codon, A>T | 0.26-0.99, p=0.05) | TATGAAATTAGATTGGAATGGATGTA(A/T)CCGTGT |
| | | | with prostate cancer | ATATTCATACCCTTGTAGATAGATAGATGGGTAGATA |
| | | | in the European | GATGATAGATAGGTAACAGATAGATGACAGATAATGA |
| | | | American population | GATAGATAGATGTAAATGTA |
| 3 | CYP3A4_SNP6 | 945 base pairs after | | GGCAGGAGAATCACTTGAACCTGGGAGGCGGATGTTG |
| | | the stop codon, A>T | | AAGTGAGCTGAGATTGCACCACTGCACTCCAGTCTGG |
| | | | | GTGAGAGTGAGACTCAGTCTTAAAAA(A/T)ATATGC |
| | | | | CTTTTTGAAGCACGTACATTTTGTAACAAAGAACTGA |
| | | | | AGCTCTTATTATATTATTAGTTTTGATTTAATGTTTT |
| | | | | CAGCCCATCTCCTTTCATAT |
| 4 | CYP3A4_SNP12 | 5′ region | Possible association | AAGTCACCAGAAAGTCAGAAGGGATGACATGCAGAGG |
| | | [−747C>G]OR 12.067, 95% Cl | CCCAGCAATCTCAGCTAAGTCAACTCCACCAGCCTTT | |
| | | | 1.491-97.692, | CTAGTTGCCCACTGTGTGTACAGCAC(C/G)CTGGTA |
| | | | p=0.020) with | GGGACCAGAGCCATGACAGGGAATAAGACTAGACTAT |
| | | | increased risk of | GCCCTTGAGGAGCTCACCTCTGTTCAGGGAAACAGGC |
| | | | prostate cancer | GTGGAAACACAATGGTGGTA |
| 5 | CYP3A4_SNP13 | Intron 7 | | CTGTAGTCCAATAGATAAAGGCAAAGAGATTAGGGCA |
| | | [IVS7 −202C>T] | | TTGAATTTTGTTCCTTTTATCCTTCAAAAGATGCACA |
| | | | | AGGGGCTGCTGATCTCACTGCTGTAG(C/T)GGTGCT |
| | | | | CCTTATGCATAGACCTGCCCTTGCTCAGCCACTGGCC |
| | | | | TGAAAGAGGGGCAAAAGTCATAGAAGGAATGGCTTCC |
| | | | | AGTTGAGAACCTTGATGTCT |
| 6 | CYP3A4_SNP15 | 2204 base pairs | Possible association | GAACTATTGGAACTGATAAACACATTCAGTAAAGTTG |
| | | after the stop | (OR 0.41, 95% Cl | CAGGATACAAAATCAGCATACAAAAATCAGTAGCATT |
| | | codon, G>C | 0.22-0.79, p=0.007) | TCTATATGCCAATAGTGAACAATCTG(G/C)CAAAAA |
| | | | with prostate cancer | TAAAAAAGTAATCCCATTTACAATAGCCACAAATAAA |
| | | | | ACTAAATACCTAGAAATTAACTTAATCAAAGAAGAGA |
| | | | | AAGGTCTCTACAATGAATAC |
| 7 | CYP3A4_SNP19 | Intron 2 | | ATAAGTCATTCAGTATCCACAACACTTGGAGAGAATT |
| | | [IVS2 −132C>T] | | CAAGAGTGATTTTAAATTTCCCTTTTCAAATACCTCC |
| | | | | TCTGTTTTCTCTTATTTCCTTTATGA(C/T)GTCTCC |
| | | | | AAATAAGCTTCCTCTAACTGCCAGCAAGTCTGATTTC |
| | | | | ATTGGCTTCGACTGTTTTCATCCCAATTAGAGGCAGG |
| | | | | GTTAAGTACATTAAAAATAA |
| 8 | CYP3A4_SNP21 | Intron 1 | | AACTGCCCCTAGGATCCAATCATCTCCTACCAGGCCC |
| | | [IVS1 −868C>T] | | CACCTCCAGTATTGGGGATTGCATTTCAACATGAGAT |
| | | | | TTTGGTAGGGGCACAGATTCAGACCATAT(C/T)ACT |
| | | | | GGCACTGTGCTAATCAGATGAATATCACCAGTTGGAA |
| | | | | GGCTAGATTCCACAAGAGGAGGAATGACCTGGAAATT |
| | | | | GGTTCTTTAGTTGTGATTCT |
| 9 | CYP3A4_SNP22 | 5′ region | | GGGGTCCCCTTGCCAACAGAATCACAGAGGACCAGCC |
| | | [−847A>T] | | TGAAAGTGCAGAGACAGCAGCTGAGGCACAGCCAAGA |
| | | | | GCTCTGGCTGTATTAATGACCTAAG(A/T)AGTCACC |
| | | | | AGAAAGTCAGAAGGGATGACATGCAGAGGCCCAGCAA |
| | | | | TCTCAGCTAAGTCAACTCCACCAGCCTTTCTAGTTGC |
| | | | | CCACTGTGTGTACAGCACCC |
| 10 | CYP3A4_SNP24 | 766 base pairs | | TCAGGCACAGTGGCTCACGCCTGTAATCCTAGCAGTT |
| | | after the stop | | TGGGAGGCTGAGCCGGGTGGATCGCCTGAGGTCAGGA |
| | | codon, delT | | GTTCAAGACAAGCCTGGCCTACATGG(T/-)TGAAAC |
| | | | | CCCATCTCTACTAAAAATACACAAATTAGCTAGGCAT |
| | | | | GGTGGACTCGCCTGTAATCTCACTACACAGGAGGCTG |
| | | | | AGGCAGGAGAATCACTTGAA |
| 11 | CYP3A4_SNP25 | 1454 base pairs | Possible association | TGGGTGTGGGAGTCCAAGCAAGCAGAGAAGGGGTCGA |
| | | after the stop | (OR 6.54, 95% Cl | CGCAGAGGGGTGGCTTGCAAGAGCAGCCAGAGCCTAA |
| | | codon, C>T | 0.99-43.10, p=0.05) | ATAGGGTATGGAGAACCCACATGAGG(C/T)GAGGAG |
| | | | with prostate cancer | GGCATCCATGAGTGGGAGGGGTTGGGTGAGGTTTGGC |
| | | | | TACATAAAGGGGATTGATCAAATAAGTAAATGTATTA |
| | | | | AGGATGATAGAAGCCAGGCT |
| 12 | CYP3A4_SNP26 | Intron 3 | | TTGCATTTCTCTAATGACCAGTGATGATGAGCATTTT |
| | | [IVS3 +1992T>C] | | TTCACATGTCTGTTGGCTGCATAGATGTCTTCTTTTG |
| | | | | AGAAGTGTCTGTTCATATCCTTTGCC(T/C)ATTTTT |
| | | | | TGATGGGGTTGTTTGCTTTTTTTCTTGTAAATTTGTT |
| | | | | TAAGTTCTTTGTAGATTCTGGATGTTAGCCCTTCGTC |
| | | | | AGATGGATAGATTGCAAAAA |
| 13 | CYP3A4_SNP27 | Intron 9 | | TAACTATTGGTTCTAGAGAGCAGGACTGGGCTTACTC |
| | | [IVS9 +841T>G] | | CAGCATACTGCTTTAAATATATCCATGTCTACATCCA |
| | | | | CTTTTGTCTGTATGTCTATGTATCTA(T/G)CTATGT |
| | | | | ATCTATCTAGCTATGTATCTATCTATCTATCTATCTA |
| | | | | TCATCTATCTATCTATCTATCATCTATCCATCTATCA |
| | | | | TCTATCATTTATCCATCTAT |
| 14 | CYP3A4_SNP28 | Intron 12 | | CTTCCCATCTTTACACTGGATGGGTTCAATTGGGAGG |
| | | [IVS12 −473T>G] | | AATTACTGGACTCTGGAAGTTGAAGACTGTCCATATA |
| | | | | ATTAAAATGTACAATAACTACCCAGG(T/G)TTACCT |
| | | | | TGCAAGTTTCAACATACACAAAATTAACTTTATATGA |
| | | | | CTCTTCAAAAACAGTTTGCCATCATACCTAATAATCT |
| | | | | GGTTTAAATTTTAAAAACTC |
| 15 | CYP3A4_SNP29 | Intron 12 | | TGCCCAGAGTGTGGCTTTAAAAGCTTCCCCATTGCTT |
| | | [IVS12 +581C>T] | | CTCATGTGAAGCCAAGGTTGAGAATGACTAATTTAAG |
| | | | | GCATTTCTGGTGGATATAAAGGACTA(C/T)CACAGT |
| | | | | CCAAGGCCATCCTGACTGACCTCACCTTCCAGGTGCC |
| | | | | TAGCTCCATCCAGCTGGGCTCCTTTTCAACCCAATTA |
| | | | | TAACTCTATTAATGTTGTTC |
| 16 | CYP3A4_SNP30 | Intron 12 | | AGAGTGTGGCTTTAAAAGCTTCCCCATTGCTTCTCAT |
| | | [IVS12 +586G>A] | | GTGAAGCCAAGGTTGAGAATGACTAATTTAAGGCATT |
| | | | | TCTGGTGGATATAAAGGACTACCACA(G/A)TCCAAG |
| | | | | GCCATCCTGACTGACCTCACCTTCCAGGTGCCTAGCT |
| | | | | CCATCCAGCTGGGCTCCTTTTCAACCCAATTATAACT |
| | | | | CTATTAATGTTGTTCCCAGC |
| 17 | CYP3A4_SNP31 | Intron 12 | | TAATTTAAGGCATTTCTGGTGGATATAAAGGACTACC |
| | | [IVS12 +646C>2A] | | ACAGTCCAAGGCCATCCTGACTGACCTCACCTTCCAG |
| | | | | GTGCCTAGCTCCATCCAGCTGGGCTC(C/A)TTTTCA |
| | | | | ACCCAATTATAACTCTATTAATGTTGTTCCCAGCCAG |
| | | | | GCATGGTGGCTCATGCCTGTAATCCCAGCACTTTGGG |
| | | | | AGGCCGAAGCAGGCGGATCA |
| 18 | CYP3A4_SNP32 | Intron 3 | | CTAATTTGATTGCACTGTGGTCTGAGAGACAGTTTGT |
| | | [IVS3 −734G>A] | | TATGATTTCTGTTCTTTTACATTTGCTGAGGAGTGCT |
| | | | | TTACTTCCAATTATGTGGTCAATTTT(G/A)GAATAA |
| | | | | GTGCGATGTGGTGCTGAGAAGAATGTATATTCTGTTG |
| | | | | ATTTGGGGTGGAGAGTTCTGTAGATGTCTATTAGGTC |
| | | | | CGCTTGGTGCAGAGCTGAGT |
| 19 | CYP17_SNP1 | Intron 1 | | GGACAGGCATAGTTTAGAGAGTTTATCCCATCCAGAG |
| | | [IVS1 −271A>C] | | TTGCCTTCTGTGGTCAGAAACTGATGAGCAAAAAGAA |
| | | | | GCCCAGAGGGCACCCTGTCAGCGAA(A/C)AGAACCC |
| | | | | CAATGCTGCTGCATTCTAATTAAGGGTTCTTTCTTTC |
| | | | | TCCTTGATCTACTGTATTTCTGAAGGAATTGGGAGTA |
| | | | | GGAGGCCTTAGGGTCTGTC |
| 20 | CYP17_SNP3 | Intron 5 | | TGGCCTTCCTGCTGCACAATCCTCAGGTGTGCTTCCC |
| | | [IVS5 +75C>G] | | CCTCATTGATCCTAGACCCCAGCCAGCCCAATCTCTG |
| | | | | GGCTCCAGAGAAAGGGAGAGCCAATT(C/G)TCTCAG |
| | | | | GCTTTCTGTGCAGGAAGACTAGGCCTGCCCTGCTCCT |
| | | | | TACCCAAGCAGTAGTTGGCTTTGACCCCAGAGTAGAG |
| | | | | CTGCCCCATCTTCTGGAAGC |
| 21 | CYP17_SNP4 | Intron 1 | | CAGCACTTAGCCTAGCACCCAGCACAGTAAGTGCCCC |
| | | [IVS1 +426G>A] | | TTATACAGCCAGGATTCATGTTACTTTTCATGGAAAA |
| | | | | TGGGGGCAGTGACTACTGTCCTCCAT(G/A)AAAGCT |
| | | | | GCTGGGGAGAATTAGCCTAGCTATTGCAGGCTGGGAT |
| | | | | TGCTGCTTTCCTGGTGCTATTTCCAGCTACTCAGGCT |
| | | | | CACAGGGGCAGTTTTCTACA |
| 22 | CYP17_SNP6 | Intron 1 | Possible association | ATTGGGAGTAGGAGGCCTTAGGGTCTGTCCTACCAAG |
| | | [IVSI −99C>T](OR 2.130, 95% Cl | TCCTTGCAGTCATGGTGGAGTGCAGTGGGGCTGTGCC | |
| | | | 1.141-3.977, | CACATGGGAGTCAGCATGCCAGGTAC(C/T)TGCCTT |
| | | | p=0.018) with | CTCCTCCAGGAAGGAAAGCAGGGACCAGAGGTGTAAG |
| | | | increased risk of | GGCAAGAGTGGGGTGGATGGTGTGAGATTCCTACAGC |
| | | | prostate cancer | CTTGCCTGCTCTCTAAAGGC |
| 23 | CYP17_SNP8 | Intron 1 | | GCCACTGTGCCCTGCCAGCCTCTCAGCTTTGATCAAG |
| | | [IVS1 −700C>G] | | CCAAGGGTTGGTTTATTTTTTCTTGGACCAATCAGCC |
| | | | | AGGTCTGCTGACCAACTACCTAGCTC(C/G)CACCTC |
| | | | | TGCTGGCTTCCTCCCGGGGGCAGAGAAGATGGAGAAG |
| | | | | GCTAGTCATGTGGATCTTCAGGGTCAGGAAATGGAAA |
| | | | | AGGGAGGCTTTGGACCCTTT |
| 24 | CYP17_SNP11 | Intron 1 | | ATGGAGAAGGCTAGTCATGTGGATCTTCAGGGTCAGG |
| | | [IVS1 −565G>A] | | AAATGGAAAAGGGAGGCTTTGGACCCTTTTGCTTTGG |
| | | | | GGGGCACCTCTAGGAGGAGGCAGCTC(G/A)GCCCAA |
| | | | | GTCCAGACTGGGTAGACAAAACATCTGCACTCTCCAA |
| | | | | ATGTGGGCTTGTGGCTGGGTATGCAGGCTTGCAATGG |
| | | | | AAGGGTAAACCTGAGTGAGG |
| 25 | CYP17_SNP12 | Intron 3 | | CTGACATTGTCCCCAATCTTCCTTCCTTTTTACTTCC |
| | | [IVS3 +141A>T] | | CTGCTCCAGCCGCAATGACCCATCTTTTTCCTGATTA |
| | | | | CCTCCGCCACCTCTACCTCCTCTGCC(A/T)CTTAAA |
| | | | | ACCTTTGCCATTTCTCTGCAGAGATAGATTTAGCCTT |
| | | | | TTAATTATGCACCTTAGTACTCCAGATAATGACCTTC |
| | | | | ATTTCTTTTTCCAATTACCAT |
| 26 | CYP17_SNP18 | 5′ region | | ATTTTTAGGGAACAAGGGAAAACAACCATAAGGTCTG |
| | | [−1488C>G] | | ACTGCCTGCAGGGTCGGGCAGAAAGAGCCATATTTTC |
| | | | | CTTCTTGAGAGAGGCTATAAATGGA(C/G)ATGCAAG |
| | | | | TAGGGAAGATATCACTAAATTCTTTTCCTAGCAAGGA |
| | | | | GTATTATTATTAATACCCTGGGAAAGGAATGCATTCC |
| | | | | TGGGGGGAGGTCTATAAACA |
| 27 | CYP17_SNP19 | 5′ region | | TAGGGTGGGGAAAAACTCCGCCCTGGTAAATTTGTGG |
| | | [−1204C>T] | | TCAGACCGGTTCTCTGCTGTCGAACCCTGTTTGCTGT |
| | | | | TGTTTAAGGTGTTTTATCAAGACAGTA(C/T)GTGCA |
| | | | | CCGCTGAACATAGACCCTCATCTGTAGTTCTGCTTTT |
| | | | | GCCCTTTGCCTTGTGATCTTTGTTGGACCCTTATCAG |
| | | | | TGGTTCTGCTTTTGCCCTTTG |
| 28 | CYP17_SNP20 | Intron 1 | | TACAGCCAGGATTCATGTTACTTTTCATGGAAAATGG |
| | | [IVS1 +466G>A] | | GGGCAGTGACTACTGTCCTCCATAAAAGCTGCTGGGG |
| | | | | AGAATTAGCCTAGCTATTGCAGGCTG(G/A)GATTGC |
| | | | | TGCTTTCCTGGTGCTATTTCCAGCTACTCAGGCTCAC |
| | | | | AGGGGCAGTTTTCTACAATGACATTTCAGGGTTGCTG |
| | | | | ATGAGCCTCCCACTCAGCAG |
| 29 | CYP17_SNP42 | 712 base pairs | | CTGGAGGATTTTAAGTATGTAAGTGGAACAATCTGTT |
| | | after the stop | | TTTTTGTTTTTGTTTTTGTTTGAGAAGGAGTTTCGCT |
| | | codon, G>A | | CTTGTTGCCCTGGCTGGAGTGCAATG(G/A)CATGAT |
| | | | | CTTGGCTCACTGCAACCCCTGCCTCCTGAGTTCAAGT |
| | | | | GATTCTCCTGCCTCAGCCTCCAAAATAGCTGGGATTG |
| | | | | CAGGCGTGTGCCACCATGCC |
| 30 | SRD5A2_SNP2 | 1356 base pairs | | TCTTGTGAAGGGGTCACCCCAGCATGAGTGCTGAGAT |
| | | after the stop | | ATGGACTCTCTA(A/C)GGAAGGGGCCGAACGCTTGT |
| | | codon (3′ UTR), | | AATTGGAATACATGGAAATATTTGTCTTCTCAGGCCT |
| | | A>C | | ATGTTTGCGGAATGCATTGTCAATATTTAGCAAACTG |
| | | | | TTTTGA |
| 31 | SRD5A2_SNP4 | 849 base pairs | | CGAGAACAGTTTTACAATAGACATTGCAAACTCTCAT |
| | | after the stop | | GTTTTTGGAAACT(A/G)GTGGCAATATCCAAATAAT |
| | | codon (3′ UTR), | | GAGTAGTGTAAAACAAAGAGAATTAATGATGAGGTTA |
| | | A>G | | CATGCTGCTTGCCTCCACCAGATGTCCACAACAATAT |
| | | | | GAAGTAC |
| 32 | SRD5A2_SNP30 | 5′ region | | GTCTGCGTGTATGACGGCTAGACAGGAGTTCAGAGAA |
| | | [−870G>A] | | CAGCGGGGTCGCCAGGCCACCACCTGATGGGCCACGG |
| | | | | CTCATTGGCTCTAGGAGCTGGGAAAG(G/A)GCATCC |
| | | | | CAGGAAAGAAGCCCTAGACTTTAGCCTGAGTCTGGGC |
| | | | | CACTCTAGGGGACCGGGAGTGGGGTGGCGGGAGAGGA |
| | | | | CGCGCAGAATCTCGACTTCT |
| 33 | SRD5A2_SNP31 | 5′ region | | AGCTAATTGTTATAATAGTGGAGAAAAGATCATGAGG |
| | | [−2036(A)7-8] | | ACAAAAAGTGGGCAGAGTCGGAAGAAAAGAGAGGAAG |
| | | | | AAATTGAGACAGAAGACATTTCATTT[A₇/A₈]TATT |
| | | | | CCATTGAGCTGGGTTTGAAATAGTGCACTGCCTGTTC |
| | | | | TCCTAATGCTGTATGGTGTCATGAAATCTATTGTTTA |
| | | | | CTGAGTCTATGAGCC |
| 34 | SRD5A2_SNP32 | 545 base pairs | | AACTCTGAAGCCACAAAGACCCAGAGCAAACCCACTC |
| | | after the stop | | CCAAATGAAAACCCCAGTCATGGCTTCCTTTTTTCTT |
| | | codon (3′ UTR), | | GGTTAATTAGGAAAGATGAGAAATTAT(T/C)AGGTA |
| | | T>C | | GACCTTGAATACAGGAGCCCTCTCCTCATAGTGCTGA |
| | | | | AAAGATACTGATGCATTGACCTCATTTCAAATTTGTG |
| | | | | CAGTGTCTTAGTTGATGAGTG |

TABLE 2(b)
Public SNPs

| SNP | | | | |
|---|---|---|---|---|
| Present | | | | |
| in Seq | | | | |
| ID | | | | |
| Number | Public SNPs | Location | Comment | Sequence |
| 35 | SRD5A2_SNP12 | Intron 2 | Possible association | AAAGAAACATTGTTTCTTAAAACAATGTTTTAAGAAA |
| | (NCBI | [IVS2 +626C>T] | (OR 3.006, 95% Cl | GTGTACGAATTTGTGTCAGGCCACATCCAAAACTGTC |
| | ss#543530; | | 1.231-7.343, | CTGGGCTGCATGTGGCCCACAGGCTG(C/T)GGGTTG |
| | rs#413836) | | p=0.016) with | GACAAGCCTGGCCTAGAAGGCTTTGCCCCCATGTATT |
| | | | increased risk for | CATGGGGGTTGGTTCCTCACTTTATTTAGTTCCCTAC |
| | | | progression of | CAATTTGCACCTCCTCAAAGGGACTTTCCC |
| | | | prostate cancer | |
| 36 | SRD5A2_SNP17 | 5′ region | Possible association | GGGAACTCACAGTTTTTTGGCTGTCTCATAGAGTTTG |
| | (NCBI | [−8029C>T] | (OR 0.308, 95% Cl | CAACAGTAAAACTGCTTCTTTCAAAGGGTCTGTGAAT |
| | ss#1037918; | | 0.126-0.750, | TCTTTCAGTTTTCCTGGTATGTTCCCATGGTAGTTCT |
| | rs#545303) | | p=0.010) with | TGCAGCAAAAG(C/T)TCACAGTGTGAGTCTCCACAC |
| | | | increased risk for | ACTGTTCTGTCCATTCCAAGCAGGAGCTGCATGTTAG |
| | | | progression of | TTCTGTCTGCTATCCACCATTTTCCAATTTTG |
| | | | prostate cancer | |
| 42 | CYP3A4_SNP17 | Intron 7 | Possible association | CCCTTTGTGGAAAACACCAAGAAGCTTTTAAGATTTG |
| | (NCBI | [IVS7 +34T>G] | (OR 0.21,95% Cl | ATTTTTTGGATCCATTCTTTCTCTCAATAAGTATGTG |
| | ss#6903779; | | 0.05-0.86, p=0.03) | GACTACTATTTCCTTTTATTTATCTT(T/G)CTCTCT |
| | rs#2687116) | | with prostate cancer | TAAAAATAACTGCTTTATTGAGATATAAATCACCATG |
| | | | | TAATTCATCCACTTAAAATATACAGTTCAGTGATTTG |
| | | | | TAGTACATTTGAAGATATGT |
| 43 | CYP3A4_SNP47 | 5′ region | Possible association | TTGGGTGTGTGGCGGGTGTGTCCGCGTTTTAAAAAGC |
| | (NCBI | [−1232C>T](OR 0.19, 95% Cl | GCCGCACGCTTTGAACTCCAATTCCACCCCCAAGAGG | |
| | ss#2723639; | | 0.06-0.62, p=0.006) | CTGGGACCATCTTA(C/T)TGGAGTCCTTGATGCTGT |
| | rs#1851426) | | with prostate cancer | GTGACCTGCAGTGACCACTGCCCCATCATTGCTGGCT |
| | | | | GAGGTGGTTGGGGTCCATCTGGCTATCTGGGCAGCTG |
| | | | | TTCTCTTC |
| 44 | SRD5A2_SNP26 | 5′ region | Possible association | ACTATTCTCCTGCCCTAATCAGCCAGGTCCAGGTAAC |
| | (NCBI | [−3001G>A] | (OR 1.57, 95% Cl | AGAAAAGTAAAGACAGCCGCTGTACCCCAGAGCCTGC |
| | ss#1037925; | | 1.08-2.30, p=0.02) | TAAAAGTATTCAAACGAGCTAATCCTAAGCCTGATTA |
| | rs#676033) | | with prostate cancer | CCTTGTCATGCCCACTCTTTCCTGCAGAAACTACAGT |
| | | | | AAAGGCTCTTGCCCACCTTGACCCCTCACTCC(G/A) |
| | | | | GCTGCCTCCTAACACTGGTGCTTCTCCATGTGGTCTT |
| | | | | GGGTGGTGTGCTGTGTCTTCTGTTTGTAGGGATCTGT |
| | | | | CGATATAAACCTTTTCCTTCACGATA |
| 45 | SRD5A2_SNP1 | 1552 bp after | Possible association | GGTACTAAGCACAGAAACTCACTATATAAGTCACATA |
| | (NCBI | the stop codon, | (OR 0.52, 05% CI | GGAAACTTGAAAGGTCTGAGGATGATGTAGATTACTG |
| | ss#4403959; | G>A | 0.27-1.00, p=0.05) | AAAAAT(G/A)CAAATTGCAATCATATAAATAAGTGT |
| | rs#1042578) | | in the European | TTTTGTTGTTCATTAAATACCTTTAAATCATGGATGT |
| | | | American population | AAGCAGTTTGTTGATA |

#SNP was discovered in the Coriell Diversity Set and was not present in the 276 individuals from prostate cancer sibships (still obviously a real SNP since it's seen in the Diversity Set)
@ambiguous genotyping results; SNP was excluded from all further analyses. However, most likely real SNPs
The numbering system for the location of SNPs is according to the common mutation nomenclature (den Dunnen and Antonarakis (2000) Human Mut 15, 7-12; http://www.dmd.nl/mutnomen.html#DNA).

TABLE 3
The origins, nucleotide changes and allele frequencies of single nucleotide polymorphisms
(SNPs) in CYP17, CYP3A4, and SRD5A2 observed in the Coriell Diversity set (CDS),
European Americans, and African-Americans.

| | | | | | Allele Frequency | | | | |
|---|---|---|---|---|---|---|---|---|---|
| | | | | | | European | | African- | |
| | | Nucleotide | | | | Americans | | Americans | |
| SNP | Origin^{a} | Change^{b} | ^{c} | ^{d} | CDS | Ctrls | Cases | Ctrls | Cases |

CYP17

| | | | | | | | | | |
|---|---|---|---|---|---|---|---|---|---|
| SNP18 | Novel(C+C) | −1488C > G | B | I | .26 | NA^{e} | NA | NA | NA |
| SNP19 | Novel(C+C) | −1204C > T | B | I | .10 | NA | NA | NA | NA |
| SNP29 | dbSNP/HGVbase(−) | −34T > C | 1, 2, 3 | II | .44 | .40 | .38 | .33 | .38 |
| SNP30 | GeneSNPs/dbSNP(−) | C22W (66C > G) | B | I | — | NA | NA | NA | NA |
| SNP31 | GeneSNPs/dbSNP/HGVbase(−) | H46H (138C > T) | | I | .43 | .43 | .36 | .41 | .35 |
| SNP32 | GeneSNPs/dbSNP/HGVbase(−) | S65S (195G > T) | | I | .46 | .44 | .33 | .40 | .32 |
| SNP4 | Novel(C+C) | IVS1 +426G > A | 1 | II | .27 | .40 | .39 | .30 | .36 |
| SNP20 | Novel(C+C) | IVS1 +466G > A | 4 | II | .06 | .03 | .01 | .02 | .02 |
| SNP8 | Novel(C+C) | IVS1 −700C > G | | I | .19 | .14 | .15 | .21 | .08 |
| SNP26 | GeneSNPs/dbSNP(+<) | IVS1 −679C > T | D | I | — | .06 | .02 | .04 | .01 |
| SNP11 | Novel(CDS) | IVS1 −565G > A | A | I | .04 | — | — | — | — |
| SNP1 | Novel(C+C) | IVS1 −271A > C | | I | .44 | .46 | .40 | .40 | .43 |
| SNP6 | Novel(C+C) | IVS1 −99C > T | 2 | II | .38 | .29 | .28 | .11 | .15 |
| SNP23 | HGMD(*) | S106P (316T > C) | A | I | — | — | — | — | — |
| SNP25 | HGMD(*) | IVS2 +5G > T | A | I | — | — | — | — | — |
| SNP7 | dbSNP(R) | IVS2 +105A > C | 1 | II | .46 | .29 | .28 | .13 | .15 |
| SNP22 | dbSNP(+<) | IVS2 −83C > T | 1 | II | .04 | .002 | .0008 | .06 | .09 |
| SNP24 | HGMD(+<) | E194X (580G > T) | D | I | — | — | — | — | .01 |
| SNP5 | dbSNP(R) | IVS3 +35T > C | | I | .06 | .16 | .16 | .20 | .07 |
| SNP12 | Novel(C+C) | IVS3 +141A > T | | I | .04 | .04 | .02 | — | .01 |
| SNP21 | GeneSNPs/dbSNP(*) | D283D (849C > T) | A | I | — | — | — | — | — |
| SNP3 | Novel(C+C) | IVS5 +75C > G | 1 | II | .33 | .40 | .39 | .20 | .23 |
| SNP33 | HGMD(−) | IVS7 +5G > A | D | I | — | — | .02 | — | — |
| SNP34 | HGMD(*) | F417C (1250T > G) | A | I | — | — | — | — | — |
| SNP35 | GeneSNPs/dbSNP/HGVbase(−) | P428P (1284G > A) | B | I | — | NA | NA | NA | NA |
| SNP36 | HGMD(*) | R440H (1319G > A) | A | I | — | — | — | — | — |
| SNP37 | HGMD(*) | R496C (1486C > T) | A | I | — | — | — | — | — |
| SNP42 | Novel(CAP) | stop +712G > A | | I | — | .28 | .18 | .18 | .21 |
| SNP16 | GeneSNPs(R) | stop +2074G > A | D | I | .06 | .01 | .02 | — | .03 |

CYP3A4

| | | | | | | | | | |
|---|---|---|---|---|---|---|---|---|---|
| SNP48 | GeneSNPs(*) | −8086G > A | A | I | — | — | — | — | — |
| SNP49 | GeneSNPs(−) | −6790G > A | B | I | .50 | NA | NA | NA | NA |
| SNP47 | dbSNP(R) | −1232C > T | 1 | II | .19 | .05 | .04 | .56 | .56 |
| SNP22 | Novel(CDS) | −847A > T | | I | .06 | — | — | .20 | .15 |
| SNP12 | Novel(CAP) | −747C > G | 1, 2 | II | — | .08 | .08 | .01 | .04 |
| SNP11 | HCANC(+) | −392A > G | 3 | II | .13 | .04 | .04 | .58 | .54 |
| SNP45 | HGVbase(*) | −290A > G | A | I | — | — | — | — | — |
| SNP50 | GeneSNPs(*) | −26G > A | A | I | — | — | — | — | — |
| SNP21 | Novel(CDS) | IVS1 −868C > T | | I | NA | — | .009 | .18 | .17 |
| SNP20 | dbSNP(R) | IVS2 +671T > A | | I | .15 | .07 | .07 | .42 | .44 |
| SNP19 | Novel(CDS) | IVS2 −132C > T | D | I | .02 | — | .009 | — | — |
| SNP26 | Novel(C+C) | IVS3 +1992T > C | B | I | .40 | NA | NA | NA | NA |
| SNP32 | Novel(CDS) | IVS3 −734G > A | B | I | NA | NA | NA | NA | NA |
| SNP33 | GeneSNPs(+<) | IVS4 −172G > A | A | I | .02 | — | — | — | — |
| SNP17 | HCANC(*) | S222P (664T > C) | A | I | — | — | — | — | — |
| SNP1 | dbSNP(R) | IVS7 +34T > G | 1 | II | .17 | .06 | .05 | .62 | .56 |
| SNP14 | dbSNP(R) | IVS7 +526C > T | | I | .02 | — | .02 | .11 | .11 |
| SNP13 | Novel(C+C) | IVS7 −202C > T | 1 | II | .31 | .14 | .13 | .66 | .72 |
| SNP2 | Novel(C+C) | IVS9 +187C > G | B | I | .08 | NA | NA | NA | NA |
| SNP27 | Novel(C+C) | IVS9 +841T > G | | I | .06 | — | .01 | .07 | .08 |
| SNP46 | HGVbase(+<) | M318I (954G > A) | B | I | — | NA | NA | NA | NA |
| SNP10 | dbSNP(R) | IVS10 +12G > A | | I | .42 | .16 | .14 | .67 | .66 |
| SNP34 | GeneSNPs/dbSNP(*) | I431T (1292T > C) | A | I | — | — | — | — | — |
| SNP18 | HCANC(*) | M445T (1334T > C) | A | I | — | — | — | — | — |
| SNP29 | Novel(CDS) | IVS12 +581C > T | A | I | .02 | — | — | — | — |
| SNP30 | Novel(CDS) | IVS12 +586G > A | D | I | .02 | .04 | .01 | — | .01 |
| SNP31 | Novel(CDS) | IVS12 +646C > A | A | I | .02 | — | — | — | — |
| SNP28 | Novel(C+C) | IVS12 −473T > G | | I | .08 | .006 | .01 | .24 | .27 |
| SNP24 | Novel(C+C) | stop +766delT; | 1 | I | .33 | .14 | .13 | .52 | .53 |
| | | T > G | | | | | | | |
| SNP6 | Novel(CAP) | stop +945A > T | D | I | — | .02 | .02 | — | — |
| SNP25 | Novel(CDS) | stop +1454C > T | 1 | II | .08 | .003 | .006 | .23 | .28 |
| SNP5 | Novel(C+C) | stop +1639A > T | 1 | II | .63 | .17 | .16 | .61 | .62 |
| SNP15 | Novel(C+C) | stop +2204G > C | 1 | II | .13 | .13 | .11 | .24 | .20 |

SRD5A2

| SNP17 | GeneSNPs(R) | −8029C > T | 1, 2 | II | .33 | .46 | .46 | .46 | .37 |
|---|---|---|---|---|---|---|---|---|---|
| SNP18 | GeneSNPs(*) | −7819G > C | A | I | — | — | — | — | — |
| SNP26 | GeneSNPs(+) | −3001G > A | 1 | II | .30 | .29 | .30 | .27 | .39 |
| SNP28 | GeneSNPs(*) | −2851A > T | A | I | — | — | — | — | — |
| SNP31 | Novel(C+C) | −2036(A)7-8, A > T | C | I | NA | .29 | .28 | .43 | .33 |
| SNP5 | GeneSNPs(R) | −1971G > A | B | I | .48 | NA | NA | NA | NA |
| SNP30 | Novel(CAP) | −870G > A | D | I | — | .01 | .02 | — | .01 |
| SNP21 | HGMD(*) | G34R (100G > A) | A | I | — | — | — | — | — |
| SNP22 | GeneSNPs/dbSNP/HGVbase(−) | A49T (145G > A) | 1, 3 | II | NA | .04 | .04 | .01 | .03 |
| SNP20 | GeneSNPs/dbSNP/HGVbase(−) | V89L (265G > C) | 1, 3 | II | NA | .29 | .29 | .32 | .34 |
| SNP23 | GeneSNPs/dbSNP(−) | IVS1 +15C > T | B | I | .46 | NA | NA | NA | NA |
| SNP11 | GeneSNPs/dbSNP(−) | IVS1 +24664G > T | | I | .48 | .24 | .27 | .19 | .22 |
| SNP12 | GeneSNPs/dbSNP(−) | IVS2 +626C > T | 1, 2 | II | .48 | .41 | .40 | .27 | .30 |
| SNP7 | HGMD(*) | G183S (547G > A) | A | I | — | — | — | — | — |
| SNP8 | HGMD(*) | N193S (578A > G) | A | I | — | — | — | — | — |
| SNP9 | HGMD(*) | P212R (635C > G) | A | I | — | — | — | — | — |
| SNP10 | HGMD(*) | IVS4 +1G > T | A | I | — | — | — | — | — |
| SNP32 | Novel(CAP) | stop +545T > C | D | I | — | — | .005 | — | — |
| SNP4 | Novel(C+C) | stop +849A > G | | I | .13 | .11 | .12 | .16 | .23 |
| SNP2 | Novel(C+C) | stop +1356A > C | D | I | .02 | .006 | .009 | — | — |
| SNP1 | GeneSNPs(R) | stop +1552G > A | 1 | II | .16 | .12 | .12 | .19 | .23 |
| SNP13 | GeneSNPs(+) | stop +3059G > A | 1 | II | .13 | .09 | .09 | .13 | .14 |
| SNP14 | GeneSNPs(−) | stop +5179A > C | D | I | .02 | .01 | .005 | — | — |
| SNP15 | GeneSNPs(−) | stop +9301G > C | 1 | II | .46 | .26 | .27 | .21 | .23 |
| SNP16 | GeneSNPs(−) | stop +9502C > T | D | I | — | .006 | — | — | — |
| ^{a}Explanations: (*), SNP did not show up in our study population; (R), rediscovered; (+), we had sequence coverage but did not rediscover the SNP; (+<), we had sequence coverage but did not rediscover the SNP, most likely due to the low minor allele frequency; (−), we did not have sequence coverage explaining why we did not rediscover the SNP; (CDS), novel SNP discovered originally in the | | | | | | | | | |
| # Coriell Diversity Set; (CAP), novel SNP discovered originally in the prostate cancer sibships; (C+C), novel SNP discovered originally in both populations | | | | | | | | | |
| ^{b}Underlined bases indicate the allele for which frequencies are given | | | | | | | | | |
| ^{c}Excluded from haplotyping in Phase I and from consideration for Phase II based on (A) being monoallelic in the prostate cancer sibships, (B) yielding ambiguous genotyping results, (C) low success rate, (D) allele frequency <5%. Included in Phase II association analyses based on (1) being a haplotype tagging SNP, (2) case-control difference in Phase I, (3) previous publications supporting association, (4) SNP conveniently | | | | | | | | | |
| # located within the same PCR fragment as another included SNP | | | | | | | | | |

^{d}I, allele frequencies based on 276 samples; II, allele frequencies based on 1117 samples
^{e}NA, data not available

TABLE 4A
All non-stratified association results between CYP17, CYP3A4, and SRD5A2 variants
and risk of prostate cancer among cases and sibling controls^{a}

| | | All Subjects | | European Americans | | African-Americans | |
|---|---|---|---|---|---|---|---|
| | | (n = 886 . . . 920) | | (n = 781 . . . 834) | | (n = 74 . . . 76) | |
| | Genotype | | p- | | p- | | p- |
| Genes | Comparison^{b} | OR (95% Cl) | value | OR (95% Cl) | value | OR (95% Cl) | value |

CYP17

| | | | | | | | |
|---|---|---|---|---|---|---|---|
| SNP29 | CC or TC vs. TT | 0.91 (0.65-1.29) | 0.61 | 0.86 (0.59-1.25) | 0.42 | 1.96 (0.72-5.31) | 0.19 |
| SNP4 | AA or GA vs. GG | 0.88 (0.62-1.25) | 0.47 | 0.82 (0.56-1.19) | 0.30 | 1.96 (0.72-5.31) | 0.19 |
| SNP20 | AA or GA vs. GG | 0.57 (0.25-1.31) | 0.19 | 0.52 (0.21-1.28) | 0.15 | 1.87 (0.55-6.35) | 0.31 |
| SNP6 | TT or CT vs. CC | 0.90 (0.64-1.27) | 0.56 | 0.81 (0.57-1.17) | 0.27 | 2.38 (0.71-7.92) | 0.16 |
| SNP7 | CC or AC vs. AA | 0.84 (0.59-1.19) | 0.33 | 0.77 (0.53-1.11) | 0.16 | 2.00 (0.59-6.72) | 0.27 |
| SNP22 | TT or CT vs. CC | 1.99 (0.67-5.86) | 0.21 | NA^{c} | NA | 1.69 (0.43-6.68) | 0.45 |
| SNP3 | GG or CG vs. CC | 0.90 (0.63-1.27) | 0.54 | 0.81 (0.56-1.19) | 0.28 | 2.23 (0.76-6.54) | 0.14 |

CYP3A4

| | | | | | | | |
|---|---|---|---|---|---|---|---|
| SNP47 | TT or CT vs. CC | 0.59 (0.31-1.09) | 0.09 | 0.60 (0.29-1.23) | 0.16 | 0.56 (0.17-1.86) | 0.34 |
| SNP12 | GG or CG vs. CC | 1.51 (0.92-2.50) | 0.11 | 1.44 (0.86-2.38) | 0.16 | NA | NA |
| SNP11 | GG or AG vs. AA | 0.76 (0.43-1.36) | 0.36 | 0.83 (0.41-1.66) | 0.59 | 0.61 (0.23-1.63) | 0.32 |
| SNP1 | GG or TG vs. TT | 0.53 (0.29-0.99) | 0.05 | 0.57 (0.28-1.18) | 0.13 | 0.44 (0.13-1.57) | 0.21 |
| SNP13 | TT or CT vs. CC | 0.79 (0.51-1.22) | 0.29 | 0.71 (0.45-1.12) | 0.14 | 2.33 (0.42-12.84) | 0.33 |
| SNP24 | GG or TG vs. TT | 0.95 (0.62-1.44) | 0.79 | 0.88 (0.56-1.36) | 0.56 | 1.81 (0.45-7.25) | 0.40 |
| SNP25 | TT or CT vs. CC | 1.59 (0.58-4.39) | 0.37 | NA | NA | 1.21 (0.37-3.98) | 0.75 |
| SNP5 | TT or AT vs. AA | 0.86 (0.56-1.31) | 0.47 | 0.74 (0.48-1.15) | 0.19 | 4.48 (0.67-30.07) | 0.12 |
| SNP15 | CC or GC vs. GG | 0.69 (0.46-1.05) | 0.09 | 0.68 (0.44-1.05) | 0.08 | 0.82 (0.22-3.03) | 0.77 |

SRD5A2

| | | | | | | | |
|---|---|---|---|---|---|---|---|
| SNP17 | TT or CT vs. CC | 0.87 (0.58-1.29) | 0.48 | 0.93 (0.61-1.41) | 0.74 | 0.21 (0.04-1.12) | 0.07 |
| SNP26 | AA or GA vs. GG | 1.57 (1.08-2.30) | 0.02 | 1.59 (1.08-2.34) | 0.02 | 1.00 (0.19-5.31) | 1.00 |
| SNP22 | AA or GA vs. GG | 0.84 (0.38-1.85) | 0.66 | 0.90 (0.40-2.02) | 0.79 | NA | NA |
| SNP20 | CC or GC vs. GG | 1.56 (1.08-2.25) | 0.02 | 1.47 (1.00-2.16) | 0.05 | 2.29 (0.81-6.50) | 0.12 |
| SNP12 | TT or CT vs. CC | 1.00 (0.69-1.46) | 0.98 | 0.98 (0.67-1.44) | 0.94 | 0.94 (0.18-4.97) | 0.94 |
| SNP1 | AA or GA vs. GG | 0.81 (0.53-1.24) | 0.33 | 0.83 (0.53-1.31) | 0.43 | 1.20 (0.23-6.21) | 0.83 |
| SNP13 | AA or GA vs. GG | 0.94 (0.61-1.47) | 0.80 | 0.98 (0.61-1.55) | 0.92 | 1.64 (0.25-10.54) | 0.61 |
| SNP15 | CC or GC vs. GG | 1.14 (0.79-1.63) | 0.49 | 1.14 (0.79-1.65) | 0.49 | 0.77 (0.15-3.94) | 0.75 |

^{a}From conditional logistic regression, with matching on family, and a variance estimator that incorporates sibling correlations.
^{b}All results are from dominant models that compare homozygous and heterozygous carriers of variant versus the homozygous wildtype (OR = 1.0).
^{c}NA, data not available

TABLE 4B
Statistically significant allele associations obtained from analysis stratified by aggressiveness^{a}

| | | All Subjects | | European Americans | | African-Americans | |
|---|---|---|---|---|---|---|---|
| | | (n = 443 . . . 465) | | (n = 394 . . . 418) | | (n = 39) | |
| SNP | Stratification | OR (95% Cl) | p-value | OR (95% Cl) | p-value | OR (95% Cl) | p-value |

CYP3A4

| | | | | | | | |
|---|---|---|---|---|---|---|---|
| SNP47 | Low TNM and grade | 0.19 (0.06-0.62) | 0.006 | 0.07 (0.01-0.53) | 0.10 | 0.66 (0.14-3.04) | 0.59 |
| SNP11 | Low TNM and grade | 0.20 (0.06-0.67) | 0.009 | 0.08 (0.01-0.59) | 0.13 | 0.66 (0.14-3.04) | 0.59 |
| SNP1 | Low TNM and grade | 0.21 (0.05-0.86) | 0.03 | 0.16 (0.03-0.82) | 0.03 | 0.65 (0.03-16.26) | 0.80 |
| SNP25 | Low TNM and grade | 6.54 (0.99-43.10) | 0.05 | NA^{b} | NA | 6.57 (1.26-34.17) | 0.03 |
| SNP5 | Low TNM and grade | 0.57 (0.30-1.10) | 0.09 | 0.51 (0.26-0.99) | 0.05 | NA | NA |
| SNP15 | Low TNM and grade | 0.41 (0.22-0.79) | 0.007 | 0.52 (0.27-1.01) | 0.06 | NA | NA |

SRD5A2

| | | | | | | | |
|---|---|---|---|---|---|---|---|
| SNP1 | Low TNM and grade | 0.59 (0.32-1.10) | 0.09 | 0.52 (0.27-1.00) | 0.05 | 1.41 (0.18-10.79) | 0.74 |

^{a}From conditional logistic regression, with matching on family, and a variance estimator that incorporates sibling correlation.
^{b}NA, data not available

TABLE 5A
All non-stratified haplotype association results for CYP17, CYP3A4, and SRD5A2^{a}.

| | All Subjects | | European Americans | | African-Americans | |
|---|---|---|---|---|---|---|
| | (n = 920) | | (n = 834) | | (n = 76) | |
| Haplotype | OR (95% Cl) | p-value | OR (95% Cl) | p-value | OR (95% Cl) | p-value |

CYP17

| | | | | | | |
|---|---|---|---|---|---|---|
| Hap1 | 1.0 | — | 1.0 | — | 1.0 | — |
| Hap2 | 0.83 (0.61-1.12) | 0.22 | 0.80 (0.58-1.10) | 0.17 | 2.63 (0.45-15.33) | 0.28 |
| Hap3 | 1.07 (0.67-1.70) | 0.78 | 1.09 (0.65-1.83) | 0.74 | 1.41 (0.49-4.08) | 0.52 |
| Hap4 | 0.85 (0.56-1.31) | 0.47 | 0.84 (0.51-1.40) | 0.51 | 1.02 (0.43-2.42) | 0.97 |

CYP3A4

| | | | | | | |
|---|---|---|---|---|---|---|
| Hap1 | 1.0 | — | 1.0 | — | 1.0 | 1 |
| Hap2 | 1.25 (0.74-2.08) | 0.41 | 1.16 (0.69-1.96) | 0.57 | NA^{b} | NA |
| Hap3 | 1.20 (0.70-2.03) | 0.51 | 1.07 (0.62-1.82) | 0.82 | 3.34 (0.49-22.89) | 0.22 |
| Hap4 | 0.46 (0.21-1.01) | 0.05 | 0.44 (0.20-0.96) | 0.04 | 0.99 (0.06-1 6.37) | 0.99 |
| Hap5 | 1.08 (0.78-1.50) | 0.66 | 1.05 (0.74-1.51) | 0.77 | 1.86 (0.60-5.75) | 0.28 |

SRD5A2

| | | | | | | |
|---|---|---|---|---|---|---|
| Hap1 | 1.0 | — | 1.0 | — | 1.0 | — |
| Hap2 | 1.14 (0.82-1.60) | 0.43 | 1.12 (0.80-1.58) | 0.50 | 2.57 (0.43-15.52) | 0.30 |
| Hap3 | 0.76 (0.48-1.21) | 0.25 | 0.81 (0.51-1.30) | 0.39 | NA | NA |
| Hap4 | 1.13 (0.72-1.77) | 0.61 | 1.03 (0.64-1.66) | 0.90 | NA | NA |
| Hap5 | 1.59 (0.78-3.24) | 0.20 | 1.58 (0.79-3.19) | 0.20 | NA | NA |
| Hap6 | 1.27 (0.60-2.68) | 0.52 | 2.16 (0.87-5.37) | 0.10 | 0.64 (0.10-4.00) | 0.63 |
| Hap7 | 0.74 (0.50-1.09) | 0.13 | 0.80 (0.51-1.23) | 0.30 | 1.11 (0.29-4.27) | 0.88 |

^{a}From conditional logistic regression, with matching on family, and a variance estimator that incorporates sibling correlation.
^{b}NA, data not available

TABLE 5B
Statistically significant haplotype associations obtained from analysis stratified by high aggressiveness
(i.e., high TNM stage or Gleason score) and low aggressiveness (i.e., low TNM stage and Gleason score)^{a}

| | | All Subjects | | European Americans | | African-Americans | |
|---|---|---|---|---|---|---|---|
| | | (n = 395 . . . 465) | | (n = 362 . . . 418) | | (n = 33 . . . 39) | |
| Haplotype | Stratification | OR (95% Cl) | p-value | OR (95% Cl) | p-value | OR (95% Cl) | p-value |

CYP3A4

| | | | | | | | |
|---|---|---|---|---|---|---|---|
| Hap4 | Low TNM and | 0.06 (0.008-0.50) | 0.009 | 0.09 (0.01-0.68) | 0.02 | NA^{b} | NA |
| | grade | | | | | | |

SRD5A2

| | | | | | | | |
|---|---|---|---|---|---|---|---|
| Hap3 | High TNM or grade | 0.52 (0.29-0.91) | 0.02 | 0.53 (0.30-0.95) | 0.03 | NA | NA |

^{a}From conditional logistic regression, with matching on family, and a variance estimator that incorporates sibling correlation.
^{b}NA, data not available

TABLE 6
Annotation of CYP3A4, CYP17 and SRD5A2 genomic sequences

| | | | Sub | |
|---|---|---|---|---|
| Gene | Annotation | Base pairs | annotation | Base pairs |
| CYP3A4 | 5′ region | 1-10481 | | |
| | Exon 1 | 10482-10642 | | |
| | | | 5′ UTR | 10482-10571 |
| | | | Start codon | 10572-10574 |
| | | | Translated | 10572-10642 |
| | | | region | |
| | Intron 1 | 10643-14574 | | |
| | Exon 2 | 14575-14668 | | |
| | Intron 2 | 14669-16579 | | |
| | Exon 3 | 16580-16632 | | |
| | Intron 3 | 16633-22072 | | |
| | Exon 4 | 22073-22172 | | |
| | Intron 4 | 22173-24526 | | |
| | Exon 5 | 24527-24640 | | |
| | Intron 5 | 24641-24905 | | |
| | Exon 6 | 24906-24994 | | |
| | Intron 6 | 24995-26259 | | |
| | Exon 7 | 26260-26408 | | |
| | Intron 7 | 26409-27502 | | |
| | Exon 8 | 27503-27630 | | |
| | Intron 8 | 27631-28314 | | |
| | Exon 9 | 28315-28381 | | |
| | Intron 9 | 28382-30736 | | |
| | Exon 10 | 30737-30897 | | |
| | Intron 10 | 30898-32482 | | |
| | Exon 11 | 32483-32709 | | |
| | Intron 11 | 32710-33768 | | |
| | Exon 12 | 33769-33931 | | |
| | Intron 12 | 33932-36520 | | |
| | Exon 13 | 36521-37073 | | |
| | | | Translated | 36521-36613 |
| | | | region | |
| | | | Stop codon | 36614-36616 |
| | | | 3′ UTR | 36617-37073 |
| | 3′ region | 37074-39071 | | |
| CYP17 | 5′ region | 1-9992 | | |
| | Exon 1 | 9993-10337 | | |
| | | | 5′ UTR | 9993-10040 |
| | | | Start codon | 10041-10043 |
| | | | Translated | 10041-10337 |
| | | | region | |
| | Intron 1 | 10338-12009 | | |
| | Exon 2 | 12010-12148 | | |
| | Intron 2 | 12149-12387 | | |
| | Exon 3 | 12388-12617 | | |
| | Intron 3 | 12618-13279 | | |
| | Exon 4 | 13280-13366 | | |
| | Intron 4 | 13367-14193 | | |
| | Exon 5 | 14194-14409 | | |
| | Intron 5 | 14410-14721 | | |
| | Exon 6 | 14722-14891 | | |
| | Intron 6 | 14892-15790 | | |
| | Exon 7 | 15791-15894 | | |
| | Intron 7 | 15895-16416 | | |
| | Exon 8 | 16417-16872 | | |
| | | | Translated | 16417-16697 |
| | | | region | |
| | | | Stop codon | 16698-16700 |
| | | | 3′ UTR | 16701-16872 |
| | 3′ region | 16873-26865 | | |
| SRD5A2 | 5′ region | 1-9995 | | |
| | Exon 1 | 9996-10307 | | |
| | | | 5′ UTR | 9996-10026 |
| | | | Start codon | 10027-10029 |
| | | | Translated | 10027-10307 |
| | | | region | |
| | Intron 1 | 10308-57160 | | |
| | Exon 2 | 57161-57324 | | |
| | Intron 2 | 57325-59454 | | |
| | Exon 3 | 59455-59556 | | |
| | Intron 3 | 59557-61469 | | |
| | Exon 4 | 61470-61620 | | |
| | Intron 4 | 61621-64664 | | |
| | Exon 5 | 64665-66344 | | |
| | | | Translated | 64665-64728 |
| | | | region | |
| | | | Stop codon | 64729-64731 |
| | | | 3′ UTR | 64732-66344 |
| | 3′ region | 66345-76341 | | |

1. An isolated polynucleotide selected from the group consisting of a nucleotide sequence comprising one or more polymorphic sequences of SEQ ID NOS: 1-34.

2. A fragment of said isolated polynucleotide of claim 1, wherein said fragment comprises a polymorphic site in the polymorphic sequence.

3. An isolated polynucleotide comprising a sequence complementary to one or more of the polymorphic sequences of claim 1.

4. A fragment of said complementary nucleotide sequence of claim 3, wherein said fragment comprises a polymorphic site in the polymorphic sequence.

5. The isolated polynucleotide of claim 1, wherein said polynucleotide is selected from the group consisting of DNA, RNA, cDNA and mRNA.

6. The isolated polynucleotide of claim 1, wherein at least one single nucleotide polymorphism is at a position selected from the group consisting of position [CYP3A4_IVS9 +187] of SEQ ID NO: 1, position [CYP3A4, 1639 base pairs after the stop codon] of SEQ ID NO: 2, position [CYP3A4, 945 base pairs after the stop codon] of SEQ ID NO: 3, position [CYP3A4_{—}5′ region −747] of SEQ ID NO: 4, position [CYP3A4_IVS7 −202] of SEQ ID NO: 5, position [CYP3A4, 2204 base pairs after the stop codon] of SEQ ID NO: 6, position [CYP3A4_IVS2 −132] of SEQ ID NO: 7, position [CYP3A4_IVS1 −868] of SEQ ID NO: 8, position [CYP3A4_{—}5′ region −847] of SEQ ID NO: 9, position [CYP3A4, 766 base pairs after the stop codon] of SEQ ID NO: 10, position [CYP3A4, 1454 base pairs after the stop codon] of SEQ ID NO: 11, position [CYP3A4_IVS3 +1992] of SEQ ID NO: 12, position [CYP3A4_IVS9 +841] of SEQ ID NO: 13, position [CYP3A4_IVS12 −473] of SEQ ID NO: 14, position [CYP3A4_IVS12 +581] of SEQ ID NO: 15, position [CYP3 A4_IVS12 +586] of SEQ ID NO: 16, position [CYP3A4_IVS 12 +646] of SEQ ID NO: 17, position [CYP3A4_IVS3 −734] of SEQ ID NO: 18, position [CYP17_IVS1 −271] of SEQ ID NO: 19, position [CYP17_IVS5 +75] of SEQ ID NO: 20, position [CYP17_IVS1 +426] of SEQ ID NO: 21, position [CYP17 IVS1 −99] of SEQ ID NO: 22, position [CYP17_IVS1 −700] of SEQ ID NO: 23, position [CYP17_IVS1 −565] of SEQ ID NO: 24, position [CYP17_IVS3 +141] of SEQ ID NO: 25, position [CYP17_{—}5′ region −1488] of SEQ ID NO: 26, position [CYP17_{—}5′ region −1204] of SEQ ID NO: 27, position [CYP17_IVS1 +466] of SEQ ID NO: 28, position [CYP17, 712 base pairs after the stop codon] of SEQ ID NO: 29, position [SRD5A2, 1356 base pairs after the stop codon (3′ UTR)] of SEQ ID NO: 30, position [SRD5A2, 849 base pairs after the stop codon (3′ UTR)] of SEQ ID NO: 31, position [SRD5A2_{—}5′ region −870] of SEQ ID NO: 32, position [SRD5A2_{—}5′ region between −2036 and −2030] of SEQ ID NO: 33 and position [SRD5A2, 545 base pairs after the stop codon (3′ UTR)] of SEQ ID NO: 34.

7. The isolated polynucleotide of claim 6, wherein at least one single nucleotide polymorphism is selected from the group consisting of [CYP3A4_IVS9 +187C>G] of SEQ ID NO: 1, [CYP3A4, 1639 base pairs after the stop codon, A>T] of SEQ ID NO: 2, [CYP3A4, 945 base pairs after the stop codon, A>T] of SEQ ID NO: 3, [CYP3A4_{—}5′ region −747C>G] of SEQ ID NO: 4, [CYP3A4_IVS7 −202C>T] of SEQ ID NO: 5, [CYP3A4, 2204 base pairs after the stop codon, G>C] of SEQ ID NO: 6, [CYP3A4_IVS2 −132C>T] of SEQ ID NO: 7, [CYP3A4_IVS1 −868C>T] of SEQ ID NO: 8, [CYP3A4_{—}5′ region −847A>T] of SEQ ID NO: 9, [CYP3A4, 766 base pairs after the stop codon, delT] of SEQ ID NO: 10, [CYP3A4, 1454 base pairs after the stop codon, C>T] of SEQ ID NO: 11, [CYP3A4_IVS3 +1992T>C] of SEQ ID NO: 12, [CYP3A4_IVS9 +841T>G] of SEQ ID NO: 13, [CYP3A4_IVS 12 −473T>G] of SEQ ID NO: 14, [CYP3A4_IVS12 +581 C>T] of SEQ ID NO: 15, [CYP3A4_IVS12 +586G>A] of SEQ ID NO: 16, [CYP3A4_IVS12 +646C>A] of SEQ ID NO: 17, [CYP3A4_IVS3 −734G>A] of SEQ ID NO: 18, [CYP 17_IVS1 −271A>C] of SEQ ID NO: 19, [CYP17_IVS5 +75C>G] of SEQ ID NO: 20, [CYP17_IVS1 +426G>A] of SEQ ID NO: 21, [CYP17_IVS1 −99C>T] of SEQ ID NO: 22, [CYP17_IVS1 −700C>G] of SEQ ID NO: 23, [CYP17_IVS1 −565G>A] of SEQ ID NO: 24, [CYP17_IVS3 +141A>T] of SEQ ID NO: 25, [CYP17_{—}5′ region −1488C>G] of SEQ ID NO: 26, [CYP17_{—}5′ region −1204C>T] of SEQ ID NO: 27, [CYP17_IVS1 +466G>A] of SEQ ID NO: 28, [CYP17, 712 base pairs after the stop codon, G>A] of SEQ ID NO: 29, [SRD5A2, 1356 base pairs after the stop codon (3′ UTR), A>C] of SEQ ID NO: 30, [SRD5A2, 849 base pairs after the stop codon (3′ UTR), A>G] of SEQ ID NO: 31, [SRD5A2_{—}5′ region −870G>A] of SEQ ID NO: 32, [SRD5A2_{—}5′ region −2036(A)7-8] of SEQ ID NO: 33 and [SRD5A2, 545 base pairs after the stop codon (3′ UTR), T>C] of SEQ ID NO: 34.

8. The complement of any of the isolated polynucleotides of claim 7.

9. The isolated polynucleotide of claim 1, wherein the nucleotide comprises part of the CYP17 gene, the CYP3A4 gene or the SRD5A2 gene.

10. A polypeptide encoded by the polynucleotide of claim 1.

11. An antibody to the polypeptide of claim 10.

12. The isolated polynucleotide of claim 1, further comprising a detectable label.

13. The isolated polynucleotide of claim 12, wherein said detectable label is selected from the group consisting of fluorophore, radionuclide, peptide, enzyme, antibody and antigen.

14. The isolated polynucleotide of claim 13, wherein said fluorophore is a fluorescent compound selected from the group consisting of Hoechst 33342, Cy2, Cy3, Cy5, CypHer, coumarin, FITC, DAPI, Alexa 633, DRAQ5 and Alexa 488.

15. A method for diagnosing a genetic susceptibility for a disease, condition or disorder related to prostate or breast cancer in a subject, said method comprising analysing a biological sample containing nucleic acid obtained from said subject to detect the presence or absence of one or more single nucleotide polymorphisms at a position selected from the group consisting of position [CYP3A4_IVS9 +187] of SEQ ID NO: 1, position [CYP3A4, 1639 base pairs after the stop codon] of SEQ ID NO: 2, position [CYP3A4, 945 base pairs after the stop codon] of SEQ ID NO: 3, position [CYP3A4_{—}5′ region −747] of SEQ ID NO: 4, position [CYP3A4_IVS7 −202] of SEQ ID NO: 5, position [CYP3A4, 2204 base pairs after the stop codon] of SEQ ID NO: 6, position [CYP3A4_IVS2 −132] of SEQ ID NO: 7, position [CYP3A4_IVS1 −868] of SEQ ID NO: 8, position [CYP3A4_{—}5′ region −847] of SEQ ID NO: 9, position [CYP3A4, 766 base pairs after the stop codon] of SEQ ID NO: 10, position [CYP3A4, 1454 base pairs after the stop codon] of SEQ ID NO: 11, position [CYP3A4_IVS3 +1992] of SEQ ID NO: 12, position [CYP3A4_IVS9 +841] of SEQ ID NO: 13, position [CYP3A4_IVS12 −473] of SEQ ID NO: 14, position [CYP3A4_IVS12 +581] of SEQ ID NO: 15, position [CYP3A4_IVS12 +586] of SEQ ID NO: 16, position [CYP3A4_IVS12 +646] of SEQ ID NO: 17, position [CYP3A4_IVS3 −734] of SEQ ID NO: 18, position [CYP17_IVS1 −271] of SEQ ID NO: 19, position [CYP17_IVS5 +75] of SEQ ID NO: 20, position [CYP17_IVS1 +426] of SEQ ID NO: 21, position [CYP17_IVS1 −99] of SEQ ID NO: 22, position [CYP17_IVS1 −700] of SEQ ID NO: 23, position [CYP17_IVS1 −565] of SEQ ID NO: 24, position [CYP17_IVS3 +141] of SEQ ID NO: 25, position [CYP17_{—}5′ region −1488] of SEQ ID NO: 26, position [CYP17_{—}5′ region −1204] of SEQ ID NO: 27, position [CYP17_IVS1 +466] of SEQ ID NO: 28, position [CYP 17, 712 base pairs after the stop codon] of SEQ ID NO: 29, position [SRD5A2, 1356 base pairs after the stop codon (3′ UTR)] of SEQ ID NO: 30, position [SRD5A2, 849 base pairs after the stop codon (3′ UTR)] of SEQ ID NO: 31, position [SRD5A2_{—}5′ region −870] of SEQ ID NO: 32, position [SRD5A2_{—}5′ region between −2036 and −2030] of SEQ ID NO: 33, position [SRD5A2, 545 base pairs after the stop codon (3′ UTR)] of SEQ ID NO: 34, position [SRD5A2_IVS2 +626] of SEQ ID NO: 35, position [SRD5A2_{—}5′ region −8029] of SEQ ID NO: 36, position [CYP3A4_IVS7 +34] of SEQ ID NO: 42, position [CYP3A4_{—}5′ region −1232] of SEQ ID NO: 43, position [SRD5A2_{—}5′ region −3001] of SEQ ID NO: 44 and position [SRD5A2, 1552 base pairs after the stop codon] of SEQ ID NO: 45.

16. The method of claim 15, wherein said nucleic acid is selected from the group consisting of DNA, RNA, cDNA and mRNA.

17. The method of claim 15, wherein said single nucleotide polymorphism is selected from the group consisting of [CYP3A4_IVS9 +187C>G] of SEQ ID NO: 1, [CYP3A4, 1639 base pairs after the stop codon, A>T] of SEQ ID NO: 2, [CYP3A4, 945 base pairs after the stop codon, A>T] of SEQ ID NO: 3, [CYP3A4_{—}5′ region −747C>G] of SEQ ID NO: 4, [CYP3A4_IVS7 −202C>T] of SEQ ID NO: 5, [CYP3A4, 2204 base pairs after the stop codon, G>C] of SEQ ID NO: 6, [CYP3A4_IVS2 −132C>T] of SEQ ID NO: 7, [CYP3A4_IVS1 −868C>T] of SEQ ID NO: 8, [CYP3A4_{—}5′ region −847A>T] of SEQ ID NO: 9, [CYP3A4, 766 base pairs after the stop codon, delT] of SEQ ID NO: 10, [CYP3A4, 1454 base pairs after the stop codon, C>T] of SEQ ID NO: 11, [CYP3A4_IVS3 +1992T>C] of SEQ ID NO: 12, [CYP3A4_IVS9 +841 T>G] of SEQ ID NO: 13, [CYP3A4_IVS12 −473T>G] of SEQ ID NO: 14, [CYP3A4_IVS12 +581 C>T] of SEQ ID NO: 15, [CYP3A4_IVS12 +586G>A] of SEQ ID NO: 16, [CYP3A4_IVS12 +646C>A] of SEQ ID NO: 17, [CYP3A4_IVS3 −734G>A] of SEQ ID NO: 18, [CYP17_IVS1 −271A>C] of SEQ ID NO: 19, [CYP17_IVS5 +75C>G] of SEQ ID NO: 20, [CYP17_IVS1 +426G>A] of SEQ ID NO: 21, [CYP17_IVS1 −99C>T] of SEQ ID NO: 22, [CYP17_IVS1 −700C>G] of SEQ ID NO: 23, [CYP17_IVS1 −565G>A] of SEQ ID NO: 24, [CYP17_IVS3 +141A>T] of SEQ ID NO: 25, [CYP17_{—}5′ region −1488C>G] of SEQ ID NO: 26, [CYP17_{—}5′ region −1204C>T] of SEQ ID NO: 27, [CYP17_IVS1 +466G>A] of SEQ ID NO: 28, [CYP 17, 712 base pairs after the stop codon, G>A] of SEQ ID NO: 29, [SRD5A2, 1356 base pairs after the stop codon (3′ UTR), A>C] of SEQ ID NO: 30, [SRD5A2, 849 base pairs after the stop codon (3′ UTR), A>G] of SEQ ID NO: 31, [SRD5A2_{—}5′ region −870G>A] of SEQ ID NO: 32, [SRD5A2_{—}5′ region −2036(A)7-8] of SEQ ID NO: 33, [SRD5A2, 545 base pairs after the stop codon (3′ UTR), T>C] of SEQ ID NO: 34, [SRD5A2_IVS2 +626C>T] of SEQ ID NO: 35, [SRD5A2_{—}5′ region −8029C>T] of SEQ ID NO: 36, [CYP3A4_IVS7 +34T>G] of SEQ ID NO: 42, [CYP3A4_{—}5′ region −1232C>T] of SEQ ID NO: 43, [SRD5A2_{—}5′ region −3001G>A] of SEQ ID NO: 44 and [SRD5A2, 1552 base pairs after the stop codon, G>A] of SEQ ID NO: 45.

18. The method of claim 15, wherein said single nucleotide polymorphism is selected from the complement of any of the single nucleotide polymorphisms.

19. The method of claim 15, wherein said analysis is accomplished by a process selected from the group consisting of sequencing, genotyping, fragment analysis, hybridisation, restriction fragment analysis, oligonucleotide ligation and allele specific PCR.

20. The method of claim 19, wherein the analysis is accomplished by hybridisation comprising the steps of
i) contacting said nucleic acid with an oligonucleotide that hybridises to one or more isolated polynucleotide polymorphic sequence selected from the group consisting of SEQ ID NOS: 1-36 and SEQ ID NOS: 42-45 or its complement;
ii) determining whether the nucleic acid and said oligonucleotide hybridize;
whereby hybridisation of the nucleic acid to the oligonucleotide indicates the presence of the polymorphic site in the nucleic acid.

21. A method for diagnosing a genetic susceptibility for a disease, condition or disorder related to prostate or breast cancer in a subject, or predicting an individual's response to a drug, said method comprising adding an antibody to a polypeptide present in a biological sample obtained from said subject which polypeptide is encoded by a polynucleotide selected from the group consisting of SEQ ID NOS: 1-36 and SEQ ID NOS: 42-45, or the complement thereof, and detecting specific binding of said antibody to said polypeptide.

22. A kit comprising at least one isolated polynucleotide of at least 5 contiguous nucleotides of SEQ ID NOS: 1-36 or SEQ ID NOS: 42-45, or the complement thereof, and containing at least one single nucleotide polymorphic site associated with a disease, condition or disorder related to prostate or breast cancer, together with instructions for the use thereof for detecting the presence or the absence of said at least single nucleotide polymorphism in said nucleic acid.

23. An oligonucleotide array comprising at least one oligonucleotide capable of hybridising to a first polynucleotide at a polymorphic site encompassed therein, wherein the first polynucleotide comprises a nucleotide sequence comprising one or more polymorphic sequences of SEQ ID NOS: 1-36 or SEQ ID NOS: 42-45.

24. The oligonucleotide array of claim 23, wherein said first polynucleotide comprises a fragment of any of said nucleotide sequences, said fragment comprising a polymorphic site in said polymorphic sequence.

25. The oligonucleotide array of claim 23. wherein the first polynucleotide is a complementary nucleotide sequence comprising a sequence complementary to one or more polymorphic sequences of SEQ ID NOS: 1-36 or SEQ ID NOS: 42-45.

26. The oligonucleotide array of claim 25, wherein the first polynucleotide comprises a fragment of said complementary sequence, said fragment comprising a polymorphic site in said polymorphic sequence.

27. The kit of claim 22, wherein the position of said polymorphic site is at a position selected from the group consisting of position [CYP3A4_IVS9 +187] of SEQ ID NO: 1, position [CYP3A4, 1639 base pairs after the stop codon] of SEQ ID NO: 2, position [CYP3A4, 945 base pairs after the stop codon] of SEQ ID NO: 3, position [CYP3A4_{—}5′ region −747] of SEQ ID NO: 4, position [CYP3A4_IVS7 −202] of SEQ ID NO: 5, position [CYP3A4, 2204 base pairs after the stop codon] of SEQ ID NO: 6, position [CYP3A4_IVS2 −132] of SEQ ID NO: 7, position [CYP3A4_IVS1 −868] of SEQ ID NO: 8, position [CYP3A4_{—}5′ region −847] of SEQ ID NO: 9, position [CYP3A4, 766 base pairs after the stop codon] of SEQ ID NO: 10, position [CYP3A4, 1454 base pairs after the stop codon] of SEQ ID NO: 11, position [CYP3A4_IVS3 +1992] of SEQ ID NO: 12, position [CYP3A4_IVS9 +841] of SEQ ID NO: 13, position [CYP3A4_IVS12 −473] of SEQ ID NO: 14, position [CYP3A4_IVS12 +581] of SEQ ID NO: 15, position [CYP3A4_IVS12 +586] of SEQ ID NO: 16, position [CYP3A4_IVS12 +646] of SEQ ID NO: 17, position [CYP3A4_IVS3 −734] of SEQ ID NO: 18, position [CYP17_IVS1 −271] of SEQ ID NO: 19, position [CYP17_IVS5 +75] of SEQ ID NO: 20, position [CYP17_IVS1 +426] of SEQ ID NO: 21, position [CYP17_IVS1 −99] of SEQ ID NO: 22, position [CYP17_IVS1 −700] of SEQ ID NO: 23, position [CYP17_IVS1 −565] of SEQ ID NO: 24, position [CYP17_IVS3 +141] of SEQ ID NO: 25, position [CYP17_{—}5′ region −1488] of SEQ ID NO: 26, position [CYP17_{—}5′ region −1204] of SEQ ID NO: 27, position [CYP17_IVS1 +466] of SEQ ID NO: 28, position [CYP17, 712 base pairs after the stop codon] of SEQ ID NO: 29, position [SRD5A2, 1356 base pairs after the stop codon (3′ UTR)] of SEQ ID NO: 30, position [SRD5A2, 849 base pairs after the stop codon (3′ UTR)] of SEQ ID NO: 31, position [SRD5A2_{—}5′ region −870] of SEQ ID NO: 32, position [SRD5A2_{—}5′ region between −2036 and −2030] of SEQ ID NO: 33, position [SRD5A2, 545 base pairs after the stop codon (3′ UTR)] of SEQ ID NO: 34, position [SRD5A2_IVS2 +626] of SEQ ID NO: 35, position [SRD5A2_{—}5′ region −8029] of SEQ ID NO: 36, position [CYP3A4_IVS7 +34] of SEQ ID NO: 42, position [CYP3A4_{—}5′ region −1232] of SEQ ID NO: 43, position [SRD5A2_{—}5′ region −3001] of SEQ ID NO: 44 and position [SRD5A2, 1552 base pairs after the stop codon] of SEQ ID NO: 45.

28. The kit of claim 22, wherein at least one single nucleotide polymorphism is selected from the group consisting of [CYP3A4_IVS9 +187C>G] of SEQ ID NO: 1, [CYP3A4, 1639 base pairs after the stop codon, A>T] of SEQ ID NO: 2, [CYP3A4, 945 base pairs after the stop codon, A>T] of SEQ ID NO: 3, [CYP3A4_{—}5′ region −747C>G] of SEQ ID NO: 4, [CYP3A4_IVS7 −202C>T] of SEQ ID NO: 5, [CYP3A4, 2204 base pairs after the stop codon, G>C] of SEQ ID NO: 6, [CYP3A4_IVS2 −132C>T] of SEQ ID NO: 7, [CYP3A4_IVS1 −868C>T] of SEQ ID NO: 8, [CYP3A4_{—}5′ region −847A>T] of SEQ ID NO: 9, [CYP3A4, 766 base pairs after the stop codon, delT] of SEQ ID NO: 10, [CYP3A4, 1454 base pairs after the stop codon, C>T] of SEQ ID NO: 11, [CYP3A4_IVS3 +1992T>C] of SEQ ID NO: 12, [CYP3A4_IVS9 +841T>G] of SEQ ID NO: 13, [CYP3A4_IVS12 −473T>G] of SEQ ID NO: 14, [CYP3A4_IVS12 +581C>T] of SEQ ID NO: 15, [CYP3A4_IVS12 +586G>A] of SEQ ID NO: 16, [CYP3A4_IVS12 +646C>A] of SEQ ID NO: 17, [CYP3A4_IVS3 −734G>A] of SEQ ID NO: 18, [CYP17_IVS1 −271A>C] of SEQ ID NO: 19, [CYP17_IVS5 +75C>G] of SEQ ID NO: 20, [CYP17_IVS1 +426G>A] of SEQ ID NO: 21, [CYP17_IVS1 −99C>T] of SEQ ID NO: 22, [CYP17_IVS1 −700C>G] of SEQ ID NO: 23, [CYP17_IVS1 −565G>A] of SEQ ID NO: 24, [CYP17_IVS3 +141A>T] of SEQ ID NO: 25, [CYP17_{—}5′ region −1488C>G] of SEQ ID NO: 26, [CYP17_{—}5′ region −1204C>T] of SEQ ID NO: 27, [CYP17_IVS1 +466G>A] of SEQ ID NO: 28, [CYP17, 712 base pairs after the stop codon, G>A] of SEQ ID NO: 29, [SRD5A2, 1356 base pairs after the stop codon (3′ UTR), A>C] of SEQ ID NO: 30, [SRD5A2, 849 base pairs after the stop codon (3′ UTR), A>G] of SEQ ID NO: 31, [SRD5A2_{—}5′ region −870G>A] of SEQ ID NO: 32, [SRD5A2_{—}5′ region −2036(A)7-8] of SEQ ID NO: 33, [SRD5A2, 545 base pairs after the stop codon (3′ UTR), T>C] of SEQ ID NO: 34, [SRD5A2_IVS2 +626C>T] of SEQ ID NO: 35, [SRD5A2_{—}5′ region −8029C>T] of SEQ ID NO: 36, [CYP3A4_IVS7 +34T>G] of SEQ ID NO: 42, [CYP3A4_{—}5′ region −1232C>T] of SEQ ID NO: 43, [SRD5A2_{—}5′ region −3001G>A] of SEQ ID NO: 44 and [SRD5A2, 1552 base pairs after the stop codon, G>A] of SEQ ID NO: 45.

29. The kit of claim 28, wherein at least one single nucleotide polymorphism is the complement of any of the single nucleotide polymorphisms.

30. The kit of claim 22, wherein said oligonucleotide further comprises a detectable label.

31. The kit of claim 30, wherein said label is selected from the group consisting of fluorophore, radionuclide, peptide, enzyme, antibody and antigen.

32. The kit of claim 30, wherein said fluorophore is a fluorescent compound selected from the group consisting of Hoechst 33342, Cy2, Cy3, Cy5, CypHer, coumarin, FITC, DAPI, Alexa 633 DRAQ5 and Alexa 488.

33. A method of treatment or prophylaxis of a subject comprising the steps of
i) analysing a biological sample containing nucleic acid obtained from said subject to detect the presence or absence of at least one single nucleotide polymorphism in SEQ ID NOS: 1-36 or SEQ ID NOS: 42-45, or the complement thereof, associated with a disease, condition or disorder related to prostate or breast cancer; and
ii) treating the subject for said disease, condition or disorder if step i) detects the presence of at least one single nucleotide polymorphism in SEQ ID NOS: 1-36 or SEQ ID NOS: 42-45, or the complement thereof.

34. The method of claim 33, wherein said nucleic acid is selected from the group consisting of DNA, RNA and mRNA.

35. The method of claim 33, wherein the sample is analysed to detect the presence or absence of at least one single nucleotide polymorphism at a position selected from the group consisting of position [CYP3A4_IVS9 +187] of SEQ ID NO: 1, position [CYP3A4, 1639 base pairs after the stop codon] of SEQ ID NO: 2, position [CYP3A4, 945 base pairs after the stop codon] of SEQ ID NO: 3, position [CYP3A4_{—}5′ region −747] of SEQ ID NO: 4, position [CYP3A4_IVS7 −202] of SEQ ID NO: 5, position [CYP3A4, 2204 base pairs after the stop codon] of SEQ ID NO: 6, position [CYP3A4_IVS2 −132] of SEQ ID NO: 7, position [CYP3A4_IVS1 −868] of SEQ ID NO: 8, position [CYP3A4_{—}5′ region −847] of SEQ ID NO: 9, position [CYP3A4, 766 base pairs after the stop codon] of SEQ ID NO: 10, position [CYP3A4, 1454 base pairs after the stop codon] of SEQ ID NO: 11, position [CYP3A4_IVS3 +1992] of SEQ ID NO: 12, position [CYP3A4_IVS9 +841] of SEQ ID NO: 13, position [CYP3A4_IVS12 −473] of SEQ ID NO: 14, position [CYP3A4_IVS12 +581] of SEQ ID NO: 15, position [CYP3A4_IVS12 +586] of SEQ ID NO: 16, position [CYP3A4_IVS12 +646] of SEQ ID NO: 17, position [CYP3A4_IVS3 −734] of SEQ ID NO: 18, position [CYP17_IVS1 −271] of SEQ ID NO: 19, position [CYP17_IVS5 +75] of SEQ ID NO: 20, position [CYP17_IVS1 +426] of SEQ ID NO: 21, position [CYP17_IVS1 −99] of SEQ ID NO: 22, position [CYP17_IVS1 −700] of SEQ ID NO: 23, position [CYP17_IVS1 −565] of SEQ ID NO: 24, position [CYP17_IVS3 +141] of SEQ ID NO: 25, position [CYP17_{—}5′ region −1488] of SEQ ID NO: 26, position [CYP17_{—}5′ region −1204] of SEQ ID NO: 27, position [CYP17_IVS1 +466] of SEQ ID NO: 28, position [CYP17, 712 base pairs after the stop codon] of SEQ ID NO: 29, position [SRD5A2, 1356 base pairs after the stop codon (3′ UTR)] of SEQ ID NO: 30, position [SRD5A2, 849 base pairs after the stop codon (3′ UTR)] of SEQ ID NO: 31, position [SRD5A2_{—}5′ region −870] of SEQ ID NO: 32, position [SRD5A2_{—}5′ region between −2036 and −2030] of SEQ ID NO: 33, position [SRD5A2, 545 base pairs after the stop codon (3′ UTR)] of SEQ ID NO: 34, position [SRD5A2_IVS2 +626] of SEQ ID NO: 35, position [SRD5A2_{—}5′ region −8029] of SEQ ID NO: 36, position [CYP3A4_IVS7 +34] of SEQ ID NO: 42, position [CYP3A4_{—}5′ region −1232] of SEQ ID NO: 43, position [SRD5A2_{—}5′ region −3001] of SEQ ID NO: 44 and position [SRD5A2, 1552 base pairs after the stop codon] of SEQ ID NO: 45.

36. The method of claim 35, wherein at least one single nucleotide polymorphism is selected from the group consisting of [CYP3A4_IVS9 +187C>G] of SEQ ID NO: 1, [CYP3A4, 1639 base pairs after the stop codon, A>T] of SEQ ID NO: 2, [CYP3A4, 945 base pairs after the stop codon, A>T] of SEQ ID NO: 3, [CYP3A4_{—}5′ region −747C>G] of SEQ ID NO: 4, [CYP3A4_IVS7 −202C>T] of SEQ ID NO: 5, [CYP3A4, 2204 base pairs after the stop codon, G>C] of SEQ ID NO: 6, [CYP3A4_IVS2 −132C>T] of SEQ ID NO: 7, [CYP3A4_IVS1 −868C>T] of SEQ ID NO: 8, [CYP3A4_{—}5′ region −847A>T] of SEQ ID NO: 9, [CYP3A4, 766 base pairs after the stop codon, delT] of SEQ ID NO: 10, [CYP3A4, 1454 base pairs after the stop codon, C>T] of SEQ ID NO: 11, [CYP3A4_IVS3 +1992T>C] of SEQ ID NO: 12, [CYP3A4_IVS9 +841T>G] of SEQ ID NO: 13, [CYP3A4_IVS12 −473T>G] of SEQ ID NO: 14, [CYP3A4_IVS12 +581C>T] of SEQ ID NO: 15, [CYP3A4_IVS12 +586G>A] of SEQ ID NO: 16, [CYP3A4_IVS12 +646C>A] of SEQ IDS NO: 17, [CYP3A4_IVS3 −734G>A] of SEQ ID NO: 18, [CYP17_IVS1 −271A>C] of SEQ ID NO: 19, [CYP17_IVS5 +75C>G] of SEQ ID NO: 20, [CYP17_IVS1 +426G>A] of SEQ ID NO: 21, [CYP17_IVS1 −99C>T] of SEQ ID NO: 22, [CYP17_IVS1 −700C>G] of SEQ ID NO: 23, [CYP17_IVS1 −565G>A] of SEQ ID NO: 24, [CYP17_IVS3 +141A>T] of SEQ ID NO: 25, [CYP17_{—}5′ region −1488C>G] of SEQ ID NO: 26, [CYP17_{—}5′ region −1204C>T] of SEQ ID NO: 27, [CYP17_IVS1 +466G>A] of SEQ ID NO: 28, [CYP17, 712 base pairs after the stop codon, G>A] of SEQ ID NO: 29, [SRD5A2, 1356 base pairs after the stop codon (3′ UTR), A>C] of SEQ ID NO: 30, [SRD5A2, 849 base pairs after the stop codon (3′ UTR), A>G] of SEQ ID NO: 31, [SRD5A2_{—}5′ region −870G>A] of SEQ ID NO: 32, [SRD5A2_{—}5′ region −2036(A)7-8] of SEQ ID NO: 33, [SRD5A2, 545 base pairs after the stop codon (3′ UTR), T>C] of SEQ ID NO: 34, [SRD5A2_IVS2 +626C>T] of SEQ ID NO: 35, [SRD5A2_{—}5′ region −8029C>T] of SEQ ID NO: 36, [CYP3A4_IVS7 +34T>G] of SEQ ID NO: 42, [CYP3A4_{—}5′ region −1232C>T] of SEQ ID NO: 43, [SRD5A2_{—}5′ region −3001G>A] of SEQ ID NO: 44, and [SRD5A2, 1552 base pairs after the stop codon, G>A] of SEQ ID NO: 45.

37. The method of claim 36, wherein at least one single nucleotide polymorphism is the complement of any of the single nucleotide polymorphisms.

38. The method of claim 33, wherein said method counteracts the effect of said at least one single nucleotide polymorphism detected.

39. The method of claim 33, wherein the method comprises treatment with a polynucleotide selected from the group consisting of polymorphic sequences SEQ ID NOS: 1-36 and SEQ ID NOS: 42-45, or their complement, provided that the polymorphic sequence, or the complement, does not contain at least one single nucleotide polymorphism at a position selected from the group consisting of position [CYP3A4_IVS9 +187] of SEQ ID NO: 1, position [CYP3A4, 1639 base pairs after the stop codon] of SEQ ID NO: 2, position [CYP3A4, 945 base pairs after the stop codon] of SEQ ID NO: 3, position [CYP3A4_{—}5′ region −747] of SEQ ID NO: 4, position [CYP3A4_IVS7 −202] of SEQ ID NO: 5, position [CYP3A4, 2204 base pairs after the stop codon] of SEQ ID NO: 6, position [CYP3A4_IVS2 −132] of SEQ ID NO: 7, position [CYP3A4_IVS1 −868] of SEQ ID NO: 8, position [CYP3A4_{—}5′ region −847] of SEQ ID NO: 9, position [CYP3A4, 766 base pairs after the stop codon] of SEQ ID NO: 10, position [CYP3A4, 1454 base pairs after the stop codon] of SEQ ID NO: 11, position [CYP3A4_IVS3 +1992] of SEQ ID NO: 12, position [CYP3A4_IVS9 +841] of SEQ ID NO: 13, position [CYP3A4_IVS12 −473] of SEQ ID NO: 14, position [CYP3A4_IVS12 +581] of SEQ ID NO: 15, position [CYP3A4_IVS12 +586] of SEQ ID NO: 16, position [CYP3A4_IVS12 +646] of SEQ ID NO: 17, position [CYP3A4_IVS3 −734] of SEQ ID NO: 18, position [CYP17_IVS1 −271] of SEQ ID NO: 19, position [CYP17_IVS5 +75] of SEQ ID NO: 20, position [CYP17_IVS1 +426] of SEQ ID NO: 21, position [CYP17_IVS1 −99] of SEQ ID NO: 22, position [CYP17_IVS1 −700] of SEQ ID NO: 23, position [CYP17_IVS1 −565] of SEQ ID NO: 24, position [CYP17_IVS3 +141] of SEQ ID NO: 25, position [CYP17_{—}5′ region −1488] of SEQ ID NO: 26, position [CYP17_{—}5′ region −1204] of SEQ ID NO: 27, position [CYP17_IVS1 +466] of SEQ ID NO: 28, position [CYP17, 712 base pairs after the stop codon] of SEQ ID NO: 29, position [SRD5A2, 1356 base pairs after the stop codon (3′ UTR)] of SEQ ID NO: 30, position [SRD5A2, 849 base pairs after the stop codon (3′ UTR)] of SEQ ID NO: 31, position [SRD5A2_{—}5′ region −870] of SEQ ID NO: 32, position [SRD5A2_{—}5′ region between −2036 and −2030] of SEQ ID NO: 33, position [SRD5A2, 545 base pairs after the stop codon (3′ UTR)] of SEQ ID NO: 34, position [SRD5A2_IVS2 +626] of SEQ ID NO: 35, position [SRD5A2_{—}5′ region −8029] of SEQ ID NO: 36, position [CYP3A4_IVS7 +34] of SEQ ID NO: 42, position [CYP3A4_{—}5′ region −1232] of SEQ ID NO: 43, position [SRD5A2_{—}5′ region −3001] of SEQ ID NO: 44 and position [SRD5A2, 1552 base pairs after the stop codon] of SEQ ID NO: 45.

40. The method of claim 39, wherein the polymorphic sequence does not contain at least one single nucleotide polymorphism selected from the group consisting of [CYP3A4_IVS9 +187C>G] of SEQ ID NO: 1, [CYP3A4, 1639 base pairs after the stop codon, A>T] of SEQ ID NO: 2, [CYP3A4, 945 base pairs after the stop codon, A>T] of SEQ ID NO: 3, [CYP3A4_{—}5′ region −747C>G] of SEQ ID NO: 4, [CYP3A4_IVS7 −202C>T] of SEQ ID NO: 5, [CYP3A4, 2204 base pairs after the stop codon, G>C] of SEQ ID NO: 6, [CYP3A4_IVS2 −132C>T] of SEQ ID NO: 7, [CYP3A4_IVS1 −868C>T] of SEQ ID NO: 8, [CYP3A4_{—}5′ region −847A>T] of SEQ ID NO: 9, [CYP3A4, 766 base pairs after the stop codon, delT] of SEQ ID NO: 10, [CYP3A4, 1454 base pairs after the stop codon, C>T] of SEQ ID NO: 11, [CYP3A4_IVS3 +1992T>C] of SEQ ID NO: 12, [CYP3A4_IVS9 +841T>G] of SEQ ID NO: 13, [CYP3A4_IVS12 −473T>G] of SEQ ID NO: 14, [CYP3A4_IVS12 +581C>T] of SEQ ID NO: 15, [CYP3A4_IVS12 +586G>A] of SEQ ID NO: 16, [CYP3A4_IVS 12 +646C>A] of SEQ ID NO: 17, [CYP3A4_IVS3 −734G>A] of SEQ ID NO: 18, [CYP17_IVS1_{—}271A>C] of SEQ ID NO: 19, [CYP17_IVS5 +75C>G] of SEQ ID NO: 20, [CYP17_IVS1 +426G>A] of SEQ ID NO: 21, [CYP17_IVS1 −99C>T] of SEQ ID NO: 22, [CYP17_IVS1 −700C>G] of SEQ ID NO: 23, [CYP17_IVS1 −565G>A] of SEQ ID NO: 24, [CYP17_IVS3 +141A>T] of SEQ ID NO: 25, [CYP17_{—}5′ region −1488C>G] of SEQ ID NO: 26, [CYP17_{—}5′ region −1204C>T] of SEQ ID NO: 27, [CYP 17_IVS1 +466G>A] of SEQ ID NO: 28, [CYP17, 712 base pairs after the stop codon, G>A] of SEQ ID NO: 29, [SRD5A2, 1356 base pairs after the stop codon (3′ UTR), A>C] of SEQ ID NO: 30, [SRD5A2, 849 base pairs after the stop codon (3′ UTR), A>G] of SEQ ID NO: 31, [SRD5A2_{—}5′ region −870G>A] of SEQ ID NO: 32, [SRD5A2_{—}5′ region −2036(A)7-8] of SEQ ID NO: 33, [SRD5A2, 545 base pairs after the stop codon (3′ UTR), T>C] of SEQ ID NO: 34, [SRD5A2_IVS2 +626C>T] of SEQ ID NO: 35, [SRD5A2_{—}5′ region −8029C>T] of SEQ ID NO: 36, [CYP3A4_IVS7 +34T>G] of SEQ ID NO: 42, [CYP3A4_{—}5′ region −1232C>T] of SEQ ID NO: 43, [SRD5A2_{—}5′ region −3001G>A] of SEQ ID NO: 44, and [SRD5A2, 1552 base pairs after the stop codon, G>A] of SEQ ID NO: 45.

41. The method of claim 40, wherein the polymorphic sequence does not contain the complement of any of the single nucleotide polymorphisms.

42. The method of claim 33, wherein said method comprises treatment with a polypeptide which is encoded by a polynucleotide selected from the group consisting of polymorphic sequences SEQ ID NOS: 1-36 and SEQ ID NOS: 42-45 or their complement, provided that the polymorphic sequence, or the complement, does not contain at least one single nucleotide polymorphism at a position selected from the group consisting of position [CYP3A4_IVS9 +187] of SEQ ID NO: 1, position [CYP3A4, 1639 base pairs after the stop codon] of SEQ ID NO: 2, position [CYP3A4, 945 base pairs after the stop codon] of SEQ ID NO: 3, position [CYP3A4_{—}5′ region −747] of SEQ ID NO: 4, position [CYP3A4_IVS7 −202] of SEQ ID NO: 5, position [CYP3A4, 2204 base pairs after the stop codon] of SEQ ID NO: 6, position [CYP3A4_IVS2 −132] of SEQ ID NO: 7, position [CYP3A4_IVS1 −868] of SEQ ID NO: 8, position [CYP3A4_{—}5′ region −847] of SEQ ID NO: 9, position [CYP3A4, 766 base pairs after the stop codon] of SEQ ID NO: 10, position [CYP3A4, 1454 base pairs after the stop codon] of SEQ ID NO: 11, position [CYP3A4_IVS3 +1992] of SEQ ID NO: 12, position [CYP3A4_IVS9 +841] of SEQ ID NO: 13, position [CYP3A4_IVS12 −473] of SEQ ID NO: 14, position [CYP3A4_IVS12 +581] of SEQ ID NO: 15, position [CYP3A4_IVS12 +586] of SEQ ID NO: 16, position [CYP3A4_IVS12 +646] of SEQ ID NO: 17, position [CYP3A4_IVS3 −734] of SEQ ID NO: 18, position [CYP17_IVS1 −271] of SEQ ID NO: 19, position [CYP17_IVS5 +75] of SEQ ID NO: 20, position [CYP17_IVS1 +426] of SEQ ID NO: 21, position [CYP17_IVS1 −99] of SEQ ID NO: 22, position [CYP17_IVS1 −700] of SEQ ID NO: 23, position [CYP17_IVS1 −565] of SEQ ID NO: 24, position [CYP17_IVS3 +141] of SEQ ID NO: 25, position [CYP17_{—}5′ region −1488] of SEQ ID NO: 26, position [CYP17_{—}5′ region −1204] of SEQ ID NO: 27, position [CYP17_IVS1 +466] of SEQ ID NO: 28, position [CYP17, 712 base pairs after the stop codon] of SEQ ID NO: 29, position [SRD5A2, 1356 base pairs after the stop codon (3′ UTR)] of SEQ ID NO: 30, position [SRD5A2, 849 base pairs after the stop codon (3′ UTR)] of SEQ ID NO: 31, position [SRD5A2_{—}5′ region −870] of SEQ ID NO: 32, position [SRD5A2_{—}5′ region between −2036 and −2030] of SEQ ID NO: 33, position [SRD5A2, 545 base pairs after the stop codon (3′ UTR)] of SEQ ID NO: 34, position [SRD5A2_IVS2 +626] of SEQ ID NO: 35, position [SRD5A2_{—}5′ region −8029] of SEQ ID NO: 36, position [CYP3A4_IVS7 +34] of SEQ ID NO: 42, position [CYP3A4_{—}5′ region −1232] of SEQ ID NO: 43, position [SRD5A2_{—}5′ region −3001] of SEQ ID NO: 44, and position [SRD5A2, 1552 base pairs after the stop codon] of SEQ ID NO: 45.

43. The method of claim 42, wherein the polymorphic sequence does not contain at least one single nucleotide polymorphism selected from the group consisting of [CYP3A4_IVS9 +187C>G] of SEQ ID NO: 1, [CYP3A4, 1639 base pairs after the stop codon, A>T] of SEQ ID NO: 2, [CYP3A4, 945 base pairs after the stop codon, A>T] of SEQ ID NO: 3, [CYP3A4_{—}5′ region −747C>G] of SEQ ID NO: 4, [CYP3A4_IVS7 −202C>T] of SEQ ID NO: 5, [CYP3A4, 2204 base pairs after the stop codon, G>C] of SEQ ID NO: 6, [CYP3A4_IVS2 −132C>T] of SEQ ID NO: 7, [CYP3A4_IVS1 −868C>T] of SEQ ID NO: 8, [CYP3A4_{—}5′ region −847A>T] of SEQ ID NO: 9, [CYP3A4, 766 base pairs after the stop codon, delT] of SEQ ID NO: 10, [CYP3A4, 1454 base pairs after the stop codon, C>T] of SEQ ID NO: 11, [CYP3A4_IVS3 +1992T>C] of SEQ ID NO: 12, [CYP3A4_IVS9 +841T>G] of SEQ ID NO: 13, [CYP3A4_IVS12 −473T>G] of SEQ ID NO: 14, [CYP3A4_IVS12 +581C>T] of SEQ ID NO: 15, [CYP3A4_IVS12 +586G>A] of SEQ ID NO: 16, [CYP3A4_IVS12 +646C>A] of SEQ ID NO: 17, [CYP3A4_IVS3 −734G>A] of SEQ ID NO: 18, [CYP17_IVS1 −271A>C] of SEQ ID NO: 19, [CYP17_IVS5 +75C>G] of SEQ ID NO: 20, [CYP17_IVS1 +426G>A] of SEQ ID NO: 21, [CYP17_IVS1 −99C>T] of SEQ ID NO: 22, [CYP17_IVS1 −700C>G] of SEQ ID NO: 23, [CYP17_IVS1 −565G>A] of SEQ ID NO: 24, [CYP17_IVS3 +141A>T] of SEQ ID NO: 25, [CYP17_{—}5′ region −1488C>G] of SEQ ID NO: 26, [CYP17_{—}5′ region −1204C>T] of SEQ ID NO: 27, [CYP17_IVS1 +466G>A] of SEQ ID NO: 28, [CYP17, 712 base pairs after the stop codon, G>A] of SEQ ID NO: 29, [SRD5A2, 1356 base pairs after the stop codon (3′ UTR), A>C] of SEQ ID NO: 30, [SRD5A2, 849 base pairs after the stop codon (3′ UTR), A>G] of SEQ ID NO: 31, [SRD5A2_{—}5′ region −870G>A] of SEQ ID NO: 32, [SRD5A2_{—}5′ region −2036(A)7-8] of SEQ ID NO: 33, [SRD5A2, 545 base pairs after the stop codon (3′ UTR), T>C] of SEQ ID NO: 34, [SRD5A2_IVS2 +626C>T] of SEQ ID NO: 35, [SRD5A2_{—}5′ region −8029C>T] of SEQ ID NO: 36, [CYP3A4_IVS7 +34T>G] of SEQ ID NO: 42, [CYP3A4_{—}5′ region −1232C>T] of SEQ ID NO: 43, [SRD5A2_{—}5′ region −3001G>A] of SEQ ID NO: 44, and [SRD5A2, 1552 base pairs after the stop codon, G>A] of SEQ ID NO: 45.

44. The method of claim 43, wherein the polymorphic sequence does not contain the complement of any of the single nucleotide polymorphisms.

45. The method of claim 33, wherein said method comprises treatment with an antibody that binds specifically with a polypeptide encoded by a polynucleotide selected from the group consisting of SEQ ID NOS: 1-36 and SEQ ID NOS: 42-45, or the complement thereof.

46. A method for predicting the genetic ability of a subject or an organism to metabolise a chemical, said method comprising analysing a biological sample containing nucleic acid obtained from said subject or organism to detect the presence or absence of one or more single nucleotide polymorphisms at a position selected from the group consisting of position [CYP3A4_IVS9 +187] of SEQ ID NO: 1, position [CYP3A4, 1639 base pairs after the stop codon] of SEQ ID NO: 2, position [CYP3A4, 945 base pairs after the stop codon] of SEQ ID NO: 3, position [CYP3A4_{—}5′ region −747] of SEQ ID NO: 4, position [CYP3A4_IVS7 −202] of SEQ ID NO: 5, position [CYP3A4, 2204 base pairs after the stop codon] of SEQ ID NO: 6, position [CYP3A4_IVS2 −132] of SEQ ID NO: 7, position [CYP3A4_IVS1 −868] of SEQ ID NO: 8, position [CYP3A4_{—}5′ region −847] of SEQ ID NO: 9, position [CYP3A4, 766 base pairs after the stop codon] of SEQ ID NO: 10, position [CYP3A4, 1454 base pairs after the stop codon] of SEQ ID NO: 11, position [CYP3A4_IVS3 +1992] of SEQ ID NO: 12, position [CYP3A4_IVS9 +841] of SEQ ID NO: 13, position [CYP3A4_IVS12 −473] of SEQ ID NO: 14, position [CYP3A4_IVS12 +581] of SEQ ID NO: 15, position [CYP3A4_IVS12 +586] of SEQ ID NO: 16, position [CYP3A4_IVS12 +646] of SEQ ID NO: 17, position [CYP3A4_IVS3 −734] of SEQ ID NO: 18, position [CYP17_IVS1 −271] of SEQ ID NO: 19, position [CYP17_IVS5 +75] of SEQ ID NO: 20, position [CYP17_IVS1 +426] of SEQ ID NO: 21, position [CYP17_IVS1 −99] of SEQ ID NO: 22, position [CYP17_IVS1 −700] of SEQ ID NO: 23, position [CYP17_IVS1 −565] of SEQ ID NO: 24, position [CYP17_IVS3 +141] of SEQ ID NO: 25, position [CYP17_{—}5′ region −1488] of SEQ ID NO: 26, position [CYP17_{—}5′ region −1204] of SEQ ID NO: 27, position [CYP17_IVS1 +466] of SEQ ID NO: 28, position [CYP17, 712 base pairs after the stop codon] of SEQ ID NO: 29, position [SRD5A2, 1356 base pairs after the stop codon (3′ UTR)] of SEQ ID NO: 30, position [SRD5A2, 849 base pairs after the stop codon (3′ UTR)] of SEQ ID NO: 31, position [SRD5A2_{—}5′ region −870] of SEQ ID NO: 32, position [SRD5A2_{—}5′ region between −2036 and −2030] of SEQ ID NO: 33, position [SRD5A2, 545 base pairs after the stop codon (3′ UTR)] of SEQ ID NO: 34, position [SRD5A2_IVS2 +626] of SEQ ID NO: 35, position [SRD5A2_{—}5′ region −8029] of SEQ ID NO: 36, position [CYP3A4_IVS7 +34] of SEQ ID NO: 42, position [CYP3A4_{—}5′ region −1232] of SEQ ID NO: 43, position [SRD5A2_{—}5′ region −3001] of SEQ ID NO: 44 and position [SRD5A2, 1552 base pairs after the stop codon] of SEQ ID NO: 45, wherein the presence of a polymorphism at one or more of said positions is indicative of the subject's or organism's ability or inability to metabolise said chemical.

47. The method of claim 46, wherein said analysis comprises detecting the presence or absence of one or more single nucleotide polymorphisms selected from the group consisting of [CYP3A4_IVS9 +187C>G] of SEQ ID NO: 1, [CYP3A4, 1639 base pairs after the stop codon, A>T] of SEQ ID NO: 2, [CYP3A4, 945 base pairs after the stop codon, A>T] of SEQ ID NO: 3, [CYP3A4_{—}5′ region −747C>G] of SEQ ID NO: 4, [CYP3A4_IVS7 −202C>T] of SEQ ID NO: 5, [CYP3A4, 2204 base pairs after the stop codon, G>C] of SEQ ID NO: 6, [CYP3A4_IVS2 −132C>T] of SEQ ID NO: 7, [CYP3A4_IVS1 −868C>T] of SEQ ID NO: 8, [CYP3A4_{—}5′ region −847A>T] of SEQ ID NO: 9, [CYP3A4, 766 base pairs after the stop codon, delT] of SEQ ID NO: 10, [CYP3A4, 1454 base pairs after the stop codon, C>T] of SEQ ID NO: 11, [CYP3A4_IVS3 +1992T>C] of SEQ ID NO: 12, [CYP3A4_IVS9 +841T>G] of SEQ ID NO: 13, [CYP3A4_IVS12 −473T>G] of SEQ ID NO: 14, [CYP3A4_IVS12 +581C>T] of SEQ ID NO: 15, [CYP3A4_IVS12 +586G>A] of SEQ ID NO: 16, [CYP3A4_IVS12 +646C>A] of SEQ ID NO: 17, [CYP3A4_IVS3 −734G>A] of SEQ ID NO: 18, [CYP17_IVS 1 −271 A>C] of SEQ ID NO: 19, [CYP17_IVS5 +75C>G] of SEQ ID NO: 20, [CYP17_IVS1 +426G>A] of SEQ ID NO: 21, [CYP17_IVS1 −99C>T] of SEQ ID NO: 22, [CYP17_IVS1 −700C>G] of SEQ ID NO: 23, [CYP17_IVS1 −565G>A] of SEQ ID NO: 24, [CYP17_IVS3 +141A>T] of SEQ ID NO: 25, [CYP17_{—}5′ region −1488C>G] of SEQ ID NO: 26, [CYP17_{—}5′ region −1204C>T] of SEQ ID NO: 27, [CYP17_IVS1 +466G>A] of SEQ ID NO: 28, [CYP17, 712 base pairs after the stop codon, G>A] of SEQ ID NO: 29, [SRD5A2, 1356 base pairs after the stop codon (3′ UTR), A>C] of SEQ ID NO: 30, [SRD5A2, 849 base pairs after the stop codon (3′ UTR), A>G] of SEQ ID NO: 31, [SRD5A2_{—}5′ region −870G>A] of SEQ ID NO: 32, [SRD5A2_{—}5′ region −2036(A)7-8] of SEQ ID NO: 33, [SRD5A2, 545 base pairs after the stop codon (3′ UTR), T>C] of SEQ ID NO: 34, [SRD5A2_IVS2 +626C>T] of SEQ ID NO: 35, and [SRD5A2_{—}5′ region −8029C>T] of SEQ ID NO: 36, [CYP3A4_IVS7 +34T>G] of SEQ ID NO: 42, [CYP3A4_{—}5′ region −1232C>T] of SEQ ID NO: 43, [SRD5A2_{—}5′ region −3001 G>A] of SEQ ID NO: 44, [SRD5A2, 1552 base pairs after the stop codon, G>A] of SEQ ID NO: 45.

48. The method of claim 46, wherein the method further comprises predicting the response of the subject to the chemical by their ability or inability to metabolise the chemical.

49. The method of claim 46, wherein said chemical is a drug or a xenobiotic.

50. The method of claim 46, wherein said organism is selected from the group consisting of bacterium, fungus, protozoa, alga, fish, plant, insect and mammal.

51. A vector comprising a polynucleotide selected from the group consisting of a nucleotide sequence comprising one or more polymorphic sequences of SEQ ID NOS: 1-36 or SEQ ID NOS: 42-45.

52. A host cell transformed with the vector of claim 51.

53. The host cell of claim 52, wherein said host cell is selected from the group consisting of bacterium, fungus, protozoa, alga, fish, plant, insect and mammal.

54. The host cell of claim 53, wherein said mammal cell is a human cell.

55. A method of metabolising a chemical using the host cell of claim 52.

56. A method for making a host cell resistant to a chemical, said method comprising transforming said cell with any of the polynucleotides of claim 1.

57. An isolated haplotype selected from the group consisting of CYP3A4_Hap4 and SRD52_Hap3.

58. The isolated CYP3A4_Hap4 haplotype of claim 57, wherein said haplotype comprises Allele T at [CYP3A4_{—}5′ region −1232C>T], Allele C at [CYP3A4_{—}5′ region −747C>G], Allele G at [CYP3A4_{—}5′ region −392A>G], Allele G at [CYP3A4_IVS7 +34T>G], Allele T at [CYP3A4_IVS7 −202C>T], Allele G at [CYP3A4_stop +766T>G], Allele C at [CYP3A4_stop +1454C>T], Allele T at [CYP3A4_stop +1639A>T] and Allele C at [CYP3A4_stop +2204G>C].

59. The isolated SRD52_Hap3 haplotype of claim 57, wherein said haplotype comprises Allele C at [SRD5A2_{—}5′ region −8029C>T], Allele G at [SRD5A2_{—}5′ region −3001G>A], Allele G at [SRD5A2_{—}145G>A], Allele G at [SRD5A2_{—}265G>C], Allele T at [SRD5A2_IVS2 +626C>T], Allele G at [SRD5A2_stop +1552G>A], Allele G at [SRD5A2_stop +3059G>A] and Allele G at [SRD5A2_stop +9301G>C].

60. A method for diagnosing a genetic susceptibility for a disease, condition or disorder related to prostate or breast cancer in a subject, said method comprising analysing a biological sample obtained from said subject to detect the presence or absence of the haplotype of claim 57.

61. A method of diagnosing a genetic susceptibility for a disease, condition or disorder related to prostate or breast cancer in a subject, said method comprising adding an antibody to a polypeptide present in a sample obtained from said subject which polypeptide is encoded by the haplotype of claim 57, or the complement thereof, and detecting specific binding of said antibody to said polypeptide.

62. A method of treatment or prophylaxis of a subject comprising the steps of
i) analysing a sample of biological material containing a nucleic acid obtained from said subject to detect the presence or absence of at least one haplotype of claim 57, or the complement thereof, associated with a disease, condition or disorder related to prostate or breast cancer; and
ii) treating the subject for said disease, condition or disorder if step i) detects the presence of at least one said haplotype, or the complement thereof.

63. The method of claim 62, wherein the method comprises treatment with a portion of an isolated CYP3A4_Hap4 haplotype wherein said portion of said haplotype does not consist of at least one allele selected from the group consisting of Allele T at [CYP3A4_{—}5′ region −1232C>T], Allele C at [CYP3A4_{—}5′ region −747C>G], Allele G at [CYP3A4_{—}5′ region −392A>G], Allele G at [CYP3A4_IVS7 +34T>G], Allele T at [CYP3A4_IVS7 −202C>T], Allele G at [CYP3A4_stop +766T>G], Allele C at [CYP3A4_stop +1454C>T], Allele T at [CYP3A4_stop +1639A>T] and Allele C at [CYP3A4_stop +2204G>C].

64. The method of claim 62, wherein the method comprises treatment with a portion of an isolated SRD5A2_Hap3 haplotype wherein said portion of said haplotype does not comprise of at least one allele selected from the group consisting of Allele C at [SRD5A2_{—}5′ region −8029C>T], Allele G at [SRD5A2_{—}5′ region −3001G>A], Allele G at [SRD5A2_{—}145G>A], Allele G at [SRD5A2_{—}265G>C], Allele T at [SRD5A2_IVS2 +626C>T], Allele G at [SRD5A2_stop +1552G>A], Allele G at [SRD5A2_stop +3059G>A] and Allele G at [SRD5A2_stop +9301G>C].


SEQUENCE LISTING (45 sequences)

SEQ ID NO: 1 - DNA, 201 bp, Homo sapiens
  SNP (101)..(101): Alternative allele: G.
atgagaatttctgccacatagcagaacgacacatgtttgaatgttataagtggtagttggaggcactttctagaggcatgcaggcatagatagccatgttctaagagtaaagggcaaccctaagcaaacctggcatgctagaaagtcagtctgcggtctgtggatcacctacatcagatcaaatgccaattctcagcctcc

SEQ ID NO: 2 - DNA, 201 bp, Homo sapiens
  SNP (101)..(101): Alternative allele: T.
tgatagaagccaggcttctcacctttgcagaagggagtcatggattcagaaagggagaaaactagcatgaatcctatgaaattagattggaatggatgtaaccgtgtatattcatacccttgtagatagatagatgggtagatagatgatagataggtaacagatagatgacagataatgagatagatagatgtaaatgta

SEQ ID NO: 3 - DNA, 201 bp, Homo sapiens
  SNP (101)..(101): Alternative allele: T.
ggcaggagaatcacttgaacctgggaggcggatgttgaagtgagctgagattgcaccactgcactccagtctgggtgagagtgagactcagtcttaaaaaaatatgcctttttgaagcacgtacattttgtaacaaagaactgaagctcttattatattattagttttgatttaatgttttcagcccatctcctttcatat

SEQ ID NO: 4 - DNA, 201 bp, Homo sapiens
  SNP (101)..(101): Alternative allele: G.
aagtcaccagaaagtcagaagggatgacatgcagaggcccagcaatctcagctaagtcaactccaccagcctttctagttgcccactgtgtgtacagcaccctggtagggaccagagccatgacagggaataagactagactatgcccttgaggagctcacctctgttcagggaaacaggcgtggaaacacaatggtggta

SEQ ID NO: 5 - DNA, 201 bp, Homo sapiens
  SNP (101)..(101): Alternative allele: T.
ctgtagtccaatagataaaggcaaagagattagggcattgaattttgttccttttatccttcaaaagatgcacaaggggctgctgatctcactgctgtagcggtgctccttatgcatagacctgcccttgctcagccactggcctgaaagaggggcaaaagtcatagaaggaatggcttccagttgagaaccttgatgtct

SEQ ID NO: 6 - DNA, 201 bp, Homo sapiens
  SNP (101)..(101): Alternative allele: C.
gaactattggaactgataaacacattcagtaaagttgcaggatacaaaatcagcatacaaaaatcagtagcatttctatatgccaatagtgaacaatctggcaaaaataaaaaagtaatcccatttacaatagccacaaataaaactaaatacctagaaattaacttaatcaaagaagagaaaggtctctacaatgaatac

SEQ ID NO: 7 - DNA, 201 bp, Homo sapiens
  SNP (101)..(101): Alternative allele: T.
ataagtcattcagtatccacaacacttggagagaattcaagagtgattttaaatttcccttttcaaatacctcctctgttttctcttatttcctttatgacgtctccaaataagcttcctctaactgccagcaagtctgatttcattggcttcgactgttttcatcccaattagaggcagggttaagtacattaaaaataa

SEQ ID NO: 8 - DNA, 201 bp, Homo sapiens
  SNP (104)..(104): Alternative allele: T.
aactgcccctaggatccaatcatctcctaccaggccccacctccagtattggggattgcatttcaacatgagattttggtaggggcacagattcagaccatatcactggcactgtgctaatcagatgaatatcaccagttggaaggctagattccacaagaggaggaatgacctggaaattggttctttagttgtgattct

SEQ ID NO: 9 - DNA, 201 bp, Homo sapiens
  SNP (100)..(100): Alternative allele: T.
ggggtccccttgccaacagaatcacagaggaccagcctgaaagtgcagagacagcagctgaggcacagccaagagctctggctgtattaatgacctaagaagtcaccagaaagtcagaagggatgacatgcagaggcccagcaatctcagctaagtcaactccaccagcctttctagttgcccactgtgtgtacagcaccc

SEQ ID NO: 10 - DNA, 201 bp, Homo sapiens
  SNP (101)..(101): Alternative allele: base deleted.
tcaggcacagtggctcacgcctgtaatcctagcagtttgggaggctgagccgggtggatcgcctgaggtcaggagttcaagacaagcctggcctacatggttgaaaccccatctctactaaaaatacacaaattagctaggcatggtggactcgcctgtaatctcactacacaggaggctgaggcaggagaatcacttgaa

SEQ ID NO: 11 - DNA, 201 bp, Homo sapiens
  SNP (101)..(101): Alternative allele: T.
tgggtgtgggagtccaagcaagcagagaaggggtcgacgcagaggggtggcttgcaagagcagccagagcctaaatagggtatggagaacccacatgaggcgaggagggcatccatgagtgggaggggttgggtgaggtttggctacataaaggggattgatcaaataagtaaatgtattaaggatgatagaagccaggct

SEQ ID NO: 12 - DNA, 201 bp, Homo sapiens
  SNP (101)..(101): Alternative allele: C.
ttgcatttctctaatgaccagtgatgatgagcattttttcacatgtctgttggctgcatagatgtcttcttttgagaagtgtctgttcatatcctttgcctattttttgatggggttgtttgctttttttcttgtaaatttgtttaagttctttgtagattctggatgttagcccttcgtcagatggatagattgcaaaaa

SEQ ID NO: 13 - DNA, 201 bp, Homo sapiens
  SNP (101)..(101): Alternative allele: G.
taactattggttctagagagcaggactgggcttactccagcatactgctttaaatatatccatgtctacatccacttttgtctgtatgtctatgtatctatctatgtatctatctagctatgtatctatctatctatctatctatcatctatctatctatctatcatctatccatctatcatctatcatttatccatctat

SEQ ID NO: 14 - DNA, 201 bp, Homo sapiens
  SNP (101)..(101): Alternative allele: G.
cttcccatctttacactggatgggttcaattgggaggaattactggactctggaagttgaagactgtccatataattaaaatgtacaataactacccaggtttaccttgcaagtttcaacatacacaaaattaactttatatgactcttcaaaaacagtttgccatcatacctaataatctggtttaaattttaaaaactc

SEQ ID NO: 15 - DNA, 201 bp, Homo sapiens
  SNP (101)..(101): Alternative allele: T.
tgcccagagtgtggctttaaaagcttccccattgcttctcatgtgaagccaaggttgagaatgactaatttaaggcatttctggtggatataaaggactaccacagtccaaggccatcctgactgacctcaccttccaggtgcctagctccatccagctgggctccttttcaacccaattataactctattaatgttgttc

SEQ ID NO: 16 - DNA, 201 bp, Homo sapiens
  SNP (101)..(101): Alternative allele: A.
agagtgtggctttaaaagcttccccattgcttctcatgtgaagccaaggttgagaatgactaatttaaggcatttctggtggatataaaggactaccacagtccaaggccatcctgactgacctcaccttccaggtgcctagctccatccagctgggctccttttcaacccaattataactctattaatgttgttcccagc

SEQ ID NO: 17 - DNA, 201 bp, Homo sapiens
  SNP (101)..(101): Alternative allele: A.
taatttaaggcatttctggtggatataaaggactaccacagtccaaggccatcctgactgacctcaccttccaggtgcctagctccatccagctgggctccttttcaacccaattataactctattaatgttgttcccagccaggcatggtggctcatgcctgtaatcccagcactttgggaggccgaagcaggcggatca

SEQ ID NO: 18 - DNA, 201 bp, Homo sapiens
  SNP (101)..(101): Alternative allele: A.
ctaatttgattgcactgtggtctgagagacagtttgttatgatttctgttcttttacatttgctgaggagtgctttacttccaattatgtggtcaattttggaataagtgcgatgtggtgctgagaagaatgtatattctgttgatttggggtggagagttctgtagatgtctattaggtccgcttggtgcagagctgagt

SEQ ID NO: 19 - DNA, 200 bp, Homo sapiens
  SNP (100)..(100): Alternative allele: C.
ggacaggcatagtttagagagtttatcccatccagagttgccttctgtggtcagaaactgatgagcaaaaagaagcccagagggcaccctgtcagcgaaaagaaccccaatgctgctgcattctaattaagggttctttctttctccttgatctactgtatttctgaaggaattgggagtaggaggccttagggtctgtc

SEQ ID NO: 20 - DNA, 201 bp, Homo sapiens
  SNP (101)..(101): Alternative allele: G.
tggccttcctgctgcacaatcctcaggtgtgcttccccctcattgatcctagaccccagccagcccaatctctgggctccagagaaagggagagccaattctctcaggctttctgtgcaggaagactaggcctgccctgctccttacccaagcagtagttggctttgaccccagagtagagctgccccatcttctggaagc

SEQ ID NO: 21 - DNA, 201 bp, Homo sapiens
  SNP (101)..(101): Alternative allele: A.
cagcacttagcctagcacccagcacagtaagtgccccttatacagccaggattcatgttacttttcatggaaaatgggggcagtgactactgtcctccatgaaagctgctggggagaattagcctagctattgcaggctgggattgctgctttcctggtgctatttccagctactcaggctcacaggggcagttttctaca

SEQ ID NO: 22 - DNA, 201 bp, Homo sapiens
  SNP (101)..(101): Alternative allele: T.
attgggagtaggaggccttagggtctgtcctaccaagtccttgcagtcatggtggagtgcagtggggctgtgcccacatgggagtcagcatgccaggtacctgccttctcctccaggaaggaaagcagggaccagaggtgtaagggcaagagtggggtggatggtgtgagattcctacagccttgcctgctctctaaaggc

SEQ ID NO: 23 - DNA, 201 bp, Homo sapiens
  SNP (101)..(101): Alternative allele: G.
gccactgtgccctgccagcctctcagctttgatcaagccaagggttggtttattttttcttggaccaatcagccaggtctgctgaccaactacctagctcccacctctgctggcttcctcccgggggcagagaagatggagaaggctagtcatgtggatcttcagggtcaggaaatggaaaagggaggctttggacccttt

SEQ ID NO: 24 - DNA, 201 bp, Homo sapiens
  SNP (101)..(101): Alternative allele: A.
atggagaaggctagtcatgtggatcttcagggtcaggaaatggaaaagggaggctttggacccttttgctttggggggcacctctaggaggaggcagctcggcccaagtccagactgggtagacaaaacatctgcactctccaaatgtgggcttgtggctgggtatgcaggcttgcaatggaagggtaaacctgagtgagg

SEQ ID NO: 25 - DNA, 201 bp, Homo sapiens
  SNP (101)..(101): Alternative allele: T.
ctgacattgtccccaatcttccttcctttttacttccctgctccagccgcaatgacccatctttttcctgattacctccgccacctctacctcctctgccacttaaaacctttgccatttctctgcagagatagatttagccttttaattatgcaccttagtactccagataatgaccttcatttcttttccaattaccat

SEQ ID NO: 26 - DNA, 201 bp, Homo sapiens
  SNP (100)..(100): Alternative allele: G.
atttttagggaacaagggaaaacaaccataaggtctgactgcctgcagggtcgggcagaaagagccatattttccttcttgagagaggctataaatggacatgcaagtagggaagatatcactaaattcttttcctagcaaggagtattattattaataccctgggaaaggaatgcattcctggggggaggtctataaaca

SEQ ID NO: 27 - DNA, 201 bp, Homo sapiens
  SNP (101)..(101): Alternative allele: T.
tagggtggggaaaaactccgccctggtaaatttgtggtcagaccggttctctgctgtcgaaccctgtttgctgttgtttaaggtgtttatcaagacagtacgtgcaccgctgaacatagaccctcatctgtagttctgcttttgccctttgccttgtgatctttgttggacccttatcagtggttctgcttttgccctttg

SEQ ID NO: 28 - DNA, 201 bp, Homo sapiens
  SNP (101)..(101): Alternative allele: A.
tacagccaggattcatgttacttttcatggaaaatgggggcagtgactactgtcctccataaaagctgctggggagaattagcctagctattgcaggctgggattgctgctttcctggtgctatttccagctactcaggctcacaggggcagttttctacaatgacatttcagggttgctgatgagcctcccactcagcag

SEQ ID NO: 29 - DNA, 201 bp, Homo sapiens
  SNP (101)..(101): Alternative allele: A.
ctggaggattttaagtatgtaagtggaacaatctgtttttttgtttttgtttttgtttgagaaggagtttcgctcttgttgccctggctggagtgcaatggcatgatcttggctcactgcaacccctgcctcctgagttcaagtgattctcctgcctcagcctccaaaatagctgggattgcaggcgtgtgccaccatgcc

SEQ ID NO: 30 - DNA, 150 bp, Homo sapiens
  SNP (50)..(50): Alternative allele: C.
tcttgtgaaggggtcaccccagcatgagtgctgagatatggactctctaaggaaggggccgaacgcttgtaattggaatacatggaaatatttgtcttctcaggcctatgtttgcggaatgcattgtcaatatttagcaaactgttttga

SEQ ID NO: 31 - DNA, 151 bp, Homo sapiens
  SNP (51)..(51): Alternative allele: G.
cgagaacagttttacaatagacattgcaaactctcatgtttttggaaactagtggcaatatccaaataatgagtagtgtaaaacaaagagaattaatgatgaggttacatgctgcttgcctccaccagatgtccacaacaatatgaagtac

SEQ ID NO: 32 - DNA, 201 bp, Homo sapiens
  SNP (101)..(101): Alternative allele: A.
gtctgcgtgtatgacggctagacaggagttcagagaacagcggggtcgccaggccaccacctgatgggccacggctcattggctctaggagctgggaaagggcatcccaggaaagaagccctagactttagcctgagtctgggccactctaggggaccgggagtggggtggcgggagaggacgcgcagaatctcgacttct

SEQ ID NO: 33 - DNA, 200 bp, Homo sapiens
  SNP (101)..(107): Alternative allele: eight A′s instead of seven after base 100.
agctaattgttataatagtggagaaaagatcatgaggacaaaaagtgggcagagtcggaagaaaagagaggaagaaattgagacagaagacatttcatttaaaaaaatattccattgagctgggtttgaaatagtgcactgcctgttctcctaatgctgtatggtgtcatgaaatctattgtttactgagtctatgagcc

SEQ ID NO: 34 - DNA, 201 bp, Homo sapiens
  SNP (101)..(101): Alternative allele: C.
aactctgaagccacaaagacccagagcaaacccactcccaaatgaaaaccccagtcatggcttcctttttcttggttaattaggaaagatgagaaattattaggtagaccttgaatacaggagccctctcctcatagtgctgaaaagatactgatgcattgacctcatttcaaatttgtgcagtgtcttagttgatgagtg

SEQ ID NO: 35 - DNA, 211 bp, Homo sapiens
  SNP (101)..(101): Alternative allele: T.
  ref <308>: DATABASE ACCESSION NUMBER: NCBI SNP CLUSTER ID: rs413836
  ref <309>: DATABASE ENTRY DATE: 2003-03-07
aaagaaacattgtttcttaaaacaatgttttaagaaagtgtacgaatttgtgtcaggccacatccaaaactgtcctgggctgcatgtggcccacaggctgcgggttggacaagcctggcctagaaggctttgcccccatgtattcatgggggttggttcctcactttatttagttccctaccaatttgcacctcctcaaagggactttccc

SEQ ID NO: 36 - DNA, 213 bp, Homo sapiens
  SNP (123)..(123): Alternative allele: T.
  ref <308>: DATABASE ACCESSION NUMBER: NCBI SNP CLUSTER ID: rs545303
  ref <309>: DATABASE ENTRY DATE: 2003-03-07
gggaactcacagttttttggctgtctcatagagtttgcaacagtaaaactgcttctttcaaagggtctgtgaattctttcagttttcctggtatgttcccatggtagttcttgcagcaaaagctcacagtgtgagtctccacacactgttctgtccattccaagcaggagctgcatgttagttctgtctgctatccaccattttccaattttg

SEQ ID NO: 37 - DNA, 20 bp, Homo sapiens
tggccttgtacgtcgcgaag

SEQ ID NO: 38 - DNA, 21 bp, Homo sapiens
agcagggcagtgcgctgcact

SEQ ID NO: 39 - DNA, 39071 bp, Homo sapiens
  misc_feature (37334)..(37334): Unknown base.
  ref <308>: DATABASE ACCESSION NUMBER: http://www.genome.utah.edu/genesnps/
  ref <309>: DATABASE ENTRY DATE: 2002-06-05
  ref <313>: RELEVANT RESIDUES: (1)..(37073)
ggatccctactcactgtcttctctctacagaacgagatgtctctggagtccatagaaagcccaggagcctggctgggcacggtggctcctgcctgtaatcccagcactttgggaggccgaggcaggcagatcacctgagctcaggagttcaagaccagcctgggcaacatggcaaaaccccatctctactaaaaatacaaaaaattagctgggcgtggtggtgcatgcctctaatcccagctacttgggaggctgaggcacaagaattgcttgagcccaggaggcagcagttgcagtgagctgagattgtgccagtgcactccagcctgggcaacagagcaagattccatttcaaaaacaaaaacaaacacaaacaaacaaacaaaaatagaaagcccagggaccacctgcgtcaggttcccagccacacctttttcttgtcctcctctgtctctggcatcttctcacaggttcctaattgtttgtggttgcacaaattcaaaatcccagaaaaattaccacttcacacccactcagatggctattttttttttgaaggaagataacaagtgttgacaagaacatggagaaattggaattctcacccattgctggtgagaatgtaatacggtgctgctgctatggaaaacagcttggagtttcctcaaaaagttcaacagaatttcaatgtgacccagcaattcccctctaagttatagatctgagaggattaaaaacagttactaaaatacacggactcacatatttctaacagtccaattcacaagggccaaaaggtgctaatagcccacatgtccatcgatggatggataaataaattgtggtctatccatacaatggaatattattcggccataaatggaatgaagtactgacgcatgctacagaatggatgaaccgcaaaaaaaatggatgaacacatgctacagaatggatagcctcactttactatgaagtgaaggccagaaacgaagtccatatattgcatcatacaaaatatccagaagagggaagcccacagagacagaatgtgcaatggtggatgccagggtctggggagaggggagagtggggagaaactgctcaactggtacaggctttattttggaatgatgggaacattttgcaactagatagaggtagtgattgcagaacacagaatgtactgaattccactgatttttttcaccttaaaatggttaattttcagtcctgagattggataatcataaaaaaatggttaattttatgttatgtgaatttcatccctatacatattttaaacctcagaaatatacactagcaggcatggaacaggtcactgtggtgcctgccaagcccggtgatgttatctggggtccccggccagccttaagcctcttgctgaccggtggagggcagaacctttgccctaaaagtataatatccacatgctggcatgattcctggccagatggcttctttattagcagtaattgaaactgcctcgatacagacactgtaccttgcaaccaaaaaatgactcaacaatgataataagggttaagctgggcctttctctctttgccagttaaattatatttattatagcttgacatgaaaaacaaagcaactccaacaggtatcacaagggcaaaggacatgaacattttatcaaagaagaaatgcagctgtcaaaaatacagaaatattcaaccttgttcataataaagtggctgggctcagtggttcatgcctgtaatcccagtgctttgcaaggctgagacaggaggatcatttgaagccagaagttcaagaccatcctaggcaagtcagttcaataccagacttcatgtctacaaaacatcaaaaaattagccaggcatggtgatgcatgcctgttgtcccagctactcaggaggctgaggcaggagaattgcttgagcctgggaggctgcggtggcggtgagccatgattgtgccattgtactccagcctgggcaatgcagcaagactgtctaaataacaaaaataatagtaaagaaaaggattgggatgccatttacttgcgtattcaatacacagagttaaaagtaatttctacgttttctatttttttattactaaaaaaagctggaccattctcacagcctgaaatgcttctcactttcccttcttctgtccaaacacttctctatgataatgcaaacagtcactcctttaggaagacttcaccccaggtagttccagatccccttatctctgccttcccagaactcctggtgtctctccagttccctccgtgtggtgaagtaccctacctagggtttcagtatggctctgtctgcaaaggtcttgttcacaccttcccttatggttctgttgccctgtgttgtgtcatagcacagggcacagtggagaacccattcacactgatagagagggccccatggtcctggagataaccatgtaaccgatcagaataaggcattgagggctgggtgtcaggcgtgggctgcacttgggtgggcaggtcccctggaaagtcactgggtttggcaagcttcctagtaacatgtctctctggggtcccccttggaacttcatgcaaaaatgctggttgctggtttattctagagagatggttcattcctttcatttgattatcaaagaaactcatgtcccaattaaaggtcataaagcccagtttgtaaactgagatgatctcagctgaatgaacttgctgaccctctgctttcctccagcctctcggtgcccttgaaatcatgtcggttcaagcagcctcatgaggcattacaaagtttaattatttcagtgattattaaaccttgtcctgtgttgaccccaggtgaatcacaagctgaacttctgacaagaacaagctatcatattcttttcaattacagaaaaaagtaagttaattgataggattttttttgtttaaaaaaaatgttactagttttgaaaaggtaatatgtgcacatggtaaacactaagaaggtataagagcataatgcttttatactactaagaataatgttttctctaagttttttttggtagatgctttcatcagattaagaaaattccctgctattagttgttgaaggtttttatatcataaatgaaagttgaatattattatcatatattattaatatattgttattgaactatcaaagccttttcctaaaaccattgagatgatcttataaccattctcctttaacctgttgacgagatcattggtatttatactatttctctgttaaccattcttgagtctcaggtttaaattcaacttggtcatggtgtgtcatctttgatcattgctgtctgtggcttgctactgttttgtttaggatttttgcactgatgctcatcaatgagactggcatgccatcttcctttgcagtcctgatttttttctgatttggatcatgtggttatggccctcatggaatgagttgggcatgatgcctttttttcatgtctctggattgatgggacactttggattctctccagatggccctcaatggtccctgcctcctcattgttaggcccctgggcaagcccttctcatttctggtaggcccaggaacctgtgggggttttgtttgtttgtttgtttcttgagtcggagtctcactctgtcacccaggctggagttggagtgcaatggcccgatcttggctcactgcaacctccacctcccagattcaagcaattctcctgcctcagcctcctgagtagctggaattacaggcacccaccgacacaccctgctaatttttgtatttttagtacagatggggtttcacaatattggccaagctggtctcgaactcctgatctcatgatctgcccggcttggcctcccaaagtgttgagattacaagcatgagccaccacacccagtgaacctgtggtttttagaagctccccatgcatgtgaatgctgtgagcatcccaggatgacagccactgtgtgttcagctgttggaactgtgagaaagcaccagtgggaccttctccagcacctgcctgctgagttcatggaagaggcttgttggggagatgatgccctggctgactcctgaaggatggttaggaatgcaccagatggaagctgggttggacccactctatgctgaagaacagcttgtgtggacacaaggagacacggatatgtcatttttgtagagcctgaggagtgtccaatcacaccatttgcttaaaacatcatgcacacttggaaaagtggactgagaccgaatgaagaagctaacagtggccagatcagaaagggtcttgtgttacttcctagagatacttagattttatcctgtgggtgataggagcagttggagggactgaagacaaggaaagaaacatgtttcaagatctatgtttttcaagacgcttttctggtggctgagtagggaattccctggataagtcctgcccagggtcaggcaaaacaagttagggggttactgaaataaggagtatgagaaatggtgtaggttgtgctgacgttttgtaacacatctcatgatgatcttcatttccttcactaatttcctgtttcattaattcccttccacgtgctcttctgaaatttgcctcacattctctgatttctcttttacctgttggtttcatcaccttttactttttgctttcctggaaacacaaatgattctgattgtgacatgtcagaattatttgcaacatttgcctttctgctgaaaccatgagttcactgaatacacaatttagtaaagtgtaggatgcacatgtcgttttcgtggtcacaaccagctctgtagcattttataactacactggcagtgtgctgggaggtgtagagagaaatatttatcacatgtgtggctgacacaacctgccaagttattttaggagcctccttggaatcccagcaagaatgctaccggcacaatttgtaatcacagcatcctgctccatgccttggcttcatggcatagtcacttctgcaagtctctttccagctgtctgttcccatgtctataaagtatgagttaaatcatcctaacactactcatcttacaaagttttcttgctgatgttaagagagttgggaaagaactgtataaactgtgaagtgccatggagatgttagtggttactttatcaagaaatagacactctagaatggagtagaaagccaacagttatgattgagtcctcctcctcttcttctttttattaatttataaagaaaagaggtttaattgactcacagttccatatggctggggaggcctcgggaaactctcagtcatagcaggaggcaaaggggaagaaggcaccttcttcacaaggcggcaggagagagagagctcctgttcttttttgtcataaagtctacagaagtgcttatacttcaggacaagggcaggcagagagaaggaaggacattgcttcaccccagccctcactgacgagtttgctaggggacctcactttgtcccagagtagggcagaactctggccactacccattcagaaggcctgggctgcactgctagttcctcactaactctgtgtggccttgggcaaggttgggcctgtgttaacagattatgaccctgggctctcaagctagaggatctaaatttgaatcctggctctgctaaagcaattagtgatgtaaactttaatgggtcagttaaccttcctgtggcttagtttgctcatctgtaaaatagggatcataacagtatcaataccacatgattgttggacagattgaatcagttaatgcaggggaagtacttagcatgacacgtattcactatcatttcctggagtaagagctgtgtgtgagtgggtgtgagcatgtgtgaaaccttttctctgcaatctcagttaagaaaccaatccagaatttaaagttcagggcctaaatgggtggttatcttctcccagttccatcctatcccacctttgctcttcctcccgcccacaggagctgttggtccttgattgggctggaagacctggtggaccctaagtgatctataagaggagaatagagaacagggaatgtcttcaaaaatctagagggacacagaggctgagaggcaggcagtcctgcagggtcttctgattgggacaaggagaaccttggtcttcacaggccaattctggtcagtttcccccatggacagatgaggaaacaggcccaggaatatccaaggtctcacacttcccatctgtcaagtcttgttgattctgttgtattcatgtctctcaaagggagatagagtttagggaagaaagaaggatcaactgtgtctgataccactgggagcttaagtaaagggttcttttacttcatagcatttatcccaatttgtaattcagtattatttgtgtggctgtttggtgtctctttctcctatatgagtgctagcttcataagggcaaggattttgattctttaatatttagtgcttgccacatgccctgaacacagcaggcatacaggctaaccaacatacagtggcatgaaagtcatgaaagtgagacacctacctcctccagtgccaagagagcataaccatgcacctgtcactctcctcaacaccacccccaagcatgaggcccaaaagcattagctaatcccctcctccagccactaaaacttaaaggccaggtgtggtggctcccatctgaaatcccagaacttcaggagacagcagcaggaggatcacttgaggccaggagtttgagatcagcctgggcaacatagctaggtcccatctgtactaaaaattagctgggcgttgttgcatgcctgtagtcccagctactaaggaggctgaggtgggaggatcacttgagcccaggaggtggaaacaacagtaagctataatcacagcactgaactctagcctgggcaacagagtgacaccctgcctcaaaacaattttaaaaataaataagagcaaaacttagataccacgtggtcaccccaacatgcaaaatcaagttttcccctactgagaagaatggggacttgacagctgagttacagagagataatcttcttcttctttttttttttttggtttacatcctcaagatcatgacttgtgaaatttgaatcgaatacacatgtaattccagagcaatgttgcctccgcataccatcagcaattcacttggctactggaagtcaggataagcttcccagaagagaggtaccacttgggctaccaatataaaaggatgaaaatatcagagtgatggtgttctttacaacgttgagtccctggacagcctgtccactgatgctgatatctgagcctaatgcttctctgaatgttgagattgaactttgatccaatgaaactagaacgagaaagaagataagtctttcattgttgataaggacattatgtttctcatacttgtatgattatttttccttagctgtactataattatctgcttatttgtctctgctctatgtgcttagggtacaaagttgaccaagaccaactttggttggaagcatagtactaagagcacagtactgagagcacagtattgagagcacagctttaaaaaacatgatgaaggctttaatacaggaaatgagcaggggagaggcatgtggtggttggatgtatcttccttgacacagtcagtgcagctctcagtagtcaagtccctacatgttagaagatgttaccttctgtggaattaagtggcagaacttgccttcaattattttcctttgcagaacaacaccaactgcattagttaggacacagtgctggctgcatttaagtcccaagcgatgattagtctctcactgttggtatagattcaaaccaatcagaccacctcctaaagtttgtagggcaggtaaatcctcatcttagaataaaaatcatcttaccaagtatgtgttttagaggcaagaagaaaacatatttgtttctgtaagagttttgtttaaaaaaaatataagaaaggctctcggtttaggtgaggtaatgaagttgttgatagttatcagatgacactggaatctttacttctctgaacgtgttctgtgcatctctcagtgtgggaacatagagagggagatcctccagcaatgccactgatatggtcagaaactgcatctttctttctccctgctgagatgagatggagtcctttgttctagaagacccatggtggtgccgctgggagtaacccttgagacaggaacacaaatcccaaccaatttgtggttgcagccttgagtctcactatttcccatagtgatgcgtagcagggaatggcaggtgcaccagagcaggagaggacctaatatctcccttcctgttagctttttataaagttttattgtgatcagtagcagttgggaagctacttgcagtcactgagcctcagtttctacatctgtaaactggggatagtagcatggcccctacttaatgtgctcagcaaagccactgaaaggagacagaaatgtatctaaattaccctggacttttatcctacctctcttggggattgtcaccaccttcccatgtttgtcctttttggtttgatgcttgctgtcacttctttccttaggtgcctctctgtacggctcttttatcccagggattccagagttacagcacatgcataccaccatccaagcatgtttatttgtctcctgcttcactaggctgtccccaaggaacatgtggctcccggcacacacctggcacaacactgcacatgacattcacccacttggccttgaatctgacaaggaatctggcatgatgttcacccactcaggccaggtgccgagcagccctggaggcttaggggccagagggatgggaaaaggtgtctttctggggtgagtatcagtttctgcaggagggctgaatgtgagaaagaataaagagagaaggaagcgaacaagcacagcttaaacatcgcctatttctattgagttttaagaacgctgtgattttgtttgtcatgcaatccattcatcaggccaggcagacacagaacttgggtgtgagtgacgataatgagctgatataattttcacaccctcatcactgagatctctcccatcaggaatgggtcagggagctcacaggtggcagcaactgctattacaggcctcatctctaccagctcctggggcctgccctcctcccattagaaaatcctccacttgtcaaaaaggaagccatttgctttgaactccaattccacccccaagaggctgggaccatcttactggagtccttgatgctgtgtgacctgcagtgaccactgccccatcattgctggctgaggtggttggggtccatctggctatctgggcagctgttctcttctctcctttctctcctgtttccagacatgcagtatttccagagagaaggggccactctttggcaaagaacctgtctaacttgctatctatggcaggacctttgaagggttcacaggaagcagcacaaattgatactattccaccaagccatcagctccatctcatccatgccctgtctctcctttaggggtccccttgccaacagaatcacagaggaccagcctgaaagtgcagagacagcagctgaggcacagccaagagctctggctgtattaatgacctaagaagtcaccagaaagtcagaagggatgacatgcagaggcccagcaatctcagctaagtcaactccaccagcctttctagttgcccactgtgtgtacagcaccctggtagggaccagagccatgacagggaataagactagactatgcccttgaggagctcacctctgttcagggaaacaggcgtggaaacacaatggtggtaaagaggaaagaggacaataggattgcatgaaggggatggaaggtgcccaggggaggaaatggttacatctgtgtgaggagtttggtgaggaaagactctaagagaaggctctgtctgtctgggtttggaaggatgtgtaggagtcttctagggggcacaggcacactccaggcataggtaaagatctgtaggtgtggcttgttgggatgaatttcaagtattttggaatgaggacagccatagagacaagggcaagagagaggcgatttaatagattttatgccaatggctccacttgagtttctgataagaacccagaacccttggactccccagtaacattgattgagttgtttatgatacctcatagaatatgaactcaaaggaggtcagtgagtggtgtgtgtgtgattctttgccaacttccaaggtggagaagcctcttccaactgcaggcagagcacaggtggccctgctactggctgcagctccagccctgcctccttctctagcatataaacaatccaacagcctcactgaatcactgctgtgcagggcaggaaagctccatgcacatagcccagcaaagagcaacacagagctgaaaggaagactcagaggagagagataagtaaggaaagtagtgatggctctcatcccagacttggccatggaaacctggcttctcctggctgtcagcctggtgctcctctatctgtgagtaactgtccaggctcctcttctctgtttccttggacttggggcgctaatcacgcctctctttcccttatctggattgaagatcaaaaaagatgttcaggccgggcgtggtggcttacacctgtaatcccagcactttgggaggctaaggcaagtggactgcctgaggtcaagagtccaagaccagcctggctaacatggcgatactctgtctctactaaaaatacaaaaattagctgggcatggtggtgcacgcctgtattcccagttacttgggaggctgaggcaggagaattgcttgaacccggcaggcggaggttgcagtgagctgagatcatgccagtgcacttcagcctgggtgacagagtgaggctgtcttaaaaaaaaaaaaaaaaaaaaaaagatgttcaaggagcagtagcttaagtgttggatgctacaaacatatagaggttattgtagatcttatgcagctctataaaggaataaataagcatcttccccatccatctttagtggcaagaagggttttgggatagcattgattgaggatgatctacttgacaatagtttggacccaaggaggataaggaaggaaagtagtgacggatctcattccaaacttggctgtggaaacctggcttctccttactaaactagaatttggattttacattttcccctttatgttgcagtagaagaggatgaatcctctcactggtgggatcctgccatcctagagcaggtagagagaagagtcactccccactgtgggtagtggaggcttctcacatgtcacatttcacttctacctcaatttcactcttactaagatttgggaatcataatgacaggaaaatagaaaatataaacctcattttaattctttcacagaaaggttagaaattcagtgagttgtggcaacatattttccatcttctgaccttttaacactaattgatatggcttaaattcattctattttaaaccagatttttttggagatagtctatttccaacatgttccttctaggtgacaaatgagggctgttagttcagtatttgttacaataaatgtgtgtaaaataacctcacctttccagaatcatgtcaggaatatgaatctaatgcacaaatgtataactctatgacaagattgcatatatcttttaaaatataccttcccaacgttcattttaatacccctatttcaaacaaacctgcttagcaggttatgttaaacgctcagggcagaggagtaagcaagactgtgagccagtgatgacagcaaaagcatccaggtaggatcaaaatggagtaagaaaatattcctcatccctcagggtagaactccaaagagatattcatgggtcctggccccgtagtggaggtcactcaaaggacaaacatgtttgcatctcatctgcttgaagcctggacacagaggcaccatctgtgtcactctgtgtgtggtctgccatgttgtggggtggtcactacagactcacagactaggcagctgggcagacaataccttagccttagatgatgctgatgcagcccaggagtcagaaactgtagtgcagacaatgccctccttaggccaacacaattaagtgcaatagatgactggcttttctgttagcctcttcattggaaccaaaagcagcattactctaccaaacagaggggagctggaaagaaactacacagtttgcccagcctagcctctgccttgacacggaaccatgtgagtctagacattcacctagatcattccttggggaccaatgctactgacacattaactcaatagtttgtcctggcctgagaggtcatgtaacttgtagaaagtttagaagcagagattagtgtcatttatttgccatggctgtgacaacaaaggaaggaacaggagtgggaaaacccaaggccaccctggttttggtagatggtgcacacgcttccactagctgttctggggcaaagatccaaatgcactattgggcctggctatgctgcttctgctgggtcccccaaacatgagcctccacgccatttctcagttgtattttaccacatattatcacagtcaccggatttgtacagaatatttggaacctatactgtcttaagggctaccctttaaagaagagaaaacaaggttttaattcaactgtctggaacattttatgtttacttatgtggaatactacatcttttgttataaacaggagggaatgtggacattcgaaggcccctaccttttagctaaaagcccatatgaagcatatggatccatttatacacaccatgcttttcagctacattttcctaatttgcctctctggggccaaccttgtgggactagcagattcatggttgagttgaaggatggtgacctcttccatcagcttttcttcttcctccagtcttccaaccctcagtaacatcagactgggaaggtcttcagacatccagaaaccccagttcggggagttcatacatgacccatcaaagatgagttgcaagcaggcctgccttagggagcaccagccttaatgggttttcctacagagatagttgatgggcagatgcaataaactgactgctttgtgattgaccaccttgagaaaaatagcatgtctggctatgttagtcttttcttgcattgttataaagaaataccagagtctgggtatttataaagaaaagggatttaattgtctcatggttctgcaggctgtacagaaagcatagtgacttcttcctctagggaggtctcgggaaatttacaatcatggcagaaggtgaaggagaagcagacagatcttacatggctggagcagaagcaagagaggctggggagaaggcgccacacattcaaaccaccagatctcataaaaactcattgtcacgaggacaacacctaaggcgggatggtgtgaaacgatgagaaactgcccctaggatccaatcatctcctaccaggccccacctccagtattggggattgcatttcaacatgagattttggtaggggcacagattcagaccatatcactggcactgtgctaatcagatgaatatcaccagttggaaggctagattccacaagaggaggaatgacctggaaattggttctttagttgtgattcttctgcacactgtcattcagggaaatatgagtcaatcatccttcccaataggtcaaatcaaccagatcatctgatcatagagactgaggtgtagctgaaagctgctcacatttctatgaggtcaatggaagccttgagcacagttgtcaatctgtagaaataaggactctgtgactcctccaagacctctctgtgaatgacggtttaagaagaaccagatcctaaaacagggtcagagcttagagggaagggaaagcataaaagcctctgagcaaattctaaagacagggtcaccataggctctcagtgaccctctgtgactgagtggctgcagtgatgcaaaatctcatcatcactgcggaagacaaaaaaatgtcaccctttctacctaggatgagaatccccaaatttggggagaggccacttactaaatagacgtaaaggaacaaagtgacctggaagaattcctgcctgaacctctcaggatcattcacatttgagaacatttatcaaatattcattccaggactgggaccatgaagacttcagctgctttgagctaatcattgtaactttttggtgtctcatggtggaggcaggaaaggacctgatgaacaaatataatcattgccgtcagagttactgttattatttcttgccttaatgttacctcgttctcttgagcattccagttcctcagtcaatgactcatcaacccctatatctataaagtcacaatcgctgtgacttgatttctgtttcactttgtagatatggaacccattcacatggactttttaagaagcttggaattccagggcccacacctctgccttttttgggaaatattttgtcctaccataaggtgagtgtttttgagctcactcttttgcttcttatgattgccaagagcagcttagttccatcagtaaaaatgcttctcctcagggggaaggtctgaagttttacactttcagaaacagtgtgtaggcatcacccagaacatggcaatgtttacccaagggctctcttgctaactctcaggaacctcaggtttgcctcagttgaacagcccaaatctcaggtagatcagcaacctgatgctcagaacttgatgtgcaaactttgtgagcgcgccataaagaaggttttctttttgctctatgcaggttcccaggaaggtacagtcatacttagttagtattaaaagtaggaaaaggaccccctgattgtgacttgttatctgtgtacatgagagtcaaactttcctctctgtagcaagagctcttgagtagccttttttcccttccttctggacagctttgaaactcaatttattagggagcccaggtatttacctttgggtcatttgcacctgcttcaatcctctccgaaaaatgctaaattccctgagggtatctccatggctctaccccagctcttactccccatgacatcttttgcagctttgagagacagaacaaaggggttctgctttatttcttttccttcaaaggctgcaccttatgggtgagactgatcacagactggatataactgagatgagacagtggtggtcaatcaaactcagatatttaagcacaaaatgagtttgtggggtttttttttacacaaaggcggaatcacatgcaaaatcactagaaaggggatgatttgatgaaattattaaatatttaattttgctgaatagaatataatatgtgccacgtggaccgtgaactttggccgagcctttgtgcctaacctagtctgatgctctgcccagggtctgggccctggatggaaaattaggagcccatgtccacatggccagccgcagcagtcagctctttggcttacatcatctttcccacatacctgaggcttgtttctcagattctaattctctcaggtgagggctttgttgtctaattactatccaggaatttcatattttttccctgtgcaaaagcaataatttccccgccaccttttccaggtcaactctttagtagatgtacccccaagatgcacattcctgggacctttgtttgcacagttaaaatgtcacccctgaaatgtcgatacaggaaggtttgtttttaagtttcagtgaaaactctgagcaagtgttgtaatttgctgtgtccgatgtgtagaggggacattttctcagaacttccatgttaagctggaaaactggaaagtgagttcactttgtcattctgtcactcgttcattttctcactcaacaacatgcctcatacttacctaaatctgctagactaaaggagttccctggtgtctgtactttccaattctgctagaactctagagcgagctcatgaaataaatgaaaaggatgacaaagagataaaacactgtgcattctcttctgatgctaattcactttcccttggcctcagtttccccatgtgcccctggaggtgatcattcagggattcatgagattttcaagacaacacatgaaaaagcaaaaagacatcagaaagacaaagaggtacttagtatttatacacaaggataagtcattcagtatccacaacacttggagagaattcaagagtgattttaaatttcccttttcaaatacctcctctgttttctcttatttcctttatgacgtctccaaataagcttcctctaactgccagcaagtctgatttcattggcttcgactgttttcatcccaattagaggcagggttaagtacattaaaaataataatcaaatattattttgtttctcctcccagggcttttgtatgtttgacatggaatgtcataaaaagtatggaaaagtgtgggggtgagtattctggaaacttccattggatagacttgtttctatgatgagtttaccccactgcacagaggacagtctcagcccaaagcctcttgggatgaagctcttgtcaacctaactacaaacagagagaagttctctgaaagaagaagatatttatttgggtgtagagtattgcaatgggaatctgcatgcctttataaactatgtgcaaattcagggaagtaaagcaagacaaagaggctccaaggaaaatatgaggaggatttcttatcagttttgaaataattatccttcgctacaaagatcagtaacaagggtgacgcctcaccaaggttggacaggcagttgctgggcaggtgtccttgcagaaatattttttttaatgttgggatggcctttgtgcaagcttgtagttttgcggagtcttttgtgatagttttgttatcaggcacacaagcatgagaatcctctcttcatagccttctttgatttatttgtcagggtttttacacacacacacacacacacacacacacacacaactagtgacatcattttggttctaacaacattcacactggttattgtaaaacttttcgaaggttgtcctaccaaggatcccatgtgtcaccaggtgtcaagttctacagtctgaactaggctgggagcattgtgattacttttctccagactttggtggcccagggactcacagcatcatgctctgtccagtgtctgcctattcccctcttctttttttttttccttaggtgcccttttattacatgtgttgtctcagacccttctaatatgtgctcataaatacatcatatcatctccttcccacatcaattcactttcaattaaaagccaaaactctttcatttagactttggatttaaagtgcttttgaatgaagggttgagagataatagagaaatagattggcaaaccatttatactctgctgttgttgttttttaattttatctgcaagtgtggaacttttcattctgttttgttattaaatttaagccaagactttttaatagaagggtatataagcatttctttgtctataccttcctgctgaatttgaagaaatgctgaatattcttaaccactggcgggctgatggactgtgattttattttattttttatttttagttttttaaattataccttaagttctgggttacatgcatagaatgtgtagttttgttacataggtatacacgtgccatggtggtttgctgcacccatcaacctgtcacctacattaggtatttctcctaatgctatctctcccctagccccccacccaacaacaggccccagtgtgtgatgtttccctccccgtgtccatgtgttctcattgttcaactcctacttaggagtgagaacatgtggtgttgagttttctgatcttgtgatagtttgctgagaatgatggtttccagcttcatccttgtccctgcaaaggacatgaactcattgtttttttatggctgcatagtattccatggggtatacgtggcacattttctttatccagtctgtcactgatggacatttgggttggttccaagtctttggtattgtgaatagttctgcaataaacatatgcgtgcatgtgtctttatcatagaatgatttatgctttgggtatatgcccagtaatgggattgctgggtcaaatggtatttctagttctagatccttgaggaatcaccacactgtcttccacaatggttgaactaatttacactcccaccaacagtgtaaaagtgttcctatttctccacatcctctccagcatctgttgtttcctgactttttaatgatcaccataccacctggcatgagatggtatctcattgtggttttgatttgcatttctctaatgaccagtgatgatgagcattttttcacatgtctgttggctgcatagatgtcttcttttgagaagtgtctgttcatatcctttgcctattttttgatggggttgtttgctttttttcttgtaaatttgtttaagttctttgtagattctggatgttagcccttcgtcagatggatagattgcaaaaattttctcccattctgtaggttgcctgtttgctctgatgatagtttcttttgctgtgtagaagctctttagtttaatcatatcccatttgtcaattttggcttttgttgccattgcttttggtgttatatttatgaagcctttgcccatgcctgtgtcctgaatggtattgcccaggttttcttctaggatttttatggtcctaggtcttacatttaagtctttaatccatcttgagttaatttttgtataaggtgtaaggaaggggtccagtttcaattttctgcatatggctaggcagtttcaccaacaccatttattaaataggaaatcttttccccattgcttttgtgtgtcaggtttgtcaaacatcagatggtagtagatgcatggtgttatttctgaggcctctgttctgttccattgatctatatttctgttttggtacctgtaccatgctgttttggttactgtagccttttagtataatttgaagtcaggtagcgtgatgcctccagttttgttctttttgcttaggattgtcttgtctatgtgggctcttttttggttccatatgaactttaaagtagttttttccaattctatgaggaaagtcagtggtagcttgatggaaatagcattgaatctataaattaccttgggcagtatggccattttcatgatatggagtcttcctacccatgagcatggaatgttcttccatttgtttgtgtcctcttttatttcattgagcagcggtttgtagttctccttgaagaggagaacttcacatcccttgtaagctggattcctagatattttattctctttgtagtaattgtgaatgggagttcactcatgatttggctctctgtttgtctattattggtgtgtaggaatgcttgtgatttttgtaaattgattttgtatcctgaggctttcctgaagttgcttatcagcttaaggagttttggggctgagacgatggggttttctaaatatacaatcatgtcatctgcaaacagagacaatttgacttcctcttttcctaattgaatatccatttctttctcttgcctgattgccctattcagaacttccgacactatgttgaataggagtggtgagagaggacatccttgtcttgtgccggttttcaaagggaatgcttctagtttttgcccattcagtatgatattggctgtaggtttgtcataaatagctcttactattttgagatacgttccattgatacctagtttattcagagtttttagcatgaaaggctgttgaattttgtcaaaggccttttctgcatctattgaggtaattatgtggtttttgtcattggttctgtttatgtgatggattacatttattgatttggtattttgaacccagccttgcatcccagggatgatgctgacttgatcctggtggataagttttttgatgttttgctggatttgatttgccagtattttattgaggattttcgcatcgatattcattagggatattggactaaaattctctttttttgttgtgtctctgtcaggctttggtatcaggagatactggcctcataaaatgagttagggaggattccctccttttctattgttcagaaaaatttcagaaggaatgataacagctcctctttgtatctctggtagaattcagctgtgaatccatttggtcctggactttttttggttggtagcctattaataattgcctcaattcaaaacctattattggtctattcagagattcaacttcttcctggtttagtcttgggagggggcatgtgtccaggaatttatctatttcttctagattttctagtttatttgcatagaggtgtttatagtattctctgatggtaatttgcatttctgtgggatcagtggtgatatcctttttatcattatttatcgcatctatttgattctgcttttttctttattagtcttgctagcagtctatcatattttgttgacctttttcaaaaaaccagctcctggcttctctcattttttgaagggtttgttgtgtctcaatctccttctgttctgctctgatcttagggctcagtcaacgtagcatagccacagtaactcccgcctctcgatggtaaggggaacgttacagagtcaggaatcgacctatcctgcattctctggggggcaatatcttagttatttcttgtcttctgctagcttttgaatttgtttactcttgcttctctagttctgttaattgtgatgttactgtgtcaattttagatctttcctgctttctctagtgggcatttaatgctataaatttccctctacacactgctttaaatgtgtcccagagattctggtacattgtgcctttgttctcattggtttcatagaacatctttatttctgccttcacttccttatttacccggtagtcattcaggagcaagttgttcagtttccatgtagttgtgtgattttgagtcactttcttaatcatgagttgtaatttgatttcactgtggtctgagagacagtttgttgtgatttcttttcttttacatttgctgaggagtatattacttccaattatgtggtcaattttagaataagtgcgatgtggtgctgagaagaatgtatattctgttaatttggggtggagatttctgtagatgtctattaggtcctcttggtccagagctgagttcaagttctgaatatccttgttaattttctgtcttgttgatctgtctaatattgacagtggggtgttaaaatctcccattattattgtgtggaaatcttagtctctttgtaggtctctaagaacttgctttatgaatctgggtgctcctgtatttgggtgcatatatatttaggatatttagctcttcctgttgaattgatcccatcaccattatgtaatgcccatctttgtctcttttgatctttgttggtttaatattctttttaaaataaaatattttaaacatatgaaacattatggagaatggcatgggaaatatccacgtatgcaccacccagcttaacaaatgctctactgtcatttctaaccatggtctctttgaagagctcttttgtctttcaatgtctcttccttgtttggcccacattatccttcatcatatgaagacttgggtggctcctgtgtcagactcttgctgtgtgtcatacctaatgaactagaacctaagattactgtgtattgtacaactaagggattatgtaaagtcaggatcaaagtctggcttcctgggttgggctccagctgtagaataaggctgttgatgtttaatcaactctgtttttttcacacagcttttatgatggtcaacagcctgtgctggctatcacagatcctgacatgatcaaaacagtgctagtgaaagaatgttattctgtcttcacaaaccggagggtaagcattcatgtgttgaaattaaaatactgattgattaaatttatattttgaaattcttatatattcatagacagttgcctaaaaaatgtccaggaaggttccacgtccacttcatcctgtcccccccgaatggtaacatcttgcaatcttgcataactataaatacagtatattcatgttactatatagtacaggaaattgacattgatacagttcacagagcttattcagatttcaccagctttatgtgccctcatttctgttctatgcaaatttatcacaatcatagatttgtgcaatgaccagcatgatcaaaacgcagaacccatttgtgttctatacaaatttatcacatcattagtagatttgtgcaatgaccagcatgatcaagatgcagatccactctgtctccatgtggctccctcctgctatcctacagtcacaagtctttttctctgacagggtccatatcagagcaaactattttttattttgaggtgatcaatgtattaatatttccttttctggattgtacttttggtgccatgtttgaaagttctttgcctagccctccttcctatattgctgtttttttctaaaagtcataaagtttaacgctttactaaatgtagctctataatcaattttgtgttattttttgtataaggtctgagatttagatcaaggttcattttttttgtggcttatggctgtccaattcctccaacgccatttttggaaagatgggtatattgttaaaaatcaactgggcatacttctttgcatctctcacgtactactgtgtcccattgatcactgtgtttattattccaccaataccacactgtgttgaccctagtagctgtacactaactcttaacctcgtgtagagtgattcttcccacttttattgacttattttttcagatttgttttaactcttcctggtcctctgcttttccataaaaattcagaatgagtttctcagtgtctacaaataaacgtgctgttattttgacaagaattgaattaaatatatagaccagtttatggaaaatgtgtatctttactgtttgatctttcaattcatgaacatagtatgcctctccatttccttagattttctttgattgcttttaacatcttgtagttttcagcatagagatcctgtacatgttttgttagatttacagctaaactatttcatttttgttggaatgaccgtaaatgatattatgtttttcttgtattttcagatattgattgttgttacatagaaatgtgctttgattttgtgtgttgatctggtatcctagaaccttgcagaactgacatagtagttctagaagtctctttgtatatgccttgagattttacacattgtcagttatgtcacttgaaaatagagacaattcttttatttcctttccaatctgtatgccttttatttcttttcttttgtctattgcactaggacttcccagtataatgttgagtaaatgtggtgaatttttgaattgttcctaatcttaggagggaagccttcagttctttcatcattaagcatcatttagacgaagtgggctgtttttttgtagattctctttatcaaattgaggaattttctctccctagtttgctgagtttttatcataaatgaatgctggaagtactcattataaaaaaaatggctatggaagatgaaagaaagtttcaagcatgttggcttgataggccagttccaagttggcaaaaataattatctcttttttctttctatccatgaaataaaaaattaagagccaagaatgtttatggaattgcattatttcttcaaaatatgttcctagttttaaaggtattacctactatttcttttaaaaccatcacattgaggcacttctttttcacgttgcccatgctgcaggagaacataaagacagcttgtctgaggcaacatacaatccaccaaagtcacctgcttgtctgtctccactccgtctctacactgcagaagtgctaggtcttgattctgtttattgtactggaagaacacattctctaccacgtggataatttgcatgtaaaagaagactgggatatagaggctggagacgacatcagggtctcctgaacactgccgccacccccccgccccgccccacacaaatacatcccaggacactgggcatctgggataaatctctattgagcatctagcatagggcccatcacccagtagacagtcactaaatagttgttgaataagtgttcctgtttaacacattttctacaaccatggagacctccacaactgatgtaggacaaaatgtttctgctttgaactctagccttttggtccagtgggatttatgaaaagtgccatctctatagctgaggatgaagaatggaagagattacgatcattgctgtctccaaccttcaccagtggaaaactcaaggaggtatgaaaataacatgagttttaataagaaacttaaagaatgaatctggtggggacaggtataaaataagatcacagtccctttccaaggggtagtccactgaatttgagctgcctaaaaatggtcttttatctttatgtacagaaaacacatcacaaaattcattataaaatgtcacttactgctccatgctggggaaagccatgtccttctgggactagagtctgcacatttaactatgggtggtgttgtgttttgtgcttagatggtccctatcattgcccagtatggagatgtgttggtgagaaatctgaggcgggaagcagagacaggcaagcctgtcaccttgaaagagtaagtagaagcgcagccatggggttctgagctgtcatgaacccctccagctgcctgccatggagctgatattcctgctgttgggttattccagtgaccagacaaaaggagggctgtggtaatgcaacttcaatgggtctcccaagatggggcagctccgatgaggaggtggggcagctggaggaaaaggatcttctcccctgtgcacaggggccagggtttacatatccattaaattgtcaccttggatattctagaagactaaatatatcctttagggggaaaaagtgtgattgtaccaaagttttaagcatggagtgtatgggatggtggaaggggaaggcacttggtatctgttggttggcagtgagtaggttgggagagttataatggagaacttagaataactttgatcatttcatgtttttttctgaggatatcagtagaatactaaatattaaaattcctaccatttctttttcctccagtctcaaagagagagggtggtaaaaacactataggtagggcaagcctattatttgctatctacacttatgcagtaaaaacaggtgtaatctgagtttgtcctgggcagaccagggatatgtggtcactcactatagaaatttccaaatcaaattttgagagatttttttttaaccaggacattattggtcattatattttacaaaaataattctgctgtcagggcaacctcagctcaccacagctggggatagtggaattttccaaagcttgagcagggagtatagagaataaggatgatatttctaggagctcagaacagggtactgttgctttgtaaagtgctgaagaggaatcggctctgggcatagagtctgcagtcaggcaatatcacctgtcttgagccccttaggaagagttaattattctactcttgttctgctgaagcacagtgcttacccatcttgtatcatccacaatcaatacatgctactgtagttgtctgatagtgggtctctgtcttcctatgatgggctccttgatctcagaggtaggtctaattcagttcagtgtctccatcacacccagcgtagggccagctgcatcactggcacctgataacaccttctgatggagtgtgatagaaggtgatctagtagatctgaaagtctgtggctgtttgtctgtcttgactggacatgtgggtttcctgttgcatgcatagaggaaggatggtaaaaaggtgctgattttaattttccacatctttctccactcagcgtctttggggcctacagcatggatgtgatcactagcacatcatttggagtgaacatcgactctctcaacaatccacaagacccctttgtggaaaacaccaagaagcttttaagatttgattttttggatccattctttctctcaataagtatgtggactactatttcctttaatttatctttctctcttaaaaataactgctttattgagatataaatcaccatgtaattcatccacttaaaatatacagttcagtgatttgtagtacatttgaagatatgtgtgaccatcatcattttaaactttaaaactttttttgtcaatctagagacctcatacatttttagctatcagccccctgtcacaaaccctgtcatcatatgcaaccactaatcaactttctgcttctatggatttgcctattctggacacttcatagaaatgatattaattcatcagggttttttattctctagttcatgaatttgtactttagtctgtatcattttctttcttctgctggcttcaggcttagtttgcccttcttcgtttactatgttgtggcatgaacatagattactgatttgtgatttttttgttcctctaaatttagacattacagctgtaactttccctctgagcacttcctttgctaaatcccatgagattgtggcctatcacatcttagttttgttcacctcaaaacagtttctatttgccctttgggtttctactttgactcattgggtacttaaatgtttattatttaacttccacatatgtgtgagtttctcaattttctttcccttattgattttatctttattccatgataggtgacagagatatgctgtgttatttctatcttgactacctactatttcttgaacagcaagattaattttgagcttcagattatgatttgggttattctaggagactgtagtccaatagataaaggcaaagagattagggcattgaattttgttccttttatccttcaaaagatgcacaaggggctgctgatctcactgctgtagcggtgctccttatgcatagacctgcccttgctcagccactggcctgaaagaggggcaaaagtcatagaaggaatggcttccagttgagaaccttgatgtcttttactcttctggttggtagagaaaactagaattgctccaggtaaattttgcacattcacaatgaatttctttttctgtttttgttttgtttttcctacagcagtctttccattcctcatcccaattcttgaagtattaaatatctgtgtgtttccaagagaagttacaaattttttaagaaaatctgtaaaaaggatgaaagaaagtcgcctcgaagatacacaaaaggtaaaatgtggtggtagttataggaggatgtttagtttttcataattttttagataatatacatatgatcagtgcagttacctgtatgtttttaaagaatgcttttaacatgaagactgctcatgtttagagcaagagaattcatttggtagaaatgcagaaagtggggtttggggaaggagatatgagaatgagtcagagacagcacatgaaatttgatatcagacacaacaattagtatgccacggcataaattttattgagataaaactttaccactttacttcccttcaataaattgtcagaggataaacattactgtttggaaatatattttactgatattatgctttccccagatcataaattggtaaagactcatttcaagtacaaacctgttattttacacattctcaaatgaaagtccctatcaggccacctgctgaagtgtacgtgtgttaaatttgagccatctcacatgatagccagatttgtttcaggaaaacatcctgctttccaaggatttagaagggtatgtttttcactggtgattcaggcaacatgcatcagatttctggtcttcaaggagccatattctcagaagggagatcaaggaccacgctgtgattacttctgacttcaggagccactttctgtcagtgaaatttctctttttgcttctagcaccgagtggatttccttcagctgatgattgactctcagaattcaaaagaaactgagtcccacaaaggtaaccagagtgtttctgagggctacttgtggggcactcagagggaaggccttgttctgaaaatgtgcaggaagtattccaggatgatgagaatttctgccacatagcagaacgacacatgtttgaatgttataagtggtagttggaggcactttctagaggcatgcaggcatagatagccatgttctaagagtaaagggcaaccctaagcaaacctggcatgctagaaagtcagtctgcggtctgtggatcacctacatcagatcaaatgccaattctcagcctccttcagatccactgaataaaaatttctgcctagaaatttattaggttgctgcaaaattaattgtgggtttttccattacttttaattgcaaaaaaatgcaattaaaagtaatggcaaaaaccacaataacttttgcagctacctaatacatctaacatccaatcaggagccacgctgttccaaaggggtgattttaactggcagtacttgtgaaagtgtgttcaccaggtaatctactgcaagtattttcactttggaatggatagtaactgtggggaaaacctctgagaccgtgtaaatatcctctttgtcttcaatgttccacctacgagttttacaacccatgtatgttttttactgaagtcactattactatgacagtggcaaaatgatgaccatggtcaattacaagccaccaagacttggcaaccatctcacaaaattcctgaatatttaactattggttctagagagcaggactgggcttactccagcatactgctttaaatatatccatgtctacatccacttttgtctgtatgtctatgtatctatctatgtatctatctagctatgtatctatctatctatctatcatctatctatctatcatctatccatctatcatctatcatttatccatctatcatctatctacttatatattatctatctatcatctaactattcatctatctatcccaatataacttgctgtgataaaggaaatagtctattgttttactgtttcatatagaaatcactagacacatatggctattgagcactggatatgtggctagtgccattgaagatcaattttaaatggtatttcagtttaatttaataaaatttgattttaaatagccactagtgggtagtggctaccatattggacagcagagctctaaactttgagattatagttcaatttcacatcagtatcatcaggttcatgataactgaatactatgattaggtagttgaatttacttataactgcatcacagaagtcttcactggtaaatcacagctctgtcgacctttctcacactcctttcatattggttttggttgtgaattacatggttggagcaggcattatatttatttctatgttccaggtctctaaaggtcctaatccagtcctgatcaaacagaccagtgatggaccatcctgagcttctctcaggagagaaatcaagaggggcccaacttgtaatcataggagcttatgctattttaatgccatccatcagactacaatcaattaccactcatctagctttttgtccatctctcattcttgtacatcctgagatagtcaattctgagaactgtagcctagatctatcacctgatgcctctcaaagatataatccgtgcttctcaagctaggctatgcacacaaatcactgcatcttgtgaaagttcagattttgaatcagtagttcaagggtggggtttgagattttgcatttctaaatgagctctcaagatgcttctgacccatggaccacactttgaataccaagaagtggtctgtagaccaatattggtcccttaagttccctcaaacatatcttcgggaaacgtcctttgattttccctacatttaaccattagtgttgcaaattctctcaaagtttgtcaagatatattgtagctaaaataaattacatttttcttgggggagagtactacctcatattaacttacaataaagtacttttaggatcattcaaggaacacacccataacactgagtatgttatgcggaaatgctctctctggaaattacacagctgtgcaggtggcgggggtggcatgaggaggagtggatggcccacattctcgaagaccttggggaaaactggattaaaatgatttgccttattctggttctgtaagatacacatcagaatgaaaccacccccagtgtacctctgaattgcttttctattcttttcccttagggatttgagggcttcacttagatttctcttcatctaaactgtgatgccctacattgatctgatttacctaaaatgtctttcctctcctttcagctctgtccgatctggagctcgtggcccaatcaattatctttatttttgctggctatgaaaccacgagcagtgttctctccttcattatgtatgaactggccactcaccctgatgtccagcagaaactgcaggaggaaattgatgcagttttacccaataaggtgagtggatggtacatggagaaggagggaggaggtgaaaccttagcaaaaatgcctcctcaccacttcccaggagaatttttataaaaagcataatcactgattctttcactgactctatgtaggaaggctctgaaaagaaaaagaaagaaacatagcaaatggttgctactggcagaagcgtaagatctttgtaaaacgtgctggctctggttcatctgctttctattactacaataatgctaagtaaaaaacctccaaaaacctcagtggcatctaacaataagcatttgttgctcacactcatttcaattggttttggttgtgaattacatgtttgcagcaggcaccatagtggtgtgtgatgtccccttagctgtatccacatatggacacaggaattggctctttttatctctttttattttcttggttacagacatgtgacttttttttttgaaaggtaacaatcactttctcatatgttatttgatgctagtggtcatagcctatagtcacatttgtttcaatgagaaagaaaaaccagtacacggttatgctaaggatttcagtccctggggtgagagccgtctcgaatgtctccccacttcataactcctccacacatcatagttggatagtgagctctgctgatattggcaggacttgctctggtctggctgtagtctgacggagcctggccctgggtgtgctgtgcaggctgactcagctctccccacacctatctcatgttccagtcaggcagtaactggtgaagaagccaagctaggaaccaggatatctggctcctgagctaaagtcttaaaacactatcatattgccttccaaatataacaccaaatactaggtgcatatcaccctcactgttttcagacctctgccaaaattgggattctttgtggtatgaagagacacggctttggggctggcccggctgtgacagtgaggtgaacacaaagggatgttcttcagagattacagtccagccctgaagcaacaactaggagactgattgagcaagtgaggacagggctgtgtggggttctatcctctttataacttccactagcactgaaatcgtgttctctggttacatccaaccagagccttctttgtcatttttgggatagaaagggactagtttatcctcaaattatttatggagattttatataatatagtgtttctctccacattttctgtatataacaaaagtcctccttttagtgtgtgtatacacatatatatacacatatatatgtgtgtgtgtatgtgtgtgtgtatatatatatacacatatatacacggttatgctaaggatttcagtccctggggtgagagccttcccgaatgcttcccaccttcataactcctccacacatctcagtgggccactgagcacagcaatgggcatgacagttattaaaacactttataaatgcttcgatcctttaccagtatgagttagtctctggagctcctaatacttcattagtactgcatggactgagttaaaagttaattcaaaatctcaatttatccaaatctgtttcgttctttccaggcaccacccacctatgatactgtgctacagatggagtatcttgacatggtggtgaatgaaacgctcagattattcccaattgctatgagacttgagagggtctgcaaaaaagatgttgagatcaatgggatgttcattcccaaaggggtggtggtgatgattccaagctatgctcttcaccgtgacccaaagtactggacagagcctgagaagttcctccctgaaaggtacaaggcccctgggaagggagccctccctgaaccagcctggttcaagcatattctgcctctcttaatctacaggacagtcatgtggttgtataattatttgcttgtatttttatatttagagatttttttaatcatcaaattgattattgtcacactttacaaaccatagactagaaaaaagaaaactacagtcatccacaattccaacaacttacgatgaaggtcatcagttatgtccttatgggtcatcagtgtccaaaatgtaaggactcttttaaaaacacatgatcacaatgctattattatgtcccccaaatgaatatttttttcataaatataatcaaatatttaggaataacattttaataaaaaacatgcatctaatcttcaaagaattttatagcttactggaacagatagacaaagaaagcagggatgatactgcattacatcggtacagtagcatcatatgcctgtgtaaattatctgacttcaactattctatggaggtgtgggggagaaagagggagagatggagagaagaagaaggaggagaaggagggagaagacaaggtaaggaggagaaggaggagaattagaaaaacaagagaggagatgagaaggaaagtgcaaaataacaattttgaaatagtacaagacaatttccccttctccttcctcatgaccaatgtaagtgtgacttgaggcaggaacctacttttccatcagtcagtcccatcacttatgtgccttttatagtgtggacacatcaccaccctgaatataatttcagtgtttagaaataagtattctttgcaacactatttatctcatctcaacaagactgaaagctcctatagtgtcaggagagtagaaaggatctgtagcttacaattctcatagcaaaataagcatagcaggatttcaatgaccagcccacaaaagtatcctgtgtactactagttgaggggtggcccctaagtaagaaaccctaacatgtaactcttaggggtattatgtcattaactttttaaaaatctaccaacgtggaaccagattcagcaagaagaacaaggacaacatagatccttacatatacacaccctttggaagtggacccagaaactgcattggcatgaggtttgctctcatgaacatgaaacttgctctaatcagagtccttcagaacttctccttcaaaccttgtaaagaaacacaggttagtcaattttctataaaaataatgttgtattaataattcttttaactgagtggtctgtattttttaaaaagaatatgcttgtttaatcttttactaatttgttctctgggccaaagaatcaattaggcccatctgtgatctttaagggtgcttcagttctggagttcaaaagctgtagcatcaaaaacatcatgtaaagcccatgtagattagcatggcataattatctgcagtctccttgaacttgagcaaagttacattcatttcaagtcgattggaaagatggggaagtatttttgcacagtcatgaagtgtaatgattaccttgttgtgacttttgaatgctgctcttcccaaccagaacttggagaagctttctcatgagagtggctcccaaccactagctgtacattggaatcaccagggagctttaaaaattcatgatgtctgggatatcacagaaattctaaactaatttgcccagagtgtggctttaaaagcttccccattgcttctcatgtgaagccaaggttgagaatgactaatttaaggcatttctggtggatataaaggactaccacagtccaaggccatcctgactgacctcaccttccaggtgcctagctccatccagctgggctccttttcaacccaattataactctattaatgttgttcccagccaggcatggtggctcatgcctgtaatcccagcactttgggaggccgaagcaggcggatcatgaggtcaggagatcgagaccatcctggctaacacggtgaaaccccgtctctactaaaaatacaaaaaattaccctggagaggtggcaggcacctgtagtcccagctactctggaggctgaggcaggagaatggcatgaaccccagggagcggagcctgcagtgagccgagattgggccactgcactccagcctgggtgacagcgagactctgtctcaaaaaaaaaaaaatgttgttccctttctcctcattttgttcttatctttcaagtcctagttcaatccccaagcccctccaaagtgtcttctcctcctagtccagggcccatttacttctctgctctgttattggatactggaggctattatcataaatttgacaatttgccattaaatcattgagttttattctctatattttctttgtatctaaaatgtcttcccccctccattaacaatatcctctcattttattcctttttaaaatatcccagtggtgccttgcaagggactgtatctaatgcaagcatttggtaaatgtttaaggagtgatgtgcagttgatggcttgcatacatatattaagctatttaatgtgaacctttaaacaaatgccattcgtgcatatgcatgtgtgtgtgtgtgtgtgcacatgtgggcatgcatgtctgtctgcagtgaaaatatattcagggtttttgaaaatttttaaataataaggtattatatttatagaaagatttgaaatattttctctgaagaagttaaagaacagacgtcattgattcatattaaacaataccctataaatcttatttctaggtctcatgtatttattattcaattccaccccttaagtgggctttctatataggagaggaagaagacagaaatagtttccatattatttccatattccatattatttgtggctttaggccagcagtgtagctgtattatatgtgcccagacaggggactcagccctgaataaaagtggtcctctggcacacctgggatggggaaggtactccttggtaagctcccaacctggcacttcttgatctccctggcaattttcttgcccattactccatggagatcagaatatcactctgttgtgtcccctcaacacggaaggagtgtctcaataagaatggggctaaaagttgagtccaaacactgtaggaattgagaggttccccaccttgcactacccttggaagccaagagaagatgttaaaaataaaatggtaatgcttcctgaaggtgtcttcccatctttacactggatgggttcaattgggaggaattactggactctggaagttgaagactgtccatataattaaaatgtacaataactacccaggtttaccttgcaagtttcaacatacacaaaattaactttatatgactcttcaaaaacagtttgccatcatacctaataatctggtttaaattttaaaaactcatccattttacttaaaatttaaatcaaaaaagaacacaggtttccatgaatttgtctcaggcctggcacagaatagtactccataaatattttgttaaatgatagatgatgaatgctctcactgtccaatcttcacacatcttatagactaagtataaagaatccaagatttatagtgctgaaagtagtttttatatgtttacaaagcattattgtcattactgcattttttttgcccattactccatagagatcagaatatcactctgttgtgtcccctcaacactgaaggagtgtctcactcactttgatgctatactttctacttttgtttatttaatgcttctcaatatgcttgtttaactgttgcagatccccctgaaattaagcttaggaggacttcttcaaccagaaaaacccgttgttctaaaggttgagtcaagggatggcaccgtaagtggagcctgaattttcctaaggacttctgctttgctcttcaagaaatctgtgcctgagaacaccagagacctcaaattactttgtgaatagaactctgaaatgaagatgggcttcatccaatggactgcataaataaccggggattctgtacatgcattgagctctctcattgtctgtgtagagtgttatacttgggaatataaaggaggtgaccaaatcagtgtgaggaggtagatttggctcctttgcttctcacgggactatttccaccacccccagttagcaccattaactcctcctgagctctgataagagaatcaacatttctcaataatttcctccacaaattattaatgaaaataagaattattttgatggctctaacaatgacatttatatcacatgttttctctggagtattctataagttttatgttaaatcaataaagaccactttacaaaagtattatcagatgctttcctgcacattaaggagaaatctatagaactgaatgagaaccaacaagtaaatatttttggtcattgtaatcactgttggcgtggggcctttgtcagaactagaatttgattattaacataggtgaaagttaatccactgtgactttgcccattgtttagaaagaatattcatagtttaattatgccttttttgatcaggcacagtggctcacgcctgtaatcctagcagtttgggaggctgagcgangtggatcgcctgaggtcaggagttcaagacaagcctggcctacatggttgatacccatctctactaaaatacacaattagctaggcatggtggactcgcctgtaatctcactacacaggaggctgaggcaggagaatcacttgaacctgggagcgggatgttgaagtgagctgagattgcaccactgcactccagtctgggtgagagtgagactcagtcttaaaaaaatatgcctttttgaagcacgtacattttgtaacaaagaactgaagctcttattatattattagttttgatttaatgttttcagcccatctcctttcatatttctgggagacagaaaacatgtttccctacacctcttgcattccatcctcaacacccaactgtctcgatgcaatgaacacttaataaaaaacagtcgattggtcaattgattgagcaataagcctaaaagcactcattccttttcttttccaaattcttcctttattttcccttcctgaataatttagtcctaaagccattaggtgggtggcagccagatggtggccacacattaaggtagagaagagagagtcatggtggctccaagtcagagaccaaggaggttcatgtggagagacatcctggcctgggtgtgggagtccaagcaagcagagaaggggtcgacgcagaggggtggcttgcaagagcagccagagcctaaatagggtatggagaacccacatgaggcgaggagggcatccatgagtgggaggggttgggtgaggtttggctacataaaggggattgatcaaataagtaaatgtattaaggatgatagaagccaggcttctcacctttgcagaagggagtcatggattcagaaagggagaaaactagcatgaatcctatgaaattagattggaatggatgtaaccgtgtatattcatacccttgtagatagatagatgggtagatagatgatagataggtaacagatagatgacagataatgagatagatagatgtaaatgtatgtctgtatttgtgtgtgtgtacaaaaaacatatattccctacttctctccactgatagggctaggtaacaatgacatttcaatagcaatgaacacacttagtgccccgatcttggcttatgaataccattttccactgaagggaaccagagctctttagagaaatggctgatgccagggcaagaattaagaatgttcaagataagtttaggacacattttgtgccaggaagcaagaaaattttcaaataatttccaagtaatctttggaaatgatacttgaaaatgacttccaaatgacatttccaaatgacttgcaaacgatttccaaatgatatttggaaaaacctaaagactccatcaaagaactattggaactgataaacacattcagtaaagttgcaggatacaaaatcagcatacaaaaatcagtagcatttctatatgccaatagtgaacaatctggcaaaaataaaaaagtaatcccatttacaatagccacaaataaaactaaatacctagaaattaacttaatcaaagaagagaaaggtctctacaatgaatactgtaaaacattgatgaaaggaattgaagacacaaaaaaggatatttcatgtttatatattgtgagaatcaacattgttaaaaatgtccacactactcaaagcaatgtacagattcaatgcaatccctcaaaataccaatgacattcttcaa

SEQ ID NO: 40 - DNA, 26865 bp, Homo sapiens
  ref <308>: DATABASE ACCESSION NUMBER: http://www.genome.utah.edu/genesnps/
  ref <309>: DATABASE ENTRY DATE: 2002-06-05
ggctgcaaatggccttaaaaatctgaacaacatttgaaaattctgagtaatgggcaaccaggcagtctggcttatgaggcctggccagctcttcttcatcatgaggccccctacctgctgccttcctgcctaattccagcagccttcactccatccttactaaacttgcagacagaccactcccagagagtgggacagatgccatcagatgccatctcccacctctgtgagcctcaactaacaactcctgtttggcccatctcttctataatgattgacaccaagaatgtctgctaccttggggctgtgatttcatctggtaatataaaaaaaattttttttttgagagtctcactctgtcatccaggctggagtgcagtggcatgatctcagctcactgcaacctgtacctcccgggttcaagcgattctcctgcctcagcctcccaagtagctgggattgcagatgcacgccaccatgaccagctaatttgtgtatttttagtagagatggggttttggcgtgtcagtcaggctggtcttgaactcctggcctcatgtgatccacctgcattggcctcccaaagtgctgggattataggcgtgggccactgcgcccagcccgagaggtaaattttttgaaataagctttagtggaacaatcctgaagaataacacaagaggaataacaggttaccagtaaggtgtcagccaatttgttccagtcacttttgaatccatgtcctataatctaaaatttagttctctttccctaagctgagagctttctatcatgtcagtatgtatgttatgaagaaaaggagacttaggtgagatgtttgtatttatcacaactgctgcatcaattgcctaggacttcaacagcttcatggaagtctgggaaatgttcaggcataaggttattgccttagctgactgaaaattgccccatacagtggtacatatcaacccttagtgaggccttttaaaaaacaaacaggttgaaaaatagattaaagtaggcaaatacagcatctgtctttagagctatcaactcaggaattctctcaatttttttttttttttttttttttttgagatggagtcttgctctgtcacccaggctggagtgcagtggtgcaatctcggctcactacaagctctgcctcccaggttcacaccattctcctgcctcagtctccggagtagctgggactacaggtgcccgccaccacgcccggctaattttttgtacttttagtagagacggagtttcaccgtgttagccaggatggtctcgatctcctgaccttgtgatctgctggcgtcagcctcccaaagtgctgggattacaggcgtgagccaccatgcccggccaggaattctctcaattatgaaatcttgcagagaagttatttttctttctcaaaatccagatgatgacaatattccttactccagatctggcattttttcatcatcactgtcttgtgaatgatcatctgctccatctgctaaatctacatcctcttcaccacccatgtggttcatcatctcagagaaataatcaaaatcagacatggcttcatctgaaccatcttcccagtctttccaatgattggagtccacactaagccagttaagctttgccctttgttttgttaaccttggccatgactggccccattttcctttttgtaaacaaacaaaaccgatctgtccattctcttatgcttgaaatcatttggatcaatacagtgaaaaagattgatgccatataaatgcttaaaatgatcacttccaggaagtggcagtacaagggcttctgcacggatagcagagcagtggtcccgaccacagtgcatgccctgagctctccgcctcccctgcctgtctgcgaggtggcagcagtgccacaagcagcccacggcagggtggaacacccacatcgacaacctcgtggtggaagggacctgtaggatgcagccatcgtgggctacaaggacttgccttccatccaggccaccatccctgggaaaaccttcatcaacgtcaagccagctgaggttgatgtcctggttggcaaagactggtcaagtttttttgtgaatgaggtgacacttgggggccagaaatgttctgtgatctgggacctactgctgcaggatggggaactgacctatctttataccaagagcaccgatggagcccccaccttcaatgtcactgtcaccatgactgctaggatgctagtgttgtaggaagtcagggacccaaatggagggatgggctggaggcatggcagaggaacagaaattgtgaagatttcattttaatatggacatttatcagttcccaaataatacttttataatttcttatgcctgtctttaatctcttaatcctgttatcttcataagctgagggtgtatgtcacctcaggaccactgtgataattgtgttaactgtacaaattgattgtaaaacatgtgtgtttgaacaatatgaaatcagtgcaccttgaaaaagaacagaataatagcgatttttagggaacaagggaagacaaccataaggtctgactgcctgtggggtcgggcaaaaagagccatattttctttcttgcagagaggatataaatggacgtgcaagtaggaaagatattgctaaattcttttcctagcaaggaatattaatattaataccctgtgaaaggaatgcattcctccggggaggtctataaacggccactctgggaatgtctgtcttgtacagttgagataaggactgagatatgccctggtctcctgcagtaccctcaggcttactagggtggggaaaaactctgccctggtaaatttgtggtcagactggttctctgctctcgaaccctgttttctgttgtttaagatgtttctcaagacaatatgtgcaccgctgaacatagaccctcatctgtagttctgcttttgccctttgccttgtgatctttgttggacccttatcagtggttctgcttttgccctttgtcctgttccctgagaagcatgtgatctttgttagacccatattagtagttctgctttttgccctttgaagcatgtgatctttgtacctactccctgttcttacaccccctcccctttttgaaacccttaataaaaacttgctggtttgaggctcaggcgggcatcacagtcctactgatatgtgatgccacccccggtggcctagctgtaaaattcctctctttatactgtctctctttatttctcagccagccgacacttatggaaaatagaaagaacctatgttgaaatattgggggctggttcccccaatatctggcacaccaatgtggttttctttctcctaattgcatgtgggaacctgattccctttggtaggtgcggagaaacgttcattggtctggtccacagaaatgcttgttcagctctctgatgattggtaagttgtctgtgtattgtccaggctaactttgggtcactcagagtctaaacattatgcttatctctgccatattaaactcctgttaaaacaggggggagtttgggtacccatggaaaatatggtcaccctattcagggcagtggaagaacattgtcctttgtttcctgaaaagggaacattagacgtggaactatgggatcgtgttggtgcaaaattccgggaactggtcccaacaggaaattatgttcccgtcactgtttggggtgactgggccttggtatgtgccgtcctaatgacataccaatcccatcaccccctgcagttaccacagttttctgaatctggaaaccctccacctcttcctcaaccttcctctcccgcacggccttcgttacctgatcagcctctcccttcgcctactccttccccacctgaggatattgaagattcaatatctaactccagtgactttggcttaacgtcaccccctgatgatcttatttcttttcacgaacagccagtacttgtagctcccacggccccgacttggacagcccaggactgtatctatgctaactcttccctcttcaaacctttgcagcctttgcctccggagccatctaatggctccggaactaaactacaatttacctgtaattctgcaggccctcccccatccaccacagcccctcaccctcctgtcatttcggttcctcaactggtcactttgccatccactcaacctgcttctctgtacccttcttcacatgtggacacccctgccactctggttgcacaggattgggcaataatcaccagtatgcttctgcctcttctgctcctctggtgcccctttctcacgctctcatactggtccaacctcctcagcctcagtttcccttatctacacataattttcctgtcacttctgtgccaactctgtctcatgtgcctgttgttgaaatttccatgcaatgcttattacgccaaaacaaacaagtggattagaggcgtgggcttatccgttcatgctggaaccttgcaatgctcaaggggtacaagtgcatcgataggcgctgctcaatcttacctttttaaaagaattcaaggatgcttgtactcagtatggtcctacttctccatatgttaaaatggtattacagactctttgtactgaggtcattttgcttcctttagactgggacttttggcaaaagctgttctaactccatctcaacatttacaattctgtacctggtggtcacgggaggcccatctgcaggctcagctaaatcgggctgatggcattctaattactcaggctcagctcacaggctccaataattactctgacactactgcccaattaggctttgatgctctcaccgtggaacaagtaacaaaggtgtgtatgagagcttgggataaattacgcgccccaggccaagctcctgtttcttttactactgttaaacagggtcacaatgaattatatcctgattttttagctaaattacaagatgctgttgaaaaatctgtctctgatgagcacgctcaaggtattcttcttcgaatgttagcttttgagaatgcgaaccatgagtgtaaaatggccatgcattccgtccaacaacaaaatttacctgatcgtgaggtgttgcctgagtatattaaatatgaaggcattggatcagacacaaagctattctgtgcccacaaagctattctgtgggcacgggccatgaaggacggcaatcaaactggctcgactgattcttttcttggagcctgctataattgtggtcaacttggtcatactcgaaaaaattgcacagttaaaaacttaaaggcagccaagccggctcaaaaaacacagccaaatgctcctgctactgtttgcccttgttgttgtaaaggtaaacattgggcaagtacttgccactctaagtctgatatagatggaaatcccttgccacagaaccagggaaatgggaaatggggccagtcctaggccccaatttcaaatgggacacctcagactcagacgaatgttgcctttctgcttcaatcggtcccaacgcagcccccagcacaaacaaatttacctacagccaacccagatgtgtcccagcctcttcttctgtctcagtacaacgcttatctatgctccagagtaaggggaggggcggtcaatctctgtagtaccattcctctaaatttactacctaattctttgcctttaattcccagaccaatacctggaaacctggaacgattataacatggggaagagggtatgcttgtgtttcaccaggagatcatcaatcccctgtctgggtgcccactagaagacttaaacttcatgtgaatactgacaataaaaaccacagggaagagacgtccacgtcagagaccaccctcatacctggtgagatctgtgccaactcctcagaaactggcacgccaaatcaaaatgggtctggttcaatcctccctaatggcaacagaaacccctctaactaatcccacttctcctaattccctttctttttctccttacgaacctaaaaatctcaccatttctattagcctgaaaataacatccccttattcttctattcctccttcagcactggatcttgcttacaataggttttatttaataattctcttccttatactttctgtctcaccagtttcccctcacattgatttacctgctacacaaaattattcttgctgggcttatgtgccttttcctccacttattcaacctctcacctggatggatgctcctgcggaaatctatactaatgatagtgtgtggatgcctggagctacagatgaccgttgccccactcaaccaggaaaagaaggcactgcatttaatgttcctacggttataaataccctcctctgtgcctcggacatgcaccttgttgtatccatctagaaactcaagtctgggctgcgtaccttctggagagatcagccacaggggaacggggacatttggtctccagcctctccctttctcctttaagacaaatgaaagggggagtaataggagataccccctactttcaatataaacctgtaggaaaaccatgtcctaaaatttttgagggcccatctaaatctttaatttgggaagattgtgttaattcacatgcagtactattaaaaaatgactcatatggtttagtaagagactgggcaccaaagggctatttgaaaagcagttgctcttctgctggaagggaatgcctggaggctacttgttttatttcttattgggaggacgaggatcatcatcctactttgcataggaggttcagctcattctgtcccttaaaatgggaagataagagcattaccccctgccacccgaggcctcttatgatatcccccattctgagcccagaacacccagaactttggaaattggctattgccatgtccagactgcgagtatgggaaggggaaacttttctttctgttgtccccactaccacccctcacatccgtgattctgaaacccatgataaatcccctttggaccattttcctctttttgatgccagtcctcctttatgggactctgattggcattatgataattcttctggacccaggtatgtccccctacctcttcagaatccccgggcacctcggattgcttctttacggcatcaaacattgggcgttgccaccgccactcctccccctcggtatcaacgtagattcaaacattctgctttgtttacctccagcctgactattatacagagttgtgttaagcctccttacatgctgttagtgggaaatatcaaaatttggatgaacaatcaaaccgtccaatgcattaattgtcatttatccacttgtgttaactcccgttttgactcaggaaaagtgtaatgttggttcgagcttgagaaggaatctggataccagtaactttacccagaacttgggaatcttccccctcagtacatttaattaatgaagtgttacaacgaattctcaaaagatcaagagatttgttttcactttaatcactgtgatcatgggcctaattacagtcactgcactggccaccactgccggagtggcattacaccaatctattcaaatggctaattttgttaatgattggtaaggcaattccacccaaatgtggaatcctcaacaaggcattgatcaaaaattagctaatgatttaagacagtctgttatttggcttggggaccagctaatgagtctcgaacatcacatgcaaatgcagttcgattgcaacacttctgatttctatagcacaccatattcctacaacgagactgatcatgggaaatggtcaaaggacaccttctgggtagggaagataatttatccttggacataactaaattaaagaaacaaatttttgaagcctctcaagctcatttatccattgtgcctggagctgaggcgttagatcaggtggcagaaagtctttctggactaaactccacaacttggattaagtctactgggggctccactgtaggaaattttggaatcatgtttctctgtttaatcggcttgtttttagtgtgccagaccagtcaaagaatcctgcatcaaaaccgagagaacgagcaagccttcatcgccacggcacatttatataaaaagaaagggagagatgttgcgggaagtcagggaccctgaatggagggactggctggagccatggcagaggaacataaattgtgaagatttcattttaatatggacatttatcagttcccaaataatacttttataatttcttatgcctgtctttactttaatctcttaatcctgttatctttgtaagctgaggatgtttgtcacttcaggaccactgtgataattgtgttaactgtacaaattgattgtaagacatgtgtttgaacaatatgaaattagtgcaccttgaaaaagaacagaataagagcaatttttagggaacaagggaaaacaaccataaggtctgactgcctgcagggtcgggcagaaagagccatattttccttcttgagagaggctataaatggacatgcaagtagggaagatatcactaaattcttttcctagcaaggagtattattattaataccctgggaaaggaatgcattcctggggggaggtctataaacagccgctctgggaatgtctatcttgtgcagttgagataaggactgagatacgccctggtctcctgcagtaccctcaggcttactagggtggggaaaaactccgccctggtaaatttgtggtcagaccggttctctgctgtcgaaccctgtttgctgttgtttaaggtgtttatcaagacagtatgtgcaccgctgaacatagaccctcatctgtagttctgcttttgccctttgccttgtgatctttgttggacccttatcagtggttctgcttttgccctttgccttgtgatctttgttggacccttatcggtagttctgcttttgccctttgtcctttccctcagaagcatgtgatctttgttagacacttattagtagttctgcttttcgccctttgaagcatgtgatctttgtacctactccctgttcttacaccccctcccctttttgaaacccttaataaaaacttgctggtttgaggctcaggtgggtatcacagtcctaccgatatgtgatgccacccccggcggcccagctgtaaaattcctctctttatactgtctctctttatttctcagccggctgacacttatagaaagaacctacgttgaaatattgggggtgggttcccccagtacgctagtcctgctgatgagcaaagaaggtgttgatggcattttgatcaacaagaaatgttatgaaacggcctcccacctctggcattcctagtactgacctatctctcccttcccttccaccgctccccacagcttagcacccctttcgttcccatacacatgtacatttttattttggggaccattaacccacagcccttatcgctgccaaaaccacatgggctggagggccagggctgcatggacagtcacaccactgcacaccagcctggtgatggagcaagactctgaaaaaaaaaaaaagaagcataaaagacctttaacagtccctgctacttgtgaccctcctgaatctgtcatctgtccagtgattttgattttgcaacatggaaagttccaagccttgactcctgagcccagataccattcgcactctggagtcattcaagcatggggagctcctcagagggtgatcaactgacctcccttacctagctcctcctccggaggtttgccctggagttgagccagcccttgaggaggccttcactcccaccgcctctctcccttctggatatgagctcaggcctggctgggctccaggagaatctttccacaaggcaagagataacacaaagtcaaggtgaagatcagggtagccctttaaaaggcctccttgtgccctagagttgccacagctcttctactccactgctgtctatcttgcctgccggcacccagccaccatgtgggagctcgtggctctcttgctgcttaccctagcttatttgttttggcccaagagaaggtgccctggtgccaagtaccccaagagcctcctgtccctgcccctggtgggcagcctgccattcctccccagacacggccatatgcataacaacttcttcaagctgcagaaaaaatatggccccatctattcggttcgtatgggcaccaagactacagtgattgtcggccaccaccagctggccaaggaggtgcttattaagaagggcaaggacttctctgggcggcctcaaatggtaagtggtgcccatctcctccctgcccccttcaccaccccctgggattgttcaggtcttcagaccatgcctagaatggggcttccagctccaacaacctgtaattcttcccaaagtagactagtgggatgatgtgaagggagtgaaccttcagacccccacccagcctctaaattaggcaggaagaattaaaggaatgtctcctctaccaggctgcccaggaggtgggatgggaggaccacagtggtctgaggggtcaaggaatcctcactccactcctctctctgagcctagacttctctaatggactgagaaggtgcatgtacagcacttagcctagcacccagcacagtaagtgccccttatacagccaggattcatgttacttttcatggaaaatgggggcagtgactactgtcctccatgaaagctgctggggagaattagcctagctattgcaggctgggattgctgctttcctggtgctatttccagctactcaggctcacaggggcagttttctacaatgacatttcagggttgctgatgagcctcccactcagcagggcccccagcctctcagcattttttttttttttttttttttttgagacagagtctctctctgtcgcccagcctggagtgcagtggccaatctcagctcactgcaacctctgcctcccgggttcaagtgattctcctgcctcagcctcctgagtacctgggactacaggagcatgccaccatgcccgctaattttttgtatttttagtagagatggtgtttcaccatgttagccaggatggtctcgatctcctgacctcgtgatcctcccaccttagcctcccaaagtgctaggattataggcgtaagccactgtgccctgccagcctctcagctttgatcaagccaagggttggtttattttttcttggaccaatcagccaggtctgctgaccaactacctagctcccacctctgctggcttcctcccgggggcagagaagatggagaaggctagtcatgtggatcttcagggtcaggaaatggaaaagggaggctttggacccttttgctttggggggcacctctaggaggaggcggctcggcccaagtccagactgggtagacaaaacatctgcactctccaaatgtgggcttgtggctgggtatgcaggcttgcaatggaagggtaaacctgagtgaggtgagctgtgcctttagctcagctaagggctcagggaaaagcagagatctgtgcagtcctcagcctctacaagctcacctgctccctaccctctggacaggcatagtttagagagtttatcccatccagagttgccttcctgtggtcagaaactgatgagcaaaaagaagcccagagggcaccctgtcagcgaaaagaaccccaatgctgctgcattctaattaagggttctttctttctccttgatctactgtatttctgaaggaattgggagtaggaggccttagggtctgtcctaccaagtccttgcagtcatggtggagtgcagtggggctgtgcccacatgggagtcagcatgccaggtacctgccttctcctccaggaaggaaagcagggaccagaggtgtaagggcaagagtggggtggatggtgtgagattcctacagccttgcctgctctctaaaggcaactctagacatcgcgtccaacaaccgtaagggtatcgccttcgctgactctggcgcacactggcagctgcatcgaaggctggcgatggccacctttgccctgttcaaggatggcgatcagaagctggagaagatcagtgagtgccaggctggcccctggggctggggctggatcccacaggagctgctggagggagaggagggttgggcaggggtaagggttaggactagagcgcaatgcagccttttcttggctactgctgccatctagtggccatctgctatctgtcccccgctcctgttaagaggcaactggtacagagagggggtaagggtgctgattcatttcccaccctcatgccccctctcccttcagtttgtcaggaaatcagtacattgtgtgatatgctggccacccacaacggacagtccatagacatctcctttcctgtcttcgtggcggtaaccaatgtcatctccttgatctgcttcaatacctcctacaagaatggggaccctgagttgaatgtcatacagaattacaatgaaggcatcatagacaacctgagcaaagacagcctggtggacctagtcccctggttgaaggtgagatgctgccagccctgccttcaggttctagtagaccctgacattgtccccaatcttccttcctttttacttccctgctccagccgcaatgacccatctttttcctgattacctccgccacctctacctcctctgccacttaaaacctttgccatttctctgcagagatagatttagccttttaattatgcaccttagtactccagataatgaccttcatttcttttccaattaccatgtgccagtactaagcattctatacgcattcatcgctgaattccccttggaagtaggttttattatccccattgtgcaggtgagaagcaggcttagcggggttaaggagcttgtctgagccttcaggctcatgtctctctcactcctaagggctggacacatagcagagtcagcgcttgatgtttgattgaatggggaaggagaggtggagaccacgccctcctcccttgtttagaattgtcttcgtcgtcatgataaacccgttctgtgtccccatcttgccttccattctggctgaaggtcaggggtggagtaggaacttccagagacagaaagctaagatccgcctccaggagagactctggcagctggagaagcaaaatggaagaagggtggatttaacatttctttttatttcccagattttccccaacaaaaccctggaaaaattaaagagccatgttaaaatacgaaatgatctgctgaataaaatacttgaaaattacaaggtaggtgatagagcagaagagaatatgagttaggctaaaagtaatcacaagagcagggtggagtccattctacacactgtagaagcttcaaaaccaagcagagaacctggcacatagtaggtgtacaataaaaactgacttaagggctgggcgcggtggctcacgcctgtaatcccaacactttgggaggccgaggtgggcagatcaattgaggtcaggagttcgagatgagcctggccaatatggtgaaaccccgtctgtactaaaaatacaaaaactaggccaggcaagatggctcacacctgtaatcccaacattttgggaggccaaggtgggcagatcacctgaggttgggagttcgagaccagcctgaccaacatggagaaactctgtctctactaaaaatacaaaattagccgggcacggtggcgcatgcctataatcccagctatttgggaggctgaggcaggagactcacttgaacccgagaagcagaggttgcagtgagctgagatcatgccattgcactccagcctgggcattgcaccaagattctgtctcaaaaaaaaaaaaaaaactgactcaaggagttgggatcgaaaagtgaggaactgaagaggatcctagaagagacctaacctctccaccaaatttaaagagggcccggggctgcctcctacctccacaagttcgtaggtcctgcccagacttgctctacttccaagtggaaggagccttgttatctctagtcagggacagaagtatggcaggagtgtcacagatggggctccttccttattaatgtctcccaacctcacccaacccaggagaaattccggagtgactctatcaccaacatgctggacacactgatgcaagccaagatgaactcagataatggcaatgctggcccagatcaagactcagagctgctttcagataaccacattctcaccaccataggggacatctttggggctggcgtggagaccaccacctctgtggttaaatggaccctggccttcctgctgcacaatcctcaggtgtgcttccccctcattgatcctagaccccagccagcccaatctctgggctccagagaaagggagagccaattctctcaggctttctgtgcaggaagactaggcctgccctgctccttacccaagcagtagttggctttgaccccagagtagagctgccccatcttctggaagccgggcctgggccccagagccactactgggaagggactggacaggctcttctcgatgtcacagttggattcttctctaagcccttgcttctcctgggcttacacacactagtcacctccaacctactctggtcttcaggtgaagaagaagctctacgaggagattgaccagaatgtgggtttcagccgcacaccaactatcagtgaccgtaaccgtctcctcctgctggaggccaccatccgagaggtgcttcgcctcaggcccgtggcccctatgctcatcccccacaaggccaacgttgactccaggtgtgcctgccctcccagtgacatctagccccatgatgcattcaacactgcttgccagcccacctggctccccctacccccggcccctgctggccaacctaaagtcagtcaaccatcaactactaaaaatcatcctgccggccgggcacggtggctcacacctgtcatcccaacactttgggaggtcgaggcgggtggatcatgaggtcaggggttcaagaccagcctgaccaatatggtgaaaccccgtctctactaaaaatacaaaaattagccatgcatcgtggcgcgtgcctgtagtcccagctactcaggaggctgaggcaggagaatcacttgaaccaaggcggcggaggttgcagtgagccaagattgcaccactgcactccagcctgggtgacagagcgggactctgtctcaaaataaataattaattaattaaatataaaaatcatcctgcccccagccccgtggctccatgtctctaccacctacagacacgcattgactcatccacagatctgcctgacttcccagaggagcttcctgctgccctcagagacatgtggtctgggatgaaaggctgggagctccatgttccaaccagctgcagcacgcacataacatgcgctgcagctccaaacgcacacccacatacactgccagacaccaaagtccacagacacaggtgttcagacagaagcgcctgttaggagggaagggatggagaagggctggatttaggtttgatctggcagaagctgaggaaaacatgagtgagtgggaatgagggagtaaagggcattttcctcacggcggaagaatgagggggcatgaggctgagcaaggaagggagtacgaagtcccagacccacttttcctcttccactctggagcagcatcggtgagtttgctgtggacaagggcacagaagttatcatcaatctgtgggcgctgcatcacaatgagaaggagtggcaccagccggatcagttcatgcctggtgagtctgtcctgtcctgcgccctgggccacacagcgagcctggactctgctccaccaccccagtaccccttcacctctgccaagcttgctgctagaaaactcttggctccaactatacggacctgttgacaccctcatcctgccatagacttacccaaactcttcacagctgggtttcccacgctcttcttccaaccaactgcaaattcaccctccaagaagccctctacgcctctgtctgccattaagtctgtcccttctcccctcggatggtgctattttcataggttaattccatctcttttccatccttcctgaatatttcatttcctctgtgtcgttaagggctacctgaaagcagggctgtatctctccccaggcgggggttcccccataataaggctacatcctcagatcagggttccctgggcagggccatgtctccccctcaaccagggcagaaccatgcctctcctccctcctgccctaacccctggctgatgccactccttgcctgcagagcgtttcttgaatccagcggggacccagctcatctcaccgtcagtaagctatttgcccttcggagcaggacctcgctcctgtataggtgagatcctggcccgccaggagctcttcctcatcatggcctggctgctgcagaggttcgacctggaggtgccagatgatgggcagctgccctccctggaaggcatccccaaggtggtctttctgatcgactctttcaaagtgaagatcaaggtgcgccaggcctggagggaagcccaggctgagggtagcacctaaaggctgtaactcacagcccctgtccaccctatgtggccccacaacacagatttagagatacaaccccccacccttctccgccattcttccctactcccaacccactctgccttctttttcagcttgtggcaatgccagtgatgtgcataaacagtttttttttttccataaggtccctgagtagttcatttatgtattcatttgctcactcattctttcaacaccgattttattgagcacctactatgtgccattcactttctcaggacactcgagctccgtggtctctgaagcttacattctagtgagggctaacaagtgaataagttcagagactcgtaagtgctttgaagtcaatcacaatttgtgtgatgtgatgagaaaatggcagaggcagtgggtggctgctttgaacaaggggtcaggaaaagtgttgcatgctgagaagcaggtaaaaaggcctccaggtgggagaggtcagcatggccagtgaaggggagagtgggccctgatgaggtcagagtggacatagtggtccgttcccactgtagctgggattacaggaggccaggagggaggttgggttttattctaattatgacaggaagtcactggaggattttaagtatgtaagtggaacaatctgtttttttgtttttgtttttgtttgagaaggagtttcgctcttgttgccctggctggagtgcaatggcatgatcttggctcactgcaacccctgcctcctgagttcaagtgattctcctgcctcagcctccaaaatagctgggattgcaggcgtgtgccaccatgcccagctaattttgtatttttagtagagatgaggttttgccatgttggtcaggctggtctcgaactcctgacctcaggtgatccacccatctcggcctcccaaagtgctgggattatagacatgagccacagcgcctagcctgttttttgttttgttttgtttgggtttttttgttgttgttttttgttctgttttttgagtgacatggtctcactctgttgccaggctgattgcagtggcacaatcatggctcactgcagcctcaacctcccaggctcaagtgatcctcccacctcagcatcccaagtaactaggactacaggcacacatcaccaggcctgactaatgttttactttttgtagagatggggttttgctgtgttttctaggctggtctcaaactcctaagctcaagcgcttctcccacctcagcctcccaaagtgtttggattataggcatgagccgctgtgcccagctggtttgtatttctgaaagatcgccgtagttgctgtgtggagaatgttctgctaagggatgggagtgggagcaggagaacagttaggaaggaggctgttgtgctggtccaggaaagccagatgttgatgagttaaaccacggccacagcagtggaaacagtgagaagtggtaggattgagtttatcttttgttttctgtttgtttgtgtgtgtgtgtgtgtgtgtgtgtgtgtgtgtgtgtgtgtgtgtgtgtgttttgagacagactcttgctctttcacacaggctggactgcaatggcacaatctcagctcactacaacctccacctcccttgagcgattctgctgcctcagtctcccaagtagctggaattacaggcgcctggataatttttttatttttaccagagatggggtttcgccatgttggccaggctggtctcgaactcctgacctcaagtgatcccacctcagcctcccaaagtgccaggactacaggtgtgagccaccgagcctggcctgagtttatattttgaaagtagggcaggccgggcgtggtggctcgtgcctgtaattccagcactttgggagtccgagatgggtggatcgcctgaggtgtggagttctagactagcctgcccaacatggcaaaaccccgtctctactaaaaatacaaaaaattacccgggcgtggtggcaggcacctgtagtcccagctactcgggaggctgagacaggagaatggcgtgaaccccggaggcggagcttgcagtgagcggagatcgcgccactgcactccagcctgggcaacagttcaagactctgtctcaaaaaaaagaaaaaaggaaaagaaaaagaaagtagggcaaaagtgttgtttgttttgatccaaataggcttcactgcatgttacctcacaggttcgaccttgggggagtgtatgtgacccacagactggacctgccgttgccatccctctgtcttggtctgcaaagaccaagactggtgtgtccccctttcctcacccttccctggggtaaacactgccaggtaagggcttctacagctgtcttctgcagcagtgcctgggttccacctccttctccccactcaaaaaccccaggaagtctgtaaaagcttcctcactagggctgggtgcagtggctcacgcctgtaatcccagcactttgggaggccgaggcgggcagatcacgaggtcaggagatcgagactgtcctggctaacacgatgaaaccccgtctctactaaaaatacaaaaaattagccgggcatggtggcgggcacctatagtcctagctactcgggaggctgaggcaggagaatggcatgaacccgggagggggagcttgcagtgagctgagatcgcgccactgcactccagcctgggcaacagtgcgagactccgtctcaaaaaaaaaaaaaaaaaaacaaagcttcctcactagaaaatgaagccctgactcctttatatctggaaactggtatatttaaaccttggtcattggccagtctgcaaatgaatgtcctgagacatcccggaagaaactgacccttttagcctagagagaggccccatccccagctccctggtgccagccagaccagggtcaggtcatcaacaacccagtccttcccctctgactagacagaaggacgtcagcagagcatggctgataccctgcctagctaagtcttagagagaagacgtccctatagggagcaagacaaggttttcagttacgacactaccttcttccttataaagagctttgttaatcaggtggcagtggaccgttccctactattccctaactcggtcttggccttcatctgccatttaccctgggactttgctaaagagcctgcaggaggccaagtgtggtggcttatgcctggaattccagcactttggggggcctaggcaggaggatcccttgagcccaggagttcgagagcagcctgggcaacagagtgaggccctgtttctacaacacttaaaaaaaaattggccgggcgcggtggctcatgcctgtaatctcagcactttgggaggccgaggcaggcagatcacaagatcaggagatcgagaccatcctggctaacacgatgaaaccctgtctctactaatgatacaaaaaattagccgggcgtgctggcaggcgcctgtagtcccagctaatcgggaggctgaggcaggagaatggcgtgaacctgggaggcggagcttgcagtgagcagagatcgcgccactgtactccagcgtgggcgacagagcaagactctgtctcaaaaaaaaaaaaaaaaattacccaggcatggtggcatacctgtagtctcagctactcaggaggctgaggtgggaggattgctggaccccaggaggccgaggctgcagtgatctgagatcgtgccactgcactgcatcctagagcaagacccaatctctaaagaaaaagaaagaaagaagaagaaggattagtatttcactttcctgccattcaatgttctggttcaatagttggagaatgaaagagtgcatatgtgtgttggggggcagagtgagagagagaaagagatgaagatatatgggaacctatagacagacaaacagatatagatagatgtaatatgcaccaggtcagtatctgtcaaagtgtggctcacagaccccttgagccagactcatctgagaaacttacttaagaacacagactctggtccctatctttcatgcattgagtacactctgggagcagggcctggaaatctgcattttacaggctatccaggtgaatcttgtacacactgctgcgcaccccccccgagtcctcaagtgacattgtgtgagattcttgcaaacttgcgatgttttccatcccagttaaacccacccaccacttccacagaggacacagatcttctgctaattgcttatctcttcatcactagtctacaaaatgcctttcaggccaagcacagtggctcacacctgtaatcccaacactatgggaggccaaggtgggcacaacgcttgagctcaggaatctgagaccagcctggccaacatggtgaaacctcgtctctagctgggcatgttggcgcacacctctagttccagctactcaggaggctgaggtgggagaatcgcttgaacccaggaggtggaagttgcagtgagccaagattgtgccactgcactccaactgggtgacagagtgagtgagagagacgccgtctcaaaaaaaaaaaaaaaaaaaaaaaaaaaaagcctttcaaattctgagcaaatctgaaatacttaactttcctccagccaatcttattcccctctgctttgagctgaggatagcttgactcgatccagcacccagctgatgggggcagaggaaccgggagagggaagggacaaactccagtatagcctttcccttgggctcactgtggtgtcagcatctctcccaggctgcttgtgatgggggctggggtcagctgcccacaagctctcctacggacttcattttttttttgagacagggtctcactctgtcacccaggctggagtgcagtggtgcaatcatggctcaccgcagtctggaacacctgggctcaagagagcttcctgcctcagcttcctgggtagctgggactacaggtgtgtgccaccactctggctaatttttaagttttttgaagagatggggatcttattatgttgcccaggctggtctcaaactcatggcccctagtgatccttctgccttggcctcccaaaattctaggattacagatgtgagccactgtgtcctgtctaaaccttcattttgaaggaaactgagggccattgtgtgggctgggtatagggtatgacttgccccagatcacacagctacgtagcagcagggctgggaccagaactcaagatttcaatggtgcatggtggggagccagccatgctttcccacctgtcccctgtggtcatgcagaagggctcttgggatatttggccgggagagtacctttcaggcaagcggagtgaccttttagaaaatgactttatgaactttgactcctgcccaaactgtgctctcccgtctggaaggccttttcaccactgatctaaacccacctgccttgaggctcagacacattttttatgactcctgagtccaccatgaccatccctcttcagcactcagcttctgttccttggtactgaacctaaactgaaccttggcatctgagcatctgcctggacttgaaatgggtggggccccccacagacactgtccacctcctgtacctctggagttcaagactgagccttctcatgttctttatcctgcatatcagcacctgacaaatagaagggtagacaaacaagagtgatgcaggacaggcaagccctaaaattggggcttagcccaggagggttcatggcttcacccagaaagaattccagcaagcagcagtggtggtagccagcaagccgcagtgcacagagcagcagaggtactgctccttgtggagcagggctacccagaggcagtgtgcccagaagagcaacgcaggggcagggctgcactcatatggacatctacttttaattatatgcaaattaaggggtggattatgcagagatttccaggatgagggtagtaacttccaggtcatcaggtcgttgccacgaaaagggatggtaactcctaggttttgccatggcaatggtaaactgacatggcacactggcaggcatgtcatggaaagctgcttctgcccccctacctgttttagctagtccttaatttggttccatgtccgagccccgcctcctacctcaagaggactgaatcaaatgctctaaatcacttggttaattggcccaatgatagtaaataaattctatctccttatgcagaaggaagcccagatctttgacaaaactggaaggtctttgccaagaccccacagttaaccgtctgagcctcagactgtccttaaccagtgatgtgatgataaataaatgtttaacaaccaggtctgggaggggcgggggtgtgcggaatagccttgatttgtaatgtttgctgattactgtggtgtagatactcccaacgtgaccaattttaaactaccaatgtgatgccagtaaacagggttggaaagagacacatagtagcattatatgttctttaccgttgttattattacatagtaaatgccattatgtagtatttccaccatactgataggtaaatgtaaataacctcaagggcacagataacagtaacatgtagtaaaataactaggaaatgatcagttttgagtatctattacctttgtttttaatataactcaattgtaggtttaagttttttaaattggctgtttcatataaacagctcataaaattcctgaaaatttaaaaatcagttctagtgagctattaagagccaaccctcccaggagaaatgggccacgcttgtcttaaagaaaagcagttcctggctgggcgaagtggctcatgcctgtaatctcagcactttgggaggccgaggcaggtggatcacctgaggtcaggagtttgagaccagcctggccaacttggtgaaaccccgtctctactaaaaatacaaaaattagcggggtgtggtggcgtgcacctgtagtcccagctactcgggaggctgaggcaggagaatcactagaactaggtggaggttgcagtgagccgagattgtgccactgcactccagcctgtgagacagaacaagactccgtctcaaaacagacaaacaaaaaacgaaaaggagttcctgggctggggacagtggctcaccacaccagcactttgtaatcccaccactttgggaagtcaaggtgggtggatctcttgagctctggagttccagaccagctgggcaacatggtaaaaccccatcactacaaaaaaatacaaaaaattagccaggtgtagtggcacatacctgtggtcccagctggtcccagttacttgggaggctgaagtgggaggatcacattgaacccaggaggtcaaggctggcagtgagtgcgccactgcgctccagcctgggtgacatagcaagaacttgtctcaaaaaaaaaaaaaagaaaaaagagaaagaaaagcagttcccctgagtctccttgttgacaggagggaaatccttccctattgtgggagggtgagagtgagtgaatgttctatcagcctctggtggccacaaaacctgaggcaaatgaggagccctctctgcagtcacactcgtagatgttagcgcccttctctggagggtggggatccagtcacagaggccgtgcagtggggtgggaagcgcaacggataaattggacccagaccaccccggtgctctgccccagctcccgtttcctgaccctggatcttaggcccatcactaacttcctgagcctcaatttccccatggataatgtgggggaacctaacacctgcgctgcccatcttccgaggctctggtgaggatgagaggcagaaaagagtgtgtgggcaggagagtcccacacagacgggaatgcaaatgactcgaagtcaggttgcagagacaaatcctcccagtgttgtcatttacagactgcgtggccatggacaccttaggtggccttaagccttggttttcccatctgtaatatggcgggggcgggtggtagggaactgggatgtttgtgtcatagcgttactgtgaggatgaaatgtgaacctatgaaatacatttagtgccttgataaatattgggtttctcgtcgttgtctatatccaaacccacgaaatgagtccttctcagggttttatacaaacctccccaaaagccaccagtttggtcaggagggggagcgggtgtgtgcagatgagtccctccgcaggatggaaccctggggaacccaccctccccgttccatccctgcagggcccactacagcccctggcctgccacctgcccactccgtccccgcagagccagggctgatggactgccgggcagcgggtggagaacccaaagacaaactcacggctccagcacaggtgttccgtcacagtttctgtgggattgggtttcttgagccttcttggtgaaaaaacatgaaatgaagggaatctgaagctatggtgaggaacaggcttataacagagtaaggagaggccaggtgtggtggctcatgcccgtaatctcagcactttgggaggccgaggcgggcggatcacctgaggtcaggagttcaagaccagcctgcccaacgtggcgaaacctcgtctctactaaaaatacaaaaaattagccaggtgtgttggtgggtgcttgttaatcccagctacttgagaggctgagacaggagaatcacttgaacccgggaggcggagattgcagtgagccaagattgcgctactgcactccagcctgggcgacaagagcaaaactccatctcaaaggaagaaaaaaaagccaaaaacaaacaaaacagagtgaggagaaatttcaggtcaaacaaaaatcctcttctcccctcacaaacacagaaagctgtactttcatcaccttggaggaagaacaaaaacactctttcaaggaaaagagacaaagaagatccaaactgtaactcctgagaattttacagtgtttccaaggggttacaccgtccctggaagttgacattggtccgaatactggtggcaagcagcatcacctgtcccaggggtgacgccattttagcactgaattgctcttggaggtgaagcctggctgggtgtgggggggtctgccccctggagcaagaggagatagggtagctcctgaggaaatggtgtctgtttcatctttcaccatgttactcttagctcaatatattgttctctagttctccctcacctttgaaaagaaattacaaggctcatgcctgtaatcccagcactttgggaggccaaggcaggtggatcgcttgagcccaggagttcaagagcagcctaggcaacatggcaaaatcctgtctctacaaaaaatacagaaattagctgggcatgacggtgtgcgcctgtagtctcacctgcttggggaggatgaagtgggaagatcacttgagcctgggaagttgaggctgcagtgagctgagattgtgccactgcactccagcctgggccaaaagagaagagaagagagagaggaagaagtcacaagttcttcaggtggcttgtgagtacctacattacagctcttcactgtcctgccttctggttattacattggccttggagccattagacctggcttcaaatcctagctctgccacttattagctatgtgatcttataaagttagttaagtcctctggatgccacaatcccattaataaagcaggaataattgcagcctatatacctcagagacctgttttgaggctacatttttttatttttttgagatggagtttcgctcttgttacccaggctggagtacaatggcgtgatctcggctcactgcaacctgagccccccgggttcaagcgattctcctgcctcagcctcctgagtagctgggactacaggcatgcaccaccacacctggctaatttcgtatttttagtagagacggggtttctctgtgttggtcaggctggtctcgaactcccgacctcaggtgatccgcccacctcggcctcccaaagtgctaggattacaggtgtgagccaccgtgcccggcctgaggctaatttttttttttaaatgtagctaagatgctcacacaagactggcattgtggaaaccgtcaaccaacaaaacaataaagacaaattaaattaaaatattt

SEQ ID NO: 41 - DNA, 76341 bp, Homo sapiens
  ref <308>: DATABASE ACCESSION NUMBER: http://www.genome.utah.edu/genesnps/
  ref <309>: DATABASE ENTRY DATE: 2002-06-05
ttgtataaagtgtaaggaagggatccagtttcagctttcaacatatggctagccagttttcccagcaccagcaccatttattaaatagggaatcctttccccatttcctgtttttgtcaggtttgtcaaagatcagatagttatatatatgtggcattatttctgatggctctgttctgttccattggtctataagtatgttttggtaccagtaccatgctgttttggttactgtagccttgtagtatagtttgaagtcaggtagcatgatgcctccagctttgttcttttggcttaggattgacttggcaatgagggctcttttttggttccatatgaactttaaagtagttttttccagttctgtgaagaaagtcattggtagcttgatggggatggcattgaatctataaattaccttgggcagtatggccattttcatgatactgattcttcctacccatcatgagtgaactcccattcacaattacttcaaagagaataaaatacctaggaatcctacttacaagggacgtgaaggatctcttcaaggagaactgcaaaccactgctcagtgaaataaaagaggatacaaacaaatggaagaacattccatgctcatggaggatataatcttgccctgtgttggccaggattagtactcgggtttctcaggtgatgagcggggccatagagctcccaagagtttatgtcttttgttttcagctgccagggtgggtagagaaaaaccattgagcgcagggtgggtggggtgggggcgcaggattgggcaggtctgatgctacttgggcagggcttgctgcagccactgtggaggacagagaggtggttcttaagccaatggtgttatgttcctagggggattatgggtgcctctactgcattatacatgtcaccagggaagcgggggaaaacaggcagtgacgggcctcactcagctctcatgcagccagcaaggccactcttactcccgccatgcccctccaacagcactgagtttatatccaggcagcctttgagcagggctgagatctttccccaggctacaatccttcccactgagaaagcaagcagggctctcaggcctcccttccctgcctgcccttcccttcagctgcagcttctgcaattgtatgtgcacttcccatttgccccccaccccgtttctgcacaggaaaattcgtactcagccaaaattataacaaagatcagctaggagcttccttcaccctgtggcccctccccagttctactggctgccctcccttcctgaagaatctctgtgagatgaggccaggaatggtttccctgggctcccccaggcctcggcacaaggcctccagccctccctagagaggcagatgagagtctttccccaatggaggtgttctctcgctgagaccagggcaggtgccaaggtgtctgtggagttcctgggggcatccgggcagaggggagaaggaggacctgggagagcacaccttcctgccctcactagagaggtatttattaacttctgcgctcctggccactgactgaattcgtgtctacattatcagctcatgtaaattcattatcccaacagctgggaatgagctccactggatgaggtggtggatgagggcccgggggctggcagctatccatgcactgtacaagctctcttgacatgggcagttcagtggaagtgtctacagggctcttcccattgcttcttctacttttatgttttgcttggcatcctaaatctgtttcagctctgggtaaggttaaatccttctcctgtgatctggattttcaggttccccagtggggatgtgtgttcagaggcaaacttttccctgctcacactttgggaactcacagttttttggctgtctcatagagtttgcaacagtaaaactgcttctttcaaagggtctgtgaattctttcagttttcctggtatgttcccatggtagttcttgcagcaaaagctcacagtgtgagtctccacacactgttctgtccattccaagcaggagctgcatgttagttctgtctgctatccaccattttccaattttgtgatatagttatgattttttattggtttgttgttttgtctttctatttaggacaagaatagtgtacacactgcagttagtgttataatatttagtgtttttctgtgtacttactattaccagtttgttttgtatcttcatattacttattattacttaataagtgattacttactgctcattaatgtccttttctttttgattgaagtattccctttagcatttcttgtaggacaggtctggtgttgatgaaattcctcagcttttattttggaaagtctttatttctctcttccatgtttgcataatattttcactagatatattattctaaggtaaaagatttttccttcagcattttaaatatatcaagccattctctccttgcctgtaaggtaaggtttctactgtgaggactgctgccagatgtattggagccccattgtacgttatttgtttcttttctcttgcttgttttaggattctttctttatccttgatctttgggagtttaattatttaaatgccttgaggtagtctcttttgggttgaatctgcttaatgttctataaccttcttgtacttgaatattgatatgtttctctaggtttgagaagttttctgttattagccctttgaataaactttccacccctctctctttctctatgtcctctttagaccaataactcttagatttgcccttttgaagctattttctagatcctgtaggcatgcttcattctttttgattcttttttcttttgtctcctctgactgtatattttcaaatagcttgtcttcaagctcactaattcttctgcttgatcaattctgctatgaaaagattctgatgcattcttcaatatgccaattgcatttttagctccagaatttctgtttgattctttttaaaatttttaaaaattctactttaagttctgggatacatgtgcagaacatacaggtttgttacataggtatacatgtgccatggtagtttgctgcaccttcaacccgtcatctaggttttaagccccacatgcatttggtatttgtcctaacgctctccctccccttgactccaaccccttgacaggtcctggtgtataatgttcccctccctgtatccatgtgttctcattgttcaactgccacttatgagtgagaacatggggtgtttgattttctgttcctgtgtttgtttgctgagaatgatggcttacagcttcatccatgtccctgcaaaggacatgaactcattttttttatggctgcatagtattccattgtgtatatgtgccacattttccttatccagtctaccattgatgggcattcgggttccatgactttgctattgtaaatagtgctgccataaacatgtgtgcatgtgtctttatagtagaatgaattatgatcctttgggtacatacccagtaatgggattgctgggtcaaatggtatttctggttctagatccttaaggaatcaccacactgtcttccacaatggttgaactaatttacactcccaccaacaatgtaaaagcattcctatttctccacagctttgccagcatctgttgtttcctgactttttaatgatcagcattctaacggtgatgagataccaatgagatggtatcttattgtggttttgatttgcgtttctctaatgatcagtgatgatgagctttttttcatatgtttgttggccgcataaatgtcttcttttggggagtatcttttcatatccttcatccactttttgatggagtttctgtttgattcttttaaagtattttaatctctttgttaaatttatctgatagaattctgaatttctcctatgtgttatctttaatttctttgagatttctcaaagcagctattttgaattctctgtctgaatggtcctatgtcttgtttctccaggattggtccatggtgccttacttagttcatttggtgaagtcatgttttcctggattatcttgatacttgtaaatgttctttggtgttaggatttattgtaggcttcactgtctgtgtttttttgttcctgtcctccttgggatggattttcagatatttgaaaagaccggagtattttgatctatgctgtatctgctttagagggtaccacaatcccagtaatgctatggttcttgcagtttctagatgtactgctttgatggtcttggacaagatccaggataattctctggattaccaggcagatactcttattttctttccttacttctcccaaacagagtccctctgtcagttctgagacacctgaagctaggagtggagtgaaaccagcacccctgaggacaccaccactatgactgtgctcagccagacttgaagccagcacaacactgggtcttccctgctgtaaccactccctggccactgcctatgtttgctcaaggccttggggctctacaataaacaggtggaaaagccatccaggtctgtgcccttcccttcagggtggcaagttcccccaagccccaggtgggtccagagatgttgtctaggagtcagggactagagtaaaaaacctttgaagtctacctggcattctattgcattaaaattgagctggggggactcgggggaaagtgtgagaggggagtaggggataaaagactacacattgggtacggcgtacaccgcttgggtgatgggcaccaaaatctcagaaatcaccactaagaacttatctgtgtaaccaaacaccacctgttccccaaaaacctattgaaataataataataataaataataataaaatgaataaacaaaaacactgagctggcactcaaaccacaaaacacagtctttcccactctttcctcccctgtccaaaggcagaggagcctcactccacagcccacaagaagtactgctggattatcacaggtattcatttaaggcagaaaggctattaagtcagctggtggtgactgctgcctggcctgggactcactctttggggcagtgggcttccctgtggcccaggaaaggtctagaaatgctgttgaagagtcaactcttggaatcaagggcccccaagagcttgcttggtgctttacctccctgtggctgagccagtacctgaaaccagctagtctcagaggctcacccaaggcccttgatgtagtatctgggtatcactgctggttattcagagccaaagggcttttcagctagcaggtgatgaacgctgccatgactgggtctttcccttcaaagcagtgggttcccttctggcccagggtgtttctaggaatgtcacctggaagctagggcctggaacagggacctcattattctgactggtgccatatctgttgtggctgagctggtatccaagatgcaaggcagagtcctcctcaactctttcctctcctcttctcaagcagcaggaaggggtctcttttagagttgtgagttgtgcagcctgggattggggaggggtgatgccagcactcccttggctgccccagctggtgtctcagtatattttgcacccctcagtccactgtctctggccctagttcagtactaggacttgtctaagaattgcagttcttttggcctaaactgcctttcaggtttagtcagagatccagagcacttcagccctcagtggtgaggtttgtgggaactgaaattctgactgctggaattagtgattcccctttggctgggactggtttgaatgctccctttatgtgtgggcatcagctgaactttgtctggctttcctttttgctgtaacaggacaacactgagtttaatgcctcaccattgatgtgttctccctcacccagtgcacgaaaatgctctttgcaccacaccacagctgccagggtgtgatggaggggtggcttcagtgcttcaagactccttctgcaaccttttcagtgcctttttcagcaacacaaagttaaaagcaggtactgcaagtgctcacttgagttttggttcttgtgaaggagctttgcttgcacagatagttgttaaattggtgtccttgttggggaaacaatcagtggagccttctattccaccatcttgctccacctcccatagtactatacatctcttcttttggctagttctgtttggatatttttgctataataaaactgtagttgtaagtatagtgctttccagagttctttaattttttttagtgaattatcaagcttgaagggttagtggggattcctaaatatggtagccagctgtttagaagtataggtagcttacgtaaccttgaacttgcagctgatgtctgaagtaaggacagtgttatggaggaccatgtccttaacttttgaagtttggctcaactctagtagttgttgtcaaaagttgatgtaacatgcctagtaattttaggaatgctggatattgtaaaggctatattcttacttgtctggatttaattgacttcctttaaacagtgttggactgtgttaggttacttgtgagtcatcttgatcctttgggggattgattttatgttttgttaagaagatagcttttattctacctaattccaatactctgtagatcatttctactactaaaccatggccttctgggatctcccagctagctgtttcctggagattataaatgactctaacacatctttcatatgcaaaccaactaattcagggctcatacgttccccaaccacttcctttatcaggactctacaccctgggccactattctcctgccctaatcagccaggtccaggtaacagaaaagtaaagacagccgctgtaccccagagcctgctaaaagtattcaaacgagctaatcctaagcctgattaccttgtcatgcccactctttcctgcagaaactacagtaaaggctcttgcccaccttgacccctcactccagctgcctcctaacactggtgcttctccatgtggtcttgggtggtgtgctgtgtcttctgtttgtagggatctgtcgatataaaccttttccttcacgatagtcatttttgtgtctgcatatgttaccacattgattaaaaagagtaagttctgtatgattccacttacccgaaattcaaaagcagacaaaattaacttatggtgttagaagtcagggtagtggtgttttttttttttgctttttttttggttttttttttgctttttttttgttttttttttttttcctggaaggaggatgtaaggaggcaggagcacagatcctgatatgctgataatctgtatcttgacctaggtgcagcttgcaagagggtgttcagtttgtgcaaatcaaccaagctgtatatttataattgggcactttcttcttgcatgtaatacagaccagttgcattattggttccaattttctacctcccactatatccctgccctttaccatgtaactttacaaatagtctctcactttggctctagaatccaccatatgacttgatttggccaatacaataaataggaagtaagggtgtgcttgttcttggcctgagcctgaagaggccttgtgtgtttcttctactctcgtacttttgttattaacattatatagacatggtggagctagctcactcatcccaggatgagagcctcagctaagtttctccatccaaacacagcctggagctgggctctaacttgttacacagatgaatgagtgaatattgttaagataagtcaagcccagcccagatttctgacccacagccaacccacagatgcataagctgaagatgactggttttatcaagctaattgttataatagtggagaaaagatcatgaggacaaaaagtgggcagagtcggaagaaaagagaggaagaaattgagacagaagacatttcatttaaaaaaatattccattgagctgggtttgaaatagtgcactgcctgttctcctaatgctgtatggtatcatgaaatctattgtttactgagtctatgagccagcttcctagggaggctatggcaattgaggacagggaagaggtaacactcaggaacatagaaggagaatttgggtccaagtgggtggagggaaaaataactgggtttagttttgggtagggctggttttgaggtctctgaaggacatgtaagtggagttttccagcagggagaaaacgcagagctaaagctcatatctttgctggcaatctagatttgggcatcttcaacaagcaggttgtagctgaggaagctgggactggacttgtactctaaagccagtgcaaaaaaagcttgaaacggctatgatggctaagacctggcttttccatgaaaaatgcttcggtcagtatgagtgattccaaagtggtgatcaattaaaaactgaagtatgattagcattaattataacccaatgggaatatgataaacgactcttggtcagcaagcagagggtgccctgtagtgctaaagcatcaccatataccatgtgtgccaagaatcaggagacaccccaaacgggagcagatgaggggttgtgtctgtcattggaccagctggcctgatccagcagaagtggatggagatacactgaatggggctcctgggaggcgtgagttgaaggggaaggaagagaagagacctccaaagtaagggaagagtgaaaaatgagaaggactggggtggagccccaagtagggaccagaggagaaaacagggataaagtaatcaagggagatgggacaggaagatgaaagaatgaggtaaacagcaggtgggaagaggaggtcaacctaaaggagaaagccgggtcgaagaaagaaggaagagaagaaaagaagggttgggaaacagaggaggaggcagccaagaaagcctggaagctgaatcatagaacggaagaggtagaagacggaggggctggaggataacataaaggtgggaaacggaagagagaaagaaccgcgtctgcgtgtatgacggctagacaggagttcagagaacagcggggtcgccaggccaccacctgatgggccacggctcattggctctaggagctgggaaagggcatcccaggaaagaagccctagactttagcctgagtctgggccactctaggggaccgggagtggggtggcgggagaggacgcgcagaatctcgacttctggccccaatctgtgcatgatcacccgagctcagcggacgctcctctctgacccaggcaggcggctcagggacgcgtgcggggatgcagagagaaaccgctgaggaattagggccgggagagactggtacctgccgggggcgtgtggtggggcagagctggcactgatgctgagagtggctaaggagcgcggcgccccagagcagaagggctggcagacgctcagagagccaggatggttcagggtccaaggaaggtcctatgttgggtgggagctgtgagggagtgaaagtgcatgaggaaccggaggagatggaaagaccttggcttgggtgttcgagggtgggactgcgtggtgaccgacggcacagagggtgtgtgttggggcggaagaaccaccccagctgaatcgtccccgtggggttttcttcccgtgtcttagttccagaagtatgcgcatcagacgctaatagttgaggaacaagtcatggaaggacagcctaagcgggaggtgaatgtaaagccgtggagagggcgggcgaactaagaaggccttcgttctcctccggccaccgcggctgcatccttgagaaaggggtattgctgcgaaccgcgccagggctggacgcggcgaggtgggaggcaggatggaggggcgggagccaaggccgagggggcggacacgggtggcgtctggcgctccataaaggggttgcgggggccgcgctctcttctgggagggcagcggccaccggcgaggaacacggcgcgatgcaggttcagtgccagcagagcccagtgctggcaggcagcgccactttggtcgcccttggggcactggccttgtacgtcgcgaagccttccggctacgggaagcacacggagagcctgaagccggcggctacccgcctgccagcccgcgccgcctggttcctgcaggagctgccttccttcgcggtgcccgcggggatcctcgcccggcagcccctctccctcttcgggccacctgggacggtacttctgggcctcttctgcctacattacttccacaggtagcgtttttcccttgcgggcgcccagtgcagcgcactgccctgctcccggcgtccaggagcgcagcgtagagcgcgcaccgaggaacgccaaggaggcagcgtggggcgctgtgaggaacgcggaggccagcctgcctggcgcacctggcgggggccggggccggggcttggacgctagagagttgacaagcggctgcggcacagacgccgccttcactcccccaggacctcatccgctctcagcagctccaaatcctctggctgctcgagagaaagaggtcctgcgggcgggaggcgacctggaaagctcaggttggggagacctattggacaggactgttccttaactgcgcgcaggcggcggttacctgtggcatatcgcgccccgccccgcttccctttcttccttcaccttttccttgcagggtggggacagattagttctgggatcttgggagagtcttacgcgtttggcatctcccgagggaaactcagctcagaggttctcattgatttagtctttgcaggtctaaggaggaaggtgggtagagcccaaatccagacttcacagatggggaagagaatatagtgagatcccactgactcaagcgagggtccccgcttcttaggcccctcccaaaagggaccaagcacaccaccaattcatctacctttccaatttccctgggacttgaaatgtgagtgggtaacttacagctcaaatgttcttctagatttaaaaaatccgggttcaggagaactttgctttctgctctgggaaggaaagacaggaaagagaccaagggatgttagggttcagagaaagactgtccagcctcagtaaagtctcacagggaaagggaagaacgaacacattgttaaattgcctaccatgcccaatcgtttagtatctatggtctcacttaactctgttgtaaccttttgagagatatttattatgtccactttacaaagaaactgaggctgagtaaggttatgggactgaaagaagacaatacagctggtgcccgctaagatgcaaatctgagcttgtgaatctctgaggtttaagaccttttagccgttttaagtaggcagatattctatgcaagaagagaacagagcccataaacagagaaaagttatcaaacgaaatcaaaaccttgaaggttttgccttattttgagtagaagattcctagggtcaaaagatcaaggaagtttggattagaaattaggataaagaatgcctctggcataatctgtctcgtaaagagaaagtgatgcctcatttcgagagaagaaggtgtcagagacggatttcaaactgtctttcttaatttcctcttatcctctgaatcttctttattggtcaagatttaatatttatgcaatcttaactgaattctcactctttttaaaaggcaaatgttgtcaagtaataagaactaaactgtatcctttcaacaaatatttactaaaagcctgctgtgaacaagattctttattttacacagattttattttactcagatttagatggtttaatttgcacagaaagaaggatgttggatacaggagataagagattagttttttatttcactgatttttgaacagacatttgacagaaatgtgatttttgtcaacaattatttttatcttcaaattaactactgtatgttggcaacataagcaatatttttatgaggtcatttcatcttaatgattagtgttttgtgacaatcatattaatgtaatctttaggcttgtttttgaaatcaaaaggcactttgcacaatacattatgaccggtgcatttaaaaaaaaacccaaaggagtaattatgcataaaacaaatatctacatattgtccttcttcagaggaaattttaaaatattagtaaaggacttggagattcattctgcctgagggaaaattgcccactggaagtacaactcttgagctacttgttcctgaggcatcgggatctgatgtttgtctttcctcttttttgaattctttctttgaagccactggagtgtgtgatctgttattatttgagtgcttacagggtgttacactctgtgtgccaaacactctgagttaataatttactcatcctcataccaacccatttaggtaggcacgcccattttgcaggtggaaaactggagcaagaaaaggttaaataacttccttgcccaaggtcacacaataacgaggaatcagagttcaaacccaggcagtagactccagatctcaggctcttcaggtatgataataaactgttgtatacttgttggataaatgcgacctcattcttgctttttaaaaactatatttattcttaaaattattatgtaaaaattttttaaattataaaaacattttttcctaatgaaaaatggaaaaatcccaaaagtagaaaatactcataattacatctaagtatattaaatattaatgttttagagaatttctatcaaatctttttccttagtagattttcacacatgcttgttatgttgaatatatggttttttagttctgtttaattacctaacattttatagtattttccaacattatcacagatttcttaatatctgcttatttcatctactgtaaaaataataatttacctatatattcatttatagttggacatttaatttgtcttggggtgaggctaggttcattgaagctgtgatgtacattttttgcatctaaattcttttcaaaggttagatctattttcttaggatttccagaagtggttcaaaggttatgaacattctttagggtcttgatataatcatacatatctgtttcgaggcttaaggagtcatttggcttgatactgagggtgcattaaggaacaagcatcaggctgtcttggctttatctcgtgctgtgtgttgctaagacagagaataagacggagagacagggagaaaggatggctcaggtttggctcagactcttttgcaaaatgtccattggggactgttcaaagcagcaatatgtcccatcaaatagaagttaaaactcattgtacttttctcaatacaaggatggcacacaagaatgtagattccacagggtcaaaggtttttgtctcacttttatggctgtatccccattgtatacaaagagctggtacatagttggtactcactagaatttgttgaatgcatgaatgaactctacttacaacaaccgtacagacagaattgtaggagagctagcaattacttgggaatcataattgctgagattgcatagttaggtcaattcagataactttcctctcttcagaatctattgttgtggctgtttggattctttagccacatggaaatgactgctgcctaagcacattgcctaatcccactgccaaaggcatttgaagtcaagagttatctcacctcttttttttttttttttttttaatgtttttttttttattatactctaagttttagggtacatgtgcacattgtgcaggttagttacatatgtatacatgtgccatgctggtgcgctgcacccactaacgtgtcatctagcattaggtatatctcccaatgctataagaaaaaccaacacagagaccaccacagttcccagagtgtgatattccccttcctgtgtccatgtgatctcattgttcaattcccacctatgagtgagaatatgcggtgtttggttttttgttcttgcgatagtttactgagaatgatggtttccaatttcatccatgtccctacaaaggacatgaactcatcattttttatggctgcatagtattccatggtgtatatgtgccacattttcttaatccagtctatcattgttggacatttgggttggttccaagtctttgctattgtgaatagtgccacaataaacatatgtgtgcatgtgtctttatagcagcatgatttatagtcctttgggtatatacccagtattgggatggctgggtcaaatggtatttctagttctagatccctgaggaatcgccacactgacttccacaatggttgaactagtttacagtcccaccaacagtgtaaaagtgttcctatttctccacatcctctccagcacctgttatttcctgactttttaatgattgccattctaactggtgtgagatgatatctcatagtggttttgatttgcatttctctgatggccagtgatgatgagcatttcttcatgtgttttttggctgcataaatgtcttcttttgagaagtgtctgttcatgtccttcgcccactttttgatggggttgtttgtttttttcttgtaaatttgtttgagttcattgtagattctggatattagccctttgtcagatgagtaggttgcgaaaattttctcccattttgtaggttgcctgttcactctgatggtagtttcttttgctgtgcagaagctctttagtttaattagatcccatttgtcaattttggcttttgttgccattgcttttcgtgttttggacatgaagtccttgcccacgcctatgtcctgaatggtaatgcctaggttttcttctagggtttttatggttttaggtctaacgtttaaatctttaatccatccatctcacctcttgttgactgtgattacagctgtggaatgaccccttgtctaaactctctgattggtcttcactttcccaagaagtcttggtgttccatacagtggtcagtcacatcacttatgcatgtggtggctgatgtctttttcaagactttgatgggaccttgataactgaatgaacaagacaaagaaacatttttttttccagagaagaagtgactacattgtgaaaaggttttgggcaatgtaatttccatgatatacatctagaagcagagtataatgcacacattagagtctgtttgaattcggagagatattccttgaggtttagatgctgaaacatttttttgattagccaaataaaatgatggcttcaataaggtgctttttgctttattagtcagttcattccccagcggaatttctcctcatccttcaagattcaggtcaggcatcctttcctccaagaagccttgtctgagtcttttcttttctctaaatccagttgtgatgctgttctttgggctttcataataggcagtgtggctttcgatgaggcatttcacacactggatcataattatctgttctaaggaaacgtagttctcatctgcccagcctcttcattttacagaagcaaagcacaaaaggttgggtcagttagggggaaatcaaagagttggaacttgagtcttctcatgagcccctgccaatgcttctcttatttcacgagaggtgcagatgtctcaactattcagaagtaatcagaccttgctaaggaaactactcatctgctgagaatcagaatgagaattaatggctactttggaaacaatagacaaatctctcctcttagagattaagaaaaaaaaagcccaagcaagaaaagcaacattcctgtgatcacttaatcatgtgttaaggatgttcctgaatcataatttaaataaattaagattaattgaaggatttttttagaatgagagtcagcaaactttttctgtaaaaagccaggtcatgatctctattgcagcaactcacccctactgttatagcgggaaagcagctacagacattatgtaaacagatgaatgtggttgtattccaataaagctttatttacaaaaacaagcagtgggccatatttggcctatgggccatagtttaccaatcccttctaaagaaaactatgaatggcgtggctttacattcagtgccctggtaattctataataaaatttggttaaattctatagcaaagcagcgggcatatcttagaaaatttctgctacatgtaagtggggacttggcagtaaatattgttttctgagaattcttgggttctaagtttttatttgaacttttatgggaaaatagtaggagtcttggagttttcattctaaatggaatgacatcataagcacatctgaaaaatatatagctatgtttgccaaaaatataaaccagctttggagatcgtgaccacttcacttttaccacactgaagaaaggaccaactttttgtccatttaaaatctaatttaatgtcacttaaaaagaacaaacaaaataaagaattacgatgggaattcaaggattcagactggcaaagtgacatgtaccaagttacatggcaagaaagggcagagtggagcttgaagcctggtctcctgatctttatcagagttctataggctcagaactatgctaggtctcatgggagtcataggagtacaatacctagtgcttgccctctaggaagaaggcaggaaagaaccttcattgtgtgcttctcactctttctggtgtgaaacttgggtggcagcaacgtcagtctgacaatatcaggcaggtgggaaaatgtaaactgagggggaaagaagcctggttagcacgggcccccgccaggccactgaacattcatctgagagggccctagtgcctgtctatgtttgattgtgcatgggacagggggcagatagataggtggataggtgttgagggggcctcaaaatggcacccttcttctccattcaagttcagaactaactgagagatccaggaattaggtaactagagacatattaccaccttttattactggtaaagctggtttgggggaatgtggcttagatttttggattatatgggccttataacacagtacctcagaaataattagctatgatcatggctgggcaaccacaggcctctcacagtggtacctgttctgaaaagacagcagaagggctgagctgtacgcatcctttggcaggtcctgctcccagattgacccatcaggtaattcacatttccttgcctgaggaggaatgggtgagggacagtggaagggcttgcagtgatgctctaatgaagggccctcagccagctgtagggggagcataaatattttttgttactagatggaatacaaacgttgtctacttattgaattttgccacagcctggcaacttgtttctgcaacttggggtacttcccgttctgctgttgactccctgccatgtatggcaatggggacatcaaagtctggactcaagttgttttaaaagtttctgtgctcaacccaggagcctttaacgagactcagctcaacataggagctctaaattttaaaaacccctgaactagggccaacagtgcaatttgtcttccatttggttatagaagggagagaagggaaaccaatatttattgcgctctgggtgttccaggcattatattatgttctttgcattggctatctcattttgtccttgaggcaatccctgttattagtgctattttacaagtgattgcagtgcagagcattgatggagtgagtgggagtgaagatgggagacttgacctggtccttgaagaatggcctgttcccagatttctgggtggggacatgatgtctgttgggaaaaggggcccatttggtgatctgtgacttaaatttgccttatactttgtatactcagtcttggagtatctctctacttttagattccaaacatcttctgtaactatgccatattatccagtcctactacccaagtcttaaattaggctctgggcctctttcaatttcttttttttaatatttgctccatattgttaaggctgatcctagaagttactgtgaagaaaacagggtaatttggttttgatattttttctttctaaccacgaaacatcccaagatcttttttcatgtatagactctggttctctcttgcctctctgcctgccctcttgtatcagcagtacatgatcttacttctatcttttaaaacttaactctgtgtctcatgctgtcagtcaatgccctttacttgccaaaagttactcatgtgataaacaaatctaagttctagtgtattgggttgtttgttttcttattattgagttttaaaagttgtttatgtattctggatagaagtattttgccaggtataaacttagcaaatattttctctcagtttgtgacttgtctcttttattctcttaactgtcttttgttttaaaaaaattatttttaggttcaagttaacatatgcagttctgttatacaggtaaactacatgtctcaggggtttggtgtacagattatttcatcaccccagtaataagcatactacccgataggtattttttcaatcatcaccctccttccatcctccaccctcaagtagaccccagtgctgttgttctcttagtatccatatgtacttggtgtttagctcccatttataagtgaggatatgcagttggttttcagtttctatgttagttagctgaggataatggcctgcaaaggacatgatctcattatttttcatggctgcataatattccatggtgtatatatgccacattttctttatccagtccaccattgatggatatttaggttgagttcatgtcttgactgttgtgaatacgtgctgcaatgagcatgcacatgcatgtgtctttatggtagaacaatatatattcctttggggatatacccaataatgggattgctgagttcaatggtgattctgttttaaattatttgagaaattcctcctagcagtgtataggcgttcccttttctccataaccttgccagcatttattatttttgactttttaatagtagctattctgattgatgtgagatgatattttatggtggttttgatttgcatttctctaatgattagtgatgccgagcattttttcatatccttgttggccacatatatcttgttttgaaaagtatctgttgatgtcctttgcccacttttaattgaattgtttggtttttgcttgtaaattagtttaaattccttatagatgctggatattagacctttgtcagatgcacagtttgcagatattttttcccattctgtaagttgcatgtttactctcttgatagttttttttcctgtgcagaagctctttagtttaattaggtcctatttgtcaatttttggttacgttgcaattgcttttggtgtctttgtcatgaggtctttgccagatcctatgtccagaatggcattttctaggttttcttctagggtgtttatggttttaggttttgcatttaagtctttaattcatcttgagttgatttttgtatgtggtgtaaggtagtggtccagttccaattttctgcatatggctagtcagttatcccagctccattttgaatagggagtcctttccctgttacttgcttttgtcaactgtattaaatatcagatggttgtaggtatgcagcattgtttctgggcactctatcctgttccattggtctatatgtctgtttttgtaccagtgccacgctgttttggttactgtagccttgtagtgtagtttgaagtcaggtaatgtggtgcctccagctttgttcttttttcttaggattgcctggttatcccagctcttttttgattccatatgaattttaaaatagtttttttctaattctgtgaagaatgtcatcagtagtttgataggaatagcatttaatctgtaaatcgctttggcagtatggccattttaacaatattgattctttctatccatgagcatggaatgtttttctgtttgtgtcatctctgatttctttcagcagtgttttgtcattctcattgtagagatcttttacctctctggttagttgtattcctaggtattttattctctttgctgcaatcatgaatgggattgcattcttgatttggctgtcagtttggatgttgttggtgtataggaatgctagtaatttttgtacattgattttgtatcctgaaactttactgaagttgtttatcagaccaagaagcttttgggaagagactatggggtgttccaggtatagaatcacatcatctgcaaacaggaatagtttaacctcttctcttcctatttgtatgccttttatttctttctcttacctgattgctctggccaggatttccagtactatgttgaaaaggagtggtgagagagggtatccttatcttgttctagttttcaaggggaatgcttccagcttttgcctattcagtatgatgttagctgtgggtttgtcattgatggctcttgttattttgaattatgtttctttaatgcctcatttgttgagggtttttaacatgatgaggtgtttagttgtattgaaagccttttctgaatctattaagataatcatgtagtttttgtttttagttctgcttatgtaaatcacatttactgatttgcatatgttgactcaaccttgtatcccagggataaagcctgcttgatcctggtggattagctttttgatgtcctaccagattaagtttgccagtgttttgttagggatttttgccctgatgttcatcagggatattggcctgaagttttctttttctgttgtgtctctgtgaggttttggtatgaggatgattctggcctcatagaatgagttggggaggagtccctcttcctcaattttttggagtagtttaagtaggagtggtaccagctcttctttacacatctggtgtaatttgtcttaactgtcttttgaaaaacagaaatgtttaattttgatgaagtctaatttttccatttttctttggcttttgatgtgatatctaaaaaatttttgcctaatctaaggtcacagtgattttctcttatatttccttctacaagatttgtagttttaggttttaatttcaggtctatgacacattttgaattaatttttaaagtaggtttccaagtttgaatcaaagtttttttgttgtgtttttttttttttgttttttgttttttgcatgtggacagtcaattgttttagtaccatttgttgaaaatactactttttgtccaatgaactgcctttgcaccttaactttaaaaataggagaattatatgaacagatactttgccaaagaaaacgtatgattggcaaacaagaacatgaaaggatgctcattattagtgattagggaaatgcaaattaaaccacaatggggtatcactatgtgtctgatagaatggctcaaataaaaaatactgaccataccaagtgtttgtgaggatgtggaagagctggaactctcatacctttgatgggaatttaaatgacaaccactttggaaaatggtttgatagtttcttaaaaagtgaaacatatacctatcatataatgtagccattgcatttctggttatttttctaagaaaaataaaagcatgtgttcatacaaatggtggtagcagcagcctgactggagtggctgcagtgttagaggcatggctggggctgcgcactccacagagctggcacggccaggaacaggcatgagccccatcccctaccaagttggtggggcagaaggccagtgctccttggcgcagctgcagccacccagccatggctctggacccgggcttccctgtgctctcaggggcctgggaagcgcccctgcccctgaaggcttggaagtacctgctcccgctccttggcctcttcccactctaggcacctgctctggggtggaacaaagttgtggctgagccaaggtgctgttgctcggcctggcatgtgcaagctcagggcggcattgacacaccagccccctgccgccttggccccctccagactttgggtgctgatgagcatgggaggaggggcaagtgggggctgagggtggcttagtgcaggcttgcaagcaccccttggcactaacagcctgggcaccatggatggcatgtggatggtggcaggaggcagacaggctcctgggtggaaaggggcaggtctcgggtgaaaccccaccttcaagccagggatggcctgaagcctgggtgctgggctgtcaattctggatggagtcagtggcccagagtgacaatttatggtgctttttctgaacctgctcatggtggcccatggaccaatcagcacgcccttccttctgaggctataaaaactctggactcatccagacttgggcagacatcaggactaccagctgtgggaaggaactgcccactttgggtctcccgagagcctctctgtcactcactgaagctcctctctgccttgctcatcctccagttgtttgcgtacctcattgttcccggacacgggacaagaaatcaggacctgctgaatgcggggactgaaagagctgtaacacaaacaggtctgaaacatgcccccctgcttgtcacattgcaggcaacaagaaggagagaagagctgggacccttcggggagcccagaactaggggagccctgagccagggctatgacaccctctttggggctttgtggttcctggcatctccaagcttccaggcaccaccatgttcccttcatccagatgtgggtgcccacagtggaagccacttggggtacatctggtccaaccatagcctcacatggagccggcacttatgctgtcatctggagctgcttgccttgcccagcaaccagtgtgcctggctgtgcacagtggctggaccctgcgctcactcacacacacacccctcatcattctgtgcctggctcacccttggcaggtgtgggatccaggcctgtagcatgagccaagtgcagcctgccaggctgagtggacagattgagctcagtgggcatgagcaatactcaggcagaaggcactactggccacagaggtttccagctggtgaagtgacaccccaaggatctcgtgacacaaagacttgtatacaaatgtttgtagtagctttatttgtacaagccaaaaactaggagtacatgctttatgattatgtttatataaaattctatgaaatttaaactaatctatggtgacacaaagtacatcagtggtttcctgaggacacaagagggacaagaaggtagaactacaaagggtatgaagaaccttttgttgtgatggatgtgttcattatttttatgtcacatggcttcatgtgacatgaaatatatctaccaaactgcactggtagatatatatatatatatacacacacacacacacacacacaccaaattgcaccctttaaataggttcagttgactgaatagtaatttacttcaataaaaataagaaaactaggtaggcctgtgaaaaaattatatgtatatgtggccctgtgtcactgagaattgttcaaagtttctgtgaagttcatctggaaatgttcaacaagtgctgataagctgtttcacttttttgaggtgtgagtagaggagggccaggaattctactcaagggaaataatccaaaaggaagaaatcagtctacacaaagttatagtgttactattcacaactgtgaaacactggaagttacctaaatgctctacaattggggattgattgtaatcaatatgattaaattaccatatatatttcaaaaatattagttgcatggactatatctgtgtatggaaataaatatatggagccatttagttaaaaagtagcacataaagagtacactctgatttaatccatgttgacatgtacaaatgaaaattgaccatgattggaatataaagtgatgtaaatagctgataatttaggtttgtattttttccttttagtattattaaagtatttaattatttaactcctattataccttatagaagaaataatactgtattacaaaattttaagatgtattttacaaactgaccatgcaattaaatctcagcagagatatgacaccccataagtagttactgttctgagggattttaaaagtcacacttcgtgtgtgagatctcccccactgagctgctatgacacaacccacttatgaaattgtcatctttctatttctcctttgtatcaagatgttgatgatcccttaacatgcaatcatgttacctggcaaagtagcacttggcaaagtccaaactattatccttttaatattttgaatctgagaggatctcatctctttcatccttaatcatggtaatttatattttccactttttttccagatctttttgactagagctttagaaattgtattgatcatctcagaactagatttttatttcattgagtttcactattgattttgttttacatttaattgttttctgttttgatctttattatgttttttcttatgcttactttgaattttatttgctctcctttttctatttttattaaggtggaagccaaggttataaatggagactttctctttcttctaatatgagcatttattgctttgaatttcatcctacatattgttttgtggcacctcacaaattccgacatgttgttaccattttaatttatttcatgtattttaatttctcctttgatttattctcatatctatgggttatttagaagggtgttatttagtttccaaatgtttaggaattttccagacatgttctgtattcatttctaatttaattccactgtggatagagagcatactttgtatgacatacatccttttaaacttactgaatttaaaacttgttttatggcccagaattgggtctatcaatcgtaataattgttctgtgtaaacatgaaaagaatgtgtatactgccaagaagaggagtgttctaaaattgtccataggttattttgattgttagtgtgcttcaaatttctatatcctgatgattttctctctacctattctaccagttattgagagaagagtattgagatatctgatcaaaattgtagacttgcttttttttcttacagctctatcagtttttgcttcatgtattttgaaggtctattattacatgcatagagacagaactggtatgtcctcttgatgaattgatgcttttatcgtgatgaaacaatcttctttattcttggtaatatcttgctttgaaatctacttttgtcttatattaacttagccactacagatttctttttactagggttaacatggtatatgtatctaccatgtttattccaacattttacttttaatctgtttctgtctttagatttaatgtgtgtgtctcaaaggtggcatactgttcaatttttctttccccctgcccctgccattctgacaatctctgatttttaactgggatattagaatgtttatatttaatgagattatttatacagtttgaattatgtctatcatcttgctatttgtttccatttgtgtctctgttatttgttcctatttcctatttatattccatcttttggatttagtattattttatttcaatttatattgtcagtttattcattataactcttttgttaatttgcttaaaggtttattgcatgtatcttaaatttatcagtcttccttaaggtgatattataccacttcaagtatggtataaaaatctgaaataatgtacttccatttcttctctcttgcccttatgttattgttgttgttttacttacatatgtgctataaaagcctcaacttattactattatttttgtttaaatcgtcaattatcttttaaacagttttaaataagataataaaaaagttatatatttgcttatgtggttgccatatttgatgcttttcatttttgagggttgatttatatttctatgtggtatcattttttccttttacctgaaatcagaatttatatttctgaaagtgtttttatttcttgttttcgaagatagaaaattctaggatatagcaatccagattgacagctgctttctttcattactttaaagatgtttcactgtcgtcttgtttgcattatttccaacaagaaatctgttcttgcccttatccttgtcctctgtacttaacgtatcttcttatctttgcctacttttaatattttcatgttatcactggatttgagatgttgggttactatattccttggtgtatttatatttcttattctgggcttccttaaacttgaatccatgagcttgagtctttcatcaaaattggacgattttcatacactattcttcattccctccctacttctcctttcttttgctgaggaaaatatctatgagacctacatgtatattaaactgtcctacaactcactgatgctcttttcatttttgtggattcttttttttttctgtgtgcttcttttgggtaatttctattgctattcttcaagttcataatcttttcttctacaatgtcttatctgttgctaatcccatctctggtatttttatctcagggattgtagtttccatctctagaattttggatttcggtctttttttctctcttccatgtccctacttaattttgtgaacacatggaatgcagttataacagctgttttaatatcattgctaacctaatatctgtgtcatttcggtgtcagttttgactgattaattatttttcctataataagcagtgttttcctccctttttgcatgcctggtaatctttgattggagggtcagaaaattgtacctcattgggtgttggatatttttgtgtttctataaatattcttgagctttgttctgggatgaagttaagttacttggaaacaatttgatcatttcaggctttgatctgatttgttcagtggttccagaagagtgttaatgctagggctaattaataatttcctctgaggtaaagcctttttgagtattctacccaatgtcttatatgagcttttctatctggcttatgggtacaggctctatttgtggccatgtgtgatcactggatactgttccctttaattcttttgggtgattctttcctcacacaaatgcatgaattaacactctgctaatacttgaggaaaattctctgcaggtctccgtgtgtctctgtccttttcagcactctgtcttgaaaattctagatgtcttgatttcttgaaatctcaattttgttttctcaactcaggaagtcttttagcttctccctgggatccccttccctgtacctcagcctggaagttttctcaaaccaggaagctgggtcagttatgaggtggtgtgagtcatttttttcttgccactcactgtttttcattgcctaatatccagtgttttgtaaataattatttcatataacttaccttttttttttctagcctggaggataaatccaattcctgttattcctccttgactggaagcagaagtgtctccaatgggttttagtatttatctttgctaattggataatctgcaatactggttgatttgttggttttagtgacctctacttgtactcaatgtcatgtttttttccccaaactttccatcttcaggcttgacgtgtaatgctttgggacgtattttacttagaagactaagtgggatagcagaatgatgacttcctttacatgagtcctgtggctccatgtagctgcagctgggaaggtggctgtaggactatagagtaggagataaagagtgggagataccaacagatggttatgaatgattggtttgaaactttcacatggaaaattatgttgcagaaacctcagattgtgcttttgttctttttttgtttggtccaaatgaacaatggaatggaggtagatgtaggagggagatatgctgaggtagatcaatctttgaatgttgcctttgatcagggctactgaatccctggacatttatttatttgttttaaaaatttaattcagggggtacatgtgcaggtttgttacatgggtatattgtgtgatgctgaggtttgagcttctaatgatcccatcacgcaagtggtgaacatagtacccaataggtagtttttcaacccttgcccctctccctccttttcccttttggaatccctggtgtgtattgttcccatctttgtatcaggacaatatttaaatgtaggtggaaactttttaaaagttctcaaatttatggcaatatctaggcctcattgctgcaaaactgtacagagctgtttgcttccaaataacatcagaagacttgggattgttaatgggtacattaatatgattagattgaatgaataagttctagtatttgatacagactgactacagtcaataataatttattactcatttaaagataactaaaatagtataattggattgtttgtaacagtaaggatcaatatttgaggtggtggataccccattttaccctaatgtgattattacacattgcatccttgtatcaaaatatctcatgtaccacataaacatatacactatatacccacaaatattaaacataaaaattttttaaaaaggaaaaaaaagacctgtaaacctgggctgcactaagaagctaaacaactgaacttagaaaaccacaattttctttttgcaaacattgagaaataaaggagacaaaattatagctcagagaaatggcatgtatttatccaacagtgaaagtgcctgttatgtccttagctgctcttcataggcttgctgagctttttacttcaagctgaaaaccatcccatgtcaatatttgtattttcttgattttgtaataatcagttagcatgagtggctgagcaactgttgtttcatgatgccaaaatccacctcaagttgatcaatttatagaaaacggtaaactagttattcaaataagttgatttagttactcaggccatgaagttgatctgattctctttgattttgatcttctggggtattaaaaatttttttaaaatggagccagtggtcagtagcatgcctatgggaggtttcactccaatgatcattattctttctatcattctggggaatcctcaaagttttttaaaaccttggtaattcattagatgtttggattgaacatctattttaatgtagaggcaggaagaaagatactgagtggacagttccaaggtctcccagtattaccaggacatgccataaatacttaaaaacataagcagggaacttaaaggttttctgctgtattttagatattcacaagctaatcatctcaaagattttgcatgatttaaatggatctgccagattaggaccattagatagttaaaaccagataacatttgcatggggttgacaatgggatgtggctgccctgtcttccagcatggcagagtaagatgggaagaagtcttgagtcattgctctttggggaaaatatcattatatgtctaggaggccttaatatttgtcttactagagaaagtaattaggttaatattgctgtgctaatagagtcttggagcctgaaaactgggctaatccacactgtaaatactggaggccgtgtgtttccccagttagggcacacttggagaggagtcaggtgtgctcttttacaaatctcattgcattattattgagctcttggaaaggtgctgagtcttcattttagaaaaagggattagatgtggagggaatcactttgctttccccagggaggcatttctctattgcttttctagggttgggataggtatatagaaggtttgattctagaatttataaaatgatattctgggaactttgagcaatattttagggagggcacccatccaaaaaactagatgagttgctgcctttctctgcaacagcccttaggagactagcttctgatcttcttcctaaacttggtatccagtttattttgcttgtcttcctgcaggccattcttgtggagacaccgagtaacaaagtcttgaagtaatgtgtgacaagctctgtagggctataggattatagagcagctactcagagggcaagggtatgaagaaaaagagcaggtgcataaaaggaacagggaaaggggagtaaaaggaaatggggaaagaggtttaaaggagatacaacagtaaaaagaagaaaagctgggaaaggagaaaggaagcgcagcaaaaaaggtggaaaaagaagagagagtgctgatgcaggtttttcaggggcctatattgggcaaagcattgaacaagttacacccaaaggccaggagcctgaaatcaggtgccggttctcaaataacaagggcttattaagaatcagtgctattgcctaggatagttaaacagaaatacaaaaagtgctggagaagttcagggaactgtgagatatagagggaaaatcaggggaagatggacaggagatgggagctctagacacccttagacatcttggtttcaggttcattggtcagaactctctccccatccccacagtctatctagatgcttacatcaaaggcaatgcttgcttgacagatttctaaatagcagtagcaacaagatggaaagaagaatggaatgtcccattagaggatatagggccaggacttcagaaagagttcatatatattaatcacatgtcttagaatatcctggagcacctgtcctctcacctacctaaaatacttgattcccaatggtcaatgactttttaaaaaaagggggggggggggattggtatgtacatagatattgtgggaacgtacaattttttaaaaagtgatgataaagttctataggtaccttcacaaaccactctgttctacttaaatcttaacagttcctggaggaaactttgagattactcttcagagcttactttggtggttggggtgtgttgtatggctttgtgtgtgtgtgtgtgtgtgtgtgtgtgtgtgtgtgtgtgtgtgggtttgtttacttttacttatgggcactgatgtattggttactagaaagttcatagataaatttttgtcctattcagtagacaaacatacctgacttgttgacatagagtctaggtgctaacttcattagtcaattcaccttactttcatatctttattttcatttgcctcctttttctttaaattttaggccatcttacagcactttatacatctatgtttatggccttaaatcttttctttttctttttttaagagcaatttgggaagaactaaaataaaattgaaatacaaatcaacattttttgttttatgcaaatgtatgttaaaatattcaaagaataaagaggttttttaaagaatagtaggttctagcacggctcagttaaaatatggtatcacttagtaagaaacactttctaaaaaacaatttatattagccataattaagaaaaaaagggatattgaataagtcaaagagtggtttccagggaagtctttctttgtctggaaagataccataggattttctgcagacagcttactcatttttttcttgattgaaatctggaaaggaaaacttgcaagaaaacattccattctggactcggcagcaggtcaagccaaagtggcaacatttgcagcctggagatcacacagtctgggagctcaaacatttaggcaataaaagctgatggaagccaaaaagccacgccactggcatctggtgtcagggtagctgctgggggagatctggccatggtggtggggcccagccatataaacaagttcagaggtagaactggagctgtgtactgggttgaagcatgtctctcctaaattcatgttttctctagaaccttagactatgaccttatttggaaatagggtctttgcgatgtcatgcattgcttaacattgtggttacattctgagtaatgtgttgttaggtgatttcatcattgtccaaacatcatagagtgtactcacacaaacctagacggtacagtctcctacacacctagaccatatactatatcctattgttcctaggctgcaaacctctacagcctgttactgtactgactactgtaggcaattggggtacaataatctaatgagactataatcatatatttagtgtgccattgaccgaaatgtcattatgatgcatgactgtagttaagatgaggtcatactggattaggtaggttctaaatccaatgtgactagtgtcctcataagaggagagaaacagacacaaacacagggaaacaccgggacgatggccatctgaagacagaggcaaacattggagtgatgcaatgacaagccaaggaatgccaaggactgcaaaccattgccagaagctaggaacactcaagtaaggactcctccctagagcctttagaggaagcatggctctgccaaccccttgatctcatacttccagccttcagaactgtgagacaatacatttctattgttttaagctatccagtctgcggcaatttgttatggcagcctaggaaattaacaccaggcatacttcagagcaggacaggtcaagggataaccaaataggtctcctgttactagaattatttacaatgtcagagtagttcttgatgtttacttggccctctcttggcaaaggctctttgtattttcctacactaaaggtctggccaagtaggggttcagattctcttcaagtatagaaactccccccgatttttatgtttgctggccttccaagtaagatttccattgaagaaaatgctccatggctaaaaagctttaaaatccattggtattatcggcagatttggactctatagagcctagtttctaaagtgcttcatagtgccgatctgaaagtgagatagcactagtcctatcagaggattgttcaacctgagactctgcatcagaaggtcaggcagcactgacctgtgtcaggcctggagcaggctctaggaggctgctttcgtgatgagttcctttttgttcaggagaatataaggaagctggtcctattttagaagcaataacagcctttgagtttgctctagggcaattgcaaaatcaagagcccccagggtgctgtactccagatttctaggtcacagatatccagaccagagttgaccagaaattggccagaatgggttctggggatgtggatgaatttccatttctcatccattcctactccatctccattttatcattctccattcctactccctcaataccaaaattaaaacaaattgttgattactaggtagccttcacattgagtttttcctatggtataacccttgaaaaaaattgatcaaccagaccattacaagaaaaaaaattatggttttactgagttaagccttttactaggttttgcggtccaatttctctgtgtagacatagacttggaaaagccaaaacatctaagcaatgcaaatttaaacaaattagttaaatctgccactgggagtcattatgtcattggcttttaacctggctgcacattaaaatcatctgggggaacccttaaaatgacttctgctcaaagtcatgtgcccaaggttgtgattaatttgtcttcagggtgattcctcccctttaatatttctaaagccactcaggtgacttgcttgtgcaaccaggggtgtaatcctctgatgtggacatgtgactgcaatagaggaaaatgctagagagcagatactctggaatcagtgccacctggacttgaatcccaactctgatatctcataattatgtgaccttgggctaacttagtctcattgtgcctcagtttccttctctgtagaatgggaataataatacctatctcctagggttgttataaagattaaatgagttaatatgggcaaagtacttagaacattgcctggaacatagtacgcaatatataaatattagttgtctgtcgtcctttgctgacatttcccttactggatgtctgaactgaaattgatctcaatagtgtaatattactccaacaagttatattaacttcctgtgactgctgtaataaattaccacaaagtggcttaaaacaaaataaatcttactcttactattctgaaggccagaagtctgaaatcaaggtgtcagcagggccacacttcttctggaagctctgaaggaaaacctgctcctttcctctttcagcttctggtggctattggcattccttggcttgcggtcacatctctctttgcttcatcttcatattacttcctcccctgtgtgtctttcttctcctggtatttcttgtataagaatccatgtgtttgcatctagggcccacgtggattatacaggatgagctgcttttctaaggattcttaatgtagtcacatcttttgccatagaagataatattcacttttttttgctatcataaggtactattcataagttcctggaattaggacatagagatatctttttggggtctaccattcaacccactatacaagatggcataataaaaagtaagcttggatgggaggcatcactgactcattctgtgtttagtggacttcacatttggcctgcacatcccattctctaatatgtctaaaaccattatttacacaacaggtaccatccaaatatacaaacaagtgactatttattgtcaatgtttgtagttacttctcggcaaggacccacagaagtggagcaagtgctcagaggactggttggcccaatggcctttggcatatagagagctccaacaaggaaaggcttcctggtggttttgctccaaagagaacaattttttgagaaactctattttgtgctaaaatgtgtacccaaaatctcaccaatgatggctgttttttattttgttttcattttagagatatgatctcactatattgcccaggctggccttgaactcctgggctcaagagatcttcctactttaccctcccgagtagctaggactacaggctgcatgactacgcctggctaattttgggtttacatttggaaaagtctgatgcttattttaaaagagaacagggtgatgctacattatcaggacatgttttctgtcccaagtgatagaaaaatcaatctgaataggcttaagtgaaaccctaatttattaactctcataactggaaaattccagcttcaggtccagatcaatgcagtggttccaaagatactatcaggacctggtttctttctccagctcttggctctacttttgctggctgtagtttcagaaaggccgtccttaggtactggcaagacaactgtactaagacctacattctctcaggttcaaggcacatgaaaataaacagtcctagaaaaaaatgtaagcaaaaccttaccgagatttgttgggttgaatactcatgcctgagacaaccaatgtgatagcaccccttcctgttctgtttggccgaggaccttggaaaggatgacccctcttaatacatgggctaagactgtggaaagaggtaggtctgttctgaagaaccagtgtaggtaccatcagaagggagaatgaatattgggtggcaaatacaacagatgaccactgtagatgcaattaggtccttggactaaaatgtctgaaatccaaacctgggttttcttgagcgacatgtgggatttttttttttttctagttgtgtgtgttggtcacaatttccctttccacttggaaataagtgagttattttgaagcagaagacccacatttaaattccatctctgctgcctctttgtagctgtgtggccttgggcacattaccttatgtctgtaagctttaaattcttcactggcctggcggtaacacacacacacacatacacacacacacacacacacacacacacacacacacacacacacgatcaggatagaggccatcttaccaggactattcatcaccagggcacataaacagaataagcctacatgaacacagtctgattgataccgaagaaccatctgtccctacatatttttcatggtcctgaaacatgttggaaatttctaccatttgagtaatggaaggaatgtttaaagcaacatgtttccagagagccaggtgaaaaatgcaaattgatgccagggaaaagcaaaatgaaatcctgcatcactgactactccttatccgccaccttagaagggatgagccaaagtcaccttgtgtgatcattctcaacatgcaaggcaaccatcaagagacttcaggcaaaatgatctttctgtttttccacatttgttttaggattcttctctaactgcttctcctgaggcagtgttccctggggatatccaataccacagggaatgtccatattgaaaaactatacctagagctcttctttctccaagttgtctccagttgcaaaaatgacctcttcatctccgtaatcacagtgctcattttcacagtggagttgctattaggtagggcaagtaccatctgattatgaattgattaaagcattcatattaagcatgtgtgagtgacaggctcaacttttttttcagggagttagtgtttatcttcaatcttctagattgaggtaagacattttttgagaaagatacccgggtctttttccctttctcctagatcggttgcaagtgtactcacaacagatgttatgtggtattctacttctaaaagactgttggccgtaggttttagctatcacttggaagataacaagtagtagaattatccttcctgttatgggtaggaaggaattcctacggataggaattatctcaagatccctcacagatgtgcactatcatgctccaaaaggtttatttttcctagaaattgactagccttcaagtcttttcagttctcttatcaaaacttactactcctttgtccaaaccctttactattcctttgtccaaacctccgtaacccctgttacagcagccacagggagctaatataccttgttggagttatattagaccattgaaatcaattttagttcagacatccagtaagggaaatctcagcaaaggaccacagacagctaatatttatatattgcttactatgcttactattcctttgtccaaactcctcaaatatccttcctttagattgcagagtagtattctgtggtgtggacctacctcagttgaattggcagttttatttttcttcagctgtttgaatgtgtcattccactgtcttctggtgtgtaaggcttcttatgagaaatctgatggtattattgatgtgtccttgtatgtgacgtgtcacatttctcttgctgccttcgacaatttctttgtctctgtattttggcaatttgattgtaatgtgtcttggtctagatttctttatattcatcctatttgtggtgctttcagcttcatgaatctgagtgttcatttcccttctggaatttgagaaattttcagtgattgtttctttaaataagctttttttcctattgtctctctgctttccttctagaatcaccataatgtatatatttgttgtcttgatgatgtctcatatgtcccttaggctttcttaactctttcattcttttttcttttgcacttataactggataatttcaaataacttgtctttgagttcactgattccttcttaggtttgatcaattctgtgtttgaagctttctattgattgtttttctttcagcaattagattcctcagatgcagaatttgtctggttctttgtatagttcctatctttttactgagaatctcatttttcaggtattactttcctgatttcctttggttgttcatatgtttcctctcgtaactcattaaacttctttatgataagtattttgaattctttgtcaggcatctaaaagatctccacttctgtagggttggttgctggagatttgttttttcctttgattgtgtcatgtttcccagattttttatgtttcttgtagctttgccttggtgtctgcacatttgaaaaaacagccatctctcccagtttttatggagtggtgttgacagttaaagaccttcatcaattagcccagctagagatttggagagcctctcacatagcaagctgactgcctcttttcctttgttcttagctgcccccatcaaactatactcattctgtcagtcctttgggttgagtggaacagggactggcattcttctggcaatgtagtgaaagaccaagaggttggaggtatacaccactcttctctcttcttggtgagatgcctaggttctgcaatttctcccaatctcacagagctctgctatcctgtaacaatctgtttacgccctttccattgttgagttgaccgaggggtccaaactatggcagtactgtcaatgttccatgtggggcaagatagaaactgtccccttggacaattcactgaaagactgggatgccggatggtcattccacttctcttcccctcctaatgagagaagtgtgggttgaggcaatatcccttggcattgatctgtgctggctttggggagaggctgacataggtaaagtgtaattgctcttcttatttgttttaatgtggctgttcttcattttgtgctcatctaggatgcttgcaacttctcataaaagtagtctcacaaaggtattttggtgtgtatattattgttagcttgatgtttctgtggaataatgaggtgagggaccatttattctaccatcttgctgatatcactcccctgaagtgcaaaagttttaaattttgatgaagtccaatttatgagttttccttttatggatggtgcttttggtgtcaagtctgagacctctttgcttagtcctagatcctgaggattgtctcctgactgaaaggctaattttcctccattgatttgcttctccacctttataaaaaatcatttgagcctatttttatggctctatttctgggtcctatattctgttttgtcaatctatgtgtctatcccaccaccaacagcacatagtcttgatcactataactatttaataagtcttaaatctgataggaactctgactttttttttccaattattttagttatccaagttcctttgactttctgtatacagatggtccctgaaattcaacagttctacttaaaactttttgactatatgaaggtatgaaagcaaaatgcattcagtagaaaccatacgcagtgtccacacactgttctgtttttcacttttagcacagtactattaaataaattgcatgacatattcaacactttattataaaataggctttgtgctagattattttgctgaactgtaggttaatgtaagtgttctgagtaagtttaaggtaaagcaagctaagctgttatatttggtgggttagttgtattaaatgtattttcaacttctgatatattgaaaacaatggttttatcaggaagtaatctcattgtaagttgaggagcatgtgtacattttagaattatcttgtctatattttttaaaaatttctgggatgtaataaaaattgcattgtcattttgggggagaattaatatccttgctatattgagtcttttaatccatgaacatgatatgtcaccatttaaaaaagatctttaatttattttattaattctttggagttttcactatagaacccttgtacatgtttggttagatttacaacaaaatatggtagttttggggtcatcgtaaatttggtgatatttttaaatttcagtgtcaacatattcattagtagcaaatagaaatttaattgtgtattttgtatcctgcaaattcactgaaataatctattagaagtgaggggtttttttctgtatatttttggggatttcccacatagataatcatgtgatttgcaaatgacttttatttctccttttctaatctataaatttttttcttaccttattacactgactagaatttctagcatgttgttgaataaaagtagtaagaatgcacatctttgtcttgttcacgatcttaaggaaaaaacattcagttactgactgttcgatataatgttagctgtagatgttccttatcaagttcaggaagttttcctctactcttattttctatttcttataattttatcattaatagaaattgaattttataaaatttttaaatgattgatataatcaggttatttctctttttttattctgttaataaggtgggttacatcaattgatttttaagtataccagccttgcatctctgaactatatctccagttggtcatgctatataattcttcatatagaattactgaattctgtttgttaaatgtttttgcatcaatttttatggggactagtagcctgtaatttttttcttgttcttctttgcactgtctttatctggtttatctttaccttttttttttttggattagggtaatacaagctttataaaatgaattgagaagctcttctattttttggaagaaatcacatagaattggtattaattacttaaatgtttggtagaattctgcagtaaagccatctggacaaggatatctctattttggggatattgaaaattaagaaataaattacctagtttattttttaaagtggttacaaaaaacaaagatgaatgtagtttttaatttaccttgagacttggtctttgatccatgaattatttaaaagtatgctgtttagtttctaagttttggagaatttcttgttattattatgtctgatctctgttttgtttctattatggccaggtgacatattctgtataattttactttttaaaaatttgtctaggattgccttatgatataagatgtggtctactgtggtatatatcccatggccacttgaaaagaatgtgcattctgctgttgtttgatgctgtgtaatagaaatgttgattatatcttgttggttgatagtgttgttgagttcttttatatctttgctgattttctagttttcctagttttctggttttcctatctatggtttagaaatgggtgttgaagccttcagctttaatagtagatctatttatttttccttctggttttgcttcatataatttgccagtttgttgcttgatgcacacacatttaagacttctatatcttcatggttaacttaccattttatcattatataatgtccctctctaattctggtaattttgtttgctcagagtctactttatctgaaattagtatagtaactctacttcatttttacttttaacatttattattttttattgtggcaaaatacacataatagaacttaacatttgaaccattttgagtgacattaaatacattcttaatgttgtgcaaccatcacttctatttagctcatgactctttatcttataaaactgaaactctgtacctagtaagaaaataattcacgcctgtaatcccagcactttggaaggccgaggcgggtggaccatgaggtcaggagattgagaccaacctggctaatatggtgaaaccccctctctactaaaaatacaaaaaattagactggtgtggtggtgggcacctatagtcccagctacttgggaggctgaggcagcagaatcgcttggacctgggaggcggcggttgcagtgagccgagattgtgccactgcaccccagcctgggtgacagagtgagactccatcttaaataataataataataataataataataataataataataataataattccctctacttccagcccctggaagccaccattctactttctgtttctatgcttttgactacttttaactacctcatataagtgaatcatatggaatttgtcttctcgtgactggcttatttcacttagcacattgctctcagggttcatccatgttatagtacgtgttagaattttcttcctttataaagccattgtaagtatataccacattttgcttatccatttattcatctgtcaatagacacttgattgcttccacattttagctattgtgaataatgcttctatcaatgtgtgcacaaatattgcttcaagaccctgttttcagttcttttagatgcatattcagaagtggatttgctatgtcatgtagtaattctatttttaatttttgagtaaccaccatattgttttcctcagtggctgcaccattttacatttctactatcagggcacaagtgttccagtttctccacatctttgccaagacttgttattttttgttttcttaatagtagccatcttaattggtttggttggagatggtatctcattgtagttttgattttaatttccctaaagattagtgatgttgagcatcttttcaggttcttatgggccatttgcatagcttctttggagagacatttattcgagtccattgcccaattttttgttgttgttgttgagttttaggagtgctttatatattctggatgtcaatctcttgtcagatatatgctttgcaaatactttctcccattctgtgggttgcccttttcctctattaatagtgtctttgatgtacaattttaaagattctccatgaagttcagtttttctgttttttcttttgttggctgcacctttggtagcatatccaagaaatcactgccagatacagtatcatgatgcttttgtcctacgttttcttttaagaattttatagtttttttcttatatttaggtctttgatcaattttgagttaatttttctatatagtattaggtaagggtctaatttatttattttgcatatggatatccagttttcccagcatgatttgctgaaaagactgttctttctccactgaatggtcttgacatctgttttaatcagggttcttcagagagacagaattaataggagatatatatctatatctatatctgtatctatctaactatatctatatctatctatctatctatctatgtagagagaggcgggggagatataagaaaggatttattaggggaattgactcacttgattatgtaggctgagaagtttcataacagatcatctgtaagctggagtgccagggaagctggtagcatggctcattccaagtctgatggccttagaaccatggaagccaatgttgcaattctcagtccaggactgaaagcccgagagcctggagaattgctggtgcaagtcctaaagtccaaagaccagagaatctgaacttgtctcatgatacaaatataatctaccatggtatatagcccatgtacacttgaaaagggtagaagaaatgaattctggctccagaagacagatcaagaatttgtccttcctttacatttttattccatcacccacattgagggcagatcttctccacccagtccaccaactcaaatgccaatctcttccagaaacaccctcacagatgcacctggggtagcccaatcattagaatcaaatgctaaatcacttgggtttcccttttacagaagggagacatgatcagtttctactgaagcattgagaataatttgtactttatcagctgctgggtatcccttaatccagtcaaggtcacatccaaaatcaaccatcatagcacccttgtcaaaaatcatttaaccatatatattagggtttatttctggactctgtattgtatttcacaggtctatgtgccttttagacttttatttttatttttattttattttattttattttttgagatggagtttcactcttgttgcccaggctggagtgcaatggcgccatctcggctcaccacaacctctgcctcccaggttcaagtgattctcctgcttcaccctgcctagtaactgggattacaggcatgtgccaccacgcctggctacttttttgtatttttagtagagacagggtttctccatgttggtcaggctggtcttgaactcctgacctcaggtgatccgccctccttggcctcccaaagtgctgggattacaggtgtgagccactgtgcccagcctatttttattttttttaactttaaaataaagttgagggggcacatgtacaggtttattacatgggtaagttgcacgttgctgaggtgtggtgtacaaacgatcccatcacccagttagtgagcattgtagccaactggtagtttttcagcccttagccccctctcacccttcccactttattagtccccagtgtctgttctttttgtctttatgttcaagtgtactcaacgtttagctcctacttataagtgagaatgtgcagtatttgttttctgttcctgtattaatttggctttgaataaaggcctctagctgcatccatgttgctgcaaaggacataatttaattttttatggctgcatagtattccaagatgtatatgtaacatattttctttatccagtccttcattaatgggcatctaggtagattgcatgtctttgtctttataggcttttattgtctatatgccagtactatattgctttgattactatagtttttagtaagatttgaaatcaggaaatgagagtcctcaatattttttctcctttttcaaaattgttttggctttttgatgtctcttgagattccatttgaattttatagtggactgttttatttctgaaaaaaaaaagtcattggaattttgatagggattgtattgagtcaatagattgtgttggtagtatggacatttgaaaaatattgtcttccagtccatgaacatggaatgttttccatttttatattcttctttaacttctttcagcaatattttataattttttactgtacaagtctttcacgtctttagttaattcctaagtattttatttgttttgatgctatcgtaaatgagactttcttaatatccttttcatattgtttattgtaagtgtatagaaatgcaactgatttttgtgtgtcaactctctatcctgctactttgcttaattcgtctattaacagttttttaaaaatgtatgtggaatcgttagagtgttctagacacatgatcatatcatctgtaagcagagataattttagttccttctttctgatttgtatctctttgatttcttttccttgtctaattgctctggctaaaacttctagcactatgttgaatagaaatggcaaaagcagcacccttgccttgttcctgattttaaaagaagagctttcatctttgagtacgtttgctttgggtttttaatatacagattttattatgttgaggaagttgccttccattctcagtttggttggtgcttttatcatgaaagagtgaatttttcaaatgctttttctgcatcaattaattgaccacgtgttttaccctctgtaagcctgacaacgctatggattacactgattgatttttgtattttgagttatccttgcattctagaattaagtcttacttgatcatggtctataattctgtttatatgctgctcaatttggtttgctagtattttgtttaaaaaattgctttgatgttcataaaaaatactgctctataattttcttttctgatagtgtctgtgtctaaatttggtgtcagaataatgctgacctcatagaataagtcaggaagtgttctctctcctcttcattttttttttggaaaagtttgagaacaattggggttagttctttaaatggtagaagtatttctactattggtagaattcaccagtgaaactgttagatcaagggctttttttttcattatgagattttgatttttgattacttatgtaatctccttactagttataagtcttttcaaattttctatttctttatgatttagtcttggtaggtttagtatttctaggaatctgaactttttcatgtaggttatccaattttggggggtacaattgttcatagtgctgtcttagaattctttttattcccatagaataagtagtaatgtcccacttttatttatgattttagtaatatgagtcttgtctctatttttcttagtcaatctaagtgtagaattgtcatttttaaaaaaaatttttaaagaaggagcatttggttttattggttttacccttgtttttctattcttgatttctttgatctttgttctaatcttaattattccttctttctggtagctttgggtttagcttattcttttctagctccttaagttgtaaagttagttggctgatataggatcttccttgtttttaatataatcatttgtagtttcaaatttcatgcttaacactacttttgctgcatcctataagttttagtatgttatgttgtcattttcattcatatctaagtattttctaatttccctttgctttctcttttgatccattaatttttttaaagaatgtgttttaaaattttcacatatttatagatttttcagttttacttttgttattgatttctaactttatcctgttgtggtcagagttgttttgtatatttctgttagatctagttggtttattgtgttaagttctctatgtcattacttatattctgtctagttattctattcatttttgacagtgtggtattgaattctttaactattattgcagaaatgtctctttttacttcaattctgaagttttcactgtctttattttgatggtctgtcattaggtgcataaatgctcataagtcttctatcttcttgctatactgaaccttttaagattaagtactttttttgtttcttgtaaccatttttaaaaaaatttaaagtccattttgcctgttagtacagccacatctgctcttttttcatttccatttgcatgaaatgtcatcttccatttttccactttcaatttatttgtgtctttggagctaaagtgagtctcttgtaaacagcatatatttggatcatttaaaaaatccattctgctcatctatattggttgaagagtttaatacattttcatgtatgctattactgaaaaggaggaacttacttctataatttttatatttgttttctctacaccgtataactccccatcccttgttcccttttgtatatattctatagctattttctttgcagttaccattggaattacatttaacatcctaaatttataacattctaacttataaaccagcttaatttcaataacataaaactctgctactttaacagctccttcccatctctctttgattgttaatgtcacaaaattgcaactttatatatcatgtgtcacaaaacataaataattccttgaaatgcattaaactcttctatggaaaaaataatgagttagaaaccatagtttcagtgatattaacttttagactaataattgtttttttaaaaagtattagtttcttaaatcatgtacagaacacaaagtcaagaaacattgttataataataccaccttttacaatgtccctatgtttaccttcattgatatttttatttctttatatagattctagttactgtatagtatgctttcattttaacctgcaggactccctttaacagttttggcatggcagatgtagggcagcaagctatctcagcttttgtttagctgggaatgtcttaagtttctcctcactttcaagttttgccagatatggtgttcttgcttgacagtttcttttttatattagcactttgaaaatatcagcccactgccttcttatctctaaaatttctgatgagaaatttgtagataatcttattgcagatgcttggtatgtgacaagtcatttctatctcttgctgctttcaagattttctctttgcctttgtatttggaaagtttgattataatgtatatccatgtgggtatctatgaattcatcttacttggagttaattgagcttcttggaggtttatgttcatgtcttttttcaaattcgaagaatttttcaccattatttcttcctatattctctctactcctttctttctctctgcgttgtctgggactctcatggtgtgtatgttgagccccttgatgatgtctcacaggtctcttgggctctgctcacttttcttcaattttgttttctttttgttcctcagacttgatgatttccattgccttatctttgagttctctgattctttcttttgcctactcatatcttccctcaaagccctctagtaaatttttcatttctgttattttaatttttagctccagatgttctttttaggttttcaaaatctttatttgtattgtcattttgttttatacattgttttcttgacttcatccacatgtacttttagtttatttatttatttatttattgagacaggttcttgctctgtcatccaggctgtagtacatggttcagttagctcactgcagcctcaacctccttggctcaggtgatccttctgccttagcctcttgagtagctaggactacaggtgtgtgccatccatgcctggataaataaataaataaatttaatagagatgaggtctcactatgttgcctaggcttttaaattttattttttgggtatgtagtaggtgtatatattcatgaggtacatgaagtattttgatacaggcatgcagtgcataataatcacttctaagatacaaaataaagccctctttactctttgctttcctttccttaagcagatggtgggagtaaattttgttgtgagttgtgttgcctggtgttgcaggaggaatggcacaagcacttccttagccaccctggctggtgtccaatgttcactgattctaagcccagcacagcactaggacttgcccaggaattgcagtccttgtggcctagactgcctctcaagtttgtttaggatcccagagcacttaagcccatggtggtaagtcttgtgaaaattcaagttccagctgctgggatgggtgattcccctctggctagggctgatctatatgctccctgtgtgtgttggcattggctgagtttagcccagctttactttccattgtgacagggcagcattgagttcaatgcagagccccacaatcactgtactgtccatttcccaggtttacagattgtctctctgtgctatgtggctgctgccagagggataggggaggggcatcattggcaattcaagactatgtttcttaccttcttcagtgcctctttcagcaatatgaagttaaaccaagtactgtgattgcttatctgatttcctgtttttacgaaggtgctttttgtgtgtgtagatagtttgttaaataggtcttctgcagtggggatcattgttgcaaggttcagtttggccatcttgctccaccctccctcttttccagttcttaaagtatcttcaagacagttattttaaagtcttttttgagtagatctgctgttaggtctttttcaggggcagtttctgttgactattttttcctttgaatgggctatattttcctcttttttttttgaatgccttgtgtttttttttgttgaaagttgggtatttcaatttaatgatgtggtaactctgggatgcagactcttctttttcctcaaggttgtttgctttttgttattgttttgtttgtttataaaaattgctttagtctgtctctgtgccaaggatcagcctgaggtgtaaacataaggtctttactggtattctctgagcttgcatctttctgtggtcatgtgtgttaactttctaattttccccatatattcagttgtttgtgaatgtccttatctttaatatctggctcccaaaaggagaaaaagagaaaaatgaaaaagggagtggaaatagggtgccagccctttaaatctctggaagtcatttcagctggatggggaggggtttgcaacaatggaataaggtgtaacagcaaagacccaccacctctttgtctgtacctctctgatcagaagcagcaatcatcaatctaagcacatatgcctgatacttagaggacagggtccattttgcttatcctggctccttcaagctgtgtgcaacaagctgctgcaggaatacatgcacagctgcctgccacaggggtggggataggaaatgggtagctgttactgtgctaagagctaaaattgactgagctgaaatcaaagttaactgcaatgtaccatccaaatgtttccctgaaagttgcaagccttcaatagactccagatttccaaaacagttatatcagacatactctgtcagtgcaattattgcctaagcagggagacagattcctcgtgtttcttactcttgcagttccccaggatgctctccctaccttcctttatttaatgtttttatgatacatcttttcccctttttattacgtttaccctgactatattgttatatttgagtgagtttcttgtcatcaacctattgtttggttatgttttagtcaactcttgtcttttaattgtctttagccaactcttgtctttacaatgatttgaattatttacatttattggatttattaatgtattaatgcttaagtctgaaattttattttttgttttatatttgttctctctgtttttctatgtgttttctttttttatgcttcctggtgattatatgaatattttttagaatttcattttgatttatccttagatttgatacaagagaatctctttcatatagttttttagtgattgctcttggtgttaaattatatatgcacaatttttcatagtttaaaagtgttgtcattttaccagtttgagtgtagaaaacatacttcccgtaatgccacatttttcttcctcatttataatataattgtcttaaatattcccccttcgtacatttagaattacataatacagtgtattggcatctattgatttttaaaaattcagtttgagaccttcttggttcttggtatgttaagttattttccattgaaacctggacattttgaaacctgaacgtggaggctccttgttacttttgtgtggcggtggggtttttggctccctgttagtcctccactgatatctgtttggctgagagagttgggaatgtttcctttcacctcccaactggtttccactgacaccatggaatgcagtggaggagggaaggtggccttgttgtcactgggaaacggtgaatattccaactctccactagggcttctctgatactacccaggccagcaggaggatgtggagttaagaaatcctggctccccatgttgtctctactaacactgtggagaagtgggatgagcttgttacttcccagtaggaattcctgactccctacccagccttttctgacacagcctggcaaggggttagagtgcctataacagcttggtaagggggaagcacaggcttctgcacttccatcagactttgctagtggggtgagagtgagccagtttttctgtggtgtttgttataacaaagcaactatattctaaaagtttttgtctttctggtttttccctttttctcattccttggctagagaaagtaagattttgtttggcctttttgatctgtgatggttccattttatggttgccaacttcaatggctctaaatctggaatatatgaggcaaaatgaaaatccaggaacacactgctttgtttttcttcaaatcctaaggtttctagcccatatgccttgttctctccaacttttagaatcttctcatgtttgctttttaaataacgtccagagactcaattatatgtagcaggaggaatgtggaaaagcatctccacttcacacccccctggaagtaaaagttatgatgcatatacagtgagataaaatttgaagctctgcatgcgtatgatgtgtgccacttatagcacatgatagactctctttcattcaaaattcattccaggcctgctgtgtcccaggcactgttcaaggcattgcagatatagtgatgaacaaaatagataacatttctctctttatggtgcatacattcttgtgggaagaggtggacaataaacacagaaagtagaatatgttagtaaaaaatgctactggaaaaagtaaagtagatttatgaggctagtgagtgctggaggcattggaggttatttattttacataaggtggtcagggaagctaacatttgagcagagtggctgcagtgagatggacaaagacaaaagtggtagaagagagcagagatgtagttggggaatcagattgtctagggtcttgaaggcagtcaagaatttgggctttcatttagtcatgaaggaggttttcagagagaagtaatatgatccagtttacagctttattggtctgctatatggagaatagattgcggtggagtaaatgtggtggtcaaatgaggaggcttttacaaatacccgggagaggtttaatgagagattaaactcagatggaataatggagttagtgagatgtggccagattctgaaaacattttgcagataaagatgacagaaattgctaacagattttatgtggggtgtgagagaatgagagagagagagagatcaaagatggtctaagatctttggcttaagcaattggaagaataaacttggcatttactgagatggggaagaccgagggaagtgcaagtctggggaagaatggggagtcagaggtttgggtttgactgtgttaaaattgaaatatccattaaactaggggcatagtaggaggttgcccacaggagtctgagtttaaggactgcagatatcctgtctggagatatcaatttgggacttgctgtcctatagataattgcttttgtctgttttgcgctgctataactgaatacctgagactgggtaatttacaaagagcagaaatctatttcacacggttctagaggctgggatgtacaaggccaaggtgccagcatctgatggactccttcttggtgtattctcacgtggaggaaggcagaaggtgaaagggcaagaatgaacaaatccactccctcaagtcctatttatagcagcattaatccattcatgttggcagagccctcatgacctaaacacctcctaaaaggccccacctttcagcaccgttgcattggggattaaatttccagcacacaaattttggggaacacattcagattatatgaatgatatttaatgatatgtctagatgtgatcacatagacagagactgtaaataaagaagaaaaagagtcctgagttctgggggcactctcatatttagaggtcaggaggatgaggagaaaccagcaaaggaaaatgagaacagatcagtgaagcagaagaaccaagagagtgtggtgtgctggaagttgagtaaagaaagtgtttccagaaagagggagtgattaatttgtcaaataagaagagagttgtaaattgattcttgggtttgggaatgtgatggtctctggtggccccaccaagatttgtttccatgggaggagggggaaggaggaaagaagtggaggagaagacagagatggtgagcataagcaactctctggagttgctataaaggaaagcaggaaaatggagtagtagatgaacaaggttacagggtcaagaaaggtgtaattactatacagaacagttaaatatatacatggaaactttgtatttatctatagagtttgaatttattttgattgacaatgagtctaaaatttcatatagtgaaaagtcttcaattggcttgataatcttagcaatagcatttatatttgaagaagacttagttaaatatgagttggtctatgcaagatgtgtaattttaaagcttcttttctttggtttaatttggatttgcaatttcagttaaccctttccaatgaatccccccaaatgaatatttacttagcaatgataaaaaaactttatatttacagagtacttatcagtctctctataagaacaaacataaaagaatgaaaatttacattagaggtgtaggaggtaattaaagtgatgtggtcccaaaccagggaaactgagtctcagaccataggtggagcatgggatctgaaggggaaactcacctacaactctacagcaccaattgccagcctcttgggctaatagactctctcccgcttctgtcattgtctttgtctttttctacagtgaaacaagctttgaactatttccagctttccaatagcaaagttattcagattagatgaactttatttgttctccgggacatctagttctccaaagtcttctgtctgtaccatgtgttcaccaaacctccttctttttcctgctgggaaacccaatcagctgcatttcccagcctcccttgcagttgggtggggccaaaagaatgagggtggaatttatgccacttccagcctgaagcatggtcattaagaaaatcttctacacaatcctccttgtcctctctcttcctttgcctgctggatagatgcagaagattccttggaagggcctgggtcctgaatggctgtggagcagaagcttgaatagcatcttactgtggcatgagcaagaaatgagcttttattatgttgatcttttgatactttgtagttgtttgttacagtagtgagcctgccctgattaatacatacttctactgcaagtattacaaaaatacaactttttaaaaaatacctttctgtagcctttcaggatcctttccttctaagaagtcaccttcaggctcccaaagctttcctggagtgtgaatgcaaggagaaacaaatagcaacaatcctgtatcagttactttttcctgcatcatcttatttaatttctgtaggaatccaatgattctcataaaacagtagtccaatgagtccagcctcataatccaataaggttaataattaacctcagtttacagatgaaggagtttgtccaagatcacagagttatgaaatagtgaaatccttcgagcctgacctttttaaccacggagcccttgctctcccactacatcttcactctgttctttttccctctggggaaggaagtaaaactcatgaatctaattcacttatgcacactctggtattagagactcaatttcctttctcttgttgtgtgtatttgtgttttgaaggaggtgagaaacagagaaggtaattatttactgacatgaattgtaggcactgttgtgattgggtggggtgggaggcattctgcagacgacatagtagacctgagtccaggtgtgaaggggagagggagacagcagacagcatttagtttctagggagacagcacaggaaactaacctttctttccccttcagctttttaatacaaataattaaagatcctcatagtccattaactggccaggagagcagatggtagaggtgagaagtttcagcatcctgagaacacttgagcagcctccctgttccaacagaattgtccatattcaagccaagagaggaaagatgtggaactgctgttgccaagggggaaagtgcaggtcttcaataccaccttgatgttttatgtttataaaatttggctcagctgggcgcggtggctcatgcctgtaatcccagcactttgggaggctgaagcaggtggatcacgaggtcaagagttccagaccagcctggccaatatggtgaaactctgtctctactaaaaatacaaaaattagctgggcatggtggcaggtgcctgtagtcccagctgcttgggaggctgaggcaggagaattgcttgaaccctggaggtggaggttgcagtgagccgagatcgtgccactgcactccagcctgggcaacagaatgagactctatctcaaaaaaaaaaaaaaaattgactcacacataatgcaacacctgtctgcatacttttgccagtcactccataatttaaaatgtgcttcttcataggaagcatcaaattagataatttcatcctcacagcaattttaggaggcaacactcacatatgatccaggtaaaaaaaatggtgactcacatccctgacatgaagcccagggcatttcttagacttcctgtttttgagaaggaaatctaagacaaacctcccagactttgaaggatgggtgttcctatccaaaatgcaactgtgattatctgtgaaatctgcagatcttgtctgctctttcttgtctgcaaaagatcagatgaatgctctggagatgtgtgtggttgtctagcatgagcatgtgcgtgtgcatttgtacatgtgtgtgtgcatgcatatgtgtgtgtgtttttgtgtaagagacaaagaaagaaaaatgaatataaaatttacagaatcaaatatcagttttcaaagcttcaagatccaattgtgtgtataggattatctgccctgtttaggaaagtgttttctggagtggagctccaagtctgtgatgaatgtgccaaactaggggttccttctcggagagtcatcatatccttagtatgccatggcttgaagaaaccccacaatgagtaaatattaggtcttgtctgatgttatagatcccaagcttacttaactatggattctcttttttgagtctatcactcattcacatacagtgacagtacatctttttgaaaatgccaattcaagagtaattagaaaggtttaatgtagaaaactaggattatcagaagacctagattttgctcttactctatcactaataggctttataatttttgataaattatttttatttagttctttttcaagtatttttttctgccttgaaggaaacatttctttaaggatatttattgtacttatttaaaattgttgttctgtcatgataattccactttgcatggtacatgttattcattatttattacataaattatttatatattacatgttgttgattatttcttagtggttgtacttttcaggtatgtaattattttggcctgtgatatacatcctcctggagaagataatttgtattgggttaaggcgaaatggcagagcctggtttttgtaccttctggtgtgagcttaagaaagaggtggggatgagaccatgttcttttttaacagtgaatcctaacctttcctccctgtgtgtcccaggacatttgtgtactcactgctcaatcgagggaggccttatccagctatactcattctcagaggcactgccttctgcactggaaatggagtccttcaaggctactatctgatttactgtgctgaataccctgatgggtggtacacagacatacggtttagcttgggtaagtgaatctgcccacttctcacctacttcccagctaacaacccttcccacaatctacatcgccatggcctctccctactgcaatgacctcgtaatgatcccatcttcccctccctcacctctaccctgatcttttctctatatggtagccagagagattgtttaaacacatatcatgccatttcccatcgcaaaactctccagtagatttcctttactctcaaaataaaatacaaattccttcccactgcctttaaatcctaaatattcttggtcttttcaatttcccaacctcttttcctggctccctgcacctccttttctgtgctccagtgccagtggccttcctgctgtgccacaagctgtcaagcttgtccagccccagtgtttttgtataggactgttccctctgcctagtacaggggtgtccagtcttttggcttccctggaccacattggaaaaagaagaattgccttgggctacacatataattcactaacaccaatgatagctgataagctgaaagaaacattgtttcttaaaacaatgttttaagaaagtgtacgaatttgtgtcaggccacatccaaaactgtcctgggctgcatgtggcccacaggctgtgggttggacaagcctggcctagaaggctttgcccccatgtattcatgggggttggttcctcactttatttagttccctaccaatttgcacctcctcaaagggactttccctgaccaccctatctaaaatggttgaagacactgctgtgtgtgctgtcccccttccctccccgctcccctaccatcccctgccccagataccctttcttccaggctaatgatcttttgactaggcaaggtggaaaatccttgttttatatcaaatcaaataaggtgagaatagacttgattgtttcaactaaactgttcttgctttttgcctattttgaaaagcaccaaagtgctttgctgacaggttgggttgggcctctttgtgggacggtgagtgtttctgggctgtaataatgccttttgggatgcaggatctagcatccctgatcctggctgtcctcacctcagaacagggccctttctgtaccagtggatagtgcatgtggagacacctccaggatatgtggctaagtaagataaataaaaatgtgaacagtgtgtgtagtatgctacttttaatgtcagaatgtgtgtgtgtgtgtgtgtgtgtgtgtgtgtgtgtgtgaactggcagtgatggaagtcagtttgttagtggccacgcctccaaactagctcacgttatttcatgagagtgggtgtttcagtcaggaatgcgttcagaaaaccatataatagtgacgggaataattcagaattcatagttcttacataacaagtgtcgaagtaggtggcttctgcattcctgtggttgcttactgacaccattaataactcaggcactttgtgtctcccctattcttagcatgtttacctgtggtcctcttattcatcacttcatggttgcaagatgttgactgtagctcaaggcattacatctggcaatcaagacaggaagaaagacgaagtaggggcaccggtcttgcctgcaccttttatcagaaaagcaaacgatttccaaggaagtcccccaaccaagacttcactttacatcttattggtggaactagctcatgtgaccccctttctcagtgcaacctctgtagtgggggatggcaaaagaaaaggggctggaatcagctgttgagatatgaaacctcgatttgactgtttgtgaatgagctaaaggcttactattatgatccatgcatttgtttgtttatctatttaaatactggacaatggtgctacctcctgctctcctctcttttctaatctgtctccatcagtggtgccactgtgtactttcagcccaagtctaggaaacagcacacccagtgctccccattcaggaccaggactcattgccctccccactttctgccacgtcttaggaccattcttagatttagaaattaaatgcccctcctttcattttagcttagtttgtgaaaaaagcaccacaatctggacacatttaaaaaaaatttctcctttctctcttctttgtcaaggtgtcttcttatttattttgggaatgggaataaacattcatagtgactatatattgcgccagctcaggaagcctggagaaatcagctacaggattccacaaggtaatgtctcccctgcccccagactctcactcttccctggagcctgtcccagggatgatgctatggtagcagatggagaggggacagtgagggcacgaatgatacaatacctctcagcttgctcttgacttcccctgcaaacccagtcccatcacagtacctctgcatttctaatctgaacccagaagctccagagagctaccagagccctcaggaaggtgcgagaccgcaatacccggaaggtgggagagagtgggagatttgagtagggttggaatcggctacctacaccctcattgcagtaggtaaggttgtcatgtacagagggctgatcctttctaggttgcaaatgatgctgaatgcttggccaataaatgtgtcccctccaattgatctagacttatcttgtgagcagttaaggctagaacttgcgtatttcctgccattcatatatacttttaaaggtgtagagaactatatttacttaatcaaaacttaggatgggaggccttacaagcacaagtgcatttttggagataaaggtgagcatgtttgtaatcctaactgctccaaagaaaccagggtatgagattgacaaatatacgcagatgggagcaaatcagtccaagtcatccttcagtaggtttagatgggtatttgtgtaggtccttggcttccaccgtttttgatgtaaatacaaccctttatttacatcaaaaaatttcctcaattatgacatgatgttagtgcacattaatagtcattgattaagaaattaatgacaaagttactatgaattcagtaacagattcaaatgaatttgaatgttgttaatatcctaacagtatctacatcaaacaaaaataatttaggaggttaagatgtgccgtttactgtcttcatagaaaatttataggccctttgctttagatccgtggttcctcccagggcgtagggaatcacaggttgggaaccactgacgcagtctcatcttcaagaaagattcaactctaaggggctttgattttaaaaacattcactccataactagataaataaagaagagcagagaaagcacttgaaaaccagttgccaagattagtttccaacttttgaattataatagagaatgatttaaaacaaagtatttttatgtaactgtagaaagaacaaaattgtttcggttgaaacatccccagtttaatgtgggtaataaaatttagtgtggatatatttaatatcattagaaatatgctgatttctagatacagtcctgagagaagaaatacctggttggatagaaaatgaaacatttaaaatctagaagccgacttcaccagcctacagcaggacaaatacctgcagctcctactctctgctttaaacatgaacagaggagagtgctttgcagaagggtggggaggggcaaatcccataagagaatctaaattccttctcccgtgagttcagatttttattattttaatgtagtgtgcagtttcacattttgggactgtgaatgtgaacgtgtgtgtgtccaggaaaggagcggggaggtaagaggtggcccaaaaaccttccttaacagaacaaaacaaaaaccagacctcctacacctctcccttatcctacccaaagctagtggtccagcagttgttagagatttggatagagcccgttaagcagttgaggccactcttcgcaactccagaatgttctccaggcaacaggctgttgttgctttgtttccccttctccccaagaggattccaccaaactcctatgactatggagggagcctccagccccacatttgttcttgctgcctttgtgtattttggaacaacacagatgggtttaatgtcaaaatgattcagttgcaatgattgaccttccgattcttctgcacgcaggtggcttgtttacgtatgtttctggagccaatttcctcggtgagatcattgaatggatcggctatgccctggccacttggtccctcccagcacttgcatttgcatttttctcactttgtttccttgggctgcgagcttttcaccaccataggtaaatttttcaataaaagcggcagcattcacgtagctttcttcttctccaaacaggcttttaacccgcagaggaagattgagaaaccgaagatatttaattttttaagggaaggttgatactataatctgggttagcatattctgacttgctgggcttgtattgaagttggaagttagctggatgagagtagcagtaataaaattttttctaagaacaaacataagtccatgtaaacagaggcaactgaggtgacggccgggaaggtcctgtgcctgtcttgttgaagaaggctgtccagcgagaagtgccttgcaccttcttgggttagctgacccaggctctgtgcccaccaggcaggcttacctggagatgcttagataaagcctcaagaagcggaagaagccgcttccatttccaccttcagtttcatcccctcccaccacatttctggaagtgtgctccagaggtgggaccatggccagcactttccaaagctccacagatatttatttatttatttatttatttatttatttttgagttggagtctcactctgatgcccaggctgaagtgcaatggggcgatcttgcctcactgcagccttcacctcccaggttcaagtgattctcccacctcagccttccaaatggctgagactacaggcccaccaccatgcccagctaactttttgcatttttagtagagacaagtttcaccatgttggccaggctgtccttgaactcctgacctcaagtgatctgcctgcctcggcctcccaaagggctgggattacaggcaaagccatcacgcctggcctccacagatgttctgaggcacagcaggctagagaaccactgggaaaaagtactatcttggtgactatgtggctgctatttttggcaacaagatttggtgctaacagttcaggtaatcacagagccctggaactccagaaggattatttcccctggaatctctgcaatgccagggtttattttggtactgccttccttccctgacacccaccctctataaatcataaccaaggccaaccctggattctggggaaggtaggacaagaaatcccatgtatggggcaatcttaggactgccctatctgctgggaaatgccagggattgcaggtgtttcagaaccctccctctgaaggtctggcctaaagcttcactaatccactaacctggggtggagtggggacaaaagaattaactaaaagtattgctggaagtgaaaggtaatatattaggttgttatcaggctttaacttgccaagtgtaaatgtcagatcctttccctaaaatggcttttgtggaaagagaaagcctataattactggagactatggatactcaaacagggaatgagtggtcttggaaccaggaccctggggttatgactcaatgcctagttttgaagatttaactaagaatgagttcctttttttctgattagaattgttcgaggacaacagttctgaaatgaatgaagcaacagctagtgacctattcctaccacctcctagactctgtgaggaggggtggcagagggttcctagcccataaaagagtcataacagttcttaccatttcaggctgggtctgctgcccaccagaggcagagcgggaatgggagaagagaaggcatcactcagcacttggagggtccctgggaaggagggactctactgtgcttggttaggggagggatggtaattccaccagacgtttccagctgctgcaacaacaaaacccaaattgaggcctcatcctgtgatgtcagacctcccagagaagggccagaaggcatagtttttaataggctaacaggactttgtgaaggcttctataaaacaaagccaggcactcacatcagaggcagcacgggcggaggcagcccaccagacttctcaaatcacagtaccgaaaaccagccaccttttgtaacttattatttgccccctaattcaaactaaggtactttaatacagtgtgtcatcttgattatgccttgaggaccgccagcatcaactagttaggaagacagcctggcaacttaagcagacaggcttcttataagatgcattcgtctcctgtggctgctgcaatacattaccacaaactgggcggcttaaaacaacacagatttgtcctttcatagttctggaggctagaattttgaaatcgaggcgtaggcagggccaggcccccgtgacggctctagggaagaattcttccttgcctctttttagcttctgatggtggccggcaacccttggtgttccgtggcttatagctgcatcactctgatctctgcctccgtcttcacatggccttctctcctgtgtgtctctgtctcagtctctttgtctcttctcctcttctttttaggacacagttatattggattaagggcacaccctgctccattatgacctcatcttaattttatctgcaatggtcctagtttcagaaaaaaaaaaattaccctctcagttactgggggttaggacttcaacatatcttttgacgagacaaaattcaacccatgatacatggtgacaatttgctggcattcccaggcctcatgaagggatattttcctgggaacattgctggttctctttgcccatagtctttggttccctgtatttcccagctgaatccaaacaagtacctccttctagatactacatttttttttctggtaggatatacgtaacaaaaattttaccactttagccgtttttaattgtaaaattcagtggcattaggtacattcataatattgcacagccatcaccactaccctctgcctgttacttttcatttcccagcaaaagaagcttcaacatttacaagaaataggctgtgggaaggagaaataagaatcataagtgaccatcgaaatagtcaggcccaaaataactgtaggttgacttgtaacaaagatttggtaagagcaaacctcagctgtcagccactgctccattatatttacaattattcctttcttgtcttttaggttctacctcaagatgtttgaggactaccccaaatctcggaaagcccttattccattcatcttttaaaggaaccaaattaaaaaggagcagagctcccacaatgctgatgaaaactgtcaagctgctgaaactgtaattttcatgatataatagtcccgtatatatgtaatagtaggtctcctggcgttctgccagctggcctggggattctgagtggtgtctgcttagagtttactcctacccttccagggacccctatcctgatccccaactgaagcttcaaaaagccacttttccaaatggcgacagttgcttcttagctattgctctgagaaagtacaaacttctcctatgtctttcaccgggcaatccaagtacatgtggcttcatacccactccctgtcaatgcaggacaactctgtaatcaagaattttttgacttgaaggcagtacttatagaccttattaaaggtatgcattttatacatgtaacagagtagcagaaatttaaactctgaagccacaaagacccagagcaaacccactcccaaatgaaaaccccagtcatggcttcctttttcttggttaattaggaaagatgagaaattattaggtagaccttgaatacaggagccctctcctcatagtgctgaaaagatactgatgcattgacctcatttcaaatttgtgcagtgtcttagttgatgagtgcctctgttttccagaagatttcacaatccccggaaaactggtatggctattcttgaaggccaggttttaataaccacaaacaaaaaggcatgaacctgggtggcttatgagagagtagagaacaacatgaccctggatggctactaagaggatagagaacagttttacaatagacattgcaaactctcatgtttttggaaactagtggcaatatccaaataatgagtagtgtaaaacaaagagaattaatgatgaggttacatgctgcttgcctccaccagatgtccacaacaatatgaagtacagcagaagccccaagcaactttcctttcctggagcttcttccttgtagttctcaggacctgttcaagaaggtgtctcctaggggcagcctgaatgcctccctcaaaggacctgcaggcagagactgaaaattgcagacagaggggcacgtctgggcagaaaacctgttttgtttggctcagacatatagttttttttttttttacaaagtttcaaaaacttaaaaatcaggagattccttcataaaactctagcattctagtttcatttaaaaagttggaggatctgaacatacagagcccacatttccacaccagaactggaactacgtagctagtaagcatttgagtttgcaaactcttgtgaaggggtcaccccagcatgagtgctgagatatggactctctaaggaaggggccgaacgcttgtaattggaatacatggaaatatttgtcttctcaggcctatgtttgcggaatgcattgtcaatatttagcaaactgttttgacaaatgagcaccagtggtactaagcacagaaactcactatataagtcacataggaaacttgaaaggtctgaggatgatgtagattactgaaaaatgcaaattgcaatcatataaataagtgtttttgttgttcattaaatacctttaaatcatggatgttaagcagttttgtttgaattaaaatatcctgggacttctggagattttcacaaggtattggggtgtgcttccctgtcccgcctgtccctcattcccattcaccctgtctgtagtgtcgtttcctttcttctcttctctcttcatgaaattaggggctgtgtgctggaaataaagaagtatgacttatacagcctctcagccacaattcagaactacaaggaagtacaataaaggtgcaggaaaacgtcatgaggttagatgccactcctggtgctgaggaatacacagggggaatgaacacacttggtgaagtaaatcttcaggtgtagcttgagctctgtgacataaaagacttccagttctctccatgacattgaaaactaccttgcttaagtcctttattttactaccatgtaaaacatccaggccggtccttgagatgaaataaatatacaataaatggcagtactcagtctcaggacaaaaccgccatttggcaatttcactttttgccacaagagggcgctttctaaaatagtatttagtccaaccttgacttaattaggtccaaccccagtcctgttgcctgatcgtggacctttcaagttagcatgatcgtggaacttcttaatctgaatatagggtagagtagcgtgaactctatgaaagctgaaaccttcccttgataattcaggtattctcagtgttctagggctgttgtgttcaatgacagtttgtcattgtttagggctaggatatgtatgtggattaaatacagtaaaggtctgaacatgtgctcatctctaaacatatttcttcccacttttaaaaatagggtgcaacaaaattatagttaattgaggtttccatggactgttttttggctaaaataatctaacagtgtcctagctcacaagatactctatcttggtcttgctgaagctttcacggaagaggcgttttcctgccaaccttgaagaaaatgttgagttttgtttgtttttcagaatttagacttaaagagctgaagcagacctcagacatcagctattcctaccacttcacattataggtagtgacactgagacccacagagttaaaaggcctcctcacagctggtacagagctggtcagagcagccatctcattttgtccctttgagggtcagatgtatgcttcttctgcttaaggcacagcctttagggctgccatctggatttctacagagctcatgcaggagaaaaatagccagagcttaagtgctgttgcatagttcagtttatacacctattgtcccatgcctgtttgtgtgtgaggcttccctcgctccgtgggagcaccaggctcatgccctttcataaacctatttgacagaatagagagttttccctcattggtgtagtcaagacaacatgtgtgttcagcatggaaaaaaggtttcaggtcataaacccccttatccctaatgcttactttcataaagaacaacagtggctccactgtattctaaaaggaaaaaggagctctaatgagaaatacgcaattaaacagctatttaggcggcttagaccctttcaggaacaaataaataaatccgtggttttacaaccctgaagtttgataatagctctcttggatttttgtggttacctcatggcaatctcactaagaagcaggggagcaagctcttggcacatgaaggaataggctcatgaagattcatttcttttcccgcttggccttttggttctctaataagctgtgagctgaggtccatgcagggaaaacaaggagggtaacggcaaatgtaaagtgaatttttctggagaaggatgctgattattttatagcatctacactatcctatgatgaaagaggcatagagccgggtggcaatgtgagttcttgggtgatttaataagccatcgagtggatttaatttctaagtaagacatttgtttattctttgtacaagtccatggagttgtgatcttaaagagttctgtgttatttgcattgtgcctcatttctccccatcaaccttagcttgagatttggatgaataaaataaataactggagaaggttaaagtatgcaccaagctgtgttagtttctttagtcctgtctttttgggttgagggtttggttttgctcaatgcagtaacaatctagattctttattggagggatagcgaatatcccttccgacatggcaagctggctgggaggaggaggacaacagagaaagtggaggaaggaggggagtaatgagcgagggtctaagggagggttggcaatggaaggcccttgagagagaggggcctcccactgacagagtgggcttcctccctttcctgtgtccaggctgcacaagcctggtcccttctttatcctcccaggaaaagacctccctctgacagagagaataaacacttgttagagctccatttcataaaagaagaagctgacttttaaaaaacacaagccatttggctgtgcttcaccataaataattgtatttctgtctgctcacctcgtaaagatagttttagttctgcgtagtagtgacattgtgagaaagaggtgaagtaattgctcttagagcgtgtggcagcatccaagtcggctgaggccaatgggaagatgggagcatccagaagaataagcaactgccagggttccccttttcaaggcctttgttttcctttactccagacgcactccccaaaaaaggatccttccttatagagaagcgtttttacagtgttgactagaaagcaataccgtaaaatcaattaaattttgtcaatgtattagtgctttcatttgttacaagttgcctttcagatactatttcattaaaataattctttatgatggtttataacaggataagctctgaacagataagtaagtgaggggaagtagaaataggagtaaaaagtaaaataaagccagagataaagttaaaatgcaaaaatgcccttggaagatcatgtacagtggctaaatctaaacataaattgggtcctgagtctccaaagtacagaaggaaattaccagttgctaaatccacaacattcacaaaagaaaaacaacttgaccagggaaacacaactattaccggtactgagactcatgataaatttctacactgtgtgatgtggggaactatgttcatgagagcatctttacaataaatgcaggggtcactttgtgcagcttcttataatgtctttccacaaggacaacattctaatgcgcagctcaattctttagaagcaaaggggatgaggaggttttttttccacttagggtgtagaaagccaccagataaccattcctactctaaaaatgagaaaaaaccagatagtatataagattataactttttgagccttttaatgaactgagataggagagcaaccagaagaactgaatttcagaggaaacacacaaaccgtttcactttcagcggagtatgggaggaagaggtaatggccataagagtgggaaagaagaaaacaacagaaatttcaatgaattatcaatagctgagtggaacctagcatgagattagaatccctgggagactcagacacttggctgaacaagcatccagatctgccccaggatgaggagtttcctgggacacaggactttctgtgctcaaactggaaaagtcctaggcagaccaggatgacctggtcaccttagtctggttgccaggtcttttacccagacctccacctggagcttatgaggtgggttggggacagggaaggggatctgagagggcccttctttggtggtgcatagttacaaaagcccactgtattagttcgttttcatgctgctgataaagacatacctgaaactgggaacaaaaagaggtttaattggacttacagttccacatggctggggaggcctcagaatcatggctgtggaaagagaaaaatgaggaaaaagcaaaagcggaaatccctgataaacccatcagatctcgtgagacttattcactatcacgagaatagcatgggaaagaccagcccccatgattcaattacctgcccctgggtccctcctacaacatgtgggaattctgggagatacaattcaagttgagatttaggtggggacacagccaaaccatatcatcccacccctggcccctccaaatctcatgtcctcacatttcaaaaccaatcataccttcccaacagtcccccaaagtgttaattcatttcagcattaacccaaagtccacagtccaaatactcatttgagacaaagcaagtcccttctacctaggagcctgtaaaatcaaaagaaagctagttacttcctagatacaatgtgggtacaggtattcggtaaatacagccattctgaatgggagaaattggccaaaacaaaggggttacagggcccatgcaagtccagaatccagcaggtcagtcaaattttaaagctccaaaatgatctccgttgactccaggtctcgcatccaggtaacgctgatgcaagaggtgggttcccatggtcttgggcagctccacccctgtggctttgcagggtacagcctcctttccggctgacttcacgagctggtgttgagtatctgtggcttttccaggcacgtggtgcaagctgttggtggatctaaaattctgggatctggagaacagtggccctcttctcacagctccactaggcagtgccccagtagggactctgtgtgggtctttgaccccacatttcccttccacactgccctatcagaggttctccataagagccctgcccctgcagcaaacttgcctgggcatccaggtgtttccatacatcttctgaaatctatgcagaggttcccaaacctcagttcttggcttctgtgcacctgcaggctcaacatcacatggaaggtgccaaggcttggagtttttaccctctaaggccacattctgagctgtacattggcccctttcagccatggttggagcagctggaacacaggacaccaagtccctaggctgcacacagtgtagggaccctgggcccagcccttgaagccactttttcctcctgggcctccaggcctgtgatgggaggagctgctgtgaaggtctgtgacgtggcctagagacattttccccatggtcttggggattaacattaggctccttgctatttatgaaaatttctacagctggcttgaatatctccccagaaaatgggtttttcttttctatcacattgtcaggctgcaaattttctaaacttttgtgctctgcatcctttgtaaaactgaatgcctttaatagtacccaagtcatctcttgaatgccttgttgcttagaaatttcttccaccagatatcctaaatcatctttctcaagttccacaaatctctaggacgggggcaaaatgcctccagtctctttgctaaaacataacaagagtcacctttagtctagttcccaataagtttctcatctccatctgagaccaccccagcctggaccttattgtccacattgctatcagcattttgggcaaagccattcaacaagtttttaggaggttccaaactttcccacattttcctgtcttcttctgagccctccaaactgttccaacctctgcctgttacccagttccaattccacatttttgggtatcttttcagcaatgctccactctactgctactaatttactgtatgagttcattttcacactgctgataaagacatacctgaaagtgggaacaaaaagaggtttaattggacttacagttatatatggctagggaggcctcagaatcatggtggggggtgaaaggcattccttacatggtggtggcaagagacaaatgagaatatcactatcacgagaatagcacgggaaagactggcccccatgattcaattacctcccccgggtccctcacacagcacgtgggaattctgaaagatacaattcaagttgagattcgggtggggtcacagccaaactatatcacccaccttcttccagggctttctgttatagaaagcaaaacctgcaagcctctgggagagggatacgaaaccctccttctctaagacacaggcaaaggtccactgactctaggggaggggataaagcaaaaaagaaccagaaaagagggcattcaggaaactgccccatcccctgctatactgtcactgagaaaggggtagaaacaaaaactatttcttctaagggggtgggtggggagaaggcagcaaatccagcaactctgaccaagagccttgttccaaggagcaagcattagcaggctactgcatggacatcgggaaatggccccagtctcaggcaatgttacactgatgctgatggaggggtagatgcagaagctgtctgtcctcaaggaggggagaggaaacccaatctggacagaagcctgcagagctacaaagaagaggtctgcatcactacgggtccctcctgactaagactcccccacagtccagcatttggctgcagagtgtgggatatggtgcaggaatgctgagaattccacaccatagtggccgagccccttaggactcacctaagactgaggctggagtaggggcgcctaggaactatcctaagtccagcatacacctcacacccaatagtgagcaagagcaaagaagaaaagaagggaaatttcaacaaattatcaatagttgagtggggcctagcatgagtttagaatccctgggagcctcagatactcagttgaccaagcacccagttctgccccagactgaggggtttcctgggacacaggactttccatgcttaaactgcaaaagttccccgcaaaccgggatgagttggtctctactgctggggagcacacagagagaaccacgcatgtggcacaggcatgctgagagctgagagtagagaagaaacaaggaatagcccctctggccctccaggcctcacagtaagcacaaggcagcagcagctcatcgtggaaagaatctgaggagtgtgatgcattacaagttaattcagtaaaacaaaccccaaatctagcctgttcccccagcccccaacttatgatctggtagaaaaggcaagcttaattctgggtataaatactatttacctcagtctctactgttcctttatgtttggcgttgaataaaaagtatgaacacacatacacatacataaaacaaggcaaaaaacaaaaagataatccactgtgacgagataaagcagtcaacagaatgagacaaagaagtggtccagatattagaactatcagatagggacattaaactaattatcattaatatatagaaggatctagaggaaaaggtggtgaacatgcatgagcagatgaagaatgtcagcagaaagacaaaaactaaacaaattccaagggaaatgctgacaaatctccccacaacatcaagagataaagaattaatgggcttggcagcctggatacagtaaaaaatcagtaaacttgaagacagatcaatagatttatccaaactgaatcaaagagagaaaaaaatatgtgtaattagagtcccataagggtggggtggggcagaagggggaggggagagagagagagagataaattgagacagaagatttcttaagcaaatgcataaagagaaagcagacagttagtcctcatctggaaaactgtgaagtgggtgaaatctaatgtgagcaaatgattatcctctttcttctggcatcatactttctggtatgatgccagaagaaagaggttacctgttaaagaatatccaaactatgttagttgttgacagcacaaggtctgattgtatccaggggatatttgcatttcactgaaggaacatttgcataaagatggaaggtcttcaaggtccacattaatgaatttatggtgaaatatccaccctctgcccagctaatgggactaatttgaggtccataccttatctaagaccactcatacacatcattcgaagtggtactcacattaaaccttctaggatgaaggctggacttttatacccattgtttcagttgtgactcaaaggggtccagccacagggcagagaggggctggagctgtgagcctgcttcctgggtcagtaattttgtttctgtgctctatgagactgatgcacaaacttggaggctcaggactgtgacagtccccaacaccagacagtctagcagagcacctagagccaggaactttccctccaacagttgccctatggttgtgcttttcaaagtattgtttctggacccctccatcagaattgccaaggtgctcctattctgtctgatctactttgacatttagtcatgttctcaccacactttatcccaccttttcagagccaccactatctctcagaattgtcataaaagtttctcaactgatctccccacttccatacttgcttccccatagtgtattctcaacacagcaagcagtcagagtgatacttttataagtacttgtaaaccttttcgcatcatcttcaaaacagtatttgggctcccatctcacccagagcaaaagagtaagtctttgcagtggcctacaaagctctacagaatctgggctccattacctctctgactttatctcctgttgctctccatgtggcccactgtccactaccatgctgacttcctggaagatcctcaaaaccccctcagccctaggatctttgcacatgctcactcctctgcctagaacgctcttctttcagacactcacgtggctcacctctcacttccttcaggtgcctgctcaaataccgcgttgtaagaaaggtctttcctgcccatcctgttcaacatagtgccccactcctacccttggtacactttctgccttatccctataatggttttcaaagcacttacaccgtctaatacacaagacagttcctagtttatgtgttcattctctgtctttccccactagactataagcccccgaaggcaaggacttgtgtgttttgtcctctgctgtgtcatcagtatttgcatgggtgcctggcataaaatgagagcttagtaaatatttatactatgggaagagctagaagcaaattttcctttctctaaatgtgggggtacttggagagaaaaggactcctaaacatggctaaaacatgtacaaactttactgctgcttcccatcatcctgagacctgggaggaataaaagaaaggatggcaagctgaatgggattttaaaggctagatcttgccattctaggtaattaatacatgactcctgctcatgataaagaaggtggtggtgtttgcatcagtgtatggatctctaagggggctgggcctggtatcatccagtcaagcatagagcagagtgtccaggcaggtcctggcaggtgtggtggaagattggcagtggccactggatgaggtgcatgtcctgatgcctcaggcagtcctggacagacggtcatttggctccttctttttggcataggttggccacttgtcaagtgtggccaggccttcaggctgggctgctcggtcataagtaatacactaacctggctgggcacggtggctcatgcctgtaatcccagcactttgggaggccgagaagggcggatcacgaggtcaggagatcaagaccatcctggctaacatggtgaaaccccgtctctactaaagaaatacaaaaaattagccgggcatggtagcgggcccctgtagtcccagctactggcgaggctaaggcaggagaatggtgtgaacccgggaggcggggcttgcagtgagccgagatcgcaccactgcactccagcctgggtgacagaacaagactctatctcaaaaaaaaaaaaaaaaaaaatagtaatacactaaccctttaaacacacacacacacacacaaacacacacacacaaacaagcacagttccacatttcaactctcattctggtgcc

SEQ ID NO: 42 - DNA, 201 bp, Homo sapiens
  SNP (101)..(101): Alternative allele: G.
  ref <308>: DATABASE ACCESSION NUMBER: NCBI SNP CLUSTER ID: rs2687116
  ref <309>: DATABASE ENTRY DATE: 2003-03-07
ccctttgtggaaaacaccaagaagcttttaagatttgattttttggatccattctttctctcaataagtatgtggactactatttccttttatttatctttctctcttaaaaataactgctttattgagatataaatcaccatgtaattcatccacttaaaatatacagttcagtgatttgtagtacatttgaagatatgt

SEQ ID NO: 43 - DNA, 189 bp, Homo sapiens
  SNP (89)..(89): Alternative allele: T.
  ref <308>: DATABASE ACCESSION NUMBER: NCBI SNP CLUSTER ID: rs1851426
  ref <309>: DATABASE ENTRY DATE: 2003-03-07
ttgggtgtgtggcgggtgtgtccgcgttttaaaaagcgccgcacgctttgaactccaattccacccccaagaggctgggaccatcttactggagtccttgatgctgtgtgacctgcagtgaccactgccccatcattgctggctgaggtggttggggtccatctggctatctgggcagctgttctcttc

SEQ ID NO: 44 - DNA, 281 bp, Homo sapiens
  SNP (181)..(181): Alternative allele: A.
  ref <308>: DATABASE ACCESSION NUMBER: NCBI SNP CLUSTER ID: rs676033
  ref <309>: DATABASE ENTRY DATE: 2003-03-07
actattctcctgccctaatcagccaggtccaggtaacagaaaagtaaagacagccgctgtaccccagagcctgctaaaagtattcaaacgagctaatcctaagcctgattaccttgtcatgcccactctttcctgcagaaactacagtaaaggctcttgcccaccttgacccctcactccggctgcctcctaacactggtgcttctccatgtggtcttgggtggtgtgctgtgtcttctgtttgtagggatctgtcgatataaaccttttccttcacgata

SEQ ID NO: 45 - DNA, 160 bp, Homo sapiens
  SNP (81)..(81): Alternative allele: A.
  ref <308>: DATABASE ACCESSION NUMBER: NCBI SNP CLUSTER ID: rs1042578
  ref <309>: DATABASE ENTRY DATE: 2003-03-07
ggtactaagcacagaaactcactatataagtcacataggaaacttgaaaggtctgaggatgatgtagattactgaaaaatgcaaattgcaatcatataaataagtgtttttgttgttcattaaatacctttaaatcatggatgtaagcagtttgttgata